SEQUENCE LISTING (224 sequences)

SEQ ID NO: 1 - protein, 19 aa, Artificial Sequence
  Description of Artificial Sequence Synthetic peptide
FVKDLLVHLKKLFREGQFN

SEQ ID NO: 2 - protein, 19 aa, Artificial Sequence
  Description of Artificial Sequence Synthetic peptide
FVKDLLVHLKKLFREGRFN

SEQ ID NO: 3 - protein, 19 aa, Artificial Sequence
  Description of Artificial Sequence Synthetic peptide
FVKDLLLHLKKLFREGQFN

SEQ ID NO: 4 - protein, 19 aa, Artificial Sequence
  Description of Artificial Sequence Synthetic peptide
FVKDLLLHLKKLFREGRFN

SEQ ID NO: 5 - protein, 16 aa, Artificial Sequence
  Description of Artificial Sequence Synthetic peptide
FVKDLLVHLKKLFREG

SEQ ID NO: 6 - protein, 16 aa, Artificial Sequence
  Description of Artificial Sequence Synthetic peptide
FVKDLLLHLKKLFREG

SEQ ID NO: 7 - protein, 17 aa, Artificial Sequence
  Description of Artificial Sequence Synthetic peptide
KDLLVHLKKLFREGQFN

SEQ ID NO: 8 - protein, 17 aa, Artificial Sequence
  Description of Artificial Sequence Synthetic peptide
KDLLVHLKKLFREGRFN

SEQ ID NO: 9 - protein, 17 aa, Artificial Sequence
  Description of Artificial Sequence Synthetic peptide
KDLLLHLKKLFREGQFN

SEQ ID NO: 10 - protein, 17 aa, Artificial Sequence
  Description of Artificial Sequence Synthetic peptide
KDLLLHLKKLFREGRFN

SEQ ID NO: 11 - protein, 13 aa, Artificial Sequence
  Description of Artificial Sequence Synthetic peptide
KDLLVHLKKLFRE

SEQ ID NO: 12 - protein, 13 aa, Artificial Sequence
  Description of Artificial Sequence Synthetic peptide
KDLLLHLKKLFRE

SEQ ID NO: 13 - protein, 8 aa, Artificial Sequence
  Description of Artificial Sequence Synthetic peptide
HLKKLFRE

SEQ ID NO: 14 - protein, 112 aa, Macaca fascicularis
SPVPPSTALKELIEELVNITQNQKAPLCNGSMVWSINLTAGVYCAALESLINVSGCSAIEKTQRMLNGFCPHKVSAGQFSSLRVRDTKIEVAQFVKDLLVHLKKLFREGQFN

SEQ ID NO: 15 - protein, 10 aa, Artificial Sequence
  Description of Artificial Sequence Synthetic peptide
GFNIKDTYIH

SEQ ID NO: 16 - protein, 17 aa, Artificial Sequence
  Description of Artificial Sequence Synthetic peptide
RIDPANDNIKYDPKFQG

SEQ ID NO: 17 - protein, 11 aa, Artificial Sequence
  Description of Artificial Sequence Synthetic peptide
SEENWYDFFDY

SEQ ID NO: 18 - protein, 16 aa, Artificial Sequence
  Description of Artificial Sequence Synthetic peptide
RSSQSIVHSNGNTYLE

SEQ ID NO: 19 - protein, 7 aa, Artificial Sequence
  Description of Artificial Sequence Synthetic peptide
KVSNRFS

SEQ ID NO: 20 - protein, 9 aa, Artificial Sequence
  Description of Artificial Sequence Synthetic peptide
FQGSHIPYT

SEQ ID NO: 21 - protein, 16 aa, Artificial Sequence
  Description of Artificial Sequence Synthetic peptide
XSSQSXXHSNGNTYLX

SEQ ID NO: 22 - protein, 7 aa, Artificial Sequence
  Description of Artificial Sequence Synthetic peptide
KXSXRFS

SEQ ID NO: 23 - protein, 7 aa, Artificial Sequence
  Description of Artificial Sequence Synthetic peptide
FQXXXXP

SEQ ID NO: 24 - protein, 132 aa, Macaca fascicularis
MALLLTMVIALTCLGGFASPSPVPPSTALKELIEELVNITQNQKAPLCNGSMVWSINLTAGVYCAALESLINVSGCSAIEKTQRMLNGFCPHKVSAGQFSSLRVRDTKIEVAQFVKDLLVHLKKLFREGQFN

SEQ ID NO: 25 - protein, 16 aa, Artificial Sequence
  Description of Artificial Sequence Synthetic peptide
XSSQSXXHSXGNXYLX

SEQ ID NO: 26 - protein, 16 aa, Artificial Sequence
  Description of Artificial Sequence Synthetic peptide
XSSQSXXHSXGNXYLE

SEQ ID NO: 27 - protein, 7 aa, Artificial Sequence
  Description of Artificial Sequence Synthetic peptide
KXSXXXS

SEQ ID NO: 28 - protein, 6 aa, Artificial Sequence
  Description of Artificial Sequence Synthetic peptide
QXXXIP

SEQ ID NO: 29 - protein, 9 aa, Artificial Sequence
  Description of Artificial Sequence Synthetic peptide
FQXXXXPYT

SEQ ID NO: 30 - protein, 102 aa, Artificial Sequence
  Description of Artificial Sequence Synthetic polypeptide
DIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHIPYT

SEQ ID NO: 31 - protein, 102 aa, Artificial Sequence
  Description of Artificial Sequence Synthetic polypeptide
DVVMTQSPLSLPVTLGQPASISCRSSQSIVHSNGNTYLEWFQQRPGQSPRRLIYKVSNRFSGVPDRFSGSGSGTDFTLKISRVEAEDVGVYYCFQGSHIPYT

SEQ ID NO: 32 - protein, 102 aa, Artificial Sequence
  Description of Artificial Sequence Synthetic polypeptide
DIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHIPYT

SEQ ID NO: 33 - protein, 102 aa, Artificial Sequence
  Description of Artificial Sequence Synthetic polypeptide
DIVMTQTPLSLSVTPGQPASISCRSSQSIVHSNGNTYLEWYLQKPGQPPQLLIYKVSNRFSGVPDRFSGSGSGTDFTLKISRVEAEDVGVYYCFQGSHIPYT

SEQ ID NO: 34 - protein, 102 aa, Artificial Sequence
  Description of Artificial Sequence Synthetic polypeptide
DIVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQGSHIPYT

SEQ ID NO: 35 - protein, 102 aa, Artificial Sequence
  Description of Artificial Sequence Synthetic polypeptide
DIVMTQTPLSSPVTLGQPASISCRSSQSIVHSNGNTYLEWLQQRPGQPPRLLIYKVSNRFSGVPDRFSGSGAGTDFTLKISRVEAEDVGVYYCFQGSHIPYT

SEQ ID NO: 36 - protein, 102 aa, Artificial Sequence
  Description of Artificial Sequence Synthetic polypeptide
DIQMTQSPSSLSASVGDRVTITCRSSQSIVHSNGNTYLEWYQQKPGKAPKLLIYKVSNRFSGVPSRFSGSGSGTDFTLTISSLQPEDFATYYCFQGSHIPYT

SEQ ID NO: 37 - protein, 102 aa, Artificial Sequence
  Description of Artificial Sequence Synthetic polypeptide
DVVMTQSPLSLPVTLGQPASISCRSSQSLVYSDGNTYLNWFQQRPGQSPRRLIYKVSNRFSGVPDRFSGSGSGTDFTLKISRVEAEDVGVYYCFQGSHIPYT

SEQ ID NO: 38 - protein, 102 aa, Artificial Sequence
  Description of Artificial Sequence Synthetic polypeptide
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHIPYT

SEQ ID NO: 39 - protein, 100 aa, Artificial Sequence
  Description of Artificial Sequence Synthetic polypeptide
DIVMTQTPLSLPVTPGEPASISCXSSQSXXHSXGNXYLXWYLQKPGQSPQLLIYKXSXXXSGVPDRFSGSGSGTDFTLKISRVEAEDVGVYYCFQXXXXP

SEQ ID NO: 40 - protein, 100 aa, Artificial Sequence
  Description of Artificial Sequence Synthetic polypeptide
DVVMTQSPLSLPVTLGQPASISCXSSQSXXHSXGNXYLXWFQQRPGQSPRRLIYKXSXXXSGVPDRFSGSGSGTDFTLKISRVEAEDVGVYYCFQXXXXP

SEQ ID NO: 41 - protein, 100 aa, Artificial Sequence
  Description of Artificial Sequence Synthetic polypeptide
DIVMTQTPLSLSVTPGQPASISCXSSQSXXHSXGNXYLXWYLQKPGQSPQLLIYKXSXXXSGVPDRFSGSGSGTDFTLKISRVEAEDVGVYYCFQXXXXP

SEQ ID NO: 42 - protein, 100 aa, Artificial Sequence
  Description of Artificial Sequence Synthetic polypeptide
DIVMTQTPLSLSVTPGQPASISCXSSQSXXHSXGNXYLXWYLQKPGQPPQLLIYKXSXXXSGVPDRFSGSGSGTDFTLKISRVEAEDVGVYYCFQXXXXP

SEQ ID NO: 43 - protein, 100 aa, Artificial Sequence
  Description of Artificial Sequence Synthetic polypeptide
DIVMTQSPLSLPVTPGEPASISCXSSQSXXHSXGNXYLXWYLQKPGQSPQLLIYKXSXXXSGVPDRFSGSGSGTDFTLKISRVEAEDVGVYYCFQXXXXP

SEQ ID NO: 44 - protein, 100 aa, Artificial Sequence
  Description of Artificial Sequence Synthetic polypeptide
DIVMTQTPLSSPVTLGQPASISCXSSQSXXHSXGNXYLXWLQQRPGQPPRLLIYKXSXXXSGVPDRFSGSGAGTDFTLKISRVEAEDVGVYYCFQXXXXP

SEQ ID NO: 45 - protein, 100 aa, Artificial Sequence
  Description of Artificial Sequence Synthetic polypeptide
DIQMTQSPSSLSASVGDRVTITCXSSQSXXHSXGNXYLXWYQQKPGKAPKLLIYKXSXXXSGVPSRFSGSGSGTDFTLTISSLQPEDFATYYCFQXXXXP

SEQ ID NO: 46 - protein, 100 aa, Artificial Sequence
  Description of Artificial Sequence Synthetic polypeptide
DVLMTQTPLSLPVSLGDQASISCXSSQSXXHSXGNXYLXWYLQKPGQSPKLLIYKXSXXXSGVPDRFSGSGSGTDFTLKISRVEAEDLGVYYCFQXXXXP

SEQ ID NO: 47 - protein, 11 aa, Artificial Sequence
  Description of Artificial Sequence Synthetic peptide
FGGGTKVEIKR

SEQ ID NO: 48 - protein, 10 aa, Artificial Sequence
  Description of Artificial Sequence Synthetic peptide
GXXIKDTYXH

SEQ ID NO: 49 - protein, 17 aa, Artificial Sequence
  Description of Artificial Sequence Synthetic peptide
XIDPXNDNIKYXXKFQG

SEQ ID NO: 50 - protein, 109 aa, Artificial Sequence
  Description of Artificial Sequence Synthetic polypeptide
QVQLVQSGAEVKKPGASVKVSCKASGFNIKDTYIHWVRQAPGQGLEWMGRIDPANDNIKYDPKFQGRVTMTRDTSISTAYMELSRLRSDDTAVYYCARSEENWYDFFDY

SEQ ID NO: 51 - protein, 109 aa, Artificial Sequence
  Description of Artificial Sequence Synthetic polypeptide
QVQLVQSGAEVKKPGASVKVSCKASGFNIKDTYIHWVRQAPGQRLEWMGRIDPANDNIKYDPKFQGRVTITRDTSASTAYMELSSLRSEDTAVYYCARSEENWYDFFDY

SEQ ID NO: 52 - protein, 109 aa, Artificial Sequence
  Description of Artificial Sequence Synthetic polypeptide
QVQLVQSGAEVKKPGASVKVSCKASGFNIKDTYIHWVRQATGQGLEWMGRIDPANDNIKYDPKFQGRVTMTRNTSISTAYMELSSLRSEDTAVYYCARSEENWYDFFDY

SEQ ID NO: 53 - protein, 109 aa, Artificial Sequence
  Description of Artificial Sequence Synthetic polypeptide
QVQLVQSGAEVKKPGASVKVSCKASGFNIKDTYIHWVRQAPGQGLEWMGRIDPANDNIKYDPKFQGRVTMTTDTSTSTAYMELRSLRSDDTAVYYCARSEENWYDFFDY

SEQ ID NO: 54 - protein, 109 aa, Artificial Sequence
  Description of Artificial Sequence Synthetic polypeptide
QVQLVQSGAEVKKPGASVKVSCKVSGFNIKDTYIHWVRQAPGKGLEWMGRIDPANDNIKYDPKFQGRVTMTEDTSTDTAYMELSSLRSEDTAVYYCATSEENWYDFFDY

SEQ ID NO: 55 - protein, 109 aa, Artificial Sequence
  Description of Artificial Sequence Synthetic polypeptide
QMQLVQSGAEVKKTGSSVKVSCKASGFNIKDTYIHWVRQAPGQALEWMGRIDPANDNIKYDPKFQGRVTITRDRSMSTAYMELSSLRSEDTAMYYCARSEENWYDFFDY

SEQ ID NO: 56 - protein, 109 aa, Artificial Sequence
  Description of Artificial Sequence Synthetic polypeptide
QVQLVQSGAEVKKPGASVKVSCKASGFNIKDTYIHWVRQAPGQGLEWMGRIDPANDNIKYDPKFQGRVTMTRDTSTSTVYMELSSLRSEDTAVYYCARSEENWYDFFDY

SEQ ID NO: 57 - protein, 107 aa, Artificial Sequence
  Description of Artificial Sequence Synthetic polypeptide
QMQLVQSGPEVKKPGTSVKVSCKASGFNIKDTYIHWVRQRQRLEWIGRIDPANDNIKYDPKFQGRVTITRDMSTSTAYMELSSLRSEDTAVYYCAASEENWYDFFDY

SEQ ID NO: 58 - protein, 109 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGRFTISRDNAKNSLYLQMNSLRAEDTAVYYCARSEENWYDFFDY

SEQ ID NO: 59 - protein, 110 aa, Artificial Sequence
  Description of Artificial Sequence Synthetic polypeptide
EVQLVESGGGLVQPGRSLRLSCAASGFNIKDTYIHWVRQAPGKGLEWVSRIDPANDNIKYDPKFQGRFTISRDNAKNSLYLQMNSLRAEDTALYYCAKDSEENWYDFFDY

SEQ ID NO: 60 - protein, 109 aa, Artificial Sequence
  Description of Artificial Sequence Synthetic polypeptide
QVQLVESGGGLVKPGGSLRLSCAASGFNIKDTYIHWIRQAPGKGLEWVSRIDPANDNIKYDPKFQGRFTISRDNAKNSLYLQMNSLRAEDTAVYYCARSEENWYDFFDY

SEQ ID NO: 61 - protein, 109 aa, Artificial Sequence
  Description of Artificial Sequence Synthetic polypeptide
EVQLVESGGGLVKPGGSLRLSCAASGFNIKDTYIHWVRQAPGKGLEWVGRIDPANDNIKYDPKFQGRFTISRDDSKNTLYLQMNSLKTEDTAVYYCTTSEENWYDFFDY

SEQ ID NO: 62 - protein, 109 aa, Artificial Sequence
  Description of Artificial Sequence Synthetic polypeptide
EVQLVESGGGVVRPGGSLRLSCAASGFNIKDTYIHWVRQAPGKGLEWVSRIDPANDNIKYDPKFQGRFTISRDNAKNSLYLQMNSLRAEDTALYHCARSEENWYDFFDY

SEQ ID NO: 63 - protein, 109 aa, Artificial Sequence
  Description of Artificial Sequence Synthetic polypeptide
EVQLVESGGGLVKPGGSLRLSCAASGFNIKDTYIHWVRQAPGKGLEWVSRIDPANDNIKYDPKFQGRFTISRDNAKNSLYLQMNSLRAEDTAVYYCARSEENWYDFFDY

SEQ ID NO: 64 - protein, 109 aa, Artificial Sequence
  Description of Artificial Sequence Synthetic polypeptide
EVQLLESGGGLVQPGGSLRLSCAASGFNIKDTYIHWVRQAPGKGLEWVSRIDPANDNIKYDPKFQGRFTISRDNSKNTLYLQMNSLRAEDTAVYYCAKSEENWYDFFDY

SEQ ID NO: 65 - protein, 109 aa, Artificial Sequence
  Description of Artificial Sequence Synthetic polypeptide
QVQLVESGGGVVQPGRSLRLSCAASGFNIKDTYIHWVRQAPGKGLEWVARIDPANDNIKYDPKFQGRFTISRDNSKNTLYLQMNSLRAEDTAVYYCAKSEENWYDFFDY

SEQ ID NO: 66 - protein, 109 aa, Artificial Sequence
  Description of Artificial Sequence Synthetic polypeptide
QVQLVESGGGVVQPGRSLRLSCAASGFNIKDTYIHWVRQAPGKGLEWVARIDPANDNIKYDPKFQGRFTISRDNSKNTLYLQMNSLRAEDTAVYYCARSEENWYDFFDY

SEQ ID NO: 67 - protein, 110 aa, Artificial Sequence
  Description of Artificial Sequence Synthetic polypeptide
EVQLVESGGVVVQPGGSLRLSCAASGFNIKDTYIHWVRQAPGKGLEWVSRIDPANDNIKYDPKFQGRFTISRDNSKNSLYLQMNSLRTEDTALYYCAKDSEENWYDFFDY

SEQ ID NO: 68 - protein, 109 aa, Artificial Sequence
  Description of Artificial Sequence Synthetic polypeptide
EVQLVESGGGLVQPGGSLRLSCAASGFNIKDTYIHWVRQAPGKGLEWVSRIDPANDNIKYDPKFQGRFTISRDNAKNSLYLQMNSLRDEDTAVYYCARSEENWYDFFDY

SEQ ID NO: 69 - protein, 109 aa, Artificial Sequence
  Description of Artificial Sequence Synthetic polypeptide
EVQLVESGGGLVQPGRSLRLSCTASGFNIKDTYIHWFRQAPGKGLEWVGRIDPANDNIKYDPKFQGRFTISRDGSKSIAYLQMNSLKTEDTAVYYCTRSEENWYDFFDY

SEQ ID NO: 70 - protein, 109 aa, Artificial Sequence
  Description of Artificial Sequence Synthetic polypeptide
EVQLVESGGGLVQPGGSLRLSCAASGFNIKDTYIHWVRQAPGKGLEYVSRIDPANDNIKYDPKFQGRFTISRDNSKNTLYLQMGSLRAEDMAVYYCARSEENWYDFFDY

SEQ ID NO: 71 - protein, 109 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISRDNAKNSLYLQMNSLRAEDTAVYYCARSEENWYDFFDY

SEQ ID NO: 72 - protein, 109 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGKATISRDNAKNSLYLQMNSLRAEDTAVYYCARSEENWYDFFDY

SEQ ID NO: 73 - protein, 109 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGRFTISADNAKNSLYLQMNSLRAEDTAVYYCARSEENWYDFFDY

SEQ ID NO: 74 - protein, 109 aa, Artificial Sequence
  Description of Artificial Sequence Synthetic polypeptide
EVQLVESGGGLVQPGGSLRLSCAASGFNIKDTYIHWVRQAPGKGLEWVGRIDPANDNIKYDPKFQGRFTISRDNAKNSLYLQMNSLRAEDTAVYYCARSEENWYDFFDY

SEQ ID NO: 75 - protein, 109 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGKATISADNAKNSLYLQMNSLRAEDTAVYYCARSEENWYDFFDY

SEQ ID NO: 76 - protein, 109 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISADNAKNSLYLQMNSLRAEDTAVYYCARSEENWYDFFDY

SEQ ID NO: 77 - protein, 109 aa, Artificial Sequence
  Description of Artificial Sequence Synthetic polypeptide
EVQLVESGGGLVQPGGSLRLSCAASGFNIKDTYIHWVRQAPGKGLEWVGRIDPANDNIKYDPKFQGRFTISADNAKNSLYLQMNSLRAEDTAVYYCARSEENWYDFFDY

SEQ ID NO: 78 - protein, 109 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGRFTISRDNAKNSAYLQMNSLRAEDTAVYYCARSEENWYDFFDY

SEQ ID NO: 79 - protein, 109 aa, Artificial Sequence
  Description of Artificial Sequence Synthetic polypeptide
EVQLVESGGGLVQPGGSLRLSCAASGFNIKDTYIHWVRQAPGKGLEWVGRIDPANDNIKYDPKFQGRFTISADNAKNSAYLQMNSLRAEDTAVYYCARSEENWYDFFDY

SEQ ID NO: 80 - protein, 109 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISADNAKNSAYLQMNSLRAEDTAVYYCARSEENWYDFFDY

SEQ ID NO: 81 - protein, 109 aa, Artificial Sequence
  Description of Artificial Sequence Synthetic polypeptide
EVQLVESGGGLVQPGGSLRLSCTGSGFNIKDTYIHWVRQAPGKGLEWIGRIDPANDNIKYDPKFQGRFTISADNAKNSLYLQMNSLRAEDTAVYYCARSEENWYDFFDY

SEQ ID NO: 82 - protein, 109 aa, Artificial Sequence
  Description of Artificial Sequence Synthetic polypeptide
EVQLQQSGAELVKPGASVKLSCTGSGFNIKDTYIHWVKQRPEQGLEWIGRIDPANDNIKYDPKFQGKATITADTSSNTAYLQLNSLTSEDTAVYYCARSEENWYDFFDY

SEQ ID NO: 83 - protein, 109 aa, Artificial Sequence
  Description of Artificial Sequence Synthetic polypeptide
QVQLVQSGAEVKKPGASVKVSCKASGXXIKDTYXHWVRQAPGQGLEWMGXIDPXNDNIKYXXKFQGRVTMTRDTSISTAYMELSRLRSDDTAVYYCARSEENWYDFFDY

SEQ ID NO: 84 - protein, 109 aa, Artificial Sequence
  Description of Artificial Sequence Synthetic polypeptide
QVQLVQSGAEVKKPGASVKVSCKASGXXIKDTYXHWVRQAPGQRLEWMGXIDPXNDNIKYXXKFQGRVTITRDTSASTAYMELSSLRSEDTAVYYCARSEENWYDFFDY

SEQ ID NO: 85 - protein, 109 aa, Artificial Sequence
  Description of Artificial Sequence Synthetic polypeptide
QVQLVQSGAEVKKPGASVKVSCKASGXXIKDTYXHWVRQATGQGLEWMGXIDPXNDNIKYXXKFQGRVTMTRNTSISTAYMELSSLRSEDTAVYYCARSEENWYDFFDY

SEQ ID NO: 86 - protein, 109 aa, Artificial Sequence
  Description of Artificial Sequence Synthetic polypeptide
QVQLVQSGAEVKKPGASVKVSCKASGXXIKDTYXHWVRQAPGQGLEWMGXIDPXNDNIKYXXKFQGRVTMTTDTSTSTAYMELRSLRSDDTAVYYCARSEENWYDFFDY

SEQ ID NO: 87 - protein, 109 aa, Artificial Sequence
  Description of Artificial Sequence Synthetic polypeptide
QVQLVQSGAEVKKPGASVKVSCKVSGXXIKDTYXHWVRQAPGKGLEWMGXIDPXNDNIKYXXKFQGRVTMTEDTSTDTAYMELSSLRSEDTAVYYCATSEENWYDFFDY

SEQ ID NO: 88 - protein, 109 aa, Artificial Sequence
  Description of Artificial Sequence Synthetic polypeptide
QMQLVQSGAEVKKTGSSVKVSCKASGXXIKDTYXHWVRQAPGQALEWMGXIDPXNDNIKYXXKFQGRVTITRDRSMSTAYMELSSLRSEDTAMYYCARSEENWYDFFDY

SEQ ID NO: 89 - protein, 109 aa, Artificial Sequence
  Description of Artificial Sequence Synthetic polypeptide
QVQLVQSGAEVKKPGASVKVSCKASGXXIKDTYXHWVRQAPGQGLEWMGXIDPXNDNIKYXXKFQGRVTMTRDTSTSTVYMELSSLRSEDTAVYYCARSEENWYDFFDY

SEQ ID NO: 90 - protein, 107 aa, Artificial Sequence
  Description of Artificial Sequence Synthetic polypeptide
QMQLVQSGPEVKKPGTSVKVSCKASGXXIKDTYXHWVRQRQRLEWIGXIDPXNDNIKYXXKFQGRVTITRDMSTSTAYMELSSLRSEDTAVYYCAASEENWYDFFDY

SEQ ID NO: 91 - protein, 109 aa, Artificial Sequence
  Description of Artificial Sequence Synthetic polypeptide
EVQLVESGGGLVQPGGSLRLSCAASGXXIKDTYXHWVRQAPGKGLEWVAXIDPXNDNIKYXXKFQGRFTISRDNAKNSLYLQMNSLRAEDTAVYYCARSEENWYDFFDY

SEQ ID NO: 92 - protein, 110 aa, Artificial Sequence
  Description of Artificial Sequence Synthetic polypeptide
EVQLVESGGGLVQPGRSLRLSCAASGXXIKDTYXHWVRQAPGKGLEWVSXIDPXNDNIKYXXKFQGRFTISRDNAKNSLYLQMNSLRAEDTALYYCAKDSEENWYDFFDY

SEQ ID NO: 93 - protein, 109 aa, Artificial Sequence
  Description of Artificial Sequence Synthetic polypeptide
QVQLVESGGGLVKPGGSLRLSCAASGXXIKDTYXHWIRQAPGKGLEWVSXIDPXNDNIKYXXKFQGRFTISRDNAKNSLYLQMNSLRAEDTAVYYCARSEENWYDFFDY

SEQ ID NO: 94 - protein, 109 aa, Artificial Sequence
  Description of Artificial Sequence Synthetic polypeptide
EVQLVESGGGLVKPGGSLRLSCAASGXXIKDTYXHWVRQAPGKGLEWVGXIDPXNDNIKYXXKFQGRFTISRDDSKNTLYLQMNSLKTEDTAVYYCTTSEENWYDFFDY

SEQ ID NO: 95 - protein, 109 aa, Artificial Sequence
  Description of Artificial Sequence Synthetic polypeptide
EVQLVESGGGVVRPGGSLRLSCAASGXXIKDTYXHWVRQAPGKGLEWVSXIDPXNDNIKYXXKFQGRFTISRDNAKNSLYLQMNSLRAEDTALYHCARSEENWYDFFDY

SEQ ID NO: 96 - protein, 109 aa, Artificial Sequence
  Description of Artificial Sequence Synthetic polypeptide
EVQLVESGGGLVKPGGSLRLSCAASGXXIKDTYXHWVRQAPGKGLEWVSXIDPXNDNIKYXXKFQGRFTISRDNAKNSLYLQMNSLRAEDTAVYYCARSEENWYDFFDY

SEQ ID NO: 97 - protein, 109 aa, Artificial Sequence
  Description of Artificial Sequence Synthetic polypeptide
EVQLLESGGGLVQPGGSLRLSCAASGXXIKDTYXHWVRQAPGKGLEWVSXIDPXNDNIKYXXKFQGRFTISRDNSKNTLYLQMNSLRAEDTAVYYCAKSEENWYDFFDY

SEQ ID NO: 98 - protein, 109 aa, Artificial Sequence
  Description of Artificial Sequence Synthetic polypeptide
QVQLVESGGGVVQPGRSLRLSCAASGXXIKDTYXHWVRQAPGKGLEWVAXIDPXNDNIKYXXKFQGRFTISRDNSKNTLYLQMNSLRAEDTAVYYCAKSEENWYDFFDY

SEQ ID NO: 99 - protein, 109 aa, Artificial Sequence
  Description of Artificial Sequence Synthetic polypeptide
QVQLVESGGGVVQPGRSLRLSCAASGXXIKDTYXHWVRQAPGKGLEWVAXIDPXNDNIKYXXKFQGRFTISRDNSKNTLYLQMNSLRAEDTAVYYCARSEENWYDFFDY

SEQ ID NO: 100 - protein, 110 aa, Artificial Sequence
  Description of Artificial Sequence Synthetic polypeptide
EVQLVESGGVVVQPGGSLRLSCAASGXXIKDTYXHWVRQAPGKGLEWVSXIDPXNDNIKYXXKFQGRFTISRDNSKNSLYLQMNSLRTEDTALYYCAKDSEENWYDFFDY

SEQ ID NO: 101 - protein, 109 aa, Artificial Sequence
  Description of Artificial Sequence Synthetic polypeptide
EVQLVESGGGLVQPGGSLRLSCAASGXXIKDTYXHWVRQAPGKGLEWVSXIDPXNDNIKYXXKFQGRFTISRDNAKNSLYLQMNSLRDEDTAVYYCARSEENWYDFFDY

SEQ ID NO: 102 - protein, 109 aa, Artificial Sequence
  Description of Artificial Sequence Synthetic polypeptide
EVQLVESGGGLVQPGRSLRLSCTASGXXIKDTYXHWFRQAPGKGLEWVGXIDPXNDNIKYXXKFQGRFTISRDGSKSIAYLQMNSLKTEDTAVYYCTRSEENWYDFFDY

SEQ ID NO: 103 - protein, 109 aa, Artificial Sequence
  Description of Artificial Sequence Synthetic polypeptide
EVQLVESGGGLVQPGGSLRLSCAASGXXIKDTYXHWVRQAPGKGLEYVSXIDPXNDNIKYXXKFQGRFTISRDNSKNTLYLQMGSLRAEDMAVYYCARSEENWYDFFDY

SEQ ID NO: 104 - protein, 109 aa, Artificial Sequence
  Description of Artificial Sequence Synthetic polypeptide
EVQLVESGGGLVQPGGSLRLSCAASGXXIKDTYXHWVRQAPGKGLEWIGXIDPXNDNIKYXXKFQGRFTISRDNAKNSLYLQMNSLRAEDTAVYYCARSEENWYDFFDY

SEQ ID NO: 105 - protein, 109 aa, Artificial Sequence
  Description of Artificial Sequence Synthetic polypeptide
EVQLVESGGGLVQPGGSLRLSCAASGXXIKDTYXHWVRQAPGKGLEWVAXIDPXNDNIKYXXKFQGKATISRDNAKNSLYLQMNSLRAEDTAVYYCARSEENWYDFFDY

SEQ ID NO: 106 - protein, 109 aa, Artificial Sequence
  Description of Artificial Sequence Synthetic polypeptide
EVQLVESGGGLVQPGGSLRLSCAASGXXIKDTYXHWVRQAPGKGLEWVAXIDPXNDNIKYXXKFQGRFTISADNAKNSLYLQMNSLRAEDTAVYYCARSEENWYDFFDY

SEQ ID NO: 107 - protein, 109 aa, Artificial Sequence
  Description of Artificial Sequence Synthetic polypeptide
EVQLVESGGGLVQPGGSLRLSCAASGXXIKDTYXHWVRQAPGKGLEWVGXIDPXNDNIKYXXKFQGRFTISRDNAKNSLYLQMNSLRAEDTAVYYCARSEENWYDFFDY

SEQ ID NO: 108 - protein, 109 aa, Artificial Sequence
  Description of Artificial Sequence Synthetic polypeptide
EVQLVESGGGLVQPGGSLRLSCAASGXXIKDTYXHWVRQAPGKGLEWVAXIDPXNDNIKYXXKFQGKATISADNAKNSLYLQMNSLRAEDTAVYYCARSEENWYDFFDY

SEQ ID NO: 109 - protein, 109 aa, Artificial Sequence
  Description of Artificial Sequence Synthetic polypeptide
EVQLVESGGGLVQPGGSLRLSCAASGXXIKDTYXHWVRQAPGKGLEWIGXIDPXNDNIKYXXKFQGRFTISADNAKNSLYLQMNSLRAEDTAVYYCARSEENWYDFFDY

SEQ ID NO: 110 - protein, 109 aa, Artificial Sequence
  Description of Artificial Sequence Synthetic polypeptide
EVQLVESGGGLVQPGGSLRLSCAASGXXIKDTYXHWVRQAPGKGLEWVGXIDPXNDNIKYXXKFQGRFTISADNAKNSLYLQMNSLRAEDTAVYYCARSEENWYDFFDY

SEQ ID NO: 111 - protein, 109 aa, Artificial Sequence
  Description of Artificial Sequence Synthetic polypeptide
EVQLVESGGGLVQPGGSLRLSCAASGXXIKDTYXHWVRQAPGKGLEWVAXIDPXNDNIKYXXKFQGRFTISRDNAKNSAYLQMNSLRAEDTAVYYCARSEENWYDFFDY

SEQ ID NO: 112 - protein, 109 aa, Artificial Sequence
  Description of Artificial Sequence Synthetic polypeptide
EVQLVESGGGLVQPGGSLRLSCAASGXXIKDTYXHWVRQAPGKGLEWVGXIDPXNDNIKYXXKFQGRFTISADNAKNSAYLQMNSLRAEDTAVYYCARSEENWYDFFDY

SEQ ID NO: 113 - protein, 109 aa, Artificial Sequence
  Description of Artificial Sequence Synthetic polypeptide
EVQLVESGGGLVQPGGSLRLSCAASGXXIKDTYXHWVRQAPGKGLEWIGXIDPXNDNIKYXXKFQGRFTISADNAKNSAYLQMNSLRAEDTAVYYCARSEENWYDFFDY

SEQ ID NO: 114 - protein, 109 aa, Artificial Sequence
  Description of Artificial Sequence Synthetic polypeptide
EVQLVESGGGLVQPGGSLRLSCTGSGXXIKDTYXHWVRQAPGKGLEWIGXIDPXNDNIKYXXKFQGRFTISADNAKNSLYLQMNSLRAEDTAVYYCARSEENWYDFFDY

SEQ ID NO: 115 - protein, 109 aa, Artificial Sequence
  Description of Artificial Sequence Synthetic polypeptide
EVQLQQSGAELVKPGASVKLSCTGSGXXIKDTYXHWVKQRPEQGLEWIGXIDPXNDNIKYXXKFQGKATITADTSSNTAYLQLNSLTSEDTAVYYCARSEENWYDFFDY

SEQ ID NO: 116 - protein, 11 aa, Artificial Sequence
  Description of Artificial Sequence Synthetic peptide
WGQGTTLTVSS

SEQ ID NO: 117 - protein, 11 aa, Artificial Sequence
  Description of Artificial Sequence Synthetic peptide
WGQGTLVTVSS

SEQ ID NO: 118 - protein, 11 aa, Artificial Sequence
  Description of Artificial Sequence Synthetic peptide
QASQGTSINLN

SEQ ID NO: 119 - protein, 7 aa, Artificial Sequence
  Description of Artificial Sequence Synthetic peptide
GASNLED

SEQ ID NO: 120 - protein, 9 aa, Artificial Sequence
  Description of Artificial Sequence Synthetic peptide
LQHSYLPWT

SEQ ID NO: 121 - protein, 10 aa, Artificial Sequence
  Description of Artificial Sequence Synthetic peptide
GFSLTGYGVN

SEQ ID NO: 122 - protein, 14 aa, Artificial Sequence
  Description of Artificial Sequence Synthetic peptide
IIWGDGSTDYNSAL

SEQ ID NO: 123 - protein, 16 aa, Artificial Sequence
  Description of Artificial Sequence Synthetic peptide
DKTFYYDGFYRGRMDY

SEQ ID NO: 124 - protein, 113 aa, Homo sapiens
PGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 125 - protein, 427 aa, Homo sapiens [sequence omitted]

SEQ ID NO: 126 - protein, 101 aa, Homo sapiens
DVVMTQSPLSLPVTLGQPASISCRSSQSLVYSDGNTYLNWFQQRPGQSPRRLIYKVSNRFSGVPDRFSGSGSGTDFTLKISRVEAEDVGVYYCMQGTHWPP

SEQ ID NO: 127 - protein, 20 aa, Macaca fascicularis
MALLLTMVIALTCLGGFASP

SEQ ID NO: 128 - protein, 329 aa, Artificial Sequence
  Description of Artificial Sequence Synthetic polypeptide
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - DNA, 360 bp, Mus musculus
gaggttcagctgcagcagtctggggcagagcttgtgaagccaggggcctcagtcaagttgtcctgcacaggttctggcttcaacattaaagacacctatatacactgggtgaagcagaggcctgaacagggcctggagtggattggaaggattgatcctgcgaatgataatattaaatatgacccgaagttccagggcaaggccactataacagcagacacatcctccaacacagcctacctacagctcaacagcctgacatctgaggacactgccgtctattactgtgctagatctgaggaaaattggtacgacttttttgactactggggccaaggcaccactctcacagtctcctca

SEQ ID NO: 130 - protein, 120 aa, Mus musculus
EVQLQQSGAELVKPGASVKLSCTGSGFNIKDTYIHWVKQRPEQGLEWIGRIDPANDNIKYDPKFQGKATITADTSSNTAYLQLNSLTSEDTAVYYCARSEENWYDFFDYWGQGTTLTVSS

SEQ ID NO: 131 - protein, 19 aa, Artificial Sequence
  Description of Artificial Sequence Synthetic peptide
MKCSWVIFFLMAVVTGVNS

SEQ ID NO: 132 - DNA, 336 bp, Mus musculus
gatgttttgatgacccaaactccactctccctgcctgtcagtcttggagatcaagcctccatctcttgcaggtctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagattagcagagtggaggctgaggatctgggagtttattactgctttcaaggttcacatattccgtacacgttcggaggggggaccaagctggaaataaaa

SEQ ID NO: 133 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHIPYTFGGGTKLEIK

SEQ ID NO: 134 - protein, 19 aa, Artificial Sequence
  Description of Artificial Sequence Synthetic peptide
MKLPVRLLVLMFWIPASSS

SEQ ID NO: 135 - DNA, 429 bp, Mus musculus
atggctgtcctggcattactcttctgcctggtaacattcccaagctgtatcctttcccaggtgcagctgaaggagtcaggacctggcctggtggcgccctcacagagcctgtccatcacatgcaccgtctcagggttctcattaaccggctatggtgtaaactgggttcgccagcctccaggaaagggtctggagtggctgggaataatttggggtgatggaagcacagactataattcagctctcaaatccagactgatcatcaacaaggacaactccaagagccaagttttcttaaaaatgaacagtctgcaaactgatgacacagccaggtacttctgtgccagagataagactttttactacgatggtttctacaggggcaggatggactactggggtcaaggaacctcagtcaccgtctcctca

SEQ ID NO: 136 - protein, 124 aa, Mus musculus
QVQLKESGPGLVAPSQSLSITCTVSGFSLTGYGVNWVRQPPGKGLEWLGIIWGDGSTDYNSALKSRLIINKDNSKSQVFLKMNSLQTDDTARYFCARDKTFYYDGFYRGRMDYWGQGTSVTVSS

SEQ ID NO: 137 - protein, 124 aa, Artificial Sequence; identical to SEQ ID NO: 136
  Description of Artificial Sequence Synthetic polypeptide

SEQ ID NO: 138 - DNA, 387 bp, Mus musculus
atgaacacgagggcccctgctgagttccttgggttcctgttgctctggtttttaggtgccagatgtgatgtccagatgattcagtctccatcctccctgtctgcatctttgggagacattgtcaccatgacttgccaggcaagtcagggcactagcattaatttaaactggtttcagcaaaaaccagggaaagctcctaagctcctgatctttggtgcaagcaacttggaagatggggtcccatcaaggttcagtggcagtagatatgggacaaatttcactctcaccatcagcagcctggaggatgaagatatggcaacttatttctgtctacagcatagttatctcccgtggacgttcggtggcggcaccaaactggaaatcaaa

SEQ ID NO: 139 - protein, 107 aa, Mus musculus
DVQMIQSPSSLSASLGDIVTMTCQASQGTSINLNWFQQKPGKAPKLLIFGASNLEDGVPSRFSGSRYGTNFTLTISSLEDEDMATYFCLQHSYLPWTFGGGTKLEIK

SEQ ID NO: 140 - protein, 22 aa, Artificial Sequence
  Description of Artificial Sequence Synthetic peptide
MNTRAPAEFLGFLLLWFLGARC

SEQ ID NO: 141 - protein, 329 aa, Homo sapiens; identical to SEQ ID NO: 128

SEQ ID NO: 142 - DNA, 417 bp, Mus musculus
atgaaatgcagctgggttatcttcttcctgatggcagtggttacaggggtcaattcagaggttcagctgcagcagtctggggcagagcttgtgaagccaggggcctcagtcaagttgtcctgcacaggttctggcttcaacattaaagacacctatatacactgggtgaagcagaggcctgaacagggcctggagtggattggaaggattgatcctgcgaatgataatattaaatatgacccgaagttccagggcaaggccactataacagcagacacatcctccaacacagcctacctacagctcaacagcctgacatctgaggacactgccgtctattactgtgctagatctgaggaaaattggtacgacttttttgactactggggccaaggcaccactctcacagtctcctca

SEQ ID NO: 143 - protein, 139 aa, Mus musculus
MKCSWVIFFLMAVVTGVNSEVQLQQSGAELVKPGASVKLSCTGSGFNIKDTYIHWVKQRPEQGLEWIGRIDPANDNIKYDPKFQGKATITADTSSNTAYLQLNSLTSEDTAVYYCARSEENWYDFFDYWGQGTTLTVSS

SEQ ID NO: 144 - DNA, 393 bp, Mus musculus
atgaagttgcctgttaggctgttggtgctgatgttctggattcctgcttccagcagtgatgttttgatgacccaaactccactctccctgcctgtcagtcttggagatcaagcctccatctcttgcaggtctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagattagcagagtggaggctgaggatctgggagtttattactgctttcaaggttcacatattccgtacacgttcggaggggggaccaagctggaaataaaa

SEQ ID NO: 145 - protein, 131 aa, Mus musculus
MKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHIPYTFGGGTKLEIK

SEQ ID NO: 146 - DNA, 417 bp, Artificial Sequence
  Description of Artificial Sequence Synthetic polynucleotide
atggattggacctggcgcatcctgttcctggtggccgctgccaccggcgctcactctcaggtgcagctggtgcagtctggcgccgaggtgaagaagcctggcgcttccgtgaaggtgtcctgtaaggcctccggcttcaacatcaaggacacctacatccactgggtgcggcaggctcccggccagcggctggagtggatgggccggatcgatcctgccaacgacaacatcaagtacgaccccaagtttcagggccgcgtgaccatcacccgcgatacctccgcttctaccgcctacatggagctgtctagcctgcggagcgaggataccgccgtgtactactgcgcccgctccgaggagaactggtacgacttcttcgactactggggccagggcaccctggtgaccgtgtcctct

SEQ ID NO: 147 - protein, 144 aa, Artificial Sequence
  Description of Artificial Sequence Synthetic polypeptide
MDWTWRILFLVAAATGAHSQVQLVQSGAEVKKPGASVKVSCKASGFNIKDTYIHWVRQAPGQRLEWMGRIDPANDNIKYDPKFQGRVTITRDTSASTAYMELSSLRSEDTAVYYCARSEENWYDFFDYWGQGTLVTVSSGESCR

SEQ ID NO: 148 - DNA, 396 bp, Artificial Sequence
  Description of Artificial Sequence Synthetic polynucleotide
atgcggctgcccgctcagctgctgggcctgctgatgctgtgggtgcccggctcttccggcgacgtggtgatgacccagtcccctctgtctctgcccgtgaccctgggccagcccgcttctatctcttgccggtcctcccagtccatcgtgcactccaacggcaacacctacctggagtggtttcagcagagacccggccagtctcctcggcggctgatctacaaggtgtccaaccgcttttccggcgtgcccgatcggttctccggcagcggctccggcaccgatttcaccctgaagatcagccgcgtggaggccgaggatgtgggcgtgtactactgcttccagggctcccacatcccttacacctttggcggcggaaccaaggtggagatcaag

SEQ ID NO: 149 - protein, 132 aa, Artificial Sequence
  Description of Artificial Sequence Synthetic peptide
MRLPAQLLGLLMLWVPGSSGDVVMTQSPLSLPVTLGQPASISCRSSQSIVHSNGNTYLEWFQQRPGQSPRRLIYKVSNRFSGVPDRFSGSGSGTDFTLKISRVEAEDVGVYYCFQGSHIPYTFGGGTKVEIK

SEQ ID NO: 150 - DNA, 417 bp, Artificial Sequence
  Description of Artificial Sequence Synthetic polynucleotide
atggagctgggcctgtcttgggtgttcctggtggctatcctggagggcgtgcagtgcgaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtggcccggatcgatcctgccaacgacaacatcaagtacgaccccaagttccagggccggttcaccatctctcgcgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 151 - protein, 139 aa, Artificial Sequence
  Description of Artificial Sequence Synthetic polypeptide
MELGLSWVFLVAILEGVQCEVQLVESGGGLVQPGGSLRLSCAASGFNIKDTYIHWVRQAPGKGLEWVARIDPANDNIKYDPKFQGRFTISRDNAKNSLYLQMNSLRAEDTAVYYCARSEENWYDFFDYWGQGTLVTVSS

SEQ ID NO: 152 - DNA, 405 bp, Artificial Sequence
  Description of Artificial Sequence Synthetic polynucleotide
atggatatgcgcgtgcccgctcagctgctgggcctgctgctgctgtggctgcgcggagcccgctgcgatatccagatgacccagtccccttcttctctgtccgcctctgtgggcgatcgcgtgaccatcacctgtcggtcctcccagtccatcgtgcactccaacggcaacacctacctggagtggtatcagcagaagcccggcaaggcccctaagctgctgatctacaaggtgtccaaccgcttttccggcgtgccttctcggttctccggctccggctccggcaccgatttcaccctgaccatctcctccctccagcccgaggatttcgccacctactactgcttccagggctcccacatcccttacacctttggcggcggaaccaaggtggagatcaagcgt

SEQ ID NO: 153 - protein, 135 aa, Artificial Sequence
  Description of Artificial Sequence Synthetic polypeptide
MDMRVPAQLLGLLLLWLRGARCDIQMTQSPSSLSASVGDRVTITCRSSQSIVHSNGNTYLEWYQQKPGKAPKLLIYKVSNRFSGVPSRFSGSGSGTDFTLTISSLQPEDFATYYCFQGSHIPYTFGGGTKVEIKR

SEQ ID NO: 154 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtggatcggccggatcgatcctgccaacgacaacatcaagtacgaccccaagttccagggccggttcaccatctctcgcgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 155 - protein, 120 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISRDNAKNSLYLQMNSLRAEDTAVYYCARSEENWYDFFDYWGQGTLVTVSS

SEQ ID NO: 156 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtggcccggatcgatcctgccaacgacaacatcaagtacgaccccaagttccagggcaaggccaccatctctcgcgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 157 - protein, 120 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGKATISRDNAKNSLYLQMNSLRAEDTAVYYCARSEENWYDFFDYWGQGTLVTVSS

SEQ ID NO: 158 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtggcccggatcgatcctgccaacgacaacatcaagtacgaccccaagttccagggccggttcaccatctctgccgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 159 - protein, 120 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGRFTISADNAKNSLYLQMNSLRAEDTAVYYCARSEENWYDFFDYWGQGTLVTVSS

SEQ ID NO: 160 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtgggccggatcgatcctgccaacgacaacatcaagtacgaccccaagttccagggccggttcaccatctctcgcgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 161 - protein, 120 aa, Artificial Sequence
  Description of Artificial Sequence Synthetic polypeptide
EVQLVESGGGLVQPGGSLRLSCAASGFNIKDTYIHWVRQAPGKGLEWVGRIDPANDNIKYDPKFQGRFTISRDNAKNSLYLQMNSLRAEDTAVYYCARSEENWYDFFDYWGQGTLVTVSS

SEQ ID NO: 162 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtggcccggatcgatcctgccaacgacaacatcaagtacgaccccaagttccagggcaaggccaccatctctgccgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 163 - protein, 120 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGKATISADNAKNSLYLQMNSLRAEDTAVYYCARSEENWYDFFDYWGQGTLVTVSS

SEQ ID NO: 164 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtggatcggccggatcgatcctgccaacgacaacatcaagtacgaccccaagttccagggccggttcaccatctctgccgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 165 - protein, 120 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISADNAKNSLYLQMNSLRAEDTAVYYCARSEENWYDFFDYWGQGTLVTVSS

SEQ ID NO: 166 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtgggccggatcgatcctgccaacgacaacatcaagtacgaccccaagttccagggccggttcaccatctctgccgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 167 - protein, 120 aa, Artificial Sequence
  Description of Artificial Sequence Synthetic polypeptide
EVQLVESGGGLVQPGGSLRLSCAASGFNIKDTYIHWVRQAPGKGLEWVGRIDPANDNIKYDPKFQGRFTISADNAKNSLYLQMNSLRAEDTAVYYCARSEENWYDFFDYWGQGTLVTVSS

SEQ ID NO: 168 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtggcccggatcgatcctgccaacgacaacatcaagtacgaccccaagttccagggccggttcaccatctctcgcgacaacgccaagaactccgcctacctccagatgaactctctgcgcgccgaggataccgccgtgtactactgcgcccggagcgaggagaactggtacgacttcttcgactactggggccagggcaccctggtgaccgtgtcctct

SEQ ID NO: 169 - protein, 120 aa, Artificial Sequence
  Description of Artificial Sequence Synthetic polypeptide
EVQLVESGGGLVQPGGSLRLSCAASGFNIKDTYIHWVRQAPGKGLEWVARIDPANDNIKYDPKFQGRFTISRDNAKNSAYLQMNSLRAEDTAVYYCARSEENWYDFFDYWGQGTLVTVSS

SEQ ID NO: 170 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtgggtgggccggatcgatcctgccaacgacaacatcaagtacgaccccaagttccagggccggttcaccatctctgccgacaacgccaagaactccgcctacctccagatgaactctctgcgcgccgaggataccgccgtgtactactgcgcccggagcgaggagaactggtacgacttcttcgactactggggccagggcaccctggtgaccgtgtcctct

SEQ ID NO: 171 - protein, 120 aa, Artificial Sequence
  Description of Artificial Sequence Synthetic polypeptide
EVQLVESGGGLVQPGGSLRLSCAASGFNIKDTYIHWVRQAPGKGLEWVGRIDPANDNIKYDPKFQGRFTISADNAKNSAYLQMNSLRAEDTAVYYCARSEENWYDFFDYWGQGTLVTVSS

SEQ ID NO: 172 - DNA, 360 bp, Artificial Sequence
  Description of Artificial Sequence Synthetic polynucleotide
gaggtgcagctggtggagtctggcggcggactggtgcagcctggcggctctctgcggctgtcttgcgccgcttccggcttcaacatcaaggacacctacatccactgggtgcggcaggctcccggcaagggcctggagtggatcggccggatcgatcctgccaacgacaacatcaagtacgaccccaagttccagggccggttcaccatctctgccgacaacgccaagaactccgcctacctccagatgaactctctgcgcgccgaggataccgccgtgtactactgcgcccggagcgaggagaactggtacgacttcttcgactactggggccagggcaccctggtgaccgtgtcctct

SEQ ID NO: 173 - protein, 120 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISADNAKNSAYLQMNSLRAEDTAVYYCARSEENWYDFFDYWGQGTLVTVSS

SEQ ID NO: 174 - DNA, 360 bp, Artificial Sequence
  Description of Artificial Sequence Synthetic polynucleotide
gaggtgcagctggtggagtctggcggcggactggtgcagcctggcggctctctgcggctgtcttgcaccggctccggcttcaacatcaaggacacctacatccactgggtgcggcaggctcccggcaagggcctggagtggatcggccggatcgatcctgccaacgacaacatcaagtacgaccccaagttccagggccggttcaccatctctgccgacaacgccaagaactccctgtacctccagatgaactctctgcgcgccgaggataccgccgtgtactactgcgcccggagcgaggagaactggtacgacttcttcgactactggggccagggcaccctggtgaccgtgtcctct

SEQ ID NO: 175 - protein, 120 aa, Artificial Sequence
  Description of Artificial Sequence Synthetic polypeptide
EVQLVESGGGLVQPGGSLRLSCTGSGFNIKDTYIHWVRQAPGKGLEWIGRIDPANDNIKYDPKFQGRFTISADNAKNSLYLQMNSLRAEDTAVYYCARSEENWYDFFDYWGQGTLVTVSS

SEQ ID NO: 176 - protein, 450 aa, Artificial Sequence
  Description of Artificial Sequence Synthetic polypeptide
EVQLVESGGGLVQPGGSLRLSCAASGFNIKDTYIHWVRQAPGKGLEWIGRIDPANDNIKYDPKFQGRFTISADNAKNSAYLQMNSLRAEDTAVYYCARSEENWYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 219 aa, Artificial Sequence
  Description of Artificial Sequence Synthetic polypeptide
DIQMTQSPSSLSASVGDRVTITCRSSQSIVHSNGNTYLEWYQQKPGKAPKLLIYKVSNRFSGVPSRFSGSGSGTDFTLTISSLQPEDFATYYCFQGSHIPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 132 aa, Homo sapiens
MALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 179 - protein, 13 aa, Macaca fascicularis
MALLLTMVIALTC

SEQ ID NO: 180 - protein, 12 aa, Macaca fascicularis
LGGFASPSPVPP

SEQ ID NO: 181 - protein, 17 aa, Macaca fascicularis
SPSPVPPSTALKELIEE

SEQ ID NO: 182 - protein, 19 aa, Macaca fascicularis
TALKELIEELVNITQNQKA

SEQ ID NO: 183 - protein, 22 aa, Macaca fascicularis
NQKAPLCNGSMVWSINLTAGVY

SEQ ID NO: 184 - protein, 21 aa, Macaca fascicularis
INLTAGVYCAALESLINVSGC

SEQ ID NO: 185 - protein, 21 aa, Macaca fascicularis
SLINVSGCSAIEKTQRMINGF

SEQ ID NO: 186 - protein, 18 aa, Macaca fascicularis
GFCPHKVSAGQFSSLRVR

SEQ ID NO: 187 - protein, 20 aa, Macaca fascicularis
VRDTKIEVAQFVKDLLVHLK

SEQ ID NO: 188 - protein, 19 aa, Macaca fascicularis
FVKDLLVHLKKLFREGQFN

SEQ ID NO: 189 - DNA, 396 bp, Artificial Sequence
  Description of Artificial Sequence Synthetic polynucleotide
atgcggctgcccgctcagctgctgggcctgctgatgctgtgggtgcccggctcttccggcgacgtggtgatgacccagtcccctctgtctctgcccgtgaccctgggccagcccgcttctatctcttgccggtcctcccagtccctggtgtactccgacggcaacacctacctgaactggttccagcagagacccggccagtctcctcggcggctgatctacaaggtgtccaaccgcttttccggcgtgcccgatcggttctccggctccggcagcggcaccgatttcaccctgaagatcagccgcgtggaggccgaggatgtgggcgtgtactactgcttccagggctcccacatcccttacacctttggcggcggaaccaaggtggagatcaag

SEQ ID NO: 190 - protein, 132 aa, Artificial Sequence
  Description of Artificial Sequence Synthetic polypeptide
MRLPAQLLGLLMLWVPGSSGDVVMTQSPLSLPVTLGQPASISCRSSQSLVYSDGNTYLNWFQQRPGQSPRRLIYKVSNRFSGVPDRFSGSGSGTDFTLKISRVEAEDVGVYYCFQGSHIPYTFGGGTKVEIK

SEQ ID NO: 191 - DNA, 336 bp, Artificial Sequence
  Description of Artificial Sequence Synthetic polynucleotide
gatgttgtgatgacccaatctccactctccctgcctgtcactcctggagagccagcctccatctcttgcagatctagtcagagcattgtgcatagtaatggaaacacctacctggaatggtacctgcagaaaccaggccagtctccacagctcctgatctacaaagtttccaaccgattttctggggtcccagacaggttcagtggcagtggatcagggacagatttcacactcaagatcagcagagtggaggctgaggatgtgggagtttattactgctttcaaagttcacatgttcctctcaccttcggtcaggggaccaagctggagatcaaa

SEQ ID NO: 192 - protein, 112 aa, Artificial Sequence
  Description of Artificial Sequence Synthetic polypeptide
DVVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQSSHVPLTFGQGTKLEIK

SEQ ID NO: 193 - protein, 329 aa, Homo sapiens; identical to SEQ ID NO: 128
  MUTAGEN (116)..(116)
  MUTAGEN (119)..(119)

SEQ ID NO: 194 - protein, 132 aa, Homo sapiens; identical to SEQ ID NO: 178

SEQ ID NO: 195 - protein, 113 aa, Homo sapiens; identical to SEQ ID NO: 124

SEQ ID NO: 196 - protein, 10 aa, Mus musculus
LDGYYFGFAY

SEQ ID NO: 197 - protein, 15 aa, Mus musculus
KASESVDNYGKSLMH

SEQ ID NO: 198 - protein, 118 aa, Mus musculus
EVKLVESGGGLVKPGGSLKLSCAASGFTFISYAMSWVRQTPEKRLEWVASISSGGNTYYPDSVKGRFTISRDNARNILYLQMSSLRSEDTAMYYCARLDGYYFGFAYWGQGTTVTVSS

SEQ ID NO: 199 - protein, 111 aa, Mus musculus
DIVLTQSPASLAVSLGQRATISCKASESVDNYGKSLMHWYQQKPGQSPKLLIYRASNLESGIPARFSGSGSRTDFTLTINPVEADDVATYYCQQSNEDPWTFGGGTKLEIK

SEQ ID NO: 200 - protein, 7 aa, Mus musculus
RASNLES

SEQ ID NO: 201 - protein, 9 aa, Mus musculus
QQSNEDPWT

SEQ ID NO: 202 - protein, 6 aa, Mus musculus
ISYAMS

SEQ ID NO: 203 - protein, 16 aa, Mus musculus
SISSGGNTYYPDSVKG

SEQ ID NO: 204 - DNA, 351 bp, Mus musculus
gaagtgaagctggtggagtctgggggaggcttagtgaaacctggagggtccctgaaactctcctgtgcagcctctggattcactttcattagctatgccatgtcttgggttcgtcagactccagagaagaggctggagtgggtcgcatccattagtagtggtggtaacacctactatccagacagtgtgaagggccgattcaccatctccagagataatgccaggaacatcctatacctgcaaatgagcagtctgaggtctgaggacacggccatgtattactgtgcacgacttgatggttactactttggatttgcttactggggccaagggactctggtcgctgtctct

SEQ ID NO: 205 - DNA, 354 bp, Artificial Sequence
  Description of Artificial Sequence Synthetic partially humanized antibody heavy chain
gaggtcaagctggtggagtcagggggaggcttagtgcaacctggagggtccctgagactctcctgtgcagcctctggattcactttcattagctatgccatgtcttgggttcgtcaggctccagggaaggggctggagtgggtcgcatccattagtagtggtggtaacacctactatccagacagcgtgaagggccgattcaccatctccagagataatgccaagaacagcctatacctgcaaatgaacagtctgagggctgaggacacggccgtgtattactgtgcacgacttgatggttactactttggatttgcttactggggccaagggaccctggtcaccgtctcctca

SEQ ID NO: 206 - DNA, 354 bp, Artificial Sequence
  Description of Artificial Sequence Synthetic fully humanized antibody heavy chain
gaggtccagctggtggagtcagggggaggcttagtgcaacctggagggtccctgagactctcctgtgcagcctctggattcactttcattagctatgccatgtcttgggttcgtcaggctccagggaaggggctggagtgggtcgcatccattagtagtggtggtaacacctactatccagacagcgtgaagggccgattcaccatctccagagataatgccaagaacagcctatacctgcaaatgaacagtctgagggctgaggacacggccgtgtattactgtgcacgacttgatggttactactttggatttgcttactggggccaagggaccctggtcaccgtctcctca

SEQ ID NO: 207 - DNA, 354 bp, Artificial Sequence
  Description of Artificial Sequence Synthetic fully humanized antibody heavy chain
gaggtccagctggtggagtcagggggaggcttagtgaaacctggagggtccctgagactctcctgtgcagcctctggattcactttcattagctatgccatgtcttgggttcgtcaggctccagggaaggggctggagtgggtctcatccattagtagtggtggtaacacctactatccagacagtgtgaagggccgattcaccatctccagagataatgccaagaacagcctatacctgcaaatgaacagtctgagggctgaggacacggccgtgtattactgtgcacgacttgatggttactactttggatttgcttactggggccaagggaccacggtcaccgtctcctca

SEQ ID NO: 208 - protein, 118 aa, Mus musculus; identical to SEQ ID NO: 198

SEQ ID NO: 209 - protein, 118 aa, Artificial Sequence
  Description of Artificial Sequence Synthetic partially humanized antibody heavy chain
EVKLVESGGGLVQPGGSLRLSCAASGFTFISYAMSWVRQAPGKGLEWVASISSGGNTYYPDSVKGRFTISRDNAKNSLYLQMNSLRAEDTAVYYCARLDGYYFGFAYWGQGTLVTVSS

SEQ ID NO: 210 - protein, 118 aa, Artificial Sequence
  Description of Artificial Sequence Synthetic fully humanized antibody heavy chain
EVQLVESGGGLVQPGGSLRLSCAASGFTFISYAMSWVRQAPGKGLEWVASISSGGNTYYPDSVKGRFTISRDNAKNSLYLQMNSLRAEDTAVYYCARLDGYYFGFAYWGQGTLVTVSS

SEQ ID NO: 211 - protein, 118 aa, Artificial Sequence
  Description of Artificial Sequence Synthetic fully humanized antibody heavy chain
EVQLVESGGGLVKPGGSLRLSCAASGFTFISYAMSWVRQAPGKGLEWVSSISSGGNTYYPDSVKGRFTISRDNAKNSLYLQMNSLRAEDTAVYYCARLDGYYFGFAYWGQGTTVTVSS

SEQ ID NO: 212 - protein, 111 aa, Artificial Sequence
  Description of Artificial Sequence Synthetic fully humanized antibody light chain
DIQMTQSPSSLSASVGDRVTITCKASESVDNYGKSLMHWYQQKPGKAPKLLIYRASNLESGVPSRFSGSGSGTDFTLTISSLQPEDFATYYCQQSNEDPWTFGGGTKVEIK

SEQ ID NO: 213 - protein, 111 aa, Mus musculus; identical to SEQ ID NO: 199

SEQ ID NO: 214 - protein, 111 aa, Artificial Sequence
  Description of Artificial Sequence Synthetic partially humanized antibody light chain
DIQLTQSPSSLSASVGDRVTITCKASESVDNYGKSLMHWYQQKPGKAPKLLIYRASNLESGVPSRFSGSGSRTDFTLTISSLQPEDFATYYCQQSNEDPWTFGGGTKVEIK

SEQ ID NO: 215 - protein, 111 aa, Artificial Sequence
  Description of Artificial Sequence Synthetic fully humanized antibody light chain
DIVLTQSPASLAVSPGQRATITCKASESVDNYGKSLMHWYQQKPGQPPKLLIYRASNLESGVPARFSGSGSGTDFTLTINPVEANDTANYYCQQSNEDPWTFGGGTKVEIK

SEQ ID NO: 216 - protein, 107 aa, Homo sapiens [sequence omitted]

SEQ ID NO: 217 - DNA, 351 bp, Mus musculus; identical to SEQ ID NO: 204

SEQ ID NO: 218 - DNA, 333 bp, Mus musculus
gacattgtgctgacccaatctccagcttctttggctgtgtctctagggcagagggccaccatatcctgcaaagccagtgaaagtgttgataattatggcaaaagtttaatgcactggtaccagcagaaaccaggacagtcacccaaactcctcatctatcgtgcatccaacctagaatctgggatccctgccaggttcagtggcagtgggtctaggacagacttcaccctcaccattaatcctgtggaggctgatgatgttgcaacctattactgtcagcaaagtaatgaggatccgtggacgttcggtggaggcaccaagctggaaatcaaa

SEQ ID NO: 219 - DNA, 333 bp, Mus musculus; identical to SEQ ID NO: 218

SEQ ID NO: 220 - DNA, 333 bp, Artificial Sequence
  Description of Artificial Sequence Synthetic partially humanized antibody light chain
gacatccagctgacccagtctccatcctccctgtctgcatctgtgggagacagagtcaccatcacttgcaaagccagtgaaagtgttgataattatggcaaaagtctgatgcactggtatcagcagaaaccagggaaagctcctaagctcctgatctatcgtgcatccaacctggaatctggcgtcccatcaaggttcagtggcagtggatctcgcacagatttcactctcaccatcagcagtctgcaacctgaagattttgcaacttactactgtcagcaaagtaatgaggatccctggaccttcggcggagggaccaaggtagagatcaaa

SEQ ID NO: 221 - DNA, 333 bp, Artificial Sequence
  Description of Artificial Sequence Synthetic fully humanized antibody light chain
gacatccagatgacccagtctccatcctccctgtctgcatctgtgggagacagagtcaccatcacttgcaaagccagtgaaagtgttgataattatggcaaaagtctgatgcactggtatcagcagaaaccagggaaagctcctaagctcctgatctatcgtgcatccaacctggaatctggcgtcccatcaaggttcagtggcagtggatctggcacagatttcactctcaccatcagcagtctgcaacctgaagattttgcaacttactactgtcagcaaagtaatgaggatccctggaccttcggcggagggaccaaggtagagatcaaa

SEQ ID NO: 222 - DNA, 351 bp, Mus musculus; identical to SEQ ID NO: 204

SEQ ID NO: 223 - DNA, 333 bp, Artificial Sequence
  Description of Artificial Sequence Synthetic fully humanized antibody light chain
gacatcgtgctcactcagtctccagcttctttggctgtgtctccagggcagagggccaccataacctgcaaagccagtgaaagtgttgataattatggcaaaagtttaatgcactggtaccagcagaaaccaggacagccacccaaactcctcatctatcgtgcatccaacctagaatctggggtccctgccaggttcagtggcagtgggtctgggacagacttcaccctcaccattaatcctgtggaggctaatgatactgcaaactattactgtcagcaaagtaatgaggatccgtggacgttcggtggagggaccaaggtggaaataaaa

SEQ ID NO: 224 - protein, 227 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSNIC

(19) United States
(12) Patent Application Publication
(10) Pub. No.: US 2009/0098142 A1
(43) Pub. Date: Apr. 16, 2009

(54) Methods and compositions for treating and monitoring treatment of IL-13-associated disorders

(76) Inventors: Marion T. Kasaian, Cambridge, MA (US); Timothy A. Cook, Acton, MA (US); Samuel J. Goldman, Acton, MA (US); Donald G. Raible, Devon, PA (US)

Correspondence Address: LOWRIE, LANDO & ANASTASI, LLP;W2023, ONE MAIN STREET, SUITE 1100, CAMBRIDGE, MA, 02142, US

(21) Appl. No.: 12/001,637

(22) Filed: Dec. 11, 2007

Related U.S. Application Data
(63) Continuation-in-part of application No. 11/149,309, filed on Jun. 9, 2005.
Application No. 11/149,309 is a continuation-in-part of application No. 11/155,843, filed on Jun. 17, 2005.
Application No. 11/155,843 is a continuation-in-part of application No. 11/149,025, filed on Jun. 9, 2005.
(60) Provisional application No. 60/578,473, filed on Jun. 9, 2004.
(60) Provisional application No. 60/581,375, filed on Jun. 22, 2004.
(60) Provisional application No. 60/578,736, filed on Jun. 9, 2004.
(60) Provisional application No. 60/581,078, filed on Jun. 17, 2004.
(60) Provisional application No. 60/925,932, filed on Apr. 23, 2007.
(60) Provisional application No. 60/874,333, filed on Dec. 11, 2006.

(51) Int. Cl. A61K 39/395; G01N 33/53; A61P 37/06

(52) U.S. Cl. 424/158.1; 435/7.9

(57) ABSTRACT

Methods and compositions for treating and/or monitoring treatment of IL-13-associated disorders or conditions are disclosed.

### CROSS REFERENCE TO RELATED APPLICATIONS

This application is a continuation-in-part of U.S. Ser. No. 11/149,309, filed Jun. 9, 2005, which claims priority under 35 U.S.C. §119 to U.S. Ser. No. 60/578,473, filed on Jun. 9, 2004, U.S. Ser. No. 60/581,375, filed on Jun. 22, 2004, and U.S. Ser. No. 60/578,736, filed on Jun. 9, 2004. This application is also a continuation-in-part of U.S. Ser. No. 11/155,843, filed on Jun. 17, 2005, which claims priority under 35 U.S.C. §119 to U.S. Ser. No. 60/581,078, filed on Jun. 17, 2004, and is a continuation-in-part of U.S. Ser. No. 11/149,025, filed on Jun. 9, 2005. This application also claims priority to U.S. Ser. No. 60/874,333, filed on Dec. 11, 2006, and U.S. Ser. No. 60/925,932, filed on Apr. 23, 2007. The contents of all of the aforementioned applications are hereby incorporated by reference in their entirety. This application also incorporates by reference the International Application filed with the U.S. Receiving Office on Dec. 11, 2007, entitled “Methods and Compositions for Treating and Monitoring Treatment of IL-13-Associated Disorders” and bearing attorney docket number 16158-105WO1.

### SEQUENCE LISTING

A copy of the Sequence Listing in electronic and paper form is being submitted herewith.

### BACKGROUND

Interleukin-13 (IL-13) is a cytokine secreted by T lymphocytes and mast cells (McKenzie et al. (1993) Proc. Natl. Acad. Sci. USA 90:3735-39; Bost et al. (1996) Immunology 87:663-41). IL-13 shares several biological activities with IL-4. For example, either IL-4 or IL-13 can cause IgE isotype switching in B cells (Tomkinson et al. (2001) J. Immunol. 166:5792-5800). Additionally, increased levels of cell surface CD23 and serum CD23 (sCD23) have been reported in asthmatic patients (Sanchez-Guererro et al. (1994) Allergy 49:587-92; DiLorenzo et al. (1999) Allergy Asthma Proc. 20:119-25). In addition, either IL-4 or IL-13 can upregulate the expression of MHC class II and the low-affinity IgE receptor (CD23) on B cells and monocytes, which results in enhanced antigen presentation and regulated macrophage function (Tomkinson et al., supra). Importantly, either IL-4 or IL-13 can increase the expression of VCAM-1 on endothelial cells, which facilitates preferential recruitment of eosinophils (and T cells) to the airway tissues (Tomkinson et al., supra). Either IL-4 or IL-13 can also increase airway mucus secretion, which can exacerbate airway responsiveness (Tomkinson et al., supra). These observations suggest that although IL-13 is not necessary for, or even capable of, inducing Th2 development, IL-13 may be a key player in the development of airway eosinophilia and AHR (Tomkinson et al., supra; Wills-Karp et al. (1998) Science 282:2258-61).

### SUMMARY

Methods and compositions for treating and/or monitoring treatment of IL-13-associated disorders or conditions are disclosed. In one aspect, Applicants have discovered that a single administration of an IL-13 antagonist or an IL-4 antagonist to a subject, prior to the onset of an IL-13 associated disorder or condition, reduces one or more symptoms of the disorder or condition, relative to an untreated subject. Enhanced reduction of the symptoms of the disorder or condition is detected after co-administration of the IL-13 antagonist with the IL-4 antagonist, relative to the reduction detected after administration of the single agent. Thus, methods for reducing or inhibiting, or preventing or delaying the onset of, one or more symptoms of an IL-13-associated disorder or condition using an IL-13 antagonist alone or in combination with an IL-4 antagonist are disclosed. In other embodiments, methods for evaluating the efficacy of an IL-13 antagonist in treating or preventing an IL-13-associated disorder or condition in a subject, e.g., a human subject, are also disclosed.

Accordingly, in one aspect, the invention features a method of treating or preventing an IL-13-associated disorder or condition in a subject. The method includes administering an IL-13 antagonist and/or an IL-4 antagonist to the subject, in an amount effective to reduce one or more symptoms of the disorder or condition (e.g., in an amount effective to reduce one or more of: IgE levels, histamine release, eotaxin levels, or a respiratory symptom in the subject). In the case of prophylactic use (e.g., to prevent, reduce or delay onset or recurrence of one or more symptoms of the disorder or condition), the subject may or may not have one or more symptoms of the disorder or condition. For example, the IL-13 antagonist and/or IL-4 antagonist can be administered prior to any detectable manifestation of the symptoms, or after at least some, but not all the symptoms are detected. In the case of therapeutic use, the treatment may improve, cure, maintain, or decrease duration of, the disorder or condition in the subject. In therapeutic uses, the subject may have a partial or full manifestation of the symptoms. In a typical case, treatment improves the disorder or condition of the subject to an extent detectable by a physician, or prevents worsening of the disorder or condition.

In one embodiment, the IL-13 antagonist and/or IL-4 antagonist is administered at a single treatment interval, e.g., as a single dose, or as a repeated dose of no more than two or three doses during a single treatment interval, e.g., the repeated dose is administered within one week or less from the initial dose. For example, the IL-13 antagonist and/or the IL-4 antagonist can be administered at a single treatment interval prior to the onset or recurrence of one or more symptoms associated with the IL-13-disorder or condition, but before a full manifestations of the symptoms associated with the disorder or condition. In certain embodiments, the IL-13 antagonist and/or IL-4 antagonist is administered to the subject prior to exposure to an agent that triggers or exacerbates an IL-13-associated disorder or condition, e.g., an allergen, a pollutant, a toxic agent, an infection and/or stress. In some embodiments, the IL-13 antagonist and/or IL-4 antagonist is administered prior to, during, or shortly after exposure to the agent that triggers and/or exacerbates the IL-13-associated disorder or condition. For example, the IL-13 antagonist and/or IL-4 antagonist can be administered 1, 5, 10, 25, or 24 hours; 2, 3, 4, 5, 10, 15, 20, or 30 days; or 4, 5, 6, 7 or 8 weeks, or more before or after exposure to the triggering or exacerbating agent. Typically, the IL-13 and/or IL-4 antagonist can be administered anywhere between 24 hours and 2 days before or after exposure to the triggering or exacerbating agent. In those embodiments where administration occurs after exposure to the agent, the subject may not be experiencing symptoms or may be experiencing a partial manifestation of the symptoms. For example, the subject may have symptoms of an early stage of the disorder or condition. Each dose can be administered by inhalation or by injection, e.g., subcutaneously, in an amount of about 0.5-10 mg/kg (e.g., about 0.7-5 mg/kg, 0.9-4 mg/kg, 1-3 mg/kg, 1.5-2.5 mg/kg, 2 mg/kg).

The IL-13 antagonist and/or IL-4 antagonist can be administered to a subject having, or at risk of having, an IL-13-associated disorder or condition. Typically, the subject is a mammal, e.g., a human (e.g., a child, an adolescent or an adult) suffering from or at risk of having an IL-13-associated disorder or condition. Examples of IL-13-associated disorders or conditions include, but are not limited to, disorders chosen from one or more of: IgE-related disorders, including but not limited to, atopic disorders, e.g., resulting from an increased sensitivity to IL-13 or IL-4 (e.g., atopic dermatitis, urticaria, eczema, and allergic conditions such as allergic rhinitis and allergic enterogastritis); respiratory disorders, e.g., asthma (e.g., allergic and nonallergic asthma (e.g., asthma due to infection with, e.g., respiratory syncytial virus (RSV), e.g., in younger children)), chronic obstructive pulmonary disease (COPD), and other conditions involving airway inflammation, eosinophilia, fibrosis and excess mucus production, e.g., cystic fibrosis and pulmonary fibrosis; inflammatory and/or autoimmune disorders or conditions, e.g., skin inflammatory disorders or conditions (e.g., atopic dermatitis), gastrointestinal disorders or conditions (e.g., inflammatory bowel diseases (IBD), ulcerative colitis and/or Crohn's disease), liver disorders or conditions (e.g., cirrhosis, hepatocellular carcinoma), and scleroderma; tumors or cancers (e.g., soft tissue or solid tumors), such as leukemia, glioblastoma, and lymphoma, e.g., Hodgkin's lymphoma; viral infections (e.g., from HTLV-1); fibrosis of other organs, e.g., fibrosis of the liver (e.g., fibrosis caused by a hepatitis B and/or C virus); and suppression of expression of protective type 1 immune responses, (e.g., during vaccination).

For example, the subject can be a human allergic to a seasonal allergen, e.g., ragweed, or an asthmatic patient after exposure to a cold or flu virus or during the cold or flu season. Prior to the onset of the symptoms (e.g., allergic or asthmatic symptoms, or prior to or during an allergy, or cold or flu season), a single dose interval of the anti-IL-13 antagonist and/or IL-4 antagonist can be administered to the subject, such that the symptoms are reduced and/or the onset of the disorder or condition is delayed. Similarly, administration of the IL-13 and/or IL-4 antagonist can be effected prior to the manifestation of one or more symptoms (e.g., before a full manifestations of the symptoms) associated with the disorder or condition when treating chronic conditions that are characterized by recurring flares or episodes of the disorder or condition. An exemplary method for treating allergic rhinitis or other allergic disorders can include initiating therapy with an IL-13 and/or IL-4 antagonist prior to exposure to an allergen, e.g., prior to seasonal exposure to an allergen, e.g., prior to allergen blooms. Such therapy can include a single treatment interval, e.g., a single dose, of the IL-13 and/or IL-4 antagonist. In other embodiments, the single treatment interval of the IL-13 and/or IL-4 antagonist is administered in combination with allergy immunotherapy. For example the single treatment interval of the IL-13 and/or IL-4 antagonist is administered in combination with an allergy immunization, e.g., a vaccine containing one or more allergens, such as ragweed, dust mite, and ryegrass. The single treatment interval can be repeated until a predetermined level of immunity is obtained in the subject.

In other embodiments, the IL-13 antagonist and/or the IL-4 antagonist is administered in an amount effective to reduce or inhibit, or prevent or delay the onset of, one or more of the symptoms of the IL-13-associated disorder or condition. For example the IL-13 and/or IL-4 antagonist can be administered in an amount that decreases one or more of: (i) the levels of IL-13 in the subject; (ii) the levels of eotaxin in the subject; (iii) the levels of histamine released by basophils (e.g., blood basophils); (iv) the IgE-titers in the subject; and/or (v) one or more changes in the respiratory symptoms of the subject (e.g., difficulty breathing, wheezing, coughing, shortness of breath and/or difficulty performing normal daily activities).

In other embodiments, the IL-13 antagonist and/or the IL-4 antagonist inhibits or reduces one or more biological activities of IL-13 or IL-4, or an IL-13 receptor (e.g., an IL-13 receptor α1 or an IL-13 receptor α2) or an IL-4 receptor (e.g., an IL-4 receptor a or a receptor associated subunit thereof, e.g., γ-chain). Exemplary biological activities that can be reduced using the IL-13 or IL-4 antagonists disclosed herein include, but is not limited to, one or more of: induction of CD23 expression; production of IgE by human B cells; phosphorylation of a transcription factor, e.g., STAT protein (e.g., STAT6 protein); antigen-induced eosinophilia in vivo; antigen-induced bronchoconstriction in vivo; and/or drug-induced airway hyperreactivity in vivo. Antagonism using an antagonist of IL-13/IL-13R or IL-4/IL-4R does not necessarily indicate a total elimination of the biological activity of the IL-13/IL-13R polypeptide and/or the IL-4/IL-4R polypeptide.

For purposes of clarity, the term “IL-13 antagonist” or “IL-4 antagonist,” as used herein, collectively refers to a compound such as a protein (e.g., a multi-chain polypeptide, a polypeptide), a peptide, small molecule, or inhibitory nucleic acid that reduces, inhibits or otherwise blocks one or more biological activities of IL-13 and an IL-13R, or IL-4 and an IL-4R, respectively. In one embodiment, the IL-13 antagonist interacts with, e.g., binds to, an IL-13 or IL-13R polypeptide (also referred to herein as an “antagonistic IL-13 binding agent.” For example, the IL-13 antagonist can interact with, e.g., can bind to, IL-13 or IL-13 receptor, preferably, mammalian, e.g., human IL-13 or IL-13R (also individually referred to herein as an “IL-13 antagonist” and “IL-13R antagonist,” respectively), and reduce or inhibit one or more IL-13- and/or IL-13R-associated biological activities. In another embodiment, the IL-4 antagonist interacts with, e.g., binds to, an IL-4 or an IL-4R polypeptide (e.g., mammalian, e.g., human IL-4 or IL-4R (also individually referred to herein as an “IL-4 antagonist” and “IL-4R antagonist,” respectively)), and reduce or inhibit one or more IL-4 and/or IL-4R activities. Antagonists bind to IL-13 or IL-4, or IL-13R or IL-4R with high affinity, e.g., with an affinity constant of at least about 10⁻⁷ M⁻¹, preferably about 10⁸ M⁻¹, and more preferably, about 10⁹ M⁻¹ to 10¹⁰ M⁻¹ or stronger. It is noted that the term “IL-13 antagonist” or “IL-4 antagonist” includes agents that inhibit or reduce one or more of the biological activities disclosed herein, but may not bind to IL-13 or IL-4 directly.

The terms “anti-IL13 binding agent” and “IL-13 binding agent” are used interchangeably herein. These terms as used herein refers to any compound, such as a protein (e.g., a multi-chain polypeptide, a polypeptide) or a peptide, that includes an interface that binds to an IL-13 protein, e.g., a mammalian IL-13, particularly, a human IL-13. The binding agent generally binds with a Kd of less than 5×10⁻⁷ M. An exemplary IL-13 binding agent is a protein that includes an antigen binding site, e.g., an antibody molecule. The anti-IL13 binding agent or IL-13 binding agent can be an IL-13 antagonist that binds to IL13, or can also include IL-13 binding agents that simply bind to IL-13, but do not elicit an activity, or may in fact agonize an IL-13 activity. For example, certain IL-13 binding agents, e.g., anti-IL-13 antibody molecules, that bind to and inhibit one or more IL-13 biological activities, e.g., antibodies 13.2, MJ2-7 and C65, are also referred to herein as antagonistic IL-13 binding agents. Examples of IL-13 antagonists that are not IL-13 binding agents as defined herein include, e.g., inhibitors of upstream or downstream IL-13 signalling (e.g., STAT6 inhibitors).

Additional embodiments may include one or more of the following features:

In some embodiments, the IL-13 antagonist or the IL4 antagonist can be an antibody molecule that binds to IL-13 or an IL-13R, or IL-4 or an IL-4R. The IL-13 or the IL-4 antagonist can also be a soluble form of the IL-13R (e.g., soluble IL-13Rα2 or IL-13Rα1) or the IL-4R (e.g., IL-4Rα), alone or fused to another moiety (e.g., an immunoglobulin Fc region) or as a heterodimer of subunits (e.g., a soluble IL-13R-IL-4R heterodimer or a soluble IL-4R-γ common heterodimer). In other embodiments, the antagonist is a cytokine mutein (e.g., an IL-13 or IL-4 mutein that binds to the corresponding receptor, but does not substantially activate the receptor), or a cytokine conjugated to a toxin. In other embodiments, the IL-13 or the IL-4 antagonist is a small molecule inhibitor, e.g., a small molecule inhibitor of STAT6, or a peptide inhibitor. In yet other embodiments, the IL-13 or IL-4 antagonist is an inhibitor of nucleic acid expression. For example, the antagonist is an antisense RNA or siRNA that blocks or reduces expression of an IL-13 or IL-13R, or IL-4 or IL-4R gene.

In one embodiment, the IL-13 antagonist or binding agent (e.g., the antibody molecule, soluble receptor, cytokine mutein, or peptide inhibitor) binds to IL-13 or an IL13R and inhibits or reduces an interaction (e.g., binding) between IL-13 and an IL-13 receptor, e.g., IL-13Rα1, IL-13Rα2, and/or IL-4Rα, thereby reducing or inhibiting signal transduction. For example, the IL-13 antagonist can bind to one or more components of a complex chosen from, e.g., IL-13 and IL-13Rα1 (“IL-13/IL-13αR1”); IL-13 and IL-4Rα (“IL-13/IL-4Rα”); IL-13, IL-13Rα1, and IL-4Rα (“IL-13/IL-13Rα1/IL-4Rα”); and IL-13 and IL-13Rα2 (“IL-13/IL13Rα2”). In embodiments, the IL-13 antagonist binds to IL-13 or an IL-13R and interferes with (e.g., inhibits, blocks or otherwise reduces) an interaction, e.g., binding, between IL-13 and an IL-13 receptor complex, e.g., a complex comprising IL-13Rα1 and IL-4Rα. In other embodiments, the IL-13 antagonist binds to IL-13 and interferes with (e.g., inhibits, blocks or otherwise reduces) an interaction, e.g., binding, between IL-13 and a subunit of the IL-13 receptor complex, e.g., IL-13Rα1 or IL-4Rα, individually. In yet another embodiment, the IL-13 antagonist, e.g., the anti-IL-13 antibody or fragment thereof, binds to IL-13, and interferes with (e.g., inhibits, blocks or otherwise reduces) an interaction, e.g., binding, between IL-13/IL-13Rα1 and IL-4Rα. In another embodiment, the IL-13 antagonist, binds to IL-13 and interferes with (e.g., inhibits, blocks or otherwise reduces) an interaction, e.g., binding, between IL-13/IL-4Rα and IL-13Rα1. Typically, the IL-13 antagonist interferes with (e.g., inhibits, blocks or otherwise reduces) an interaction, e.g., binding, of IL-13/IL-13Rα1 with IL-4Rα. Exemplary antibodies inhibit or prevent formation of the ternary complex, IL-13/IL-13Rα1/IL-4Rα.

In another embodiment, the IL-4 antagonist (e.g., the antibody molecule, soluble receptor, cytokine mutein, or peptide inhibitor) binds to IL-4 or an IL4R, and inhibits or reduces an interaction (e.g., binding) between IL-4 and an IL-4 receptor, e.g., IL-4Rα and/or γ common), thereby reducing or inhibiting signal transduction. For example, the IL-4 antagonist can bind to one or more components of a complex chosen from, e.g., IL-4 and IL-4Rα (“IL-4/IL-4Rα”), IL-4 and γ common (“IL-4/γcommon”), or IL-4, IL-4Rα, and γ common (“IL-4/IL-4Rα/γ common”). In exemplary embodiments, the IL-4 antagonist binds to IL-4 and interferes with (e.g., inhibits, blocks or otherwise reduces) an interaction, e.g., binding, between IL-4 and a subunit of the IL-4 receptor complex, e.g., IL-4Rα or γ common, individually. In yet another embodiment, the IL-4 antagonist, binds to IL-4, and interferes with (e.g., inhibits, blocks or otherwise reduces) an interaction, e.g., binding, between IL-4/IL-4Rα and γ common.

In one embodiment, the IL-13/IL-13R or IL-4/IL-4R antagonist or binding agent is an antibody molecule (e.g., an antibody, or an antigen-binding fragment thereof) that binds to IL-13/IL-13R or IL-4/IL-4R. For example, the antibody molecule can be a full length monoclonal or single specificity antibody that binds to IL-13 or IL-4, or an IL-13 receptor or an IL-4 receptor (e.g., an antibody molecule that includes at least one, and typically two, complete heavy chains, and at least one, and typically two, complete light chains); or an antigen-binding fragment thereof (e.g., a heavy or light chain variable domain monomer or dimer (e.g., V_{H}, V_{HH}), an Fab, F(ab′)₂, Fv, or a single chain Fv fragment). Typically, the antibody molecule is a human, camelid, shark, humanized, chimeric, or in vitro-generated antibody to human IL-13 or IL-4, or a human IL-13 receptor or IL-4 receptor. In certain embodiments, the antibody molecule includes a heavy chain constant region chosen from, e.g., the heavy chain constant regions of IgG1, IgG2, IgG3, IgG4, IgM, IgA1, IgA2, IgD, and IgE; particularly, chosen from, e.g., the heavy chain constant regions of IgG1, IgG2, IgG3, and IgG4, more particularly, the heavy chain constant regions IgG1 (e.g., human IgG1 or a modified form thereof). In another embodiment, the antibody molecule has a light chain constant region chosen from, e.g., the light chain constant regions of kappa or lambda, preferably kappa (e.g., human kappa). In one embodiment, the constant region is altered, e.g., mutated, to modify the properties of the antibody molecule (e.g., to increase or decrease one or more of: Fc receptor binding, antibody glycosylation, the number of cysteine residues, effector cell function, or complement function). For example, the human IgG1 constant region can be mutated at one or more residues, e.g., one or more of residues 234 and 237, as described in Example 5, to decrease one or more of: Fc receptor binding, antibody glycosylation, the number of cysteine residues, effector cell function, or complement function. In embodiments, the antibody molecule includes a human IgG1 constant region mutated at one or more residues of SEQ ID NO: 193, e.g., mutated at positions 116 and 119 of SEQ ID NO: 193.

In one embodiment, the antibody molecule is a inhibitory or neutralizing antibody molecule. For example, the anti-IL13 antibody molecule can have a functional activity comparable to IL-13Rα2 (e.g., the anti-IL13 antibody molecule reduces or inhibits IL-13 interaction with IL-13Rα1). The anti-IL13 antibody molecule may prevent formation of a complex between IL-13 and IL-13Rα1, or disrupt or destabilize a complex between IL-13 and IL-13Rα1. In one embodiment, the anti-IL13 antibody molecule inhibits ternary complex formation, e.g., formation of a complex between IL 13, IL-13Rα1 and IL4-R. In one embodiment, the antibody molecule confers a post-injection protective effect against exposure to an antigen, e.g., an Ascaris antigen in a sheep model, at least 6 weeks after injection. In other embodiments, the anti-IL13 antibody molecule can inhibit one or more IL-13-associated biological activities with an IC₅₀ of about 50 nM to 5 pM, typically about 100 to 250 pM or less, e.g., better inhibition. In one embodiment, the anti-IL13 antibody molecule can associate with IL-13 with kinetics in the range of 10³ to 10⁸ M⁻¹ s⁻¹, typically 10⁴ to 10⁷ M⁻¹ s⁻¹. In one embodiment, the anti-IL13 antibody molecule binds to human IL-13 with a kₒₙ of between 5×10⁴ and 8×10⁵ M⁻¹ s⁻¹. In yet another embodiment, the anti-IL13 antibody molecule has dissociation kinetics in the range of 10⁻² to 10⁻⁶ x⁻¹, typically 10⁻² to 10⁻⁵ s⁻¹. In one embodiment, the anti-IL13 antibody molecule binds to IL-13, e.g., human IL-13, with an affinity and/or kinetics similar (e.g., within a factor 20, 10, or 5) to monoclonal antibody 13.2, MJ 2-7 or C65, or modified forms thereof, e.g., chimeric forms or humanized forms thereof. The affinity and binding kinetics of an IL-13 binding agent can be tested using, e.g., biosensor technology (BIACORE™).

In still another embodiment, the anti-IL13 antibody molecule specifically binds to an epitope, e.g., a linear or a conformational epitope, of IL-13, e.g., mammalian, e.g., human IL-13. For example, the antibody molecule binds to at least one amino acid in an epitope defined by IL-13Rα1 binding to human IL-13 or an epitope defined by IL-13Rα2 binding to human IL-13, or an epitope that overlaps with such epitopes. The anti-IL13 antibody molecule may compete with IL-13Rα1 and/or IL-13Rα2 for binding to IL-13, e.g., to human IL-13. The anti-IL13 antibody molecule may competitively inhibit binding of IL-13Rα1 and/or IL-13Rα2 to IL-13. The anti-IL13 antibody molecule may interact with an epitope on IL-13 which, when bound, sterically prevents interaction with IL-13Rα1 and/or IL-13Rα2. In embodiments, the anti-IL13 antibody molecule binds specifically to human IL-13 and competitively inhibits the binding of a second antibody to said human IL-13, wherein said second antibody is chosen from 13.2, MJ 2-7 and/or C65 (or any other anti-IL13 antibody disclosed herein) for binding to IL-13, e.g., to human IL-13. The anti-IL13 antibody molecule may competitively inhibit binding of 13.2, MJ 2-7 and/or C65 to IL-13. The anti-IL13 antibody molecule may specifically bind at least one amino acid in an epitope defined by 13.2, MJ 2-7 binding to human IL-13 or an epitope defined by C65 binding to human IL-13. In one embodiment, the anti-IL13 antibody molecule may bind to an epitope that overlaps with that of 13.2, MJ 2-7 or C65, e.g., includes at least one, two, three, or four amino acids in common, or an epitope that, when bound, sterically prevents interaction with 13.2, MJ 2-7 or C65. For example, the antibody molecule may contact one or more residues from IL-13 chosen from one or more of residues 81-93 and/or 114-132 of human IL-13 (SEQ ID NO: 194), or chosen from one or more of: Glutamate at position 68 [49], Asparagine at position 72 [53], Glycine at position 88 [69], Proline at position 91 [72], Histidine at position 92 [73], Lysine at position 93 [74], and/or Arginine at position 105 [86] of SEQ ID NO:194 [position in mature sequence; SEQ ID NO: 195]. In other embodiments, the antibody molecule contacts one or more amino acid residues from IL-13 chosen from one or more of residues 116, 117, 118, 122, 123, 124, 125, 126, 127, and/or 128 of SEQ ID NO:24 or SEQ ID NO: 178. In one embodiment, the antibody molecule binds to IL-13 irrespective of the polymorphism present at position 130 in SEQ ID NO:24.

In one embodiment, the antibody molecule includes one, two, three, four, five or all six CDR's from mAb13.2, MJ2-7, C65, or other antibodies disclosed herein, or closely related CDRs, e.g., CDRs which are identical or which have at least one amino acid alteration, but not more than two, three or four alterations (e.g., substitutions (e.g., conservative substitutions), deletions, or insertions). Optionally, the antibody molecule may include any CDR described herein. In embodiments, the heavy chain immunoglobulin variable domain comprises a heavy chain CDR3 that differs by fewer than 3 amino acid substitutions from a heavy chain CDR3 of monoclonal antibody MJ2-7 (SEQ ID NO:17), mAb13.2 (SEQ ID NO:196) or C65 (SEQ ID NO:123). In other embodiments, the light chain immunoglobulin variable domain comprises a light chain CDR1 that differs by fewer than 3 amino acid substitutions from a corresponding light chain CDR of monoclonal antibody MJ2-7 (SEQ ID NO:18), mAb13.2 (SEQ ID NO: 197) or C65 (SEQ ID NO:118). The amino acid sequence of the heavy chan variable domain of MJ2-7 has the amino acid sequence shown as SEQ ID NO:130. The amino acid sequence of the light chan variable domain of MJ2-7 has the amino acid sequence shown as SEQ ID NO: 133. The amino acid sequence of the heavy chan variable domain of monoclonal antibody 13.2 has the amino acid sequence shown as SEQ ID NO:198. The amino acid sequence of the light chan variable domain of monoclonal antibody 13.2 has the amino acid sequence shown as SEQ ID NO:199.

In certain embodiments, the heavy chain variable domain of the antibody molecule includes one or more of:

(SEQ ID NO:48)

G-(YF)-(NT)-I-K-D-T-Y-(MI)-H,
in CDR1,

(SEQ ID NO:49)

(WR)-I-D-P-(GA)-N-D-N-I-K-Y-(SD)-(PQ)-K-F-Q-G,
in CDR2, and/or

(SEQ ID NO:17)

SEENWYDFFDY,
in CDR3; or

(SEQ ID NO:15)

GFNIKDTYIH,
in CDR1,

(SEQ ID NO:16)

RIDPANDNIKYDPKFQG,
in CDR2, and/or

(SEQ ID NO:17)

SEENWYDFFDY,
in CDR3

In other embodiments, the light chain variable domain of the antibody molecule includes one or more of:

(SEQ ID NO:25)

(RK)-S-S-Q-S-(LI)-(KV)-H-S-(ND)-G-N-(TN)-Y-L-(EDNQ
YAS),
in CDR1,

(SEQ ID NO:27)

K-(LVI)-S-(NY)-(RW)-(FD)-S,
in CDR2, and/or

(SEQ ID NO:28)

Q-(GSA)-(ST)-(HEQ)-I-P,
in CDR3; or

(SEQ ID NO:18)

RSSQSIVHSNGNTYLE,
in CDR1

(SEQ ID NO:19)

KVSNRFS,
in CDR2, and

(SEQ ID NO:20)

FQGSHIPYT,
in CDR3.

In other embodiments, the antibody molecule includes one or more CDRs including an amino acid sequence selected from the group consisting of the amino acid sequence of SEQ ID NO: 197, SEQ ID NO:200, SEQ ID NO:201, SEQ ID NO:202, SEQ ID NO:203, and SEQ ID NO:196.

In yet another embodiment, the antibody molecule includes at least one, two, or three Chothia hypervariable loops from a heavy chain variable region of an antibody chosen from, e.g., mAb13.2, MJ2-7, C65, or any other antibody disclosed herein, or at least particularly the amino acids from those hypervariable loops that contact IL-13. In yet another embodiment, the antibody or fragment thereof includes at least one, two, or three hypervariable loops from a light chain variable region of an antibody chosen from, e.g., mAb13.2, MJ2-7, C65, or other antibodies disclosed herein, or at least includes the amino acids from those hypervariable loops that contact IL-13. In yet another embodiment, the antibody or fragment thereof includes at least one, two, three, four, five, or six hypervariable loops from the heavy and light chain variable regions of an antibody chosen from, e.g., mAb13.2, MJ2-7, C65, or other antibodies disclosed herein.

In one embodiment, the protein includes all six hypervariable loops from mAb13.2, MJ2-7, C65, or other antibodies disclosed herein or closely related hypervariable loops, e.g., hypervariable loops which are identical or which have at least one amino acid alteration, but not more than two, three or four alterations, from the sequences disclosed herein. Optionally, the protein may include any hypervariable loop described herein.

In still another example, the protein includes at least one, two, or three hypervariable loops that have the same canonical structures as the corresponding hypervariable loop of mAb13.2, MJ2-7, C65, or other antibodies disclosed herein, e.g., the same canonical structures as at least loop 1 and/or loop 2 of the heavy and/or light chain variable domains of mAb13.2, MJ2-7, C65, or other antibodies disclosed herein. See, e.g., Chothia et al. (1992) J. Mol. Biol. 227:799-817; Tomlinson et al. (1992) J. Mol. Biol. 227:776-798 for descriptions of hypervariable loop canonical structures. These structures can be determined by inspection of the tables described in these references.

In one embodiment, the heavy chain framework of the antibody molecule (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the heavy chain framework of one of the following germline V segment sequences: DP-25, DP-1, DP-12, DP-9, DP-7, DP-31, DP-32, DP-33, DP-58, or DP-54, or another V gene which is compatible with the canonical structure class 1-3 (see, e.g., Chothia et al. (1992) J. Mol. Biol. 227:799-817; Tomlinson et al. (1992) J. Mol. Biol. 227:776-798). Other frameworks compatible with the canonical structure class 1-3 include frameworks with the one or more of the following residues according to Kabat numbering: Ala, Gly, Thr, or Val at position 26; Gly at position 26; Tyr, Phe, or Gly at position 27; Phe, Val, Ile, or Leu at position 29; Met, Ile, Leu, Val, Thr, Trp, or Ile at position 34; Arg, Thr, Ala, Lys at position 94; Gly, Ser, Asn, or Asp at position 54; and Arg at position 71.

In one embodiment, the light chain framework of the antibody molecule (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the light chain framework of a Vκ II subgroup germline sequence or one of the following germline V segment sequences: A17, A1, A18, A2, A19/A3, or A23 or another V gene which is compatible with the canonical structure class 4-1 (see, e.g., Tomlinson et al. (1995) EMBO J. 14:4628). Other frameworks compatible with the canonical structure class 4-1 include frameworks with the one or more of the following residues according to Kabat numbering: Val or Leu or Ile at position 2; Ser or Pro at position 25; Ile or Leu at position 29; Gly at position 31d; Phe or Leu at position 33; and Phe at position 71.

In another embodiment, the light chain framework of the antibody molecule (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the light chain framework of a Vκ I subgroup germline sequence, e.g., a DPK9 sequence.

In another embodiment, the heavy chain framework of the antibody molecule (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the light chain framework of a VH I subgroup germline sequence, e.g., a DP-25 sequence or a VH III subgroup germline sequence, e.g., a DP-54 sequence.

In certain embodiments, the heavy chain immunoglobulin variable domain of the antibody molecule includes an amino acid sequence encoded by a nucleotide sequence that hybridizes under high stringency conditions to the complement of the nucleotide sequence encoding a heavy chain variable domain of V2.1 (SEQ ID NO:71), V2.3 (SEQ ID NO:73), V2.4 (SEQ ID NO:74), V2.5 (SEQ ID NO:75), V2.6 (SEQ ID NO:76), V2.7 (SEQ ID NO:77), V2.11 (SEQ ID NO:80), ch13.2 (SEQ ID NO:204), h13.2v1 (SEQ ID NO:205), h13.2v2 (SEQ ID NO:206) or h13.2v3 (SEQ ID NO:207); or includes an amino acid sequence that is at least 80%, 85%, 90%, 95%, 97%, 98%, 99% or higher identical identical to the amino acid sequence of the heavy chain variable domain of V2.1 (SEQ ID NO:71), V2.3 (SEQ ID NO:73), V2.4 (SEQ ID NO:74), V2.5 (SEQ ID NO:75), V2.6 (SEQ ID NO:76), V2.7 (SEQ ID NO:77), V2.11 (SEQ ID NO:80); ch13.2 (SEQ ID NO:208), h13.2v1 (SEQ ID NO:209), h13.2v2 (SEQ ID NO:210) or h13.2v3 (SEQ ID NO:211). In embodiments, the heavy chain immunoglobulin variable domain includes the amino acid sequence of SEQ ID NO:80, which may in turn further include a heavy chain variable domain framework region 4 (FR4) that includes the amino acid sequence of SEQ ID NO:116 or SEQ ID NO:117.

In other embodiments, the light chain immunoglobulin variable domain of the antibody molecule includes an amino acid sequence encoded by a nucleotide sequence that hybridizes under high stringency conditions to the complement of the nucleotide sequence encoding a light chain variable domain of V2.11 (SEQ ID NO:36) or h13.2v2 (SEQ ID NO:212); or includes an amino acid sequence that is at least 80%, 85%, 90%, 95%, 97%, 98%, 99% or higher identical identical to a light chain variable domain of V2.11 (SEQ ID NO:36) or h13.2v2 (SEQ ID NO:212). In embodiments, the light chain immunoglobulin variable domain includes the amino acid sequence of SEQ ID NO:36, which may in turn further include a light chain variable domain framework region 4 (FR4) that includes the amino acid sequence of SEQ ID NO:47.

In yet another embodiment, the antibody molecule includes a framework of the heavy chain variable domain sequence comprising:
(i) at a position corresponding to 49, Gly;
(ii) at a position corresponding to 72, Ala;
(iii) at positions corresponding to 48, Ile, and to 49, Gly;
(iv) at positions corresponding to 48, Ile, to 49, Gly, and to 72, Ala;
(v) at positions corresponding to 67, Lys, to 68, Ala, and to 72, Ala; and/or
(vi) at positions corresponding to 48, Ile, to 49, Gly, to 72, Ala, to 79, Ala.

In one embodiment, the anti-IL13 antibody molecule includes at least one light chain that comprises the amino acid sequence of SEQ ID NO:177 (or an amino acid sequence at least 80%, 85%, 90%, 95%, 97%, 98%, 99% or higher identical identical to SEQ ID NO: 177) and at least one heavy chain that comprises the amino acid sequence of SEQ ID NO:176 (or an amino acid sequence at least 80%, 85%, 90%, 95%, 97%, 98%, 99% or higher identical identical to SEQ ID NO: 176).

In one embodiment, the anti-IL13 antibody molecule includes two immunoglobulin chains: a light chain that includes SEQ ID NO:199, 213, 214, 212, or 215 and a heavy chain that includes SEQ ID NO:198, 208, 209, 210, or 211 (or an amino acid sequence at least 80%, 85%, 90%, 95%, 97%, 98%, 99% or higher identical identical to SEQ ID NO:199, 213, 214, 212, or 215, or SEQ ID NO:198, 208, 209, 210, or 211). The antibody molecule may further include in the heavy chain the amino acid sequence of SEQ ID NO:193 and in the light chain the amino acid sequence of SEQ ID NO:216 (or an amino acid sequence at least 80%, 85%, 90%, 95%, 97%, 98%, 99% or higher identical identical to SEQ ID NO:193 or SEQ ID NO:216).

Additional examples of anti-IL13 antibody molecules are disclosed in U.S. Ser. No. 07/012,8192 or WO 05/007699 and in Blanchard, C. et al. (2005) Clinical and Experimental Allergy 35(8):1096-1103 disclosing CAT-354; WO 05/062967, WO 05/062972 and Clinical Trials Gov. Identifier: NCT00441818 disclosing TNX-650; Clinical Trials Gov. Identifier: NCT532233 disclosing QAX-576; U.S. Ser. No. 06/014,0948 or WO 06/055638, filed in the name of Abgenix; U.S. Pat. No. 6,468,528 assigned to AMGEN; WO 05/091856 naming Centocor, Inc. as the applicant; and in Yang et al. (2004) Cytokine 28(6):224-32 and Yang et al. (2005) J Pharmacol Exp Ther: 313(1):8-15; and anti-IL13 antibodies as disclosed in WO 07/080,174 filed in the name of Glaxo, and as disclosed in WO 07/045,477 in the name of Novartis.

Additional examples of IL-13 or IL-4 antagonists include, but are not limited to, antibody molecules against IL-4 (e.g., pascolizumab and related antibodies disclosed in Hart, T. K. et al. (2002) Clin Exp Immunol. 130(1):93-100; Steinke, J. W. (2004) Immunol. Allergy Clin North Am 24(4):599-614; and in Ramanthan et al. U.S. Pat. No. 6,358,509), IL-4Rα (e.g., AMG-317 and related anti-ILAR antibodies disclosed in U.S. Ser. No. 05/011,8176, U.S. Ser. No. 05/011,2694 and in Clinical Trials Gov. Identifier: NCT00436670); IL-13Rα1 (e.g., anti-13Rα1 antibodies disclosed in WO 03/080675 which names AMRAD as the applicant); and mono- or bi-specific antibody molecules that bind to IL4 and/or IL-13 (disclosed, e.g., in WO 07/085,815).

In other embodiments, the IL-13 or IL-4 antagonist is an IL-13 or IL-4 mutein (e.g., a truncated or variant form of the cytokine that binds to the an IL-13R or an IL-4 receptor, but does not significantly increase the activity of the receptor), or a cytokine-conjugated to a toxin. IL-4 muteins are disclosed by Weinzel et al. (2007) Lancet 370:1422-31. Additional examples of IL-13/IL-4 inhibiting peptides are disclosed in Andrews, A. L. et al. (2006) J. Allergy and Clin Immunol 118:858-865. An example of a cytokine-toxin conjugate is disclosed in WO 03/047632, in Kunwar, S. et al. (2007) J. Clin Oncol 25(7):837-44 and in Husain, S. R. et al. (2003) J. Neurooncol 65(1):37-48.

In yet other embodiments, the IL13 antagonist or the IL-4 antagonist is a full length, or a fragment or modified form of an IL-13 receptor polypeptide (e.g., IL-13Rα2 or IL13Rα1) or an IL-4 receptor polypeptide (e.g., IL-4Rα). For example, the antagonist can be a soluble form of an IL-13 receptor or an IL-14 receptor (e.g., a soluble form of mammalian (e.g., human) IL-13Rα2, IL13Rα1 or IL-4Rα comprising a cytokine-binding domain; e.g., a soluble form of an extracellular domain of mammalian (e.g., human) IL-13Rα2, IL13Rα1 or IL-4Rα). Exemplary receptor antagonists include, e.g., IL-4R-IL-13R binding fusions as described in WO 05/085284 and Economides, A. N. et al. (2003) Nat Med 9(1):47-52, as well as in Borish, L. C. et al. (1999) Am J Respir Crit. Care Med 160(6):1816-23.

A soluble form of an IL-13 receptor or IL-4 receptor, or an IL-13 or IL-4 mutein can be used alone or functionally linked (e.g., by chemical coupling, genetic or polypeptide fusion, non-covalent association or otherwise) to a second moiety to facilitate expression, steric flexibility, detection and/or isolation or purification, e.g., an immunoglobulin Fc domain, serum albumin, pegylation, a GST, Lex-A or an MBP polypeptide sequence. The fusion proteins may additionally include a linker sequence joining the first moiety to the second moiety. For example, a soluble IL-13 receptor or IL-4 receptor, or an IL-13 or IL-4 mutein can be fused to a heavy chain constant region of the various isotypes, including: IgG1, IgG2, IgG3, IgG4, IgM, IgA1, IgA2, IgD, and IgE). Typically, the fusion protein can include the extracellular domain of a human soluble IL-13 receptor or IL-4 receptor, or an IL-13 or IL-4 mutein (or a sequence homologous thereto), and, e.g., fused to, a human immunoglobulin Fc chain, e.g., human IgG (e.g., human IgG1 or human IgG2, or a mutated form thereof). The Fc sequence can be mutated at one or more amino acids to reduce effector cell function, Fc receptor binding and/or complement activity.

It will be understood that the antibody molecules and soluble or fusion proteins described herein can be functionally linked (e.g., by chemical coupling, genetic fusion, non-covalent association or otherwise) to one or more other molecular entities, such as an antibody (e.g., a bispecific or a multispecific antibody), toxins, radioisotopes, cytotoxic or cytostatic agents.

In another embodiment, the IL-13 or IL-4 antagonist inhibits the expression of nucleic acid encoding an IL-13 or IL-13R, or an IL-4 or IL-4R. Examples of such antagonists include nucleic acid molecules, for example, antisense molecules, ribozymes, RNAi, siRNA, triple helix molecules that hybridize to a nucleic acid encoding an IL-13 or IL-13R, or an IL-4 or IL-4R, or a transcription regulatory region, and blocks or reduces mRNA expression of IL-13 or IL-13R, or an IL-4 or IL-4R. ISIS-369645 provides an example of an antisense nucleic acid that inhibits expression of IL-4Rα (developed by ISIS Pharmaceuticals and disclosed in, e.g., Karras, J. G. et al. (2007) Am J Respir Cell Mol Biol. 36(3):276-86). Exemplary short interference RNAs (siRNAs) that interfere with RNA encoding IL-4 or IL-13 are disclosed in WO 07/131,274.

In yet another embodiment, the IL-13 or IL-4 antagonist is an inhibitor, e.g., a small molecule inhibitor, of upstream or downstream IL-13 signalling (e.g., STAT6 inhibitors). Examples of STAT6 inhibitors are disclosed in WO 04/002964, in Canadian Patent Application: CA 2490888 and in Nagashima, S. et al. (2007) Bioorg Med Chem 15(2):1044-55; and in U.S. Pat. No. 6,207,391 and WO 01/083517.

In another embodiment, one or more IL-13 antagonists are administered in combination with one or more IL-4 antagonists. The combination therapy can include the IL-13 antagonist formulated with and/or administered with the IL-4 antagonist. The IL-13 antagonist and the IL-4 antagonist can be administered simultaneously, or sequentially. If administered sequentially, a physician can select an appropriate sequence for administering the IL-13 antagonist in combination with the IL-4 antagonist. The combination therapy can also include other therapeutic agents chosen from one or more of: inhaled steroids; beta-agonists, e.g., short-acting or long-acting beta-agonists; antagonists of leukotrienes or leukotriene receptors; combination drugs such as ADVAIR®; IgE inhibitors, e.g., anti-IgE antibodies (e.g., XOLAIR®); phosphodiesterase inhibitors (e.g., PDE4 inhibitors); xanthines; anticholinergic drugs; mast cell-stabilizing agents such as cromolyn; IL-5 inhibitors; eotaxin/CCR3 inhibitors; and antihistamines. Such combinations can be used to treat asthma and other respiratory disorders. Additional examples of therapeutic agents that can be coadministered and/or coformulated with an IL-13 binding agent include one or more of: TNF antagonists (e.g., a soluble fragment of a TNF receptor, e.g., p55 or p75 human TNF receptor or derivatives thereof, e.g., 75 kd TNFR-IgG (75 kD TNF receptor-IgG fusion protein, ENBREL™)); TNF enzyme antagonists, e.g., TNFα converting enzyme (TACE) inhibitors; muscarinic receptor antagonists; TGF-β antagonists; interferon gamma; perfenidone; chemotherapeutic agents, e.g., methotrexate, leflunomide, or a sirolimus (rapamycin) or an analog thereof, e.g., CCI-779; COX2 and cPLA2 inhibitors; NSAIDs; immunomodulators; p38 inhibitors, TPL-2, Mk-2 and NFκB inhibitors, among others. In another aspect, the application provides a method of evaluating the efficacy of an IL-13 antagonistic binding agent, e.g., an anti-IL13 antibody molecule as described herein, in treating (e.g., reducing) pulmonary inflammation in a subject, e.g., a human or non-human subject.

In yet another embodiment, the methods disclosed herein further include the step(s) of:

evaluating (e.g., detecting) a change in one or more of the following parameters in a subject after administration of the IL-13 antagonist and/or IL-4 antagonists: (i) detecting the levels of IL-13 unbound and/or bound to an IL13 binding agent in a sample, e.g., a biological sample (e.g., serum, plasma, blood) as described in the in vitro detection methods herein; (ii) measuring eotaxin levels in a sample, e.g., a biological sample (e.g., serum, plasma, blood); (iii) detecting histamine release by basophils; (iv) detecting IgE-titers; and/or (v) evaluating changes in the symptoms of the subject (e.g., difficulty breathing, wheezing, coughing, shortness of breath and/or difficulty performing normal daily activities). In embodiments, the detection of parameters (i)-(v) can be carried out before and/or after administration of the IL-13 antagonistic binding agent (after single or multiple administrations) to the subject (e.g., at selected intervals after initiating therapy). The detection and/or evaluation of the changes in one or more of (i)-(v) can be performed by a clinician or support staff. A change, e.g., a reduction, in one or more of (i)-(v) relative to a predetermined level (e.g., comparing before and after treatment) indicates that the IL-13 antagonistic binding agent is effectively reducing lung inflammation in the subjects. In embodiments, the subject is a human patient, e.g., an adult or a child.

In another aspect, the invention provides compositions, e.g., pharmaceutical compositions, or dose formulations that include a pharmaceutically acceptable carrier and at least one IL-13 antagonistic binding agent, e.g., an anti-IL-13 antibody molecule, formulated with an IL-4 antagonist. Combinations of the aforesaid antagonists and another drug, e.g., a therapeutic agent (e.g., one or more cytokine and growth factor inhibitors, immunosuppressants, anti-inflammatory agents (e.g., systemic anti-inflammatory agents), metabolic inhibitors, enzyme inhibitors, and/or cytotoxic or cytostatic agents, as described herein, can also be used.

In yet another aspect, the invention features a kit that includes an IL-13 antagonist and/or an IL-4 antagonist for use in the methods disclosed herein with instructions for administering the antagonist as a single treatment interval to treat or prevent an IL-13 associated disorder or condition (e.g., a disorder or condition as described herein).

In another aspect, the invention features a composition that includes an IL-13 antagonist and/or an IL-4 antagonist for use in the methods disclosed herein.

In yet another aspect, the invention features the use of a composition that includes an IL-13 antagonist and/or an IL-4 antagonist in the manufacture of a medicament to treat or prevent an IL-13-associated disorder or condition (e.g., a disorder or condition as described herein).

In another aspect, this application provides a method for detecting the presence of IL-13 in a sample in vitro (e.g., a biological sample, such as serum, plasma, tissue, biopsy). The subject method can be used to diagnose a disorder, e.g., an IL-13-associated disorder, or to monitor the efficacy of a treatment. The method includes: (i) contacting the sample with an IL-13 binding agent, e.g., a first IL-13 binding agent or anti-IL13 antibody molecule as described herein; and (ii) detecting the formation of a complex between the first IL-13 binding agent and IL-13 (e.g., substantially free IL-13 and/or IL-13-bound to a second anti-IL-13 binding agent or antibody molecule), in the sample. A statistically significant change in the level of IL-13 bound to the first anti-IL-13 binding agent or antibody molecule in the sample relative to a reference value or sample (e.g., a control sample) is indicative of the presence of the IL-13 in the sample.

In certain embodiments, the first anti-IL-13 binding agent or antibody molecule is immobilized to a support (e.g., a solid support, such as an ELISA plate, beads).

In other embodiments, the method further includes obtaining a sample from a subject before and/or after exposure of the subject to a second anti-IL-13 binding agent or antibody molecule. The sample can contain substantially free IL-13 and/or IL-13 bound to the second anti-IL-13 binding agent or antibody molecule. The sample is allowed to contact the immobilized first anti-IL-13 binding agent or antibody molecule, under conditions that allow binding of the IL-13 to the immobilized first anti-IL-13 binding agent or antibody molecule to occur.

In embodiments, the detection step includes detecting the presence of IL-13 (e.g., substantially free IL-13 and/or IL-13-bound to a second anti-IL-13 binding agent or antibody molecule) bound to the immobilized first anti-IL-13 binding agent or antibody molecule, e.g., using a labeled third anti-IL-13 binding agent or antibody molecule, or a labeled agent that recognizes the complex of IL-13 first or second binding agent or antibody molecule. The label can be directly or indirectly attached to the anti-IL-13 binding agent or antibody molecule, e.g., fluorescence, radioactivity, biotin-avidin, as described herein. For example, the anti-IL13 binding agent or antibody molecule is directly or indirectly labeled with a detectable substance to facilitate detection of the bound or unbound antibody. Suitable detectable substances include various enzymes, prosthetic groups, fluorescent materials, luminescent materials and radioactive materials.

In one embodiment, the first anti-IL-13 binding agent or antibody molecule binds to substantially free IL-13, and does not substantially bind to IL-13 bound to a second anti-IL-13 binding agent or antibody molecule. In other embodiments, the first anti-IL-13 binding agent or antibody molecule binds to substantially free IL-13 and IL-13 bound to a second anti-IL-13 binding agent or antibody molecule.

In another embodiment, the first, second and/or third anti-IL-13 binding agents or antibody molecules bind to different epitopes on IL-13. For example, the first anti-IL-13 antibody molecule is a mAb13.2 or a humanized version thereof (disclosed herein and in U.S. Ser. No. 06/006,3228), or an IL-13 binding agent capable of competing with mAb13.2 for binding to IL-13; the second anti-IL-13 antibody molecule is an MJ2-7 or a humanized version thereof; and/or the third anti-IL-13 antibody molecule is a C65 antibody or a humanized version thereof (disclosed herein and in U.S. Ser. No. 06/007,3148) (or an IL-13 binding agent capable of competing with mJ2-7 or C65 for binding to IL-13). Any order of anti-IL13 antibody molecules can be used in the detection methods.

In embodiment, the complex of IL-13 bound to the second IL-13 binding agent, which is immobilized to the first IL-3 binding agent, is detected by contacting the immobilized complex with an Fc binding agent (e.g., an anti-Fc antibody molecule), thereby determining the amount of IL-13 bound to the second IL-13 binding agent in a sample.

In embodiments, an increase in the level of IL-13 in the sample (e.g., a biological sample, such as serum, plasma, tissue, biopsy) of the subject relative to a predetermined level is indicative of increased inflammation in the lung.

In yet another aspect, the invention provides a method for detecting the presence of IL-13 in vivo (e.g., in vivo imaging in a subject). The subject method can be used to diagnose a disorder, e.g., an IL-13-associated disorder, or to measure the efficacy of a treatment. The method includes: (i) administering a first IL-13 binding agent, e.g., a first anti-IL-13 antibody molecule as described herein, to a subject under conditions that allow binding of the first IL-13 binding agent to IL-13 to occur; and (ii) detecting IL-13 in vivo (e.g., detecting the formation of a complex between IL-13 and the first IL-13 binding agent) using a second IL-13 binding agent detectably labeled, wherein a statistically significant change in the level of IL-13 in the subject relative to the control subject is indicative of the presence of IL-13. In embodiments, an increase in the level of IL-13 in the subject relative to a predetermined level is indicative of increased inflammation in the lung.

In one embodiment, the IL-13 binding agent and the IL-13 antagonist bind to substantially free IL-13 and/or IL-13 bound to a second IL-13 binding agent. In one embodiment, the IL-13 antagonist and the IL-13 binding agent recognize different epitopes on IL-13. For example, the IL-13 antagonist can be a mAb13.2 or a humanized version thereof (disclosed herein and in U.S. Ser. No. 06/006,3228), or an IL-13 antagonist capable of competing with mAb13.2 for binding to IL-13; the IL-13 binding agent is an MJ2-7 or a humanized version thereof; or the binding agent is a C65 antibody or a humanized version thereof (disclosed herein and in U.S. Ser. No. 06/007,3148) (or an IL-13 binding agent capable of competing with mJ2-7 or C65 for binding to IL-13). Any order of anti-IL13 antagonist or binding agents can be used in the detection methods.

In another aspect, the application provides a method of evaluating the efficacy of an IL-13 antagonistic binding agent, e.g., an anti-IL13 antibody molecule as described herein, in treating (e.g., reducing) pulmonary inflammation in a subject, e.g., a human or non-human subject. The method includes:

administering an IL-13 antagonist and/or an IL-4 antagonist to the subject;

detecting a change in one or more of the following parameters: (i) detecting the levels of IL-13 unbound and/or bound to an IL13 binding agent in a sample, e.g., a biological sample (e.g., serum, plasma, blood) as described in the in vitro detection methods herein, wherein a change in the levels of IL-13 unbound and/or bound relative to a reference value (e.g., a control sample) is indicative of the efficacy of the agent.

In embodiments, the method further includes: (i) measuring eotaxin levels in a sample, e.g., a biological sample (e.g., serum, plasma, blood); (ii) detecting histamine release, e.g., by basophils; (iii) detecting IgE-titers; and/or (iv) evaluating changes in the symptoms of the subject (e.g., difficulty breathing, wheezing, coughing, shortness of breath and/or difficulty performing normal daily activities). The detection of parameters (i)-(v) can be carried out before and/or after administration of the IL-13 antagonistic binding agent (after single or multiple administrations) to the subject (e.g., at selected intervals after initiating therapy). The detection and/or evaluation of the changes in one or more of (i)-(v) can be performed by a clinician or support staff. A change, e.g., a reduction, in one or more of (i)-(v) relative to a predetermined level (e.g., comparing before and after treatment) indicates that the IL-13 antagonistic binding agent is effectively reducing lung inflammation in the subjects. In embodiments, the subject is a human patient, e.g., an adult or a child.

In embodiments, the efficacy of an IL-13 binding agent (e.g., an anti-IL13 antibody molecule as described) in neutralizing one or more IL-13-associated activities in vivo can be evaluated in a subject, e.g., a non-human subject, such as sheep, rodent, non-human primate (e.g., a cynomolgus monkey naturally allergic to an antigen, e.g., Ascaris suum). For example, the efficacy of IL-13 binding agents can be evaluated by measuring in cynomolgus monkeys naturally allergic to Ascaris suum, before and after challenge with the Ascaris antigen in the presence or absence of the IL-13 binding agent, one or more of the following: (i) detecting inflammatory cells (e.g., eosinophils, macrophages, neutrophils) into the airways; (ii) measuring eotaxin levels; (iii) detecting in antigen-specific (e.g., Ascaris-specific) basophil histamine release; and/or (iv) detecting in antigen-specific (e.g., Ascaris-specific) IgE titers. A change, e.g., a reduction, in the level of one or more of (i)-(iv) relative to a predetermined level (e.g., comparison before and after treatment) indicates that the IL-13 binding agent is effectively reducing airway eosinophilia in the subjects.

Methods of diagnosing an IL-13-associated disorder using an IL-13 binding agent, e.g., an anti-IL13 antibody molecule as described herein are also disclosed.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The terms “proteins” and “polypeptides” are used interchangeably herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The contents of all publications, pending patent applications, published patent applications (inclusive of U.S. Ser. No. 06/007,3148 and U.S. Ser. No. 06/006,3228), and published patents cited throughout this application are hereby incorporated by reference in their entirety.

Others features, objects and advantages of the invention will be apparent from the description and drawings, and from the claims.

### BRIEF DESCRIPTION OF THE DRAWINGS

FIG. 1A is an alignment of full-length human and cynomolgus monkey IL-13, SEQ ID NO:178 and SEQ ID NO:24, respectively. Amino acid differences are indicated by the shaded boxed residues. The location of the R to Q substitution (which corresponds to the polymorphism detected in allergic patients) is boxed at position 130. The location of the cleavage site is shown by the arrow.

FIG. 1B is a list of exemplary peptides from cynomolgus monkey IL-13, (SEQ ID NOs:179-188, respectively).

FIG. 2 is a graph depicting the neutralization of NHP IL-13 activity by various IL-13 binding agents, as measured by percentage of CD23⁺ monocytes (y-axis). Concentration of MJ2-7 (Δ), C65 (♦), and sIL-13Rα2-Fc () are indicated on the x-axis.

FIG. 3 is a graph depicting the neutralization of NHP IL-13 activity by MJ2-7 (murine; ) or humanized MJ2-7 v2.11 (∘). NHP IL-13 activity was measured by phosphorylation of STAT6 (y-axis) as a function of antibody concentration (x-axis).

FIG. 4 is a graph depicting the neutralization of NHP IL-13 activity by MJ2-7 v2.11 (∘) or sIL-13Rα2-Fc (▴). NHP IL-13 activity was measured by phosphorylation of STAT6 (y-axis) as a function of antagonist concentration (x-axis).

FIG. 5 is a graph depicting the neutralization of NHP IL-13 activity by MJ2-7 (Δ), C65 (♦), or sIL-13Rα2-Fc (). NHP IL-13 activity was measured by phosphorylation of STAT6 (y-axis) as a function of antagonist concentration (x-axis).

FIG. 6A is a graph depicting induction of tenascin production (y-axis) by native human IL-13 (x-axis).

FIG. 6B is a graph depicting the neutralization of NHP IL-13 activity by MJ2-7, as measured by inhibition of induction of tenascin production (y-axis) as a function of antibody concentration (x-axis).

FIG. 7 is a graph depicting binding of MJ2-7 or control antibodies to NHP-IL-13 bound to sIL-13Rα2-Fc coupled to a SPR chip.

FIG. 8 is a graph depicting binding of varying concentrations (0.09-600 nM) of NHP IL-13 to captured hMJ2-7 V2-11 antibody.

FIG. 9 is a graph depicting the neutralization of NHP IL-13 activity by mouse MJ2-7 () or humanized Version 1 (∘), Version 2 (♦), or Version 3 (Δ) antibodies. NHP IL-13 activity was measured by phosphorylation of STAT6 (y-axis) as a function of antibody concentration (x-axis).

FIG. 10 is a graph depicting the neutralization of NHP IL-13 activity by antibodies including mouse MJ2-7 VH and VL (), mouse VH and humanized Version 2 VL (Δ), or Version 2 VH and VL (♦). NHP IL-13 activity was measured by phosphorylation of STAT6 (y-axis) as a function of antibody concentration (x-axis).

FIGS. 11A and 11B are graphs depicting inhibition of binding of IL-13 to immobilized IL-13 receptor by MJ2-7 antibody, as measured by ELISA. Binding is depicted as absorbance at 450 nm (y-axis). Concentration of MJ2-7 antibody is depicted on the x-axis. FIG. 11A depicts binding to IL-13Rα1. FIG. 11B depicts binding to IL-13Rα2.

FIG. 12 is an alignment of DPK18 germline amino acid sequence (SEQ ID NO:126) and humanized MJ2-7 Version 3 VL (SEQ ID NO:190).

FIG. 13A is an amino acid sequence (SEQ ID NO:124) of mature, processed human IL-13.

FIG. 13B shows an amino acid sequence (SEQ ID NO:125) of human IL-13Rα1.

FIG. 14A-14D shows an increase in the total number of cells/ml and percentage of inflammatory cells present in BAL fluid post-Ascaris challenge compared to pre-(baseline) samples.

FIGS. 15A-15B show total of BAL cells/ml in BAL fluids in control and antibody-treated cynomolgus monkeys pre- and post-Ascaris challenge. Control (circles (∘); MJ2-7-treated samples (open triangles (A)) and mAb 13.2-treated samples (black triangles (▴)). (Humanized versions of MJ2-7 (MJ2-7v.2) and mAb 13.2 v 2 were used in this study).

FIGS. 16A-16B show changes in eotaxin levels in concentrated BAL fluid collected from antibody-treated cynomolgus monkeys post-Ascaris challenge relative to control. FIG. 16A depicts a bar graph showing an increase in eotaxin levels (pg/ml) post-Ascaris challenge relative to a baseline, pre-challenge values. FIG. 16B depicts a decrease in eotaxin levels in concentrated BAL fluids from cynomolgus monkeys treated with mAb 13.2—(grey circles) or MJ2-7—(grey triangles) antibodies compared to a control. (Humanized versions of MJ2-7 (MJ2-7v.2) and mAb 13.2 v2 were used in this study).

FIGS. 17A-17B depict the changes in Ascaris-specific IgE-titers in control and antibody-treated samples 8-weeks post-challenge. FIG. 17A depicts representative examples showing no change in Ascaris-specific IgE titer in an individual monkey treated with irrelevant Ig (IVIG; animal 20-45; top panel), and decreased titer of Ascaris-specific IgE in an individual monkey treated with humanized MJ2-7v.2 (animal 120-434; bottom panel). FIG. 17B depicts a decrease in Ascaris-specific IgE-titers in mAb13.2 or MJ2-7 (black circles) relative to irrelevant Ig-treated cynomolgus monkeys (IVIG (grey circles)) 8-weeks post-Ascaris challenge.

FIGS. 18A-18B show the changes in Ascaris-specific basophil histamine release in control and antibody-treated samples 24-hours and 8-weeks post-challenge. FIG. 15A is a graph depicting the following samples in representative individual monkeys treated with saline (left) or humanized mAb13.2v.2 (right): pre-antibody or Ascaris challenged samples (circles); 48-hours post-antibody treatment, 24-hours post-Ascaris challenged samples (triangles); and 8 weeks post-Ascaris challenged samples (diamonds). FIG. 18B depicts a bar graph showing the changes in normalized histamine levels pre- and 8-week post-Ascaris challenge in control (black), humanized mAb13.2—(white) and humanized MJ2-7v.2—(shaded) treated cynomolgus monkeys.

FIG. 19 depicts the correlation between Ascaris-specific histamine release and Ascaris-specific IgE levels in control (open circles) and anti-IL13- or dexamethasone-treated samples (black circles).

FIG. 20 is a series of bar graphs depicting the changes in serum IL-13 levels in individual cynomolgus monkeys treated with humanized MJ2-7 (hMJ2-7v2). The label in each panel (e.g., 120-452) corresponds to the monkey identification number. The “pre” sample was collected prior to administration of the antibody. The time “0” was collected 24-hours post-antibody administration, but prior to Ascaris challenge. The remaining time points were post-Ascaris challenge.

FIG. 21 is a bar graph depicting the STAT6 phosphorylation activity of non-human primate IL-13 at 0, 1, or 10 ng/ml, either in the absence of serum (“no serum”); the presence of serum from saline or IVIG-treated animals (“control”); or in the presence of serum from anti-IL13 antibody-treated animals, either before antibody administration (“pre”), or 1-2 weeks post-administration of the indicated antibody. Serum was tested at 1:4 dilution. (Humanized versions of MJ2-7 (MJ2-7v.2) and mAb 13.2 v2 were used in this study).

FIGS. 22A-22C are linear graphs showing that levels of non-human primate IL-13 trapped by humanized MJ2-7 (hMJ2-7v2) in cynomolgus monkey serum correlate with the level of inflammation measured in the BAL fluids post-Ascaris challenge.

FIGS. 23A-23B are line graphs showing altered lung function in mice in response to human recombinant R110Q IL-13 intratracheal administration; FIG. 23A shows the changes in airway resistance (RI) in response to increasing doses of nebulized metacholine; FIG. 23B shows the changes in dynamic lung compliance (Cdyn) in response to increasing doses of nebulized metacholine.

FIGS. 24A-24B are bar graphs showing increased lung inflammation and cytokine production in mice in response to human recombinant R110Q IL-13 intranasal administration. In FIG. 24A, the percentage of eosinophils and neutrophils in bronchoalveolar lavage (BAL) were determined by differential cell counts. In FIG. 24B, the levels of cytokines, MCP-1, TNF-α, and IL-6, in BAL were determined by cytometric bead array. Data is median±s.e.m. of 10 animals per group.

FIGS. 25A-25B are dot plots showing humanized MJ2-7-11 (hMJ2-7v.2-11) antibody levels in BAL and serum following intratracheal and intravenous administration. Animals were treated with human recombinant R110Q IL-13, or an equivalent volume (20 μL) of saline, intratracheally on days 1, 2, and 3. Humanized MJ2-7v.2-11 antibody was administered on day 0 and 2 hours before each dose of human recombinant R110Q IL-13. FIG. 25A depicts the results when the antibody is administered intravenously on day 0 and intraperitoneally on days 1, 2, and 3; or intranasally on days 0, 1, 2, and 3 (shown in FIG. 25B). Total human IgG levels in BAL and serum were assayed by ELISA.

FIGS. 26A-26C show the effect of humanized MJ2-7v.2-11 antibody after intranasal administration of human recombinant R110Q IL-13-induced altered lung function. (A) FIG. 26A shows the changes in lung resistance (RI; cm H₂O/ml/sec) expressed as change from baseline. FIG. 26B shows data expressed as methacholine dose required to elicit lung resistance (RI) corresponding to a change of 2.5 ml H₂O/cm/sec from baseline. Median values are shown for each treatment group. p-values were calculated by two-tailed t-test. FIG. 26C shows the median human IgG levels in BAL and sera.

FIGS. 27A-27D show the changes in BAL and serum levels of human recombinant R110Q IL-13 administered alone (FIGS. 27A-27B) or in complex with humanized MJ2-7v.2-11 antibody (FIGS. 26C-27D) following intratracheal administration of human recombinant R110Q IL-13 and intranasal administration of humanized MJ2-7v.2-11 antibody. Median values are indicated for each group. n.d. is not detectable.

FIGS. 28A-28B are dot plots showing eosinophil (FIG. 28A) and neutrophil (FIG. 28B) infiltration into BAL levels following intranasal administration of human recombinant R110Q IL-13 and intranasal administration of 500, 100, and 20 μg of humanized MJ2-7v.2-11 and humanized 13.2v.2, saline, or 500 μg of IVIG. Eosinophil and neutrophil percentages were determined by differential cell counts. Median values for each group are indicated. p-values were determined by two-tailed test and are indicated for each antibody-treated group as compared to IVIG.

FIGS. 29A-29C are dot plots showing changes in chytokine levels, MCP-1, TNF-α, and IL-6, respectively, following intranasal administration of human recombinant R110Q IL-13 and intranasal administration of 500 μg of humanized MJ2-7v.2-11, humanized 13.2v.2, or IVIG, or saline. Dashed line indicates limit of assay sensitivity. Data represent median values for each group. p-value was ≦0.0001, according to a two-tailed t-test.

FIGS. 30A-30B are dot plots showing that human recombinant R110Q IL-13 levels are directly related to lung inflammation, as measured by eosinohilia; and inversely proportional to humanized MJ2-7v.2-11 BAL levels following intranasal administration of human recombinant R110Q IL-13 and intranasal administration of 500, 100, or 20 μg doses of humanized MJ2-7v.2-11 antibody. Humanized MJ2-7v.2-11 antibody BAL levels were measured by ELISA. Human recombinant R110Q IL-13 BAL levels were determined by cytometric bead assay. % eosinophil was determined by differential cell counting. Associations are shown between levels of; (FIG. 30A) % eosinophilic inflammation and human recombinant R110Q IL-13, including data from saline control animals, mice treated with human recombinant R110Q IL-13 alone, and mice treated with human recombinant R110Q IL-13 and 500, 100, and 20 μg of humanized MJ2-7v.2-11 antibody or 500 μg IVIG; and (FIG. 30B) humanized MJ2-7v.2-11 and IL-6, including data from mice treated with 500, 100, and 20 μg of humanized MJ2-7V2-11. r² and p-values were determined by linear regression analysis.

FIG. 31 shows the schedules for administrating sIL-13Ra2 one day before and one day after OVA challenge (Schedule 1), and sIL-13Ra2, anti-IL-4 or both one day before OVA challenge (Schedule 2).

FIGS. 32A-32C show total serum IgE (FIG. 32A), OVA-specific IgE (FIG. 32B), and OVA-specific IgG1 (FIG. 32C) following treatment with sILRa2.Rc one day before and after OVA challenge. The dashed line in FIG. 32B indicates the limit of assay sensitivity. n=20 mice/group

FIGS. 33A-33C depict show total serum IgE (FIG. 33A), OVA-specific IgE (FIG. 33B), and OVA-specific IgG1 (FIG. 33C) following single treatment with sILRa2.Fc one day before OVA challenge. The dashed line in FIG. 33B indicates the limit of assay sensitivity. n=20 mice/group.

FIGS. 34A-34B show total serum IgE (FIG. 34A) and OVA-specific IgE (FIG. 34B) following single treatment of sIL-13Ra2.Fc or anti-IL-4 treatment one day before OVA challenge. The dashed line in FIG. 34B indicates the limit of assay sensitivity. n 20 mice/group.

FIG. 35A-35B show OVA-specific IgG1 (FIG. 35A) and OVA-specific IgG3 (FIG. 35B) following single treatment one day prior to OVA challenge with combined sIL-13Ra2.Fc and anti-IL-4.

### DETAILED DESCRIPTION

Methods and compositions for treating and/or monitoring treatment of IL-13-associated disorders or conditions are disclosed. In one aspect, Applicants have discovered that a single administration of an IL-13 antagonist or an IL-4 antagonist to a subject, prior to the onset of an IL-13 associated disorder or condition, reduces one or more symptoms of the disorder or condition, relative to an untreated subject. Enhanced reduction of the symptoms of the disorder or condition is detected after co-administration of an IL-13 antagonist with an IL-4 antagonist, relative to the reduction detected after administration of the single agent. Thus, methods for reducing or inhibiting, or preventing or delaying the onset of, one or more symptoms of an IL-13-associated disorder or condition using an IL-13 antagonist, alone or in combination with an IL-4 antagonist, are disclosed. In other embodiments, methods for evaluating the efficacy of an IL-13 antagonist, in a subject, e.g., a human or non-human subject, are also disclosed.

### DEFINITIONS

For convenience, certain terms are defined herein. Additional definitions can be found throughout the specification.

The term “IL-13” includes the full length unprocessed form of the cytokines known in the art as IL-13 (irrespective of species origin, and including mammalian, e.g., human and non-human primate IL-13) as well as mature, processed forms thereof, as well as any fragment (of at least 5 amino acids) or variant of such cytokines. Positions within the IL-13 sequence can be designated in accordance to the numbering for the full length, unprocessed human IL-13 sequence. For an exemplary full-length monkey IL-13, see SEQ ID NO:24; for mature, processed monkey IL-13, see SEQ ID NO:14; for full-length human IL-13, see SEQ ID NO:178, and for mature, processed human IL-13, see SEQ ID NO:124. An exemplary sequence is recited as follows:

(SEQ ID NO:178)

MALLLTTVIALTCLGGFASPGPVPPSTALRELIEELVNITQNQKAPLCNG
SMVWSINLTAGMYCAALESLINVSGCSAIEKTQRMLSGFCPHKVSAGQFS
SLHVRDTKIEVAQFVKDLLLHLKKLFREGRFN

For example, position 130 is a site of a common polymorphism.

Exemplary sequences of IL-13 receptor proteins and soluble forms thereof (e.g., IL-13Rα1 and IL-13Rα2 or fusions thereof) are described, e.g., in Donaldson et al. (1998) J Immunol. 161:2317-24; U.S. Pat. No. 6,214,559; U.S. Pat. No. 6,248,714; and U.S. Pat. No. 6,268,480.

Exemplary sequences and characterization of IL-4, e.g., human IL-4, are disclosed in Strober et al. (1988) Pediatr. Res. 24:549; and in Ramanthan et al. U.S. Pat. No. 6,358,509.

Exemplary sequence of IL-4 receptor proteins, soluble forms and fusions thereof are described in, e.g., in Stahl et al. U.S. Pat. No. 7,083,949; Seipelt, I. et al. (1997) Biochem and Biophys Res Comm 239:534-542; Stahl, N. et al. (1999) FASEB Journal Abstract, 1457; and Harada, N. et al. (1990) Proc Natl Acad Sci USA 87:857-861. An exemplary secreted form of human IL-4 receptor is recited as follows:

(SEQ ID NO:224)

MGWLCSGLLFPVSCLVLLQVASSGNMKVLQEPTCVSDYMSISTCEWKMNG
PTNCSTELRLLYQLVFLLSEAHTCIPENNGGAGCVCHLLMDDVVSADNYT
LDLWAGQQLLWKGSFKPSEHVKPRAPGNLTVHTNVSDTLLLTWSNPYPPD
NYLYNHLTYAVNIWSENDPADFRIYNVTYLEPSLRIAASTLKSGISYRAR
VRAWAQCYNTTWSEWSPSTKWHNSNIC

The phrase “a biological activity of” IL-13/IL-13R polypeptide and/or the IL-4/IL-4R polypeptide refers to one or more of the biological activities of the corresponding mature IL-13 or IL-4 polypeptide, including, but not limited to, (1) interacting with, e.g., binding to, an IL-13R or IL-4R polypeptide (e.g., a human IL-13R or IL-4R polypeptide); (2) associating with signal transduction molecules, e.g., γ common; (3) stimulating phosphorylation and/or activation of stat proteins, e.g., STAT6; (4) induction of CD23 expression; (5) production of IgE by human B cells; (6) induction of antigen-induced eosinophilia in vivo; (7) induction of antigen-induced bronchoconstriction in vivo; (8) induction of drug-induced airway hyperreactivity in vivo; (9) induction of eotoxin levels in vivo; and/or (10) induction histamine release by basophils.

An “IL-13 associated disorder or condition” is one in which IL-13 contributes to a pathology or symptom of the disorder or condition. Accordingly, an IL-13 binding agent, e.g., an IL-13 binding agent that is an antagonist of one or more IL-13 associated activities, can be used to treat or prevent the disorder.

As used herein, a “therapeutically effective amount” of an IL-13/IL-13R antagonist or an IL-4/IL-4 antagonist refers to an amount of an agent which is effective, upon single or multiple dose administration to a subject, e.g., a human patient, at curing, reducing the severity of, ameliorating, or preventing one or more symptoms of a disorder, or in prolonging the survival of the subject beyond that expected in the absence of such treatment.

As used herein, a “prophylactically effective amount” of an IL-13/IL-13R antagonist or an IL-4/IL-4R antagonist refers to an amount of an IL-13/IL-13R antagonist or an IL-4/IL-4R antagonist which is effective, upon single or multiple dose administration to a subject, e.g., a human patient, in preventing, reducing the severity, or delaying the occurrence of the onset or recurrence of an IL-13-associated disorder or condition, e.g., a disorder or condition as described herein.

As used herein “a single treatment interval” referres to an amount and/or frequency of administration of an IL-13/IL-13R antagonist and/or IL-4/IL-4R antagonist that when administered as a single dose, or as a repeated dose of limited frequency reduces the severity of, ameliorates, prevents, or delays the occurrence of the onset or recurrence of, one or more symptoms of an IL-13-associated disorder or condition, e.g., a disorder or condition as described herein. In embodiments, the frequency of administration is limited to no more than two or three doses during a single treatment interval, e.g., the repeated dose is administered within one week or less from the initial dose.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5 M sodium phosphate, 7% SDS at 65° C., followed by one or more washes at 0.2×SSC, 1% SDS at 65° C. Very high stringency conditions (4) are the preferred conditions and the ones that are used unless otherwise specified.

The methods and compositions of the present invention encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to the sequence specified are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%. 91%, 92%, 93%, 94%, 95%, 96%, 97%, 98% or 99% identity to the sequence specified are termed substantially identical.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

### Antibody Molecules

Examples of IL-13 or IL-4 antagonists and/or binding agents include antibody molecules. As used herein, the term “antibody molecule” refers to a protein comprising at least one immunoglobulin variable domain sequence. The term antibody molecule includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes one or two heavy (H) chain variable domain sequences and/or one of two light (L) chain variable domain sequence. Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a VH or VHH domain; (vi) a dAb fragment, which consists of a VH domain; (vii) a camelid or camelized variable domain; and (viii) a single chain Fv (scFv).

The VH and VL regions can be further subdivided into regions of hypervariability, termed “complementarity determining regions” (CDR), interspersed with regions that are more conserved, termed “framework regions” (FR). The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). Generally, unless specifically indicated, the following definitions are used: AbM definition of CDR1 of the heavy chain variable domain and Kabat definitions for the other CDRs. In addition, embodiments of the invention described with respect to Kabat or AbM CDRs may also be implemented using Chothia hypervariable loops. Each VH and VL typically includes three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an IL-13 binding agent that comprises determinants that form an interface that binds to the IL-13, e.g., a mammalian IL-13, e.g., human or non-human primate IL-13, or an epitope thereof. With respect to proteins (or protein mimetics), the antigen-binding site typically includes one or more loops (of at least four amino acids or amino acid mimics) that form an interface that binds to IL-13. Typically, the antigen-binding site of an antibody molecule includes at least one or two CDRs, or more typically at least three, four, five or six CDRs.

An “epitope” refers to the site on a target compound that is bound by a binding agent, e.g., an antibody molecule. An epitope can be a linear or conformational epitope, or a combination thereof. In the case where the target compound is a protein, for example, an epitope may refer to the amino acids that are bound by the binding agent. Overlapping epitopes include at least one common amino acid residue.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)). Numerous methods are available for obtaining antibody molecules.

One exemplary method of generating antibody molecules includes screening protein expression libraries, e.g., phage or ribosome display libraries. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; and WO 90/02809. In addition to the use of display libraries, other methods can be used to obtain an anti-IL-13 antibody molecule. For example, an IL-13 protein or a peptide thereof can be used as an antigen in a non-human animal, e.g., a rodent, e.g., a mouse, hamster, or rat.

In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, published Oct. 31, 1996, and PCT Application No. PCT/US96/05928, filed Apr. 29, 1996.

In another embodiment, a monoclonal antibody is obtained from the non-human animal, and then modified, e.g., humanized or deimmunized. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies can be generated by replacing sequences of the Fv variable domain that are not directly involved in antigen binding with equivalent sequences from human Fv variable domains. Exemplary methods for generating humanized antibody molecules are provided by Morrison (1985) Science 229:1202-1207; by Oi et al. (1986) BioTechniques 4:214; and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

An antibody molecule may also be modified by specific deletion of human T cell epitopes or “deimmunization” by the methods disclosed in WO 98/52976 and WO 00/34317. Briefly, the heavy and light chain variable domains of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the V_{H} and V_{L} sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used.

Human germline sequences, e.g., are disclosed in Tomlinson, et al. (1992) J. Mol. Biol. 227:776-798; Cook, G. P. et al. (1995) Immunol. Today Vol. 16 (5): 237-242; Chothia, D. et al. (1992) J. Mol. Biol. 227:799-817; and Tomlinson et al. (1995) EMBO J. 14:4628-4638. The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Additionally, chimeric, humanized, and single-chain antibody molecules (e.g., proteins that include both human and nonhuman portions), may be produced using standard recombinant DNA techniques. Humanized antibodies may also be produced, for example, using transgenic mice that express human heavy and light chain genes, but are incapable of expressing the endogenous mouse immunoglobulin heavy and light chain genes.

Additionally, the antibody molecules described herein also include those that bind to IL-13, interfere with the formation of a functional IL-13 signaling complex, and have mutations in the constant regions of the heavy chain. It is sometimes desirable to mutate and inactivate certain fragments of the constant region. For example, mutations in the heavy constant region can be made to produce antibodies with reduced binding to the Fc receptor (FcR) and/or complement; such mutations are well known in the art. An example of such a mutation to the amino sequence of the constant region of the heavy chain of IgG is provided in SEQ ID NO:128. Certain active fragments of the CL and CH subunits (e.g., CH1) are covalently link to each other. A further aspect provides a method for obtaining an antigen-binding site that is specific for a surface of IL-13 that participates in forming a functional IL-13 signaling complex.

Exemplary antibody molecules can include sequences of VL chains as set forth in SEQ ID NOs:30-46, and/or of VH chains as set forth in and SEQ ID NOs:50-115, but also can include variants of these sequences that retain IL-13 binding ability. Such variants may be derived from the provided sequences using techniques well known in the art. Amino acid substitutions, deletions, or additions, can be made in either the FRs or in the CDRs. Whereas changes in the framework regions are usually designed to improve stability and reduce immunogenicity of the antibody molecule, changes in the CDRs are usually designed to increase affinity of the antibody molecule for its target. Such affinity-increasing changes are typically determined empirically by altering the CDR region and testing the antibody molecule. Such alterations can be made according to the methods described in Antibody Engineering, 2nd. ed. (1995), ed. Borrebaeck, Oxford University Press.

An exemplary method for obtaining a heavy chain variable domain sequence that is a variant of a heavy chain variable domain sequence described herein, includes adding, deleting, substituting, or inserting one or more amino acids in a heavy chain variable domain sequence described herein, optionally combining the heavy chain variable domain sequence with one or more light chain variable domain sequences, and testing a protein that includes the modified heavy chain variable domain sequence for specific binding to IL-13, and (preferably) testing the ability of such antigen-binding domain to modulate one or more IL-13-associated activities. An analogous method may be employed using one or more sequence variants of a light chain variable domain sequence described herein.

Variants of antibody molecules can be prepared by creating libraries with one or more varied CDRs and screening the libraries to find members that bind to IL-13, e.g., with improved affinity. For example, Marks et al. (Bio/Technology (1992) 10:779-83) describe methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. The repertoire may be combined with a CDR3 of a particular antibody. Further, the CDR3-derived sequences may be shuffled with repertoires of VH or VL domains lacking a CDR3, and the shuffled complete VH or VL domains combined with a cognate VL or VH domain to provide specific antigen-binding fragments. The repertoire may then be displayed in a suitable host system such as the phage display system of WO 92/01047, so that suitable antigen-binding fragments can be selected. Analogous shuffling or combinatorial techniques are also disclosed by Stemmer (Nature (1994) 370:389-91). A further alternative is to generate altered VH or VL regions using random mutagenesis of one or more selected VH and/or VL genes to generate mutations within the entire variable domain. See, e.g., Gram et al. Proc. Nat. Acad. Sci. USA (1992) 89:3576-80.

Another method that may be used is to direct mutagenesis to CDR regions of VH or VL genes. Such techniques are disclosed by, e.g., Barbas et al. (Proc. Nat. Acad. Sci. USA (1994) 91:3809-13) and Schier et al. (J. Mol. Biol. (1996) 263:551-67). Similarly, one or more, or all three CDRs may be grafted into a repertoire of VH or VL domains, or even some other scaffold (such as a fibronectin domain). The resulting protein is evaluated for ability to bind to IL-13.

In one embodiment, a binding agent that binds to a target is modified, e.g., by mutagenesis, to provide a pool of modified binding agents. The modified binding agents are then evaluated to identify one or more altered binding agents which have altered functional properties (e.g., improved binding, improved stability, lengthened stability in vivo). In one implementation, display library technology is used to select or screen the pool of modified binding agents. Higher affinity binding agents are then identified from the second library, e.g., by using higher stringency or more competitive binding and washing conditions. Other screening techniques can also be used.

In some embodiments, the mutagenesis is targeted to regions known or likely to be at the binding interface. If, for example, the identified binding agents are antibody molecules, then mutagenesis can be directed to the CDR regions of the heavy or light chains as described herein. Further, mutagenesis can be directed to framework regions near or adjacent to the CDRs, e.g., framework regions, particular within 10, 5, or 3 amino acids of a CDR junction. In the case of antibodies, mutagenesis can also be limited to one or a few of the CDRs, e.g., to make step-wise improvements.

In one embodiment, mutagenesis is used to make an antibody more similar to one or more germline sequences. One exemplary germlining method can include: identifying one or more germline sequences that are similar (e.g., most similar in a particular database) to the sequence of the isolated antibody. Then mutations (at the amino acid level) can be made in the isolated antibody, either incrementally, in combination, or both. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a CDR region. For example, the germline CDR residue can be from a germline sequence that is similar (e.g., most similar) to the variable domain being modified. After mutagenesis, activity (e.g., binding or other functional activity) of the antibody can be evaluated to determine if the germline residue or residues are tolerated. Similar mutagenesis can be performed in the framework regions.

Selecting a germline sequence can be performed in different ways. For example, a germline sequence can be selected if it meets a predetermined criteria for selectivity or similarity, e.g., at least a certain percentage identity, e.g., at least 75, 80, 85, 90, 91, 92, 93, 94, 95, 96, 97, 98, 99, or 99.5% identity. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may including using two germline sequences that separately contribute to the amino-terminal portion and the carboxy-terminal portion. In other implementations more than one or two germline sequences are used, e.g., to form a consensus sequence.

In other embodiments, the antibody may be modified to have an altered glycosylation pattern (i.e., altered from the original or native glycosylation pattern). As used in this context, “altered” means having one or more carbohydrate moieties deleted, and/or having one or more glycosylation sites added to the original antibody. Addition of glycosylation sites to the presently disclosed antibodies may be accomplished by altering the amino acid sequence to contain glycosylation site consensus sequences; such techniques are well known in the art. Another means of increasing the number of carbohydrate moieties on the antibodies is by chemical or enzymatic coupling of glycosides to the amino acid residues of the antibody. These methods are described in, e.g., WO 87/05330, and Aplin and Wriston (1981) CRC Crit. Rev. Biochem. 22:259-306. Removal of any carbohydrate moieties present on the antibodies may be accomplished chemically or enzymatically as described in the art (Hakimuddin et al. (1987) Arch. Biochem. Biophys. 259:52; Edge et al. (1981) Anal. Biochem. 118:131; and Thotakura et al. (1987) Meth. Enzymol. 138:350). See, e.g., U.S. Pat. No. 5,869,046 for a modification that increases in vivo half life by providing a salvage receptor binding epitope.

In one embodiment, the anti-IL-13 antibody molecule includes at least one, two and preferably three CDRs from the light or heavy chain variable domain of an antibody disclosed herein, e.g., MJ 2-7. For example, the protein includes one or more of the following sequences within a CDR region:

GFNIKDTYIH (SEQ ID NO:15),

RIDPANDNIKYDPKFQG (SEQ ID NO: 16),

SEENWYDFFDY (SEQ ID NO:17),

RSSQSIVHSNGNTYLE (SEQ ID NO: 18),

KVSNRFS (SEQ ID NO:19), and

FQGSHIPYT (SEQ ID NO:20), or a CDR having an amino acid sequence that differs by no more than 4, 3, 2.5, 2, 1.5, 1, or 0.5 alterations (e.g., substitutions, insertions or deletions) for every 10 amino acids (e.g., the number of differences being proportional to the CDR length) relative to a sequence listed above, e.g., at least one alteration but not more than two, three, or four per CDR.

For example, the anti-IL-13 antibody molecule can include, in the light chain variable domain sequence, at least one, two, or three of the following sequences within a CDR region:

RSSQSIVHSNGNTYLE (SEQ ID NO:18),

KVSNRFS (SEQ ID NO:19), and

FQGSHIPYT (SEQ ID NO:20), or an amino acid sequence that differs by no more than 4, 3, 2.5, 2, 1.5, 1, or 0.5 substitutions, insertions or deletions for every 10 amino acids relative to a sequence listed above.

The anti-IL-13 antibody molecule can include, in the heavy chain variable domain sequence, at least one, two, or three of the following sequences within a CDR region:

GFNIKDTYIH (SEQ ID NO:15),

RIDPANDNIKYDPKFQG (SEQ ID NO:16), and

SEENWYDFFDY (SEQ ID NO:17), or an amino acid sequence that differs by no more than 4, 3, 2.5, 2, 1.5, 1, or 0.5 substitutions, insertions or deletions for every 10 amino acids relative to a sequence listed above. The heavy chain CDR3 region can be less than 13 or less than 12 amino acids in length, e.g., 11 amino acids in length (either using Chothia or Kabat definitions).

In another example, the anti-IL-13 antibody molecule can include, in the light chain variable domain sequence, at least one, two, or three of the following sequences within a CDR region (amino acids in parentheses represent alternatives for a particular position):

(i)

(SEQ ID NO:25)

(RK)-S-S-Q-S-(LI)-(KV)-H-S-(ND)-G-N-(TN)-Y-L-(EDNQ
YAS)
or

(SEQ ID NO:26)

(RK)-S-S-Q-S-(LI)-(KV)-H-S-(ND)-G-N-(TN)-Y-L-E,
or

(SEQ ID NO:21)

(RK)-S-S-Q-S-(LI)-(KV)-H-S-N-G-N-T-Y-L-(EDNQYAS),
(ii)

(SEQ ID NO:27)

K-(LVI)-S-(NY)-(RW)-(FD)-S,
or

(SEQ ID NO:22)

K-(LV)-S-(NY)-R-F-S,
and
(iii)

(SEQ ID NO:28)

Q-(GSA)-(ST)-(HEQ)-I-P,

(SEQ ID NO:23)

F-Q-(GSA)-(SIT)-(HEQ)-(IL)-P,
or

(SEQ ID NO:194)

Q-(GSA)-(ST)-(HEQ)-I-P-Y-T,
or

(SEQ ID NO:29)

F-Q-(GSA)-(SIT)-(HEQ)-(IL)-P-Y-T.

In one preferred embodiment, the anti-IL-13 antibody molecule includes all six CDR's from MJ 2-7 or closely related CDRs, e.g., CDRs which are identical or which have at least one amino acid alteration, but not more than two, three or four alterations (e.g., substitutions, deletions, or insertions). The IL-13 binding agent can include at least two, three, four, five, six, or seven IL-13 contacting amino acid residues of MJ 2-7

In still another example, the anti-IL-13 antibody molecule includes at least one, two, or three CDR regions that have the same canonical structures and the corresponding CDR regions of MJ 2-7, e.g., at least CDR1 and CDR2 of the heavy and/or light chain variable domains of MJ 2-7.

In another example, the anti-IL-13 antibody molecule can include, in the heavy chain variable domain sequence, at least one, two, or three of the following sequences within a CDR region (amino acids in parentheses represent alternatives for a particular position):

(i)

(SEQ ID NO:48)

G-(YF)-(NT)-I-K-D-T-Y-(MI)-H,
(ii)

(SEQ ID NO:49)

(WR)-I-D-P-(GA)-N-D-N-I-K-Y-(SD)-(PQ)-K-F-Q-G,
and
(iii)

(SEQ ID NO:17)

SEENWYDFFDY.

In one embodiment, the anti-IL-13 antibody molecule includes at least one, two and preferably three CDR's from the light or heavy chain variable domain of an antibody disclosed herein, e.g., C65. For example, the anti-IL-13 antibody molecule includes one or more of the following sequences within a CDR region:

QASQGTSINLN (SEQ ID NO:118),

GASNLED (SEQ ID NO:119), and

LQHSYLPWT (SEQ ID NO:120)

GFSLTGYGVN (SEQ ID NO:121),

IIWGDGSTDYNSAL (SEQ ID NO:122), and

DKTFYYDGFYRGRMDY (SEQ ID NO:123), or a CDR having an amino acid sequence that differs by no more than 4, 3, 2.5, 2, 1.5, 1, or 0.5 substitutions, insertions or deletions for every 10 amino acids (e.g., the number of differences being proportional to the CDR length) relative to a sequence listed above, e.g., at least one alteration but not more than two, three, or four per CDR. For example, the protein can include, in the light chain variable domain sequence, at least one, two, or three of the following sequences within a CDR region:

QASQGTSINLN (SEQ ID NO: 118),

GASNLED (SEQ ID NO:119), and

LQHSYLPWT (SEQ ID NO:120), or an amino acid sequence that differs by no more than 4, 3, 2.5, 2, 1.5, 1, or 0.5 substitutions, insertions or deletions for every 10 amino acids relative to a sequence listed above.

The anti-IL-13 antibody molecule can include, in the heavy chain variable domain sequence, at least one, two, or three of the following sequences within a CDR region:

GFSLTGYGVN (SEQ ID NO:121),

IIWGDGSTDYNSAL (SEQ ID NO:122), and

DKTFYYDGFYRGRMDY (SEQ ID NO:123), or an amino acid sequence that differs by no more than 4, 3, 2.5, 2, 1.5, 1, or 0.5 substitutions, insertions or deletions for every 10 amino acids relative to a sequence listed above.

In embodiments, the IL-13 antibody molecule can include one of the following sequences:

(SEQ ID NO:30)

DIVMTQTPLSLPVTPGEPASISCRSSQSIVHSNGNTYLEWYLQKPGQSPQ
LLIYKVSNRFSGVPDRFSGSGSGTDFTLKISRVEAEDVGVYYCFQGSHIP
YT

(SEQ ID NO:31)

DVVMTQSPLSLPVTLGQPASISCRSSQSIVHSNGNTYLEWFQQRPGQSPR
RLIYKVSNRFSGVPDRFSGSGSGTDFTLKISRVEAEDVGVYYCFQGSHIP
YT

(SEQ ID NO:32)

DIVMTQTPLSLSVTPGQPASISCRSSQSIVHSNGNTYLEWYLQKPGQSPQ
LLIYKVSNRFSGVPDRFSGSGSGTDFTLKISRVEAEDVGVYYCFQGSHIP
YT

(SEQ ID NO:33)

DIVMTQTPLSLSVTPGQPASISCRSSQSIVHSNGNTYLEWYLQKPGQPPQ
LLIYKVSNRFSGVPDRFSGSGSGTDFTLKISRVEAEDVGVYYCFQGSHIP
YT

(SEQ ID NO:34)

DIVMTQSPLSLPVTPGEPASISCRSSQSIVHSNGNTYLEWYLQKPGQSPQ
LLIYKVSNRFSGVPDRFSGSGSGTDFTLKISRVEAEDVGVYYCFQGSHIP
YT

(SEQ ID NO:35)

DIVMTQTPLSSPVTLGQPASISCRSSQSIVHSNGNTYLEWLQQRPGQPPR
LLIYKVSNRFSGVPDRFSGSGAGTDFTLKISRVEAEDVGVYYCFQGSHIP
YT

(SEQ ID NO:36)

DIQMTQSPSSLSASVGDRVTITCRSSQSIVHSNGNTYLEWYQQKPGKAPK
LLIYKVSNRFSGVPSRFSGSGSGTDFTLTISSLQPEDFATYYCFQGSHIP
YT

(SEQ ID NO:37)

DVVMTQSPLSLPVTLGQPASISCRSSQSLVYSDGNTYLNWFQQRPGQSPR
RLIYKVSNRFSGVPDRFSGSGSGTDFTLKISRVEAEDVGVYYCFQGSHIP
YT

(SEQ ID NO:38)

DVLMTQTPLSLPVSLGDQASISCRSSQSIVHSNGNTYLEWYLQKPGQSPK
LLIYKVSNRFSGVPDRFSGSGSGTDFTLKISRVEAEDLGVYYCFQGSHIP
YT

or a sequence that has fewer than eight, seven, six, five, four, three, or two alterations (e.g., substitutions, insertions or deletions, e.g., conservative substitutions or a substitution for an amino acid residue at a corresponding position in MJ 2-7). Exemplary substitutions are at one of the following Kabat positions: 2, 4, 6, 35, 36, 38, 44, 47, 49, 62, 64-69, 85, 87, 98, 99, 101, and 102. The substitutions can, for example, substitute an amino acid at a corresponding position from MJ 2-7 into a human framework region.

The IL-13 antibody molecule may also include one of the following sequences:

(SEQ ID NO:39)

DIVMTQTPLSLPVTPGEPASISC-(RK)-S-S-Q-S-(LI)-(KV)-H-
S-(ND)-G-N-(TN)-Y-L-(EDNQYAS)WYLQKPGQSPQLLIYK-
(LVI)-S-(NY)-(RW)-(FD)-SGVPDRFSGSGSGTDFTLKISRV
EAEDVGVYYC F-Q-(GSA)-(SIT)-(HEQ)-(IL)-P

(SEQ ID NO:40)

DVVMTQSPLSLPVTLGQPASISC-(RK)-S-S-Q-S-(LI)-(KV)-H-
S-(ND)-G-N-(TN)-Y-L-(EDNQYAS)WFQQRPGQSPRRLIYK-(LV
I)-S-(NY)-(RW)-(FD)-SGVPDRFSGSGSGTDFTLKISRVEAEDVGV
YYCF-Q-(GSA)-(SIT)-(HEQ)-(IL)-P

(SEQ ID NO:41)

DIVMTQTPLSLSVTPGQPASISC-(RK)-S-S-Q-S-(LI)-(KV)-H-
S-(ND)-G-N-(TN)-Y-L-(EDNQYAS)WYLQKPGQSPQLLIYK-(LV
I)-S-(NY)-(RW)-(FD)-SGVPDRFSGSGSGTDFTLKISRVEAEDVGV
YYCF-Q-(GSA)-(SIT)-(HEQ)-(IL)-P

(SEQ ID NO:42)

DIVMTQTPLSLSVTPGQPASISC(RK)-S-S-Q-S-(LI)-(KV)-H-S-
(ND)-G-N-(TN)-Y-L-(EDNQYAS)WYLQKPGQPPQLLIYK-(LVI)-
S-(NY)-(RW)-(FD)-SGVPDRFSGSGSGTDFTLKISRVEAEDVGVYYC
F-Q-(GSA)-(SIT)-(HEQ)-(IL)-P

(SEQ ID NO:43)

DIVMTQSPLSLPVTPGEPASISC(RK)-S-S-Q-S-(LI)-(KV)-H-S-
(ND)-G-N-(TN)-Y-L-(EDNQYAS)WYLQKPGQSPQLLIYK-(LVI)-
S-(NY)-(RW)-(FD)-SGVPDRFSGSGSGTDFTLKISRVEAEDVGVYYC
F-Q-(GSA)-(SIT)-(HEQ)-(IL)-P

(SEQ ID NO:44)

DIVMTQTPLSSPVTLGQPASISC(RK)-S-S-Q-S-(LI)-(KV)-H-S-
(ND)-G-N-(TN)-Y-L-(EDNQYAS)WLQQRPGQPPRLLIYK-(LVI)-
S-(NY)-(RW)-(FD)-SGVPDRFSGSGAGTDFTLKISRVEAEDVGVYYC
F-Q-(GSA)-(SIT)-(HEQ)-(IL)-P

(SEQ ID NO:45)

DIQMTQSPSSLSASVGDRVTITC(RK)-S-S-Q-S-(LI)-(KV)-H-S-
(ND)-G-N-(TN)-Y-L-(EDNQYAS)WYQQKPGKAPKLLIYK-(LVI)-
S-(NY)-(RW)-(FD)-SGVPSRFSGSGSGTDFTLTISSLQPEDFATYYC
F-Q-(GSA)-(SIT)-(HEQ)-(IL)-P

(SEQ ID NO:46)

DVLMTQTPLSLPVSLGDQASISC(RK)-S-S-Q-S-(LI)-(KV)-H-S-
(ND)-G-N-(TN)-Y-L-(EDNQYAS)WYLQKPGQSPKLLIYK-(LVI)-
S-(NY)-(RW)-(FD)-SGVPDRFSGSGSGTDFTLKISRVEAEDLGVYYC
F-Q-(GSA)-(SIT)-(HEQ)-(IL)-P

or a sequence that has fewer than eight, seven, six, five, four, three, or two alterations (e.g., substitutions, insertions or deletions, e.g., conservative substitutions or a substitution for an amino acid residue at a corresponding position in MJ 2-7) in the framework region. Exemplary substitutions are at one or more of the following Kabat positions: 2, 4, 6, 35, 36, 38, 44, 47, 49, 62, 64-69, 85, 87, 98, 99, 101, and 102. The substitutions can, for example, substitute an amino acid at a corresponding position from MJ 2-7 into a human framework region. The sequences may also be followed by the dipeptide Tyr-Thr. The FR4 region can include, e.g., the sequence FGGGTKVEIKR (SEQ ID NO:47).

In other embodiments, the IL-13 antibody molecule can include one of the following sequences:

(SEQ ID NO:50)

QVQLVQSGAEVKKPGASVKVSCKASGFNIKDTYIHWVRQAPGQGLEWMGR
IDPANDNIKYDPKFQGRVTMTRDTSISTAYMELSRLRSDDTAVYYCARSE
ENWYDFFDY

(SEQ ID NO:51)

QVQLVQSGAEVKKPGASVKVSCKASGFNIKDTYIHWVRQAPGQRLEWMGR
IDPANDNIKYDPKFQGRVTITRDTSASTAYMELSSLRSEDTAVYYCARSE
ENWYDFFDY

(SEQ ID NO:52)

QVQLVQSGAEVKKPGASVKVSCKASGFNIKDTYIHWVRQATGQGLEWMGR
IDPANDNIKYDPKFQGRVTMTRNTSISTAYMELSSLRSEDTAVYYCARSE
ENWYDFFDY

(SEQ ID NO:53)

QVQLVQSGAEVKKPGASVKVSCKASGFNIKDTYIHWVRQAPGQGLEWMGR
IDPANDNIKYDPKFQGRVTMTTDTSTSTAYMELRSLRSDDTAVYYCARSE
ENWYDFFDY

(SEQ ID NO:54)

QVQLVQSGAEVKKPGASVKVSCKVSGFNIKDTYIHWVRQAPGKGLEWMGR
IDPANDNIKYDPKFQGRVTMTEDTSTDTAYMELSSLRSEDTAVYYCATSE
ENWYDFFDY

(SEQ ID NO:55)

QMQLVQSGAEVKKTGSSVKVSCKASGFNIKDTYIHWVRQAPGQALEWMGR
IDPANDNIKYDPKFQGRVTITRDRSMSTAYMELSSLRSEDTAMYYCARSE
ENWYDFFDY

(SEQ ID NO:56)

QVQLVQSGAEVKKPGASVKVSCKASGFNIKDTYIHWVRQAPGQGLEWMGR
IDPANDNIKYDPKFQGRVTMTRDTSTSTVYMELSSLRSEDTAVYYCARSE
ENWYDFFDY

(SEQ ID NO:57)

QMQLVQSGPEVKKPGTSVKVSCKASGFNIKDTYIHWVRQARGQRLEWIGR
IDPANDNIKYDPKFQGRVTITRDMSTSTAYMELSSLRSEDTAVYYCAASE
ENWYDFFDY

(SEQ ID NO:58)

EVQLVESGGGLVQPGGSLRLSCAASGFNIKDTYIHWVRQAPGKGLEWVAR
IDPANDNIKYDPKFQGRFTISRDNAKNSLYLQMNSLRAEDTAVYYCARSE
ENWYDFFDY

(SEQ ID NO:59)

EVQLVESGGGLVQPGRSLRLSCAASGFNIKDTYIHWVRQAPGKGLEWVSR
IDPANDNIKYDPKFQGRFTISRDNAKNSLYLQMNSLRAEDTALYYCAKDS
EENWYDFFDY

(SEQ ID NO:60)

QVQLVESGGGLVKPGGSLRLSCAASGFNIKDTYIHWIRQAPGKGLEWVSR
IDPANDNIKYDPKFQGRFTISRDNAKNSLYLQMNSLRAEDTAVYYCARSE
ENWYDFFDY

(SEQ ID NO:61)

EVQLVESGGGLVKPGGSLRLSCAASGFNIKDTYIHWVRQAPGKGLEWVGR
IDPANDNIKYDPKFQGRFTISRDDSKNTLYLQMNSLKTEDTAVYYCTTSE
ENWYDFFDY

(SEQ ID NO:62)

EVQLVESGGGVVRPGGSLRLSCAASGFNIKDTYIHWVRQAPGKGLEWVSR
IDPANDNIKYDPKFQGRFTISRDNAKNSLYLQMNSLRAEDTALYHCARSE
ENWYDFFDY

(SEQ ID NO:63)

EVQLVESGGGLVKPGGSLRLSCAASGFNIKDTYIHWVRQAPGKGLEWVSR
IDPANDNIKYDPKFQGRFTISRDNAKNSLYLQMNSLRAEDTAVYYCARSE
ENWYDFFDY

(SEQ ID NO:64)

EVQLLESGGGLVQPGGSLRLSCAASGFNIKDTYIHWVRQAPGKGLEWVSR
IDPANDNIKYDPKFQGRFTISRDNSKNTLYLQMNSLRAEDTAVYYCAKSE
ENWYDFFDY

(SEQ ID NO:65)

QVQLVESGGGVVQPGRSLRLSCAASGFNIKDTYIHWVRQAPGKGLEWVAR
IDPANDNIKYDPKFQGRFTISRDNSKNTLYLQMNSLRAEDTAVYYCAKSE
ENWYDFFDY

(SEQ ID NO:66)

QVQLVESGGGVVQPGRSLRLSCAASGFNIKDTYIHWVRQAPGKGLEWVAR
IDPANDNIKYDPKFQGRFTISRDNSKNTLYLQMNSLRAEDTAVYYCARSE
ENWYDFFDY

(SEQ ID NO:67)

EVQLVESGGVVVQPGGSLRLSCAASGFNIKDTYIHWVRQAPGKGLEWVSR
IDPANDNIKYDPKFQGRFTISRDNSKNSLYLQMNSLRTEDTALYYCAKDS
EENWYDFFDY

(SEQ ID NO:68)

EVQLVESGGGLVQPGGSLRLSCAASGFNIKDTYIHWVRQAPGKGLEWVSR
IDPANDNIKYDPKFQGRFTISRDNAKNSLYLQMNSLRDEDTAVYYCARSE
ENWYDFFDY

(SEQ ID NO:69)

EVQLVESGGGLVQPGRSLRLSCTASGFNIKDTYIHWFRQAPGKGLEWVGR
IDPANDNIKYDPKFQGRFTISRDGSKSIAYLQMNSLKTEDTAVYYCTRSE
ENWYDFFDY

(SEQ ID NO:70)

EVQLVESGGGLVQPGGSLRLSCAASGFNIKDTYIHWVRQAPGKGLEYVSR
IDPANDNIKYDPKFQGRFTISRDNSKNTLYLQMGSLRAEDMAVYYCARSE
ENWYDFFDY

(SEQ ID NO:71)

EVQLVESGGGLVQPGGSLRLSCAASGFNIKDTYIHWVRQAPGKGLEWIGR
IDPANDNIKYDPKFQGRFTISRDNAKNSLYIIQMNSLRAEDTAVYYCARS
EENWYDFFDY

(SEQ ID NO:72)

EVQLVESGGGLVQPGGSLRLSCAASGFNIKDTYIHWVRQAPGKGLEWVAR
IDPANDNIKYDPKFQGKATISRDNAKNSLYLQMNSLRAEDTAVYYCARSE
ENWYDFFDY

(SEQ ID NO:73)

EVQLVESGGGLVQPGGSLRLSCAASGFNIKDTYIHWVRQAPGKGLEWVAR
IDPANDNIKYDPKFQGRFTISADNAKNSLYLQMNSLRAEDTAVYYCARSE
ENWYDFFDY

(SEQ ID NO:74)

EVQLVESGGGLVQPGGSLRLSCAASGFNIKDTYIHWVRQAPGKGLEWVGR
IDPANDNIKYDPKFQGRFTISRDNAKNSLYLQMNSLRAEDTAVYYCARSE
ENWYDFFDY

(SEQ ID NO:75)

EVQLVESGGGLVQPGGSLRLSCAASGFNIKDTYIHWVRQAPGKGLEWVAR
IDPANDNIKYDPKFQGKATISADNAKNSLYLQMNSLRAEDTAVYYCARSE
ENWYDFFDY

(SEQ ID NO:76)

EVQLVESGGGLVQPGGSLRLSCAASGFNIKDTYIHWVRQAPGKGLEWIGR
IDPANDNIKYDPKFQGRFTISADNAKNSLYLQMNSLRAEDTAVYYCARSE
ENWYDFFDY

(SEQ ID NO:77)

EVQLVESGGGLVQPGGSLRLSCAASGFNIKDTYIHWVRQAPGKGLEWVGR
IDPANDNIKYDPKFQGRFTISADNAKNSLYLQMNSLRAEDTAVYYCARSE
ENWYDFFDY

(SEQ ID NO:78)

EVQLVESGGGLVQPGGSLRLSCAASGFNIKDTYIHWVRQAPGKGLEWVAR
IDPANDNIKYDPKFQGRFTISRDNAKNSAYLQMNSLRAEDTAVYYCARSE
ENWYDFFDY

(SEQ ID NO:79)

EVQLVESGGGLVQPGGSLRLSCAASGFNIKDTYIHWVRQAPGKGLEWVGR
IDPANDNIKYDPKFQGRFTISADNAKNSAYLQMNSLRAEDTAVYYCARSE
ENWYDFFDY

(SEQ ID NO:80)

EVQLVESGGGLVQPGGSLRLSCAASGFNIKDTYIHWVRQAPGKGLEWIGR
IDPANDNIKYDPKFQGRFTISADNAKNSAYLQMNSLRAEDTAVYYCARSE
ENWYDFFDY

(SEQ ID NO:81)

EVQLVESGGGLVQPGGSLRLSCTGSGFNIKDTYIHWVRQAPGKGLEWIGR
IDPANDNIKYDPKFQGRFTISADNAKNSLYLQMNSLRAEDTAVYYCARSE
ENWYDFFDY

(SEQ ID NO:82)

EVQLQQSGAELVKPGASVKLSCTGSGFNIKDTYIHWVKQRPEQGLEWIGR
IDPANDNIKYDPKFQGKATITADTSSNTAYLQLNSLTSEDTAVYYCARSE
ENWYDFFDY

or a sequence that has fewer than eight, seven, six, five, four, three, or two alterations (e.g., substitutions, insertions or deletions, e.g., conservative substitutions or a substitution for an amino acid residue at a corresponding position in MJ 2-7). Exemplary substitutions are at one or more of the following Kabat positions: 2, 4, 6, 25, 36, 37, 39, 47, 48, 93, 94, 103, 104, 106, and 107. Exemplary substitutions can also be at one or more of the following positions (accordingly to sequential numbering): 48, 49, 67, 68, 72, and 79. The substitutions can, for example, substitute an amino acid at a corresponding position from MJ 2-7 into a human framework region. In one embodiment, the sequence includes (accordingly to sequential numbering) one or more of the following: Ile at 48, Gly at 49, Lys at 67, Ala at 68, Ala at 72, and Ala at 79; preferably, e.g., Ile at 48, Gly at 49, Ala at 72, and Ala at 79.

Further, the frameworks of the heavy chain variable domain sequence can include: (i) at a position corresponding to 49, Gly; (ii) at a position corresponding to 72, Ala; (iii) at positions corresponding to 48, Ile, and to 49, Gly; (iv) at positions corresponding to 48, Ile, to 49, Gly, and to 72, Ala; (v) at positions corresponding to 67, Lys, to 68, Ala, and to 72, Ala; and/or (vi) at positions corresponding to 48, Ile, to 49, Gly, to 72, Ala, to 79, Ala.

The IL-13 antibody molecule may also include one of the following sequences:

(SEQ ID NO:83)

QVQLVQSGAEVKKPGASVKVSCKASG-(YF)-(NT)-I-K-D-T-Y-
(MI)-H, WVRQAPGQGLEWMG(WR)-I-D-P-(GA)-N-D-N-I-K-Y-
(SD)-(PQ)-K-F-Q-GRVTMTRDTSISTAYMELSRLRSDDTAVYYCARS
EENWYDFFDY

(SEQ ID NO:84)

QVQLVQSGAEVKKPGASVKVSCKASG-(YF)-(NT)-I-K-D-T-Y-
(MI)-H, WVRQAPGQRLEWMG(WR)-I-D-P-(GA)-N-D-N-I-K-Y-
(SD)-(PQ)-K-F-Q-GRVTITRDTSASTAYMELSSLRSEDTAVYYCARS
EENWYDFFDY

(SEQ ID NO:85)

QVQLVQSGAEVKKPGASVKVSCKASG-(YF)-(NT)-I-K-D-T-Y-
(MI)-H, WVRQATGQGLEWMG(WR)-I-D-P-(GA)-N-D-N-I-K-Y-
(SD)-(PQ)-K-F-Q-GRVTMTRNTSISTAYMELSSLRSEDTAVYYCARS
EENWYDFFDY

(SEQ ID NO:86)

QVQLVQSGAEVKKPGASVKVSCKASG-(YF)-(NT)-I-K-D-T-Y-
(MI)-H, WVRQAPGQGLEWMG(WR)-I-D-P-(GA)-N-D-N-I-K-Y-
(SD)-(PQ)-K-F-Q-GRVTMTTDTSTSTAYMELRSLRSDDTAVYYCARS
EENWYDFFDY

(SEQ ID NO:87)

QVQLVQSGAEVKKPGASVKVSCKVSG-(YF)-(NT)-I-K-D-T-Y-
(MI)-H, WVRQAPGKGLEWMG(WR)-I-D-P-(GA)-N-D-N-I-K-Y-
(SD)-(PQ)-K-F-Q-GRVTMTEDTSTDTAYMELSSLRSEDTAVYYCATS
EENWYDFFDY

(SEQ ID NO:88)

QMQLVQSGAEVKKTGSSVKVSCKASG-(YF)-(NT)-I-K-D-T-Y-
(MI)-H, WVRQAPGQALEWMG(WR)-I-D-P-(GA)-N-D-N-I-K-Y-
(SD)-(PQ)-K-F-Q-GRVTITRDRSMSTAYMELSSLRSEDTAMYYCARS
EENWYDFFDY

(SEQ ID NO:89)

QVQLVQSGAEVKKPGASVKVSCKASG-(YF)-(NT)-I-K-D-T-Y-
(MI)-H, WVRQAPGQGLEWMG(WR)-I-D-P-(GA)-N-D-N-I-K-Y-
(SD)-(PQ)-K-F-Q-GRVTMTRDTSTSTVYMELSSLRSEDTAVYYCARS
EENWYDFFDY

(SEQ ID NO:90)

QMQLVQSGPEVKKPGTSVKVSCKASG-(YF)-(NT)-I-K-D-T-Y-
(MI)-H, WVRQARGQRLEWIG(WR)-I-D-P-(GA)-N-D-N-I-K-Y-
(SD)-(PQ)-K-F-Q-GRVTITRDMSTSTAYMELSSLRSEDTAVYYCAAS
EENWYDFFDY

(SEQ ID NO:91)

EVQLVESGGGLVQPGGSLRLSCAASG-(YF)-(NT)-I-K-D-T-Y-
(MI)-H, WVRQAPGKGLEWVA(WR)-I-D-P-(GA)-N-D-N-I-K-Y-
(SD)-(PQ)-K-F-Q-GRFTISRDNAKNSLYLQMNSLRAEDTAVYYCARS
EENWYDFFDY

(SEQ ID NO:92)

EVQLVESGGGLVQPGRSLRLSCAASG-(YF)-(NT)-I-K-D-T-Y-
(MI)-H, WVRQAPGKGLEWVS(WR)-I-D-P-(GA)-N-D-N-I-K-Y-
(SD)-(PQ)-K-F-Q-GRFTISRDNAKNSLYLQMNSLRAEDTALYYCAKD
SEENWYDFFDY

(SEQ ID NO:93)

QVQLVESGGGLVKPGGSLRLSCAASG-(YF)-(NT)-I-K-D-T-Y-
(MI)-H, WIRQAPGKGLEWVS(WR)-I-D-P-(GA)-N-D-N-I-K-Y-
(SD)-(PQ)-K-F-Q-GRFTISRDNAKNSLYLQMNSLRAEDTAVYYCARS
EENWYDFFDY

(SEQ ID NO:94)

EVQLVESGGGLVKPGGSLRLSCAASG-(YF)-(NT)-I-K-D-T-Y-
(MI)-H, WVRQAPGKGLEWVG(WR)-I-D-P-(GA)-N-D-N-I-K-Y-
(SD)-(PQ)-K-F-Q-GRFTISRDDSKNTLYLQMNSLKTEDTAVYYCTTS
EENWYDFFDY

(SEQ ID NO:95)

EVQLVESGGGVVRPGGSLRLSCAASG-(YF)-(NT)-I-K-D-T-Y-
(MI)-H, WVRQAPGKGLEWVS(WR)-I-D-P-(GA)-N-D-N-I-K-Y-
(SD)-(PQ)-K-F-Q-GRFTISRDNAKNSLYLQMNSLRAEDTALYHCARS
EENWYDFFDY

(SEQ ID NO:96)

EVQLVESGGGLVKPGGSLRLSCAASG-(YF)-(NT)-I-K-D-T-Y-
(MI)-H, WVRQAPGKGLEWVS(WR)-I-D-P-(GA)-N-D-N-I-K-Y-
(SD)-(PQ)-K-F-Q-GRFTISRDNAKNSLYLQMNSLRAEDTAVYYCARS
EENWYDFFDY

(SEQ ID NO:97)

EVQLLESGGGLVQPGGSLRLSCAASG-(YF)-(NT)-I-K-D-T-Y-
(MI)-H, WVRQAPGKGLEWVS(WR)-I-D-P-(GA)-N-D-N-I-K-Y-
(SD)-(PQ)-K-F-Q-GRFTISRDNSKNTLYLQMNSLRAEDTAVYYCAKS
EENWYDFFDY

(SEQ ID NO:98)

QVQLVESGGGVVQPGRSLRLSCAASG-(YF)-(NT)-I-K-D-T-Y-
(MI)-H, WVRQAPGKGLEWVA(WR)-I-D-P-(GA)-N-D-N-I-K-Y-
(SD)-(PQ)-K-F-Q-GRFTISRDNSKNTLYLQMNSLRAEDTAVYYCAKS
EENWYDFFDY

(SEQ ID NO:99)

QVQLVESGGGVVQPGRSLRLSCAASG-(YF)-(NT)-I-K-D-T-Y-
(MI)-H, WVRQAPGKGLEWVA(WR)-I-D-P-(GA)-N-D-N-I-K-Y-
(SD)-(PQ)-K-F-Q-GRFTISRDNSKNTLYLQMNSLRAEDTAVYYCARS
EENWYDFFDY

(SEQ ID NO:100)

EVQLVESGGVVVQPGGSLRLSCAASG-(YF)-(NT)-I-K-D-T-Y-
(MI)-H, WVRQAPGKGLEWVS(WR)-I-D-P-(GA)-N-D-N-I-K-Y-
(SD)-(PQ)-K-F-Q-GRFTISRDNSKNSLYLQMNSLRTEDTALYYCAKD
SEENWYDFFDY

(SEQ ID NO:101)

EVQLVESGGGLVQPGGSLRLSCAASG-(YF)-(NT)-I-K-D-T-Y-
(MI)-H, WVRQAPGKGLEWVS(WR)-I-D-P-(GA)-N-D-N-I-K-Y-
(SD)-(PQ)-K-F-Q-GRFTISRDNAKNSLYLQMNSLRDEDTAVYYCARS
EENWYDFFDY

(SEQ ID NO:102)

EVQLVESGGGLVQPGRSLRLSCTASG-(YF)-(NT)-I-K-D-T-Y-
(MI)-H, WFRQAPGKGLEWVG(WR)-I-D-P-(GA)-N-D-N-I-K-Y-
(SD)-(PQ)-K-F-Q-GRFTISRDGSKSIAYLQMNSLKTEDTAVYYCTRS
EENWYDFFDY

(SEQ ID NO:103)

EVQLVESGGGLVQPGGSLRLSCAASG-(YF)-(NT)-I-K-D-T-Y-
(MI)-H, WVRQAPGKGLEYVS(WR)-I-D-P-(GA)-N-D-N-I-K-Y-
(SD)-(PQ)-K-F-Q-GRFTISRDNSKNTLYLQMGSLRAEDMAVYYCARS
EENWYDFFDY

(SEQ ID NO:104)

EVQLVESGGGLVQPGGSLRLSCAASG-(YF)-(NT)-I-K-D-T-Y-
(MI)-H, WVRQAPGKGLEWIG(WR)-I-D-P-(GA)-N-D-N-I-K-Y-
(SD)-(PQ)-K-F-Q-GRFTISRDNAKNSLYLQMNSLRAEDTAVYYCARS
EENWYDFFDY

(SEQ ID NO:105)

EVQLVESGGGLVQPGGSLRLSCAASG-(YF)-(NT)-I-K-D-T-Y-
(MI)-H, WVRQAPGKGLEWVA(WR)-I-D-P-(GA)-N-D-N-I-K-Y-
(SD)-(PQ)-K-F-Q-GKATISRDNAKNSLYLQMNSLRAEDTAVYYCARS
EENWYDFFDY

(SEQ ID NO:106)

EVQLVESGGGLVQPGGSLRLSCAASG-(YF)-(NT)-I-K-D-T-Y-
(MI)-H, WVRQAPGKGLEWVA(WR)-I-D-P-(GA)-N-D-N-I-K-Y-
(SD)-(PQ)-K-F-Q-GRFTISADNAKNSLYLQMNSLRAEDTAVYYCARS
EENWYDFFDY

(SEQ ID NO:107)

EVQLVESGGGLVQPGGSLRLSCAASG-(YF)-(NT)-I-K-D-T-Y-
(MI)-H, WVRQAPGKGLEWVG(WR)-I-D-P-(GA)-N-D-N-I-K-Y-
(SD)-(PQ)-K-F-Q-GRFTISRDNAKNSLYLQMNSLRAEDTAVYYCARS
EENWYDFFDY

(SEQ ID NO:108)

EVQLVESGGGLVQPGGSLRLSCAASG-(YF)-(NT)-I-K-D-T-Y-
(MI)-H, WVRQAPGKGLEWVA(WR)-I-D-P-(GA)-N-D-N-I-K-Y-
(SD)-(PQ)-K-F-Q-GKATISADNAKNSLYLQMNSLRAEDTAVYYCARS
EENWYDFFDY

(SEQ ID NO:109)

EVQLVESGGGLVQPGGSLRLSCAASG-(YF)-(NT)-I-K-D-T-Y-
(MI)-H, WVRQAPGKGLEWIG(WR)-I-D-P-(GA)-N-D-N-I-K-Y-
(SD)-(PQ)-K-F-Q-GRFTISADNAKNSLYLQMNSLRAEDTAVYYCARS
EENWYDFFDY

(SEQ ID NO:110)

EVQLVESGGGLVQPGGSLRLSCAASG-(YF)-(NT)-I-K-D-T-Y-
(MI)-H, WVRQAPGKGLEWVG(WR)-I-D-P-(GA)-N-D-N-I-K-Y-
(SD)-(PQ)-K-F-Q-GRFTISADNAKNSLYLQMNSLRAEDTAVYYCARS
EENWYDFFDY

(SEQ ID NO:111)

EVQLVESGGGLVQPGGSLRLSCAASG-(YF)-(NT)-I-K-D-T-Y-
(MI)-H, WVRQAPGKGLEWVA(WR)-I-D-P-(GA)-N-D-N-I-K-Y-
(SD)-(PQ)-K-F-Q-GRFTISRDNAKNSAYLQMNSLRAEDTAVYYCARS
EENWYDFFDY

(SEQ ID NO:112)

EVQLVESGGGLVQPGGSLRLSCAASG-(YF)-(NT)-I-K-D-T-Y-
(MI)-H, WVRQAPGKGLEWVG(WR)-I-D-P-(GA)-N-D-N-I-K-Y-
(SD)-(PQ)-K-F-Q-GRFTISADNAKNSAYLQMNSLRAEDTAVYYCARS
EENWYDFFDY

(SEQ ID NO:113)

EVQLVESGGGLVQPGGSLRLSCAASG-(YF)-(NT)-I-K-D-T-Y-
(MI)-H, WVRQAPGKGLEWIG(WR)-I-D-P-(GA)-N-D-N-I-K-Y-
(SD)-(PQ)-K-F-Q-GRFTISADNAKNSAYLQMNSLRAEDTAVYYCARS
EENWYDFFDY

(SEQ ID NO:114)

EVQLVESGGGLVQPGGSLRLSCTGSG-(YF)-(NT)-I-K-D-T-Y-
(MI)-H, WVRQAPGKGLEWIG(WR)-I-D-P-(GA)-N-D-N-I-K-Y-
(SD)-(PQ)-K-F-Q-GRFTISADNAKNSLYLQMNSLRAEDTAVYYCARS
EENWYDFFDY

(SEQ ID NO:115)

EVQLQQSGAELVKPGASVKLSCTGSG-(YF)-(NT)-I-K-D-T-Y-
(MI)-H, WVKQRPEQGLEWIG(WR)-I-D-P-(GA)-N-D-N-I-K-Y-
(SD)-(PQ)-K-F-Q-GKATITADTSSNTAYLQLNSLTSEDTAVYYCARSE
ENWYDFFDY

or a sequence that has fewer than eight, seven, six, five, four, three, or two alterations

(e.g., substitutions, insertions or deletions, e.g., conservative substitutions or a substitution for an amino acid residue at a corresponding position in MJ 2-7) in the framework region. Exemplary substitutions are at one or more of the following Kabat positions: 2, 4, 6, 25, 36, 37, 39, 47, 48, 93, 94, 103, 104, 106, and 107. The substitutions can, for example, substitute an amino acid at a corresponding position from MJ 2-7 into a human framework region. The FR4 region can include, e.g., the sequence

| | |
|---|---|
| WGQGTTLTVSS | (SEQ ID NO:116) |
| or | |
| WGQGTLVTVSS. | (SEQ ID NO:117) |

Additional examples of IL-13 antibodies, that interfere with IL-13 binding to IL-13R (e.g., an IL-13 receptor complex), or a subunit thereof, include “mAb13.2” and modified, e.g., chimeric or humanized forms thereof. The amino acid and nucleotide sequences for the heavy chain variable region of mAb13.2 are set forth herein as SEQ ID NO:198 and SEQ IUD NO:217, respectively. The amino acid and nucleotide sequences for the light chain variable region of mAb13.2 are set forth herein as SEQ ID NO:199 and SEQ ID NO:218, respectively. An exemplary chimeric form (e.g., a form comprising the heavy and light chain variable region of mAb13.2) is referred to herein as “ch13.2.” The amino acid and nucleotide sequences for the heavy chain variable region of ch13.2 are set forth herein as SEQ ID NO:208 and SEQ ID NO:204, respectively. The amino acid and nucleotide sequences for the light chain variable region of ch13.2 are set forth herein as SEQ ID NO:213 and SEQ ID NO:219, respectively. A humanized form of mAb13.2, which is referred to herein as “h13.2v1,” has amino acid and nucleotide sequences for the heavy chain variable region set forth herein as SEQ ID NO:209 and SEQ ID NO:205, respectively. The amino acid and nucleotide sequences for the light chain variable region of h13.2v1 are set forth herein as SEQ ID NO:214 and SEQ ID NO:220, respectively. Another humanized form of mAb13.2, which is referred to herein as “h13.2v2,” has amino acid and nucleotide sequences for the heavy chain variable region set forth herein as SEQ ID NO:210 and SEQ ID NO:206, respectively. The amino acid and nucleotide sequences for the light chain variable region of h13.2v2 are set forth herein as SEQ ID NO:212 and SEQ ID NO:221, respectively. Another humanized form of mAb13.2, which is referred to herein as “h13.2v3,” has amino acid and nucleotide sequences for the heavy chain variable region set forth herein as SEQ ID NO:211 and SEQ ID NO:207, respectively. The amino acid and nucleotide sequences for the light chain variable region of h13.2v3 are set forth herein as SEQ ID NO:35 and SEQ ID NO:223, respectively.

In another embodiment, the anti-IL-13 antibody molecule comprises at least one, two, three, or four antigen-binding regions, e.g., variable regions, having an amino acid sequence as set forth in SEQ ID NOs:198, 208, 209, 210, or 211 for VH, and/or SEQ ID NOs:199, 213, 214, 212, or 215 for VL), or a sequence substantially identical thereto (e.g., a sequence at least about 85%, 90%, 95%, 99% or more identical thereto, or which differs by no more than 1, 2, 5, 10, or 15 amino acid residues from SEQ ID NOs: 199, 213, 214, 212, 198, 208, 209, 210, 215, or 211). In another embodiment, the antibody includes a VH and/or VL domain encoded by a nucleic acid having a nucleotide sequence as set forth in SEQ ID NOs 222, 204, 205, 208, or 207 for VH, and/or SEQ ID NOs:218, 219, 220, 221, or 223 for VL), or a sequence substantially identical thereto (e.g., a sequence at least about 85%, 90%, 95%, 99% or more identical thereto, or which differs by no more than 3, 6, 15, 30, or 45 nucleotides from SEQ ID NOs:218, 219, 220, 221, 222, 204, 205, 206, 223, or 207). In yet another embodiment, the antibody or fragment thereof comprises at least one, two, or three CDRs from a heavy chain variable region having an amino acid sequence as set forth in SEQ ID NOs:202, 203, or 196 for VH CDRs 1-3, respectively, or a sequence substantially homologous thereto (e.g., a sequence at least about 85%, 90%, 95%, 99% or more identical thereto, and/or having one or more substitutions, e.g., conserved substitutions). In yet another embodiment, the antibody or fragment thereof comprises at least one, two, or three CDRs from a light chain variable region having an amino acid sequence as set forth in SEQ ID NOs:197, 200, or 201 for VL CDRs 1-3, respectively, or a sequence substantially homologous thereto (e.g., a sequence at least about 85%, 90%, 95%, 99% or more identical thereto, and/or having one or more substitutions, e.g., conserved substitutions). In yet another embodiment, the antibody or fragment thereof comprises at least one, two, three, four, five or six CDRs from heavy and light chain variable regions having an amino acid sequence as set forth in SEQ ID NOs:202, 203, 196 for VH CDRs 1-3, respectively; and SEQ ID NO:197, 200, or 201 for VL CDRs 1-3, respectively, or a sequence substantially homologous thereto (e.g., a sequence at least about 85%, 90%, 95%, 99% or more identical thereto, and/or having one or more substitutions, e.g., conserved substitutions).

In one embodiment, the anti-IL-13 antibody molecule includes all six CDRs from C65 or closely related CDRs, e.g., CDRs which are identical or which have at least one amino acid alteration, but not more than two, three or four alterations (e.g., substitutions, deletions, or insertions).

In still another embodiment, the IL-13 binding agent includes at least one, two or three CDR regions that have the same canonical structures and the corresponding CDR regions of C65, e.g., at least CDR1 and CDR2 of the heavy and/or light chain variable domains of C65.

In one embodiment, the heavy chain framework (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the heavy chain framework of one of the following germline V segment sequences: DP-71 or DP-67 or another V gene which is compatible with the canonical structure class of C65 (see, e.g., Chothia et al. (1992) J. Mol. Biol. 227:799-817; Tomlinson et al. (1992) J. Mol. Biol. 227:776-798).

In one embodiment, the light chain framework (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the light chain framework of DPK-1 or DPK-9 germline sequence or another V gene which is compatible with the canonical structure class of C65 (see, e.g., Tomlinson et al. (1995) EMBO J. 14:4628).

In another embodiment, the light chain framework (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the light chain framework of a Vκ I subgroup germline sequence, e.g., a DPK-9 or DPK-1 sequence.

In another embodiment, the heavy chain framework (e.g., FR1, FR2, FR3, individually, or a sequence encompassing FR1, FR2, and FR3, but excluding CDRs) includes an amino acid sequence, which is at least 80%, 85%, 90%, 95%, 97%, 98%, 99% or higher identical to the light chain framework of a VH IV subgroup germline sequence, e.g., a DP-71 or DP-67 sequence.

In one embodiment, the light or the heavy chain variable framework (e.g., the region encompassing at least FR1, FR2, FR3, and optionally FR4) can be chosen from: (a) a light or heavy chain variable framework including at least 80%, 85%, 90%, 95%, or 100% of the amino acid residues from a human light or heavy chain variable framework, e.g., a light or heavy chain variable framework residue from a human mature antibody, a human germline sequence, a human consensus sequence, or a human antibody described herein; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, a human consensus sequence; (c) a non-human framework (e.g., a rodent framework); or (d) a non-human framework that has been modified, e.g., to remove antigenic or cytotoxic determinants, e.g., deimmunized, or partially humanized. In one embodiment, the heavy chain variable domain sequence includes human residues or human consensus sequence residues at one or more of the following positions (preferably at least five, ten, twelve, or all): (in the FR of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the FR of the variable domain of the heavy chain) 2H, 4H, 24H, 36H, 37H, 39H, 43H, 45H, 49H, 58H, 60H, 67H, 68H, 69H, 70H, 73H, 74H, 75H, 78H, 91H, 92H, 93H, and/or 103H (according to the Kabat numbering).

In one embodiment, the anti-IL13 antibody molecules includes at least one non-human CDR, e.g., a murine CDR, e.g., a CDR from e.g., mAb13.2, MJ2-7, C65, and/or modified forms thereof (e.g., humanized or chimeric variansts thereof), and at least one framework which differs from a framework of e.g., mAb13.2, MJ2-7, C65, and/or modified forms thereof (e.g., humanized or chimeric variansts thereof) by at least one amino acid, e.g., at least 5, 8, 10, 12, 15, or 18 amino acids. For example, the proteins include one, two, three, four, five, or six such non-human CDRs and includes at least one amino acid difference in at least three of HC FR1, HC FR2, HC FR3, LC FR1, LC FR2, and LC FR3.

In one embodiment, the heavy or light chain variable domain sequence of the anti-IL-13 antibody molecule includes an amino acid sequence, which is at least 80%, 85%, 90%, 95%, 97%, 98%, 99% or higher identical to a variable domain sequence of an antibody described herein, e.g., mAb13.2, MJ2-7, C65, and/or modified forms thereof (e.g., humanized or chimeric variansts thereof); or which differs at least 1 or 5 residues, but less than 40, 30, 20, or 10 residues, from a variable domain sequence of an antibody described herein, e.g., mAb13.2, MJ2-7, C65, and/or modified forms thereof (e.g., humanized or chimeric variansts thereof). In one embodiment, the heavy or light chain variable domain sequence of the protein includes an amino acid sequence encoded by a nucleic acid sequence described herein or a nucleic acid that hybridizes to a nucleic acid sequence described herein or its complement, e.g., under low stringency, medium stringency, high stringency, or very high stringency conditions.

In one embodiment, one or both of the variable domain sequences include amino acid positions in the framework region that are variously derived from both a non-human antibody (e.g., a murine antibody such as mAb13.2) and a human antibody or germline sequence. For example, a variable domain sequence can include a number of positions at which the amino acid residue is identical to both the non-human antibody and the human antibody (or human germline sequence) because the two are identical at that position. Of the remaining framework positions where the non-human and human differ, at least 50, 60, 70, 80, or 90% of the positions of the variable domain are preferably identical to the human antibody (or human germline sequence) rather than the non-human. For example, none, or at least one, two, three, or four of such remaining framework position may be identical to the non-human antibody rather than to the human. For example, in HC FR1, one or two such positions can be non-human; in HC FR2, one or two such positions can be non-human; in FR3, one, two, three, or four such positions can be non-human; in LC FR1, one, two, three, or four such positions can be non-human; in LC FR2, one or two such positions can be non-human; in LC FR3, one or two such positions can be non-human. The frameworks can include additional non-human positions.

In one embodiment, an antibody molecule has CDR sequences that differ only insubstantially from those of MJ 2-7, C65, or 13.2. Insubstantial differences include minor amino acid changes, such as substitutions of 1 or 2 out of any of typically 5-7 amino acids in the sequence of a CDR, e.g., a Chothia or Kabat CDR. Typically, an amino acid is substituted by a related amino acid having similar charge, hydrophobic, or stereochemical characteristics. Such substitutions are within the ordinary skills of an artisan. Unlike in CDRs, more substantial changes in structure framework regions (FRs) can be made without adversely affecting the binding properties of an antibody. Changes to FRs include, but are not limited to, humanizing a nonhuman-derived framework or engineering certain framework residues that are important for antigen contact or for stabilizing the binding site, e.g., changing the class or subclass of the constant region, changing specific amino acid residues which might alter an effector function such as Fc receptor binding (Lund et al. (1991) J. Immunol. 147:2657-62; Morgan et al. (1995) Immunology 86:319-24), or changing the species from which the constant region is derived. Antibodies may have mutations in the CH2 region of the heavy chain that reduce or alter effector function, e.g., Fc receptor binding and complement activation. For example, antibodies may have mutations such as those described in U.S. Pat. Nos. 5,624,821 and 5,648,260. In the IgG1 or IgG2 heavy chain, for example, such mutations may be made to resemble the amino acid sequence set forth in SEQ ID NO:17. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08).

The anti-IL-13 antibody molecule can be in the form of intact antibodies, antigen-binding fragments of antibodies, e.g., Fab, F(ab′)₂, Fd, dAb, and scFv fragments, and intact antibodies and fragments that have been mutated either in their constant and/or variable domain (e.g., mutations to produce chimeric, partially humanized, or fully humanized antibodies, as well as to produce antibodies with a desired trait, e.g., enhanced IL-13 binding and/or reduced FcR binding).

The anti-IL-13 antibody molecule can be derivatized or linked to another functional molecule, e.g., another peptide or protein (e.g., an Fab fragment). For example, the binding agent can be functionally linked (e.g., by chemical coupling, genetic fusion, noncovalent association or otherwise) to one or more other molecular entities, such as another antibody molecule (e.g., to form a bispecific or a multispecific antibody molecule), toxins, radioisotopes, cytotoxic or cytostatic agents, among others.

### Additional IL-13/IL-13R or IL-4/IL-4R Binding Agents

Also provided are other binding agents, other than antibody molecules, that bind to IL-13 or IL-4 polypeptide or nucleic acid, or an IL-13R or IL-4R polypeptide or nucleic acid. In embodiments, the other binding agents described herein are antagonists and thus reduce, inhibit or otherwise diminish one or more biological activities of IL-13 and/or IL-4 (e.g., one or more biological activities of IL-13 and/or IL-4 as described herein).

Binding agents can be identified by a number of means, including modifying a variable domain described herein or grafting one or more CDRs of a variable domain described herein onto another scaffold domain. Binding agents can also be identified from diverse libraries, e.g., by screening. One method for screening protein libraries uses phage display. Particular regions of a protein are varied and proteins that interact with IL-13 or IL-4, or its receptors, are identified, e.g., by retention on a solid support or by other physical association. For example, to identify particular binding agents that bind to the same epitope or an overlapping epitope as MJ2-7, C65 or mAb 13.2 on IL-13, binding agents can be eluted by adding MJ2-7, C65 or mAb13.2 (or related antibody), or binding agents can be evaluated in competition experiments with MJ2-7, C65 or mAb13.2 (or related antibody). It is also possible to deplete the library of agents that bind to other epitopes by contacting the library to a complex that contains IL-13 and MJ2-7, C65 or mAb13.2 (or related antibody). The depleted library can then be contacted to IL-13 to obtain a binding agent that binds to IL-13 but not to IL-13 when it is bound by MJ 2-7, C65 or mAb13.2. It is also possible to use peptides from IL-13 that contain the MJ 2-7, C65 epitope, or mAb13.2 as a target.

Phage display is described, for example, in U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; WO 94/05781; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; and Barbas et al. (1991) PNAS 88:7978-7982. Yeast surface display is described, e.g., in Boder and Wittrup (1997) Nat. Biotechnol. 15:553-557. Another form of display is ribosome display. See, e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30. and Schaffitzel et al. (1999) J Immunol Methods. 231(1-2):119-35.

Binding agents that bind to IL-13 or IL-4, or its receptors, can have structural features of one scaffold proteins, e.g., a folded domain. An exemplary scaffold domain, based on an antibody, is a “minibody” scaffold has been designed by deleting three beta strands from a heavy chain variable domain of a monoclonal antibody (Tramontano et al., 1994, J. Mol. Recognit. 7:9; and Martin et al., 1994, EMBO J. 13:5303-5309). This domain includes 61 residues and can be used to present two hypervariable loops, e.g., one or more hypervariable loops of a variable domain described herein or a variant described herein. In another approach, the binding agent includes a scaffold domain that is a V-like domain (Coia et al. WO 99/45110). V-like domains refer to a domain that has similar structural features to the variable heavy (VH) or variable light (VL) domains of antibodies. Another scaffold domain is derived from tendamistatin, a 74 residue, six-strand beta sheet sandwich held together by two disulfide bonds (McConnell and Hoess, 1995, J. Mol. Biol. 250:460). This parent protein includes three loops. The loops can be modified (e.g., using CDRs or hypervariable loops described herein) or varied, e.g., to select domains that bind to IL-13 or IL-4, or its receptors. WO 00/60070 describes a β-sandwich structure derived from the naturally occurring extracellular domain of CTLA-4 that can be used as a scaffold domain.

Still another scaffold domain for an IL-13/13R or IL-4/IL-4R binding agent is a domain based on the fibronectin type III domain or related fibronectin-like proteins. The overall fold of the fibronectin type III (Fn3) domain is closely related to that of the smallest functional antibody fragment, the variable domain of the antibody heavy chain. Fn3 is a β-sandwich similar to that of the antibody VH domain, except that Fn3 has seven β-strands instead of nine. There are three loops at the end of Fn3; the positions of BC, DE and FG loops approximately correspond to those of CDR1, 2 and 3 of the VH domain of an antibody. Fn3 is advantageous because it does not have disulfide bonds. Therefore, Fn3 is stable under reducing conditions, unlike antibodies and their fragments (see WO 98/56915; WO 01/64942; WO 00/34784). An Fn3 domain can be modified (e.g., using CDRs or hypervariable loops described herein) or varied, e.g., to select domains that bind to IL-13 or IL-4, or its receptors.

Still other exemplary scaffold domains include: T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). US 20040009530 describes examples of some alternative scaffolds.

Examples of small scaffold domains include: Kunitz domains (58 amino acids, 3 disulfide bonds), Cucurbida maxima trypsin inhibitor domains (31 amino acids, 3 disulfide bonds), domains related to guanylin (14 amino acids, 2 disulfide bonds), domains related to heat-stable enterotoxin IA from gram negative bacteria (18 amino acids, 3 disulfide bonds), EGF domains (50 amino acids, 3 disulfide bonds), kringle domains (60 amino acids, 3 disulfide bonds), fungal carbohydrate-binding domains (35 amino acids, 2 disulfide bonds), endothelin domains (18 amino acids, 2 disulfide bonds), and Streptococcal G IgG-binding domain (35 amino acids, no disulfide bonds). Examples of small intracellular scaffold domains include SH2, SH3, and EVH domains. Generally, any modular domain, intracellular or extracellular, can be used.

Exemplary criteria for evaluating a scaffold domain can include: (1) amino acid sequence, (2) sequences of several homologous domains, (3) 3-dimensional structure, and/or (4) stability data over a range of pH, temperature, salinity, organic solvent, oxidant concentration. In one embodiment, the scaffold domain is a small, stable protein domains, e.g., a protein of less than 100, 70, 50, 40 or 30 amino acids. The domain may include one or more disulfide bonds or may chelate a metal, e.g., zinc.

Still other binding agents are based on peptides, e.g., proteins with an amino acid sequence that are less than 30, 25, 24, 20, 18, 15, or 12 amino acids. Peptides can be incorporated in a larger protein, but typically a region that can independently bind to IL-13, e.g., to an epitope described herein. Peptides can be identified by phage display. See, e.g., US 20040071705.

A binding agent may include non-peptide linkages and other chemical modification. For example, part or all of the binding agent may be synthesized as a peptidomimetic, e.g., a peptoid (see, e.g., Simon et al. (1992) Proc. Natl. Acad. Sci. USA 89:9367-71 and Horwell (1995) Trends Biotechnol. 13:132-4). A binding agent may include one or more (e.g., all) non-hydrolyzable bonds. Many non-hydrolyzable peptide bonds are known in the art, along with procedures for synthesis of peptides containing such bonds. Exemplary non-hydrolyzable bonds include—[CH₂NH]— reduced amide peptide bonds, —[COCH₂]— ketomethylene peptide bonds, —[CH(CN)NH]— (cyanomethylene)amino peptide bonds, —[CH₂CH(OH)]— hydroxyethylene peptide bonds, —[CH₂O]— peptide bonds, and—[CH₂S]— thiomethylene peptide bonds (see e.g., U.S. Pat. No. 6,172,043).

In another embodiment, the IL-13 or IL-4 antagonist is derived from a lipocalin, e.g., a human lipocalin scaffold.

### Soluble Receptors

A soluble form of an IL-13 or an IL-4 receptor or a modified antagonistic cytokine can be used alone or functionally linked (e.g., by chemical coupling, genetic or polypeptide fusion, non-covalent association or otherwise) to a second moiety, e.g., an immunoglobulin Fc domain, serum albumin, pegylation, a GST, Lex-A or an MBP polypeptide sequence. As used herein, a “fusion protein” refers to a protein containing two or more operably associated, e.g., linked, moieties, e.g., protein moieties. Typically, the moieties are covalently associated. The moieties can be directly associate, or connected via a spacer or linker.

The fusion proteins may additionally include a linker sequence joining the first moiety to the second moiety. For example, the fusion protein can include a peptide linker, e.g., a peptide linker of about 4 to 20, more preferably, 5 to 10, amino acids in length; the peptide linker is 8 amino acids in length. Each of the amino acids in the peptide linker is selected from the group consisting of Gly, Ser, Asn, Thr and Ala; the peptide linker includes a Gly-Ser element. In other embodiments, the fusion protein includes a peptide linker and the peptide linker includes a sequence having the formula (Ser-Gly-Gly-Gly-Gly)y wherein y is 1, 2, 3, 4, 5, 6, 7, or 8.

In other embodiments, additional amino acid sequences can be added to the N- or C-terminus of the fusion protein to facilitate expression, detection and/or isolation or purification. For example, the receptor fusion protein may be linked to one or more additional moieties, e.g., GST, His6 tag, FLAG tag. For example, the fusion protein may additionally be linked to a GST fusion protein in which the fusion protein sequences are fused to the C-terminus of the GST (i.e., glutathione S-transferase) sequences. Such fusion proteins can facilitate the purification of the receptor fusion protein. In another embodiment, the fusion protein is includes a heterologous signal sequence (i.e., a polypeptide sequence that is not present in a polypeptide encoded by a receptor nucleic acid) at its N-terminus. For example, the native receptor signal sequence can be removed and replaced with a signal sequence from another protein. In certain host cells (e.g., mammalian host cells), expression and/or secretion of receptor can be increased through use of a heterologous signal sequence.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) Current Protocols in Molecular Biology, John Wiley & Sons, 1992). Moreover, many expression vectors are commercially available that encode a fusion moiety (e.g., an Fc region of an immunoglobulin heavy chain). A receptor encoding nucleic acid can be cloned into such an expression vector such that the fusion moiety is linked in-frame to the immunoglobulin protein.

In some embodiments, fusion polypeptides exist as oligomers, such as dimers or trimers.

In other embodiments, the receptor polypeptide moiety is provided as a variant receptor polypeptide having a mutation in the naturally-occurring receptor sequence (wild type) that results in higher affinity (relative to the non-mutated sequence) binding of the receptor polypeptide to cytokine.

In other embodiments, additional amino acid sequences can be added to the N- or C-terminus of the fusion protein to facilitate expression, steric flexibility, detection and/or isolation or purification. The second polypeptide is preferably soluble. In some embodiments, the second polypeptide enhances the half-life, (e.g., the serum half-life) of the linked polypeptide. In some embodiments, the second polypeptide includes a sequence that facilitates association of the fusion polypeptide with a second BMP-10 receptor polypeptide. In embodiments, the second polypeptide includes at least a region of an immunoglobulin polypeptide. Immunoglobulin fusion polypeptide are known in the art and are described in e.g., U.S. Pat. Nos. 5,516,964; 5,225,538; 5,428,130; 5,514,582; 5,714,147; and 5,455,165. For example, a soluble form of a BMP-10 receptor or a BMP-10 antagonistic propeptide can be fused to a heavy chain constant region of the various isotypes, including: IgG1, IgG2, IgG3, IgG4, IgM, IgA1, IgA2, IgD, and IgE). Typically, the fusion protein can include the extracellular domain of a human BMP-10 receptor, or a BMP-10 propeptide (or a sequence homologous thereto), and, e.g., fused to, a human immunoglobulin Fc chain, e.g., human IgG (e.g., human IgG1 or human IgG2, or a mutated form thereof).

The Fc sequence can be mutated at one or more amino acids to reduce effector cell function, Fc receptor binding and/or complement activity. Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260). Similar type of alterations could be described which if applied to the murine, or other species immunoglobulin would reduce or eliminate these functions. For example, it is possible to alter the affinity of an Fc region of an antibody (e.g., an IgG, such as a human IgG) for an FcR (e.g., Fc gamma R1), or for C1q binding by replacing the specified residue(s) with a residue(s) having an appropriate functionality on its side chain, or by introducing a charged functional group, such as glutamate or aspartate, or perhaps an aromatic non-polar residue such as phenylalanine, tyrosine, tryptophan or alanine (see e.g., U.S. Pat. No. 5,624,821).

In embodiments, the second polypeptide has less effector function that the effector function of a Fc region of a wild-type immunoglobulin heavy chain. Fc effector function includes for example, Fc receptor binding, complement fixation and T cell depleting activity (see for example, U.S. Pat. No. 6,136,310). Methods for assaying T cell depleting activity, Fc effector function, and antibody stability are known in the art. In one embodiment, the second polypeptide has low or no detectable affinity for the Fc receptor. In an alternative embodiment, the second polypeptide has low or no detectable affinity for complement protein Clq.

It will be understood that the antibody molecules and soluble receptor or fusion proteins described herein can be functionally linked (e.g., by chemical coupling, genetic fusion, non-covalent association or otherwise) to one or more other molecular entities, such as an antibody (e.g., a bispecific or a multispecific antibody), toxins, radioisotopes, cytotoxic or cytostatic agents, among others.

### Nucleic Acid Antagonists

In yet another embodiment, the antagonist inhibits the expression of nucleic acid encoding an IL-13 or IL-13R, or an IL-4 or IL-4R. Examples of such antagonists include nucleic acid molecules, for example, antisense molecules, ribozymes, RNAi, triple helix molecules that hybridize to a nucleic acid encoding an IL-13 or IL-13R, or an IL-4 or IL-4R, or a transcription regulatory region, and blocks or reduces mRNA expression of an IL-13 or IL-13R, or an IL-4 or IL-4R.

In embodiments, nucleic acid antagonists are used to decrease expression of an endogenous gene encoding an IL-13 or IL-13R, or an IL-4 or IL-4R. In one embodiment, the nucleic acid antagonist is an siRNA that targets mRNA encoding an IL-13 or IL-13R, or an IL-4 or IL-4R. Other types of antagonistic nucleic acids can also be used, e.g., a dsRNA, a ribozyme, a triple-helix former, or an antisense nucleic acid. Accordingly, isolated nucleic acid molecules that are nucleic acid inhibitors, e.g., antisense, RNAi, to a an IL-13 or IL-13R, or an IL-4 or IL-4R-encoding nucleic acid molecule are provided.

An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire an IL-13 or IL-13R, or an IL-4 or IL-4R coding strand, or to only a portion thereof. In another embodiment, the antisense nucleic acid molecule is antisense to a “noncoding region” of the coding strand of a nucleotide sequence encoding an IL-13 or IL-13R, or an IL-4 or IL-4R (e.g., the 5′ and 3′ untranslated regions). Anti-sense agents can include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

Exemplary antisense compounds include DNA or RNA sequences that specifically hybridize to the target nucleic acid, e.g., the mRNA encoding BMP-10/BMP-10 receptor. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include N⁴—(C₁-C₁₂) alkylaminocytosines and N⁴,N⁴—(C₁-C₁₂) dialkylaminocytosines. Modified nucleobases may also include 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. Furthermore, N⁶—(C₁-C₁₂) alkylaminopurines and N⁶,N⁶—(C₁-C₁₂) dialkylaminopurines, including N⁶-methylaminoadenine and N⁶,N⁶-dimethylaminoadenine, are also suitable modified nucleobases. Similarly, other 6-substituted purines including, for example, 6-thioguanine may constitute appropriate modified nucleobases. Other suitable nucleobases include 2-thiouracil, 8-bromoadenine, 8-bromoguanine, 2-fluoroadenine, and 2-fluoroguanine. Derivatives of any of the aforementioned modified nucleobases are also appropriate. Substituents of any of the preceding compounds may include C₁-C₃₀ alkyl, C₂-C₃₀ alkenyl, C₂-C₃₀ alkynyl, aryl, aralkyl, heteroaryl, halo, amino, amido, nitro, thio, sulfonyl, carboxyl, alkoxy, alkylcarbonyl, alkoxycarbonyl, and the like. Descriptions of other types of nucleic acid agents are also available. See, e.g., U.S. Pat. Nos. 4,987,071; 5,116,742; and 5,093,246; Woolf et al. (1992) Proc Natl Acad Sci USA; Antisense RNA and DNA, D. A. Melton, Ed., Cold Spring Harbor Laboratory, Cold Spring Harbor, N.Y. (1988); 89:7305-9; Haselhoff and Gerlach (1988) Nature 334:585-59; Helene, C. (1991) Anticancer Drug Des. 6:569-84; Helene (1992) Ann. N.Y. Acad. Sci. 660:27-36; and Maher (1992) Bioassays 14:807-15.

The antisense nucleic acid molecules of the invention are typically administered to a subject (e.g., by direct injection at a tissue site), or generated in situ such that they hybridize with or bind to cellular mRNA and/or genomic DNA encoding a BMP-10/BMP-10 receptor protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′—O— methylribonucleotide (Inoue et al. (1987) Nucleic Acids Res. 15:6131-6148) or a chimeric RNA-DNA analogue (Inoue et al. (1987) FEBS Lett. 215:327-330).

siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically, the siRNA sequences are exactly complementary to the target mRNA. dsRNAs and siRNAs in particular can be used to silence gene expression in mammalian cells (e.g., human cells). siRNAs also include short hairpin RNAs (shRNAs) with 29-base-pair stems and 2-nucleotide 3′ overhangs. See, e.g., Clemens et al. (2000) Proc. Natl. Acad. Sci. USA 97:6499-6503; Billy et al. (2001) Proc. Natl. Sci. USA 98:14428-14433; Elbashir et al. (2001) Nature. 411:494-8; Yang et al. (2002) Proc. Natl. Acad. Sci. USA 99:9942-9947; Siolas et al. (2005), Nat. Biotechnol. 23(2):227-31; 20040086884; U.S. 20030166282; 20030143204; 20040038278; and 20030224432.

In still another embodiment, an antisense nucleic acid of the invention is a ribozyme. A ribozyme having specificity for an IL-13 or IL-13R, or an IL-4 or IL-4R-encoding nucleic acid can include one or more sequences complementary to the nucleotide sequence of an IL-13 or IL-13R, or an IL-4 or IL-4R cDNA disclosed herein, and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a BMP-10/BMP-10 receptor-encoding mRNA. See, e.g., Cech et al. U.S. Pat. No. 4,987,071; and Cech et al. U.S. Pat. No. 5,116,742. Alternatively, BMP-10/BMP-10 receptor mRNA can be used to select a catalytic RNA having a specific ribonuclease activity from a pool of RNA molecules. See, e.g., Bartel, D. and Szostak, J. W. (1993) Science 261:1411-1418.

an IL-13 or IL-13R, or an IL-4 or IL-4R gene expression can be inhibited by targeting nucleotide sequences complementary to the regulatory region of the an IL-13 or IL-13R, or an IL-4 or IL-4R (e.g., the an IL-13 or IL-13R, or an IL-4 or IL-4R promoter and/or enhancers) to form triple helical structures that prevent transcription of an IL-13 or IL-13R, or an IL-4 or IL-4R gene in target cells. See generally, Helene, C. (1991) Anticancer Drug Des. 6:569-84; Helene, C. i (1992) Ann. N.Y. Acad. Sci. 660:27-36; and Maher, L. J. (1992) Bioassays 14:807-15. The potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

An IL-13 or IL-13R, or an IL-4 or IL-4R nucleic acid molecule can be modified at the base moiety, sugar moiety or phosphate backbone to improve, e.g., the stability, hybridization, or solubility of the molecule. For non-limiting examples of synthetic oligonucleotides with modifications see Toulmé (2001) Nature Biotech. 19:17 and Faria et al. (2001) Nature Biotech. 19:40-44. Such phosphoramidite oligonucleotides can be effective antisense agents.

For example, the deoxyribose phosphate backbone of the nucleic acid molecules can be modified to generate peptide nucleic acids (see Hyrup B. et al. (1996) Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra and Perry-O'Keefe et al. Proc. Natl. Acad. Sci. 93: 14670-675.

PNAs can be used in therapeutic and diagnostic applications. For example, PNAs can be used as antisense or antigene agents for sequence-specific modulation of gene expression by, for example, inducing transcription or translation arrest or inhibiting replication. PNAs of nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe supra).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; W088/09810) or the blood-brain barrier (see, e.g., W0 89/10134). In addition, oligonucleotides can be modified with hybridization-triggered cleavage agents (see, e.g., Krol et al. (1988) Bio-Techniques 6:958-976) or intercalating agents (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

### Binding Agent Production

Some antibody molecules, e.g., Fabs, or binding agents can be produced in bacterial cells, e.g., E. coli cells. For example, if the Fab is encoded by sequences in a phage display vector that includes a suppressible stop codon between the display entity and a bacteriophage protein (or fragment thereof), the vector nucleic acid can be transferred into a bacterial cell that cannot suppress a stop codon. In this case, the Fab is not fused to the gene III protein and is secreted into the periplasm and/or media.

Antibody molecules can also be produced in eukaryotic cells. In one embodiment, the antibodies (e.g., scFv's) are expressed in a yeast cell such as Pichia (see, e.g., Powers et al. (2001) J Immunol Methods. 251:123-35), Hanseula, or Saccharomyces.

In one embodiment, antibody molecules are produced in mammalian cells. Typical mammalian host cells for expressing the clone antibodies or antigen-binding fragments thereof include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, and a cell from a transgenic animal, e.g., a transgenic mammal. For example, the cell is a mammary epithelial cell.

In addition to the nucleic acid sequences encoding the antibody molecule, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced.

In an exemplary system for recombinant expression of an antibody molecule, a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells can be cultured to allow for expression of the antibody heavy and light chains and intact antibody is recovered from the culture medium. Standard molecular biology techniques can be used to prepare the recombinant expression vector, transfect the host cells, select for transformants, culture the host cells and recover the antibody molecule from the culture medium. For example, some antibody molecules can be isolated by affinity chromatography with a Protein A or Protein G coupled matrix.

For antibody molecules that include an Fc domain, the antibody production system preferably synthesizes antibodies in which the Fc region is glycosylated. For example, the Fc domain of IgG molecules is glycosylated at asparagine 297 in the CH2 domain. This asparagine is the site for modification with biantennary-type oligosaccharides. It has been demonstrated that this glycosylation is required for effector functions mediated by Fcγ receptors and complement C1q (Burton and Woof (1992) Adv. Immunol. 51:1-84; Jefferis et al. (1998) Immunol. Rev. 163:59-76). In one embodiment, the Fc domain is produced in a mammalian expression system that appropriately glycosylates the residue corresponding to asparagine 297. The Fc domain can also include other eukaryotic post-translational modifications.

Antibody molecules can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody molecule and a signal sequence for secretion. The milk produced by females of such transgenic mammals includes, secreted-therein, the antibody of interest. The antibody molecule can be purified from the milk, or for some applications, used directly.

### Characterization of Binding Agents

The binding properties of a binding agent may be measured by any method, e.g., one of the following methods: BIACORE™ analysis, Enzyme Linked Immunosorbent Assay (ELISA), x-ray crystallography, sequence analysis and scanning mutagenesis. The ability of a protein to neutralize and/or inhibit one or more IL-13-associated activities may be measured by the following methods: assays for measuring the proliferation of an IL-13 dependent cell line, e.g. TFI; assays for measuring the expression of IL-13-mediated polypeptides, e.g., flow cytometric analysis of the expression of CD23; assays evaluating the activity of downstream signaling molecules, e.g., STAT6; assays evaluating production of tenascin; assays testing the efficiency of an antibody described herein to prevent asthma in a relevant animal model, e.g., the cynomolgus monkey, and other assays. An IL-13 binding agent, particularly an IL-13 antibody molecule, can have a statistically significant effect in one or more of these assays. Exemplary assays for binding properties include the following.

The binding interaction of a IL-13 or IL-4 binding agent and a target (e.g., IL-13 or IL-4) can be analyzed using surface plasmon resonance (SPR). SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (K_{d}), and kinetic parameters, including Kₒₙ and K_{off}, for the binding of a molecule to a target. Such data can be used to compare different molecules. Information from SPR can also be used to develop structure-activity relationships (SAR). For example, the kinetic and equilibrium binding parameters of different antibody molecule can be evaluated. Variant amino acids at given positions can be identified that correlate with particular binding parameters, e.g., high affinity and slow K_{off}. This information can be combined with structural modeling (e.g., using homology modeling, energy minimization, or structure determination by x-ray crystallography or NMR). As a result, an understanding of the physical interaction between the protein and its target can be formulated and used to guide other design processes.

### Respiratory Disorders

An IL-13 and/or IL-4 antagonist can be used to treat or prevent respiratory disorders including, but are not limited to asthma (e.g., allergic and nonallergic asthma (e.g., due to infection, e.g., with respiratory syncytial virus (RSV), e.g., in younger children)); bronchitis (e.g., chronic bronchitis); chronic obstructive pulmonary disease (COPD) (e.g., emphysema (e.g., cigarette-induced emphysema); conditions involving airway inflammation, eosinophilia, fibrosis and excess mucus production, e.g., cystic fibrosis, pulmonary fibrosis, and allergic rhinitis. For example, an IL-13 binding agent (e.g., an anti-IL-13 antibody molecule) can be administered in an amount effective to treat or prevent the disorder or to ameliorate at least one symptom of the disorder.

Asthma can be triggered by myriad conditions, e.g., inhalation of an allergen, presence of an upper-respiratory or ear infection, etc. (Opperwall (2003) Nurs. Clin. North Am. 38:697-711). Allergic asthma is characterized by airway hyperresponsiveness (AHR) to a variety of specific and nonspecific stimuli, elevated serum immunoglobulin E (IgE), excessive airway mucus production, edema, and bronchial epithelial injury (Wills-Karp, supra). Allergic asthma begins when the allergen provokes an immediate early airway response, which is frequently followed several hours later by a delayed late-phase airway response (LAR) (Henderson et al. (2000) J. Immunol. 164:1086-95). During LAR, there is an influx of eosinophils, lymphocytes, and macrophages throughout the airway wall and the bronchial fluid. (Henderson et al., supra). Lung eosinophilia is a hallmark of allergic asthma and is responsible for much of the damage to the respiratory epithelium (Li et al. (1999) J. Immunol. 162:2477-87).

CD4⁺ T helper (Th) cells are important for the chronic inflammation associated with asthma (Henderson et al., supra). Several studies have shown that commitment of CD4+ cells to type 2 T helper (Th2) cells and the subsequent production of type 2 cytokines (e.g., IL-4, IL-5, IL-10, and IL-13) are important in the allergic inflammatory response leading to AHR (Tomkinson et al. (2001) J. Immunol. 166:5792-5800, and references cited therein). First, CD4⁺ T cells have been shown to be necessary for allergy-induced asthma in murine models. Second, CD4⁺ T cells producing type 2 cytokines undergo expansion not only in these animal models but also in patients with allergic asthma. Third, type 2 cytokine levels are increased in the airway tissues of animal models and asthmatics. Fourth, Th2 cytokines have been implicated as playing a central role in eosinophil recruitment in murine models of allergic asthma, and adoptively transferred Th2 cells have been correlated with increased levels of eotaxin (a potent eosinophil chemoattractant) in the lung as well as lung eosinophilia (Wills-Karp et al., supra; Li et al., supra).

The methods for treating or preventing asthma described herein include those for extrinsic asthma (also known as allergic asthma or atopic asthma), intrinsic asthma (also known as non-allergic asthma or non-atopic asthma) or combinations of both, which has been referred to as mixed asthma. Extrinsic or allergic asthma includes incidents caused by, or associated with, e.g., allergens, such as pollens, spores, grasses or weeds, pet danders, dust, mites, etc. As allergens and other irritants present themselves at varying points over the year, these types of incidents are also referred to as seasonal asthma. Also included in the group of extrinsic asthma is bronchial asthma and allergic bronchopulmonary aspergillosis.

Disorders that can be treated or alleviated by the agents described herein include those respiratory disorders and asthma caused by infectious agents, such as viruses (e.g., cold and flu viruses, respiratory syncytial virus (RSV), paramyxovirus, rhinovirus and influenza viruses. RSV, rhinovirus and influenza virus infections are common in children, and are one leading cause of respiratory tract illnesses in infants and young children. Children with viral bronchiolitis can develop chronic wheezing and asthma, which can be treated using the methods described herein. Also included are the asthma conditions which may be brought about in some asthmatics by exercise and/or cold air. The methods are useful for asthmas associated with smoke exposure (e.g., cigarette-induced and industrial smoke), as well as industrial and occupational exposures, such as smoke, ozone, noxious gases, sulfur dioxide, nitrous oxide, fumes, including isocyanates, from paint, plastics, polyurethanes, varnishes, etc., wood, plant or other organic dusts, etc. The methods are also useful for asthmatic incidents associated with food additives, preservatives or pharmacological agents. Also included are methods for treating, inhibiting or alleviating the types of asthma referred to as silent asthma or cough variant asthma.

The methods disclosed herein are also useful for treatment and alleviation of asthma associated with gastroesophageal reflux (GERD), which can stimulate bronchoconstriction. GERD, along with retained bodily secretions, suppressed cough, and exposure to allergens and irritants in the bedroom can contribute to asthmatic conditions and have been collectively referred to as nighttime asthma or nocturnal asthma. In methods of treatment, inhibition or alleviation of asthma associated with GERD, a pharmaceutically effective amount of the IL-13 and/or IL-4 antagonist can be used as described herein in combination with a pharmaceutically effective amount of an agent for treating GERD. These agents include, but are not limited to, proton pump inhibiting agents like PROTONIX® brand of delayed-release pantoprazole sodium tablets, PRILOSEC® brand omeprazole delayed release capsules, ACIPHEX® brand rebeprazole sodium delayed release tablets or PREV ACID® brand delayed release lansoprazole capsules.

### Atopic Disorders and Symptoms Thereof

It has been observed that cells from atopic patients have enhanced sensitivity to IL-13. Accordingly, an IL-13 and/or IL-4 antagonist can be administered in an amount effective to treat or prevent an atopic disorder. “Atopic” refers to a group of diseases in which there is often an inherited tendency to develop an allergic reaction.

Examples of atopic disorders include allergy, allergic rhinitis, atopic dermatitis, asthma and hay fever. Asthma is a phenotypically heterogeneous disorder associated with intermittent respiratory symptoms such as, e.g., bronchial hyperresponsiveness and reversible airflow obstruction. Immunohistopathologic features of asthma include, e.g., denudation of airway epithelium, collagen deposition beneath the basement membrane; edema; mast cell activation; and inflammatory cell infiltration (e.g., by neutrophils, eosinophils, and lymphocytes). Airway inflammation can further contribute to airway hyperresponsiveness, airflow limitation, acute bronchoconstriction, mucus plug formation, airway wall remodeling, and other respiratory symptoms. An IL-13 binding agent (e.g., an IL-13 binding agent such as an antibody molecule described herein) can be administered in an amount effective to ameliorate one or more of these symptoms.

Symptoms of allergic rhinitis (hay fever) include itchy, runny, sneezing, or stuffy nose, and itchy eyes. An IL-13 and/or IL-4 antagonist can be administered to ameliorate one or more of these symptoms. Atopic dermatitis is a chronic (long-lasting) disease that affects the skin. Information about atopic dermatitis is available, e.g., from NIH Publication No. 03-4272. In atopic dermatitis, the skin can become extremely itchy, leading to redness, swelling, cracking, weeping clear fluid, and finally, crusting and scaling. In many cases, there are periods of time when the disease is worse (called exacerbations or flares) followed by periods when the skin improves or clears up entirely (called remissions). Atopic dermatitis is often referred to as “eczema,” which is a general term for the several types of inflammation of the skin. Atopic dermatitis is the most common of the many types of eczema. Examples of atopic dermatitis include: allergic contact eczema (dermatitis: a red, itchy, weepy reaction where the skin has come into contact with a substance that the immune system recognizes as foreign, such as poison ivy or certain preservatives in creams and lotions); contact eczema (a localized reaction that includes redness, itching, and burning where the skin has come into contact with an allergen (an allergy-causing substance) or with an irritant such as an acid, a cleaning agent, or other chemical); dyshidrotic eczema (irritation of the skin on the palms of hands and soles of the feet characterized by clear, deep blisters that itch and burn); neurodermatitis (scaly patches of the skin on the head, lower legs, wrists, or forearms caused by a localized itch (such as an insect bite) that become intensely irritated when scratched); nummular eczema (coin-shaped patches of irritated skin-most common on the arms, back, buttocks, and lower legs-that may be crusted, scaling, and extremely itchy); seborrheic eczema (yellowish, oily, scaly patches of skin on the scalp, face, and occasionally other parts of the body). Additional particular symptoms include stasis dermatitis, atopic pleat (Dennie-Morgan fold), cheilitis, hyperlinear palms, hyperpigmented eyelids (eyelids that have become darker in color from inflammation or hay fever), ichthyosis, keratosis pilaris, lichenification, papules, and urticaria. An IL-13 or IL-4 antagonist can be administered to ameliorate one or more of these symptoms.

An exemplary method for treating allergic rhinitis or other allergic disorder can include initiating therapy with an IL-13 and/or IL-4 antagonist prior to exposure to an allergen, e.g., prior to seasonal exposure to an allergen, e.g., prior to allergen blooms. Such therapy can include one or more doses, e.g., doses at regular intervals.

### Cancer

IL-13 and its receptors may be involved in the development of at least some types of cancer, e.g., a cancer derived from hematopoietic cells or a cancer derived from brain or neuronal cells (e.g., a glioblastoma). For example, blockade of the IL-13 signaling pathway, e.g., via use of a soluble IL-13 receptor or a STAT6−/− deficient mouse, leads to delayed tumor onset and/or growth of Hodgkins lymphoma cell lines or a metastatic mammary carcinoma, respectively (Trieu et al. (2004) Cancer Res. 64: 3271-75; Ostrand-Rosenberg et al. (2000) J. Immunol. 165: 6015-6019). Cancers that express IL-13R(2 (Husain and Puri (2003) J. Neurooncol. 65:37-48; Mintz et al. (2003) J. Neurooncol. 64:117-23) can be specifically targeted by anti-IL-13 antibodies described herein. IL-13 antagonists can be useful to inhibit cancer cell proliferation or other cancer cell activity. A cancer refers to one or more cells that has a loss of responsiveness to normal growth controls, and typically proliferates with reduced regulation relative to a corresponding normal cell.

Examples of cancers against which IL-13 antagonists (e.g., an IL-13 binding agent such as an antibody or antigen binding fragment described herein) can be used for treatment include leukemias, e.g., B-cell chronic lymphocytic leukemia, acute myelogenous leukemia, and human T-cell leukemia virus type 1 (HTLV-1) transformed T cells; lymphomas, e.g. T cell lymphoma, Hodgkin's lymphoma; glioblastomas; pancreatic cancers; renal cell carcinoma; ovarian carcinoma; AIDS-Kaposi's sarcoma, and breast cancer (as described in Aspord, C. et al. (2007) JEM 204:1037-1047). For example, an IL-13 binding agent (e.g., an anti-IL-13 antibody molecule) can be administered in an amount effective to treat or prevent the disorder, e.g., to reduce cell proliferation, or to ameliorate at least one symptom of the disorder.

### Fibrosis

IL-13 and/or IL-4 antagonists can also be useful in treating inflammation and fibrosis, e.g., fibrosis of the liver. IL-13 production has been correlated with the progression of liver inflammation (e.g., viral hepatitis) toward cirrhosis, and possibly, hepatocellular carcinoma (de Lalla et al. (2004) J. Immunol. 173:1417-1425). Fibrosis occurs, e.g., when normal tissue is replaced by scar tissue, often following inflammation. Hepatitis B and hepatitis C viruses both cause a fibrotic reaction in the liver, which can progress to cirrhosis. Cirrhosis, in turn, can evolve into severe complications such as liver failure or hepatocellular carcinoma. Blocking IL-13 activity using the IL-13 and/or IL-4 antagonists described herein can reduce inflammation and fibrosis, e.g., the inflammation, fibrosis, and cirrhosis associated with liver diseases, especially hepatitis B and C. For example, the antagonists(s) can be administered in an amount effective to treat or prevent the disorder or to ameliorate at least one symptom of the inflammatory and/or fibrotic disorder.

### Inflammatory Bowel Disease

Inflammatory bowel disease (IBD) is the general name for diseases that cause inflammation of the intestines. Two examples of inflammatory bowel disease are Crohn's disease and ulcerative colitis. IL-13/STAT6 signaling has been found to be involved in inflammation-induced hypercontractivity of mouse smooth muscle, a model of inflammatory bowel disease (Akiho et al. (2002) Am. J. Physiol. Gastrointest. Liver Physiol. 282:G226-232). For example, an IL-13 and/or IL-4 antagonist can be administered in an amount effective to treat or prevent the disorder or to ameliorate at least one symptom of the inflammatory bowel disorder.

### Pharmaceutical Compositions

The IL-13 and/or IL-4 antagonists (such as those described herein) can be used in vitro, ex vivo, or in vivo. They can be incorporated into a pharmaceutical composition, e.g., by combining the IL-13 binding agent with a pharmaceutically acceptable carrier. Such a composition may contain, in addition to the IL-13 binding agent and carrier, various diluents, fillers, salts, buffers, stabilizers, solubilizers, and other materials well known in the art. Pharmaceutically acceptable materials is generally a nontoxic material that does not interfere with the effectiveness of the biological activity of an IL-13 binding agent. The characteristics of the carrier can depend on the route of administration.

The pharmaceutical composition described herein may also contain other factors, such as, but not limited to, other anti-cytokine antibody molecules or other anti-inflammatory agents as described in more detail below. Such additional factors and/or agents may be included in the pharmaceutical composition to produce a synergistic effect with an IL-13 and/or IL-4 antagonist described herein. For example, in the treatment of allergic asthma, a pharmaceutical composition described herein may include anti-IL-4 antibody molecules or drugs known to reduce an allergic response.

The pharmaceutical composition described herein may be in the form of a liposome in which an IL-13 and/or IL-4 antagonist, such as one described herein is combined, in addition to other pharmaceutically acceptable carriers, with amphipathic agents such as lipids that exist in aggregated form as micelles, insoluble monolayers, liquid crystals, or lamellar layers while in aqueous solution. Suitable lipids for liposomal formulation include, without limitation, monoglycerides, diglycerides, sulfatides, lysolecithin, phospholipids, saponin, bile acids, and the like. Exemplary methods for preparing such liposomal formulations include methods described in U.S. Pat. Nos. 4,235,871; 4,501,728; 4,837,028; and 4,737,323.

As used herein, the term “therapeutically effective amount” means the total amount of each active component of the pharmaceutical composition or method that is sufficient to show a meaningful patient benefit, e.g., amelioration of symptoms of, healing of, or increase in rate of healing of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

Administration of an IL-13 and/or IL-4 antagonist used in the pharmaceutical composition can be carried out in a variety of conventional ways, such as oral ingestion, inhalation, or cutaneous, subcutaneous, or intravenous injection. When a therapeutically effective amount of an IL-13 and/or IL-4 antagonist is administered by intravenous, cutaneous or subcutaneous injection, the binding agent can be prepared as a pyrogen-free, parenterally acceptable aqueous solution. The composition of such parenterally acceptable protein solutions can be adapted in view factors such as pH, isotonicity, stability, and the like, e.g., to optimize the composition for physiological conditions, binding agent stability, and so forth. A pharmaceutical composition for intravenous, cutaneous, or subcutaneous injection can contain, e.g., an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition may also contain stabilizers, preservatives, buffers, antioxidants, or other additive.

The amount of an IL-13 and/or IL-4 antagonist in the pharmaceutical composition can depend upon the nature and severity of the condition being treated, and on the nature of prior treatments that the patient has undergone. The pharmaceutical composition can be administered to normal patients or patients who do not show symptoms, e.g., in a prophylactic mode. An attending physician may decide the amount of IL-13 and/or IL-4 antagonist with which to treat each individual patient. For example, an attending physician can administer low doses of antagonist and observe the patient's response. Larger doses of antagonist may be administered until the optimal therapeutic effect is obtained for the patient, and at that point the dosage is not generally increased further. For example, a pharmaceutical may contain between about 0.1 mg to 50 mg antibody per kg body weight, e.g., between about 0.1 mg and 5 mg or between about 8 mg and 50 mg antibody per kg body weight. In one embodiment in which the antibody is delivered subcutaneously at a frequency of no more than twice per month, e.g., every other week or monthly, the composition includes an amount of about 0.7-3.3, e.g., 1.0-3.0 mg/kg, e.g., about 0.8-1.2, 1.2-2.8, or 2.8-3.3 mg/kg.

The duration of therapy using the pharmaceutical composition may vary, depending on the severity of the disease being treated and the condition and potential idiosyncratic response of each individual patient. In one embodiment, the IL-13 and/or IL-4 antagonist can also be administered via the subcutaneous route, e.g., in the range of once a week, once every 24, 48, 96 hours, or not more frequently than such intervals. Exemplary dosages can be in the range of 0.1-20 mg/kg, more preferably 1-10 mg/kg. The agent can be administered, e.g., by intravenous infusion at a rate of less than 20, 10, 5, or 1 mg/min to reach a dose of about 1 to 50 mg/m² or about 5 to 20 mg/m².

In one embodiment, an administration of a an IL-13 and/or IL-4 antagonist to the patient includes varying the dosage of the protein, e.g., to reduce or minimize side effects. For example, the subject can be administered a first dosage, e.g., a dosage less than a therapeutically effective amount. In a subsequent interval, e.g., at least 6, 12, 24, or 48 hours later, the patient can be administered a second dosage, e.g., a dosage that is at least 25, 50, 75, or 100% greater than the first dosage. For example, the second and/or a comparable third, fourth and fifth dosage can be at least about 70, 80, 90, or 100% of a therapeutically effective amount.

### Inhalation

A composition that includes an IL-13 and/or IL-4 antagonist can be formulated for inhalation or other mode of pulmonary delivery. The term “pulmonary tissue” as used herein refers to any tissue of the respiratory tract and includes both the upper and lower respiratory tract, except where otherwise indicated. An IL-13 and/or IL-4 antagonist can be administered in combination with one or more of the existing modalities for treating pulmonary diseases.

In one example the an IL-13 and/or IL-4 antagonist is formulated for a nebulizer. In one embodiment, the an IL-13 and/or IL-4 antagonist can be stored in a lyophilized form (e.g., at room temperature) and reconstituted in solution prior to inhalation. It is also possible to formulate the an IL-13 and/or IL-4 antagonist for inhalation using a medical device, e.g., an inhaler. See, e.g., U.S. Pat. No. 6,102,035 (a powder inhaler) and U.S. Pat. No. 6,012,454 (a dry powder inhaler). The inhaler can include separate compartments for the IL-13 and/or IL-4 antagonist at a pH suitable for storage and another compartment for a neutralizing buffer and a mechanism for combining the IL-13 and/or IL-4 antagonist with a neutralizing buffer immediately prior to atomization. In one embodiment, the inhaler is a metered dose inhaler.

The three common systems used to deliver drugs locally to the pulmonary air passages include dry powder inhalers (DPIs), metered dose inhalers (MDIs) and nebulizers. MDIs, the most popular method of inhalation administration, may be used to deliver medicaments in a solubilized form or as a dispersion. Typically MDIs comprise a Freon or other relatively high vapor pressure propellant that forces aerosolized medication into the respiratory tract upon activation of the device. Unlike MDIs, DPIs generally rely entirely on the inspiratory efforts of the patient to introduce a medicament in a dry powder form to the lungs. Nebulizers form a medicament aerosol to be inhaled by imparting energy to a liquid solution. Direct pulmonary delivery of drugs during liquid ventilation or pulmonary lavage using a fluorochemical medium has also been explored. These and other methods can be used to deliver an IL-13 and/or IL-4 antagonist. In one embodiment, the an IL-13 and/or IL-4 antagonist is associated with a polymer, e.g., a polymer that stabilizes or increases half-life of the compound.

For example, for administration by inhalation, an IL-13 and/or IL-4 antagonist is delivered in the form of an aerosol spray from pressured container or dispenser which contains a suitable propellant or a nebulizer. The IL-13 and/or IL-4 antagonist may be in the form of a dry particle or as a liquid. Particles that include the IL-13 and/or IL-4 antagonist can be prepared, e.g., by spray drying, by drying an aqueous solution of the IL-13 and/or IL-4 antagonist with a charge neutralizing agent and then creating particles from the dried powder or by drying an aqueous solution in an organic modifier and then creating particles from the dried powder.

The IL-13 and/or IL-4 antagonist may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges for use in an inhaler or insufflator may be formulated containing a powder mix of an IL-13 and/or IL-4 antagonist and a suitable powder base such as lactose or starch, if the particle is a formulated particle. In addition to the formulated or unformulated compound, other materials such as 100% DPPC or other surfactants can be mixed with the an IL-13 and/or IL-4 antagonist to promote the delivery and dispersion of formulated or unformulated compound. Methods of preparing dry particles are described, for example, in WO 02/32406.

An IL-13 and/or IL-4 antagonist can be formulated for aerosol delivery, e.g., as dry aerosol particles, such that when administered it can be rapidly absorbed and can produce a rapid local or systemic therapeutic result. Administration can be tailored to provide detectable activity within 2 minutes, 5 minutes, 1 hour, or 3 hours of administration. In some embodiments, the peak activity can be achieved even more quickly, e.g., within one half hour or even within ten minutes. An IL-13 and/or IL-4 antagonist can be formulated for longer biological half-life (e.g., by association with a polymer such as PEG) for use as an alternative to other modes of administration, e.g., such that the IL-13 and/or IL-4 antagonist enters circulation from the lung and is distributed to other organs or to a particular target organ.

In one embodiment, the IL-13 and/or IL-4 antagonist is delivered in an amount such that at least 5% of the mass of the polypeptide is delivered to the lower respiratory tract or the deep lung. Deep lung has an extremely rich capillary network. The respiratory membrane separating capillary lumen from the alveolar air space is very thin (≦6 μm) and extremely permeable. In addition, the liquid layer lining the alveolar surface is rich in lung surfactants. In other embodiments, at least 2%, 3%, 5%, 10%, 20%, 30%, 40%, 50%, 60%, 70%, or 80% of the composition of an IL-13 and/or IL-4 antagonist is delivered to the lower respiratory tract or to the deep lung. Delivery to either or both of these tissues results in efficient absorption of the IL-13 and/or IL-4 antagonist and high bioavailability. In one embodiment, the IL-13 and/or IL-4 antagonist is provided in a metered dose using, e.g., an inhaler or nebulizer. For example, the IL-13 binding agent is delivered in a dosage unit form of at least about 0.02, 0.1, 0.5, 1, 1.5, 2, 5, 10, 20, 40, or 50 mg/puff or more. The percent bioavailability can be calculated as follows: the percent bioavailability=(AUCₙₒₙ₋ᵢₙᵥₐₛᵢᵥₑ/AUC_{i.v. or s.c.})×(dose_{i.v. or s.c.}/doseₙₒₙ₋ᵢₙᵥₐₛᵢᵥₑ)×100.

Although not necessary, delivery enhancers such as surfactants can be used to further enhance pulmonary delivery. A “surfactant” as used herein refers to a IL IL-13 and/or IL-4 antagonist having a hydrophilic and lipophilic moiety, which promotes absorption of a drug by interacting with an interface between two immiscible phases. Surfactants are useful in the dry particles for several reasons, e.g., reduction of particle agglomeration, reduction of macrophage phagocytosis, etc. When coupled with lung surfactant, a more efficient absorption of the IL-13 and/or IL-4 antagonist can be achieved because surfactants, such as DPPC, will greatly facilitate diffusion of the compound. Surfactants are well known in the art and include but are not limited to phosphoglycerides, e.g., phosphatidylcholines, L-alpha-phosphatidylcholine dipalmitoyl (DPPC) and diphosphatidyl glycerol (DPPG); hexadecanol; fatty acids; polyethylene glycol (PEG); polyoxyethylene-9-; auryl ether; palmitic acid; oleic acid; sorbitan trioleate (Span 85); glycocholate; surfactin; poloxomer; sorbitan fatty acid ester; sorbitan trioleate; tyloxapol; and phospholipids.

### Stabilization

In one embodiment, an IL-13 and/or IL-4 antagonist is physically associated with a moiety that improves its stabilization and/or retention in circulation, e.g., in blood, serum, lymph, bronchopulmonary lavage, or other tissues, e.g., by at least 1.5, 2, 5, 10, or 50 fold.

For example, an IL-13 and/or IL-4 antagonist can be associated with a polymer, e.g., a substantially non-antigenic polymers, such as polyalkylene oxides or polyethylene oxides. Suitable polymers will vary substantially by weight. Polymers having molecular number average weights ranging from about 200 to about 35,000 (or about 1,000 to about 15,000, and 2,000 to about 12,500) can be used.

For example, an IL-13 and/or IL-4 antagonist can be conjugated to a water soluble polymer, e.g., hydrophilic polyvinyl polymers, e.g. polyvinylalcohol and polyvinylpyrrolidone. A non-limiting list of such polymers includes polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextran sulfate, dextran, dextrins, glycogen, or the polysaccharide subunit of acid mucopolysaccharides, e.g. hyaluronic acid; polymers of sugar alcohols such as polysorbitol and polymannitol; heparin or heparan.

The conjugates of an IL-13 and/or IL-4 antagonist and a polymer can be separated from the unreacted starting materials, e.g., by gel filtration or ion exchange chromatography, e.g., HPLC. Heterologous species of the conjugates are purified from one another in the same fashion. Resolution of different species (e.g. containing one or two PEG residues) is also possible due to the difference in the ionic properties of the unreacted amino acids. See, e.g., WO 96/34015.

Other Uses of IL-13 and/or IL-4 Antagonists

In yet another aspect, the invention features a method for modulating (e.g., decreasing, neutralizing and/or inhibiting) one or more associated activities of IL-13 in vivo by administering an IL-13 and/or IL-4 antagonist described herein in an amount sufficient to inhibit its activity. An IL-13 and/or IL-4 antagonist can also be administered to subjects for whom inhibition of an IL-13-mediated inflammatory response is required. These conditions include, e.g., airway inflammation, asthma, fibrosis, eosinophilia and increased mucus production.

The efficacy of an IL-13 and/or IL-4 antagonist described herein can be evaluated, e.g., by evaluating ability of the antagonist to modulate airway inflammation in cynomolgus monkeys exposed to an Ascaris suum allergen. An IL-13 and/or IL-4 antagonist can be used to neutralize or inhibit one or more IL-13-associated activities, e.g., to reduce IL-13 mediated inflammation in vivo, e.g., for treating or preventing IL-13-associated pathologies, including asthma and/or its associated symptoms.

In one embodiment, an IL-13 and/or IL-4 antagonist, or a pharmaceutical compositions thereof, is administered in combination therapy, i.e., combined with other agents, e.g., therapeutic agents, that are useful for treating pathological conditions or disorders, such as allergic and inflammatory disorders. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

For example, the combination therapy can include one or more IL-13 binding agents (e.g., the IL-13 antagonist alone or in combination with the IL-4 antagonist) that bind to IL-13 and interfere with the formation of a functional IL-13 signaling complex, coformulated with, and/or coadministered with, one or more additional therapeutic agents, e.g., one or more cytokine and growth factor inhibitors, immunosuppressants, anti-inflammatory agents, metabolic inhibitors, enzyme inhibitors, and/or cytotoxic or cytostatic agents, as described in more detail below. Furthermore, one or more IL-13 binding agents (e.g., the IL-13 antagonist alone or in combination with the IL-4 antagonist) may be used in combination with two or more of the therapeutic agents described herein. Such combination therapies may advantageously utilize lower dosages of the administered therapeutic agents, thus avoiding possible toxicities or complications associated with the various monotherapies. Moreover, the therapeutic agents disclosed herein act on pathways that differ from the IL-13/IL-13-receptor pathway, and thus are expected to enhance and/or synergize with the effects of the IL-13 binding agents.

Therapeutic agents that interfere with different triggers of asthma or airway inflammation, e.g., therapeutic agents used in the treatment of allergy, upper respiratory infections, or ear infections, may be used in combination with an IL-13 binding agent (e.g., the IL-13 antagonist alone or in combination with the IL-4 antagonist). In one embodiment, one or more IL-13 binding agents (e.g., the IL-13 antagonist alone or in combination with the IL-4 antagonist) may be coformulated with, and/or coadministered with, one or more additional agents, such as other cytokine or growth factor antagonists (e.g., soluble receptors, peptide inhibitors, small molecules, adhesins), antibody molecules that bind to other targets (e.g., antibodies that bind to other cytokines or growth factors, their receptors, or other cell surface molecules), and anti-inflammatory cytokines or agonists thereof. Non-limiting examples of the agents that can be used in combination with IL-13 binding agents (e.g., the IL-13 antagonist alone or in combination with the IL-4 antagonist) include, but are not limited to, inhaled steroids; beta-agonists, e.g., short-acting or long-acting beta-agonists; antagonists of leukotrienes or leukotriene receptors; combination drugs such as ADVAIR®; IgE inhibitors, e.g., anti-IgE antibodies (e.g., XOLAIR®); phosphodiesterase inhibitors (e.g., PDE4 inhibitors); xanthines; anticholinergic drugs; mast cell-stabilizing agents such as cromolyn; IL-5 inhibitors; eotaxin/CCR3 inhibitors; and antihistamines.

In other embodiments, one or more IL-13 antagonists alone or in combination with one or more IL-4 antagonists can be co-formulated with, and/or coadministered with, one or more anti-inflammatory drugs, immunosuppressants, or metabolic or enzymatic inhibitors. Examples of the drugs or inhibitors that can be used in combination with the IL-13 binding agents include, but are not limited to, one or more of: TNF antagonists (e.g., a soluble fragment of a TNF receptor, e.g., p55 or p75 human TNF receptor or derivatives thereof, e.g., 75 kd TNFR-IgG (75 kD TNF receptor-IgG fusion protein, ENBREL™)); TNF enzyme antagonists, e.g., TNFα converting enzyme (TACE) inhibitors; muscarinic receptor antagonists; TGF-β antagonists; interferon gamma; perfenidone; chemotherapeutic agents, e.g., methotrexate, leflunomide, or a sirolimus (rapamycin) or an analog thereof, e.g., CCI-779; COX2 and cPLA2 inhibitors; NSAIDs; immunomodulators; p38 inhibitors, TPL-2, Mk-2 and NFκB inhibitors.

### Vaccine Formulations

In another aspect, the invention features a method of modifying an immune response associated with immunization. An IL-13 antagonist, alone or in combination with an IL-4 antagonist, can be used to increase the efficacy of immunization by inhibiting IL-13 activity. Antagonists can be administered before, during, or after delivery of an immunogen, e.g., administration of a vaccine. In one embodiment, the immunity raised by the vaccination is a cellular immunity, e.g., an immunity against cancer cells or virus infected, e.g., retrovirus infected, e.g., HIV infected, cells. In one embodiment, the vaccine formulation contains one or more antagonists and an antigen, e.g., an immunogen. In one embodiment, the IL-13 and/or IL-4 antagonists are administered in combination with immunotherapy (e.g., in combination with an allergy immunization with one or more immunogens chosen from ragweed, ryegrass, dust mite and the like. In another embodiment, the antagonist and the immunogen are administered separately, e.g., within one hour, three hours, one day, or two days of each other.

Inhibition of IL-13 can improve the efficacy of, e.g., cellular vaccines, e.g., vaccines against diseases such as cancer and viral infection, e.g., retroviral infection, e.g., HIV infection. Induction of CD8⁺ cytotoxic T lymphocytes (CTL) by vaccines is down modulated by CD4⁺ T cells, likely through the cytokine IL-13. Inhibition of IL-13 has been shown to enhance vaccine induction of CTL response (Ahlers et al. (2002) Proc. Natl. Acad. Sci. USA 99:13020-10325). An IL-13 antagonist can be used in conjunction with a vaccine to increase vaccine efficacy. Cancer and viral infection (such as retroviral (e.g., HIV) infection) are exemplary disorders against which a cellular vaccine response can be effective. Vaccine efficacy is enhanced by blocking IL-13 signaling at the time of vaccination (Ahlers et al. (2002) Proc. Nat. Acad. Sci. USA 99:13020-25). A vaccine formulation may be administered to a subject in the form of a pharmaceutical or therapeutic composition.

### Methods for Diagnosing, Prognosing, and Monitoring Disorders

IL-13 binding agents can be used in vitro and in vivo as diagnostic agents. One exemplary method includes: (i) administering the IL-13 binding agent (e.g., an IL-13 antibody molecule) to a subject; and (ii) detecting the IL-13 binding agent in the subject. The detecting can include determining location of the IL-13 binding agent in the subject. Another exemplary method includes contacting an IL-13 binding agent to a sample, e.g., a sample from a subject. The presence or absence of IL-13 or the level of IL-13 (either qualitative or quantitative) in the sample can be determined.

In another aspect, the present invention provides a diagnostic method for detecting the presence of a IL-13, in vitro (e.g., a biological sample, such as tissue, biopsy) or in vivo (e.g., in vivo imaging in a subject). The method includes: (i) contacting a sample with IL-13 binding agent; and (ii) detecting formation of a complex between the IL-13 binding agent and the sample. The method can also include contacting a reference sample (e.g., a control sample) with the binding agent, and determining the extent of formation of the complex between the binding agent an the sample relative to the same for the reference sample. A change, e.g., a statistically significant change, in the formation of the complex in the sample or subject relative to the control sample or subject can be indicative of the presence of IL-13 in the sample.

Another method includes: (i) administering the IL-13 binding agent to a subject; and (ii) detecting formation of a complex between the IL-13 binding agent and the subject. The detecting can include determining location or time of formation of the complex.

The IL-13 binding agent can be directly or indirectly labeled with a detectable substance to facilitate detection of the bound or unbound protein. Suitable detectable substances include various enzymes, prosthetic groups, fluorescent materials, luminescent materials and radioactive materials.

Complex formation between the IL-13 binding agent and IL-13 can be detected by measuring or visualizing either the binding agent bound to the IL-13 or unbound binding agent. Conventional detection assays can be used, e.g., an enzyme-linked immunosorbent assays (ELISA), a radioimmunoassay (RIA) or tissue immunohistochemistry. Further to labeling the IL-13 binding agent, the presence of IL-13 can be assayed in a sample by a competition immunoassay utilizing standards labeled with a detectable substance and an unlabeled IL-13 binding agent. In one example of this assay, the biological sample, the labeled standards and the IL-13 binding agent are combined and the amount of labeled standard bound to the unlabeled binding agent is determined. The amount of IL-13 in the sample is inversely proportional to the amount of labeled standard bound to the IL-13 binding agent.

Methods for Diagnosing Prognosing, and/or Monitoring Asthma

The binding agents described herein can be used, e.g., in methods for diagnosing, prognosing, and monitoring the progress of asthma by measuring the level of IL-13 in a biological sample. In addition, this discovery enables the identification of new inhibitors of IL-13 signaling, which will also be useful in the treatment of asthma. Such methods for diagnosing allergic and nonallergic asthma can include detecting an alteration (e.g., a decrease or increase) of IL-13 in a biological sample, e.g., serum, plasma, bronchoalveolar lavage fluid, sputum, etc. “Diagnostic” or “diagnosing” means identifying the presence or absence of a pathologic condition. Diagnostic methods involve detecting the presence of IL-13 by determining a test amount of IL-13 polypeptide in a biological sample, e.g., in bronchoalveolar lavage fluid, from a subject (human or nonhuman mammal), and comparing the test amount with a normal amount or range (i.e., an amount or range from an individual(s) known not to suffer from asthma) for the IL-13 polypeptide. While a particular diagnostic method may not provide a definitive diagnosis of asthma, it suffices if the method provides a positive indication that aids in diagnosis.

Methods for prognosing asthma and/or atopic disorders can include detecting upregulation of IL-13, at the mRNA or protein level. “Prognostic” or “prognosing” means predicting the probable development and/or severity of a pathologic condition. Prognostic methods involve determining the test amount of IL-13 in a biological sample from a subject, and comparing the test amount to a prognostic amount or range (i.e., an amount or range from individuals with varying severities of asthma) for IL-13. Various amounts of the IL-13 in a test sample are consistent with certain prognoses for asthma. The detection of an amount of IL-13 at a particular prognostic level provides a prognosis for the subject.

The present application also provides methods for monitoring the course of asthma by detecting the upregulation of IL-13. Monitoring methods involve determining the test amounts of IL-13 in biological samples taken from a subject at a first and second time, and comparing the amounts. A change in amount of IL-13 between the first and second time can indicate a change in the course of asthma and/or atopic disorder, with a decrease in amount indicating remission of asthma, and an increase in amount indicating progression of asthma and/or atopic disorder. Such monitoring assays are also useful for evaluating the efficacy of a particular therapeutic intervention (e.g., disease attenuation and/or reversal) in patients being treated for an IL-13 associated disorder.

Fluorophore- and chromophore-labeled binding agents can be prepared. The fluorescent moieties can be selected to have substantial absorption at wavelengths above 310 nm, and preferably above 400 nm. A variety of suitable fluorescers and chromophores are described by Stryer (1968) Science, 162:526 and Brand, L. et al. (1972) Annual Review of Biochemistry, 41:843-868. The binding agents can be labeled with fluorescent chromophore groups by conventional procedures such as those disclosed in U.S. Pat. Nos. 3,940,475, 4,289,747, and 4,376,110. One group of fluorescers having a number of the desirable properties described above is the xanthene dyes, which include the fluoresceins and rhodamines. Another group of fluorescent compounds are the naphthylamines. Once labeled with a fluorophore or chromophore, the binding agent can be used to detect the presence or localization of the IL-13 in a sample, e.g., using fluorescent microscopy (such as confocal or deconvolution microscopy).

Histological Analysis. Immunohistochemistry can be performed using the binding agents described herein. For example, in the case of an antibody, the antibody can synthesized with a label (such as a purification or epitope tag), or can be detectably labeled, e.g., by conjugating a label or label-binding group. For example, a chelator can be attached to the antibody. The antibody is then contacted to a histological preparation, e.g., a fixed section of tissue that is on a microscope slide. After an incubation for binding, the preparation is washed to remove unbound antibody. The preparation is then analyzed, e.g., using microscopy, to identify if the antibody bound to the preparation. The antibody (or other polypeptide or peptide) can be unlabeled at the time of binding. After binding and washing, the antibody is labeled in order to render it detectable.

Protein Arrays. An IL-13 binding agent (e.g., a protein that is an IL-13 binding agent) can also be immobilized on a protein array. The protein array can be used as a diagnostic tool, e.g., to screen medical samples (such as isolated cells, blood, sera, biopsies, and the like). The protein array can also include other binding agents, e.g., ones that bind to IL-13 or to other target molecules.

Methods of producing protein arrays are described, e.g., in De Wildt et al. (2000) Nat. Biotechnol. 18:989-994; Lueking et al. (1999) Anal. Biochem. 270:103-111; Ge (2000) Nucleic Acids Res. 28, e3, I-VII; MacBeath and Schreiber (2000) Science 289:1760-1763; WO 01/40803 and WO 99/51773A1. Polypeptides for the array can be spotted at high speed, e.g., using commercially available robotic apparati, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer. For example, the array can be an array of antibodies, e.g., as described in De Wildt, supra. Cells that produce the protein can be grown on a filter in an arrayed format. proteins production is induced, and the expressed protein are immobilized to the filter at the location of the cell.

A protein array can be contacted with a sample to determine the extent of IL-13 in the sample. If the sample is unlabeled, a sandwich method can be used, e.g., using a labeled probe, to detect binding of the IL-13. Information about the extent of binding at each address of the array can be stored as a profile, e.g., in a computer database. The protein array can be produced in replicates and used to compare binding profiles, e.g., of different samples.

Flow Cytometry. The IL-13 binding agent can be used to label cells, e.g., cells in a sample (e.g., a patient sample). The binding agent can be attached (or attachable) to a fluorescent compound. The cells can then be analyzed by flow cytometry and/or sorted using fluorescent activated cell sorted (e.g., using a sorter available from Becton Dickinson Immunocytometry Systems, San Jose Calif.; see also U.S. Pat. Nos. 5,627,037; 5,030,002; and 5,137,809). As cells pass through the sorter, a laser beam excites the fluorescent compound while a detector counts cells that pass through and determines whether a fluorescent compound is attached to the cell by detecting fluorescence. The amount of label bound to each cell can be quantified and analyzed to characterize the sample. The sorter can also deflect the cell and separate cells bound by the binding agent from those cells not bound by the binding agent. The separated cells can be cultured and/or characterized.

In vivo Imaging. In still another embodiment, the invention provides a method for detecting the presence of a IL-13 within a subject in vivo. The method includes (i) administering to a subject (e.g., a patient having an IL-13 associated disorder) an anti-IL-13 antibody molecule, conjugated to a detectable marker; (ii) exposing the subject to a means for detecting the detectable marker. For example, the subject is imaged, e.g., by NMR or other tomographic means.

Examples of labels useful for diagnostic imaging include radiolabels such as ¹³¹I, ¹¹¹In, ¹²³I, ^{99m}Tc, ³²P, ³³P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The binding agent can be labeled with such reagents using known techniques. For example, see Wensel and Meares (1983) Radioimmunoimaging and Radioimmunotherapy, Elsevier, New York for techniques relating to the radiolabeling of antibodies and Colcher et al. (1986) Meth. Enzymol. 121: 802-816. A radiolabeled binding agent can also be used for in vitro diagnostic tests. The specific activity of a isotopically-labeled binding agent depends upon the half-life, the isotopic purity of the radioactive label, and how the label is incorporated into the antibody. Procedures for labeling polypeptides with the radioactive isotopes (such as ¹⁴C, ³H, ³⁵S, ¹²⁵I, ^{99m}Tc, ³²P, ³³P, and ¹³¹I) are generally known. See, e.g., U.S. Pat. No. 4,302,438; Goding, J. W. (Monoclonal antibodies: principles and practice: production and application of monoclonal antibodies in cell biology, biochemistry, and immunology 2nd ed. London; Orlando: Academic Press, 1986. pp 124-126) and the references cited therein; and A. R. Bradwell et al., “Developments in Antibody Imaging”, Monoclonal Antibodies for Cancer Detection and Therapy, R. W. Baldwin et al., (eds.), pp 65-85 (Academic Press 1985).

IL-13 binding agents described herein can be conjugated to Magnetic Resonance Imaging (MRI) contrast agents. Some MRI techniques are summarized in EP-A-0 502 814. Generally, the differences in relaxation time constants T1 and T2 of water protons in different environments is used to generate an image. However, these differences can be insufficient to provide sharp high resolution images. The differences in these relaxation time constants can be enhanced by contrast agents. Examples of such contrast agents include a number of magnetic agents paramagnetic agents (which primarily alter T1) and ferromagnetic or superparamagnetic (which primarily alter T2 response). Chelates (e.g., EDTA, DTPA and NTA chelates) can be used to attach (and reduce toxicity) of some paramagnetic substances (e.g., Fe³⁺, Mn²⁺, Gd³⁺). Other agents can be in the form of particles, e.g., less than 10 μm to about 10 nm in diameter) and having ferromagnetic, antiferromagnetic, or superparamagnetic properties. The IL-13 binding agents can also be labeled with an indicating group containing the NMR active ¹⁹F atom, as described by Pykett (1982) Scientific American, 246:78-88 to locate and image IL-13 distribution.

Also within the scope described herein are kits comprising an IL-13 binding agent and instructions for diagnostic use, e.g., the use of the IL-13 binding agent (e.g., an antibody molecule or other polypeptide or peptide) to detect IL-13, in vitro, e.g., in a sample, e.g., a biopsy or cells from a patient having an IL-13 associated disorder, or in vivo, e.g., by imaging a subject. The kit can further contain a least one additional reagent, such as a label or additional diagnostic agent. For in vivo use the binding agent can be formulated as a pharmaceutical composition.

### Kits

An IL-13 binding agent, e.g., an anti-IL-13 antibody molecule, and/or the IL-4 antagonist can be provided in a kit, e.g., as a component of a kit. For example, the kit includes (a) an IL-13 binding agent, e.g., an anti-IL-13 antibody molecule, and/or the IL-4 antagonist and, optionally (b) informational material. The informational material can be descriptive, instructional, marketing or other material that relates to a method, e.g., a method described herein. The informational material of the kits is not limited in its form. In one embodiment, the informational material can include information about production of the compound, molecular weight of the compound, concentration, date of expiration, batch or production site information, and so forth. In one embodiment, the informational material relates to using the IL-13 binding agent to treat, prevent, diagnose, prognose, or monitor a disorder described herein. In one embodiment the informational material includes instructions for administration of the IL-13 binding as a single treatment interval.

In one embodiment, the informational material can include instructions to administer an IL-13 binding agent, e.g., an anti-IL-13 antibody molecule, in a suitable manner to perform the methods described herein, e.g., in a suitable dose, dosage form, or mode of administration (e.g., a dose, dosage form, or mode of administration described herein). In another embodiment, the informational material can include instructions to administer an IL-13 binding agent, e.g., an anti-IL-13 antibody molecule, to a suitable subject, e.g., a human, e.g., a human having, or at risk for, allergic asthma, non-allergic asthma, or an IL-13 mediated disorder, e.g., an allergic and/or inflammatory disorder, or HTLV-1 infection. IL-13 production has been correlated with HTLV-1 infection (Chung et al., (2003) Blood 102: 4130-36).

For example, the material can include instructions to administer an IL-13 binding agent, e.g., an anti-IL-13 antibody molecule, to a patient, a patient with or at risk for allergic asthma, non-allergic asthma, or an IL-13 mediated disorder, e.g., an allergic and/or inflammatory disorder, or HTLV-1 infection.

The kit can include one or more containers for the composition containing an IL-13 binding agent, e.g., an anti-IL-13 antibody molecule. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an IL-13 binding agent, e.g., anti-IL-13 antibody molecule. For example, the kit includes a plurality of syringes, ampules, foil packets, atomizers or inhalation devices, each containing a single unit dose of an IL-13 binding agent, e.g., an anti-IL-13 antibody molecule, or multiple unit doses.

The kit optionally includes a device suitable for administration of the composition, e.g., a syringe, inhalant, pipette; forceps, measured spoon, dropper (e.g., eye dropper), swab (e.g., a cotton swab or wooden swab), or any such delivery device. In a preferred embodiment, the device is an implantable device that dispenses metered doses of the binding agent.

The Examples that follow are set forth to aid in the understanding of the inventions but are not intended to, and should not be construed to, limit its scope in any way.

### EXAMPLES

### Example 1

### (a) Cloning of NHP-IL-13 and Homology to Human IL-13

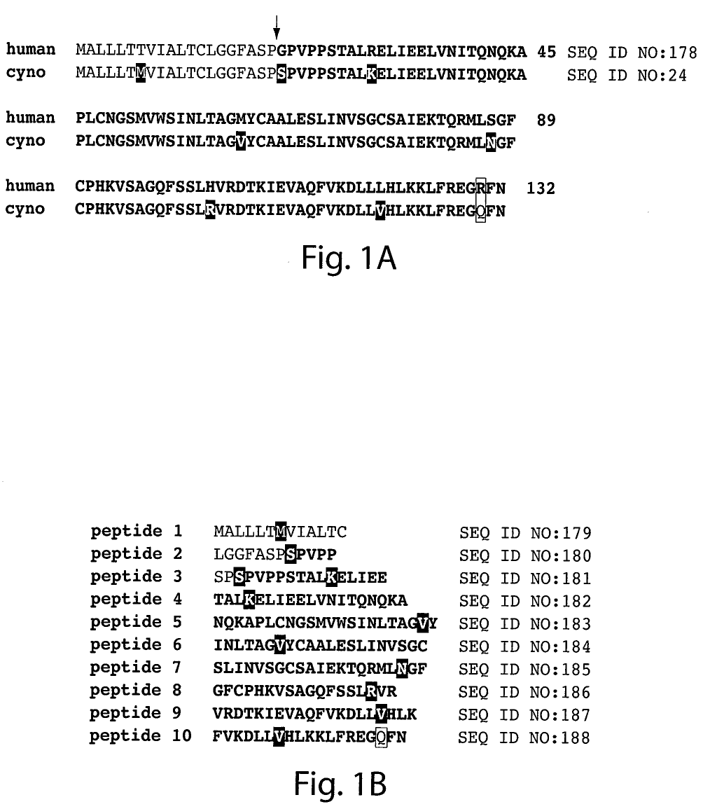

The cynomolgus monkey IL-13 (NHP IL-13) was cloned using hybridization probes. A comparison of the cynomolgus monkey IL-13 amino acid sequence to that of human IL-13 is shown in FIG. 1A. There is 94% amino acid identity between the two sequences, due to 8 amino acid differences. One of these differences, R130Q, represents a common human polymorphism preferentially expressed in asthmatic subjects (Heinzmann et al. (2000) Hum. Mol. Genet. 9:549-559).

### (b) Binding of NHP-IL-13 to Human IL13Rα2

Human IL-13 binds with high affinity to the alpha2 form of IL-13 receptor (IL13Rα2). A soluble form of this receptor was expressed with a human IgG1 Fc tail (sIL13Rα2-Fc). By binding to IL-13 and sequestering the cytokine from the cell surface IL13Rα1-IL4R signaling complex, sIL13Rα2-Fc can act as a potent inhibitor of human IL-13 bioactivity. sIL13Rα2-Fc was shown to bind to NHP-IL-13 produced by CHO cells or E. coli.

### (c) Bioactivity of NHP-IL-13 on Human Monocytes

(i) CD23 expression on human monocytes. cDNA encoding cynomolgus monkey IL-13 was expressed in E. coli and refolded to maintain bioactivity. Reactivity of human cells to cynomolgus IL-13 was demonstrated using a bioassay in which normal peripheral blood mononuclear cells from healthy donors were treated with IL-13 overnight at 37° C. This induced up-regulation of CD23 expression on the surface of monocytes. Results showed that cynomolgus IL-13 had bioactivity on primary human monocytes.

(ii) STAT6 phosphorylation on HT-29 cells. The human HT-29 epithelial cell line responds to IL-13 by undergoing STAT6 phosphorylation, a consequence of signal transduction through the IL-13 receptor. To assay the ability of recombinant NHP-IL-13 to induce STAT6 phosphorylation, HT-29 cells were challenged with the NHP-IL-13 for 30 minutes at 37° C., then fixed, permeabilized, and stained with fluorescent antibody to phospho-STAT6. Results showed that cynomolgus IL-13 efficiently induced STAT6 phosphorylation in this human cell line.

### (d) Generation of Antibodies that Bind to NHP-IL-13

Mice or other appropriate animals may be immunized and boosted with cynomolgus IL-13, e.g., using one or more of the following methods. One method for immunization may be combined with either the same or different method for boosting:

(i) Immunization with cynomolgus IL-13 protein expressed in E. coli, purified from inclusion bodies, and refolded to preserve biological activity. For immunization, the protein is emulsified with complete Freund's adjuvant (CFA), and mice are immunized according to standard protocols. For boosting, the same protein is emulsified with incomplete Freund's adjuvant (IFA).

(ii) Immunization with peptides spanning the entire sequence of mature cynomolgus IL-13. Each peptide contains at least one amino acid that is unique to cynomolgus IL-13 and not present in the human protein. See FIG. 1B. Where the peptide has a C-terminal residue other than cysteine, a cysteine is added for conjugation to a carrier protein. The peptides are conjugated to an immunogenic carrier protein such as KLH, and used to immunize mice according to standard protocols. For immunization, the protein is emulsified with complete Freund's adjuvant (CFA), and mice are immunized according to standard protocols. For boosting, the same protein is emulsified with incomplete Freund's adjuvant (IFA).

(iii) Immunization with NHP-IL-13-encoding cDNA expressed. The cDNA encoding NHP-IL-13, including leader sequence, is cloned into an appropriate vector. This DNA is coated onto gold beads which are injected intradermally by gene gun.

(iv) The protein or peptides can be used as a target for screening a protein library, e.g., a phage or ribosome display library. For example, the library can display varied immunoglobulin molecules, e.g., Fab's, scFv's, or Fd's.

(e) Selection of antibody clones cross-reactive with NHP and optionally a human IL-13, e.g., a native human IL-13.

Primary Screen

The primary screen for antibodies was selection for binding to recombinant NHP-IL-13 by ELISA. In this ELISA, wells are coated with recombinant NHP IL-13. The immune serum was added in serial dilutions and incubated for one hour at room temperature. Wells were washed with PBS containing 0.05% TWEEN®-20 (PBS-Tween). Bound antibody was detected using horseradish peroxidase (HRP)-labeled anti-mouse IgG and tetramethylbenzidene (TMB) substrate. Absorbance was read at 450 mm. Typically, all immunized mice generated high titers of antibody to NHP-IL-13.

Secondary Screen

The secondary screen was selection for inhibition of binding of recombinant NHP-IL-13 to sIL-13Rα1-Fc by ELISA. Wells were coated with soluble IL-13Rα1-Fc, to which FLAG-tagged NHP-IL-13 could bind. This binding was detected with anti-FLAG antibody conjugated to HRP. Hydrolysis of TMB substrate was read as absorbance at 450 nm. In the assay, the FLAG-tagged NHP-IL-13 was added together with increasing concentrations of immune serum. If the immune serum contained antibody that bound to NHP-IL-13 and prevented its binding to the sIL13Rα1-Fc coating the wells, the ELISA signal was decreased. All immunized mice produced antibody that competed with sIL13Rα1-Fc binding to NHP-IL-13, but the titers varied from mouse to mouse. Spleens were selected for fusion from animals whose serum showed inhibited sIL13Rα1-Fc binding to NHP-IL-13 at the highest dilution.

Tertiary Screen

The tertiary screen tested for inhibition of NHP-IL-13 bioactivity. Several bioassays were available to be used, including the TF-1 proliferation assay, the monocyte CD23 expression assay, and the HT-29 cell STAT6 phosphorylation assay. Immune sera were tested for inhibition of NHP-IL-13-mediated STAT6 phosphorylation. The HT-29 human epithelial cell line was challenged for 30 minutes at 37° C. with recombinant NHP-IL-13 in the presence or absence of the indicated concentration of mouse immune serum. Cells were then fixed, permeabilized, and stained with ALEXA™ Fluor 488-conjugated mAb to phospho-STAT6 (Pharmingen). The percentage of cells responding to IL-13 by undergoing STAT6 phosphorylation was determined by flow cytometry. Spleens of mice with the most potent neutralization activity, determined as the strongest inhibition of NHP-IL-13 bioactivity at a high serum dilution, were selected for generation of hybridomas.

Quaternary Screen

A crude preparation containing human IL-13 was generated from human umbilical cord blood mononuclear cells (BioWhittaker/Cambrex). The cells were cultured in a 37° C. incubator at 5% CO₂, in RPMI media containing 10% heat-inactivated FCS, 50 U/ml penicillin, 50 mg/ml streptomycin, and 2 mM L-glutamine. Cells were stimulated for 3 days with the mitogen PHA-P (Sigma), and skewed toward Th2 with recombinant human IL-4 (R&D Systems) and anti-human IL-12. The Th2 cells were expanded for one week with IL-2, then activated to produce cytokine by treatment with phorbol 12-myristate 13-acetate (PMA) and ionomycin for three days. The supernatant was collected and dialyzed to remove PMA and ionomycin. To deplete GM-CSF and IL-4, which could interfere with bioassays for IL-13, the supernatant was treated with biotinylated antibodies to GM-CSF and IL-4 (R&D Systems, Inc), then incubated with streptavidin-coated magnetic beads (Dynal). The final concentration of IL-13 was determined by ELISA (Biosource), and for total protein by Bradford assay (Bio-Rad). The typical preparation contains <0.0005% IL-13 by weight.

Selection of Hybridoma Clones

Using established methods, hybridomas were generated from spleens of mice selected as above, fused to the P3×63_AG8.653 myeloma cell line (ATCC). Cells were plated at limiting dilution and clones were selected according to the screening criteria described above. Data was collected for the selection of clones based on ability to compete for NHP-IL-13 binding to sIL13Rα1-Fc by ELISA. Clones were further tested for ability to neutralize the bioactivity of NHP-IL-13. Supernatants of the hybridomas were tested for competition of STAT-6 phosphorylation induced by NHP-IL-13 in the HT-29 human epithelial cell line.

### Example 2

### MJ 2-7 Antibody

Total RNA was prepared from MJ 2-7 hybridoma cells using the QIAGEN RNEASY™ Mini Kit (Qiagen). RNA was reverse transcribed to cDNA using the SMART™ PCR Synthesis Kit (BD Biosciences Clontech). The variable region of MJ 2-7 heavy chain was extrapolated by PCR using SMART™ oligonucleotide as a forward primer and mIgG1 primer annealing to DNA encoding the N-terminal part of CH1 domain of mouse IgG1 constant region as a reverse primer. The DNA fragment encoding MJ 2-7 light chain variable region was generated using SMART™ and mouse kappa specific primers. The PCR reaction was performed using DEEP VENT™ DNA polymerase (New England Biolabs) and 25 nM of dNTPs for 24 cycles (94° C. for 1 minute, 60° C. for 1 minute, 72° C. for 1 minute). The PCR products were subcloned into the pED6 vector, and the sequence of the inserts was identified by DNA sequencing. N-terminal protein sequencing of the purified mouse MJ 2-7 antibody was used to confirm that the translated sequences corresponded to the observed protein sequence.

Exemplary nucleotide and amino acid sequences of mouse monoclonal antibody MJ 2-7 which interacts with NHP IL-13 and which has characteristics which suggest that it may interact with human IL-13 are as follows:

An exemplary nucleotide sequence encoding the heavy chain variable domain includes:

(SEQ ID NO:129)

GAG GTTCAGCTGC AGCAGTCTGG GGCAGAGCTT GTGAAGCCAG
GGGCCTCAGT CAAGTTGTCC TGCACAGGTT CTGGCTTCAA
CATTAAAGAC ACCTATATAC ACTGGGTGAA GCAGAGGCCT
GAACAGGGCC TGGAGTGGAT TGGAAGGATT GATCCTGCGA
ATGATAATAT TAAATATGAC CCGAAGTTCC AGGGCAAGGC
CACTATAACA GCAGACACAT CCTCCAACAC AGCCTACCTA
CAGCTCAACA GCCTGACATC TGAGGACACT GCCGTCTATT
ACTGTGCTAG ATCTGAGGAA AATTGGTACG ACTTTTTTGA
CTACTGGGGC CAAGGCACCA CTCTCACAGT CTCCTCA

An exemplary amino acid sequence for the heavy chain variable domain includes:

(SEQ ID NO:130)

EVQLQQSGAELVKPGASVKLSCTGSGFNIKDTYIHWVKQRPEQGLEWIGR
IDPANDNIKYDPKFQGKATITADTSSNTAYLQLNSLTSEDTAVYYCARSE
ENWYDFFDYWGQGTTLTVSS

CDRs are underlined. The variable domain optionally is preceded by a leader sequence. e.g., MKCSWVIFFLMAVVTGVNS (SEQ ID NO:131). An exemplary nucleotide sequence encoding the light chain variable domain includes:

(SEQ ID NO:132)

GAT GTTTTGATGA CCCAAACTCC ACTCTCCCTG CCTGTCAGTC
TTGGAGATCA AGCCTCCATC TCTTGCAGGT CTAGTCAGAG
CATTGTACAT AGTAATGGAA ACACCTATTT AGAATGGTAC
CTGCAGAAAC CAGGCCAGTC TCCAAAGCTC CTGATCTACA
AAGTTTCCAA CCGATTTTCT GGGGTCCCAG ACAGGTTCAG
TGGCAGTGGA TCAGGGACAG ATTTCACACT CAAGATTAGC
AGAGTGGAGG CTGAGGATCT GGGAGTTTAT TACTGCTTTC
AAGGTTCACA TATTCCGTAC ACGTTCGGAG GGGGGACCAA
GCTGGAAATA AAA

An exemplary amino acid sequence for the light chain variable domain includes:

(SEQ ID NO:133)

DVLMTQTPLSLPVSLGDQASISCRSSQSIVHSNGNTYLEWYLQKPGQSPK
LLIYKVSNRFSGVPDRFSGSGSGTDFTLKISRVEAEDLGVYYCFQGSHIP
YTFGGGTKLEIK

CDRs are underlined. The amino acid sequence optionally is preceded by a leader sequence, e.g., MKLPVRLLVLMFWIPASSS (SEQ ID NO:134). The term “MJ 2-7” is used interchangeably with the term “mAb7.1.1,” herein.

### Example 3

### C65 Antibody

Exemplary nucleotide and amino acid sequences of mouse monoclonal antibody C65, which interacts with NHP IL-13 and which has characteristics that suggest that it may interact with human IL-13 are as follows:

An exemplary nucleic acid sequence for the heavy chain variable domain includes:

| (SEQ ID NO:135) | |
|---|---|
| 1 | ATGGCTGTCC TGGCATTACT CTTCTGCCTG GTAACATTCC CAAGCTGTAT |
| 51 | CCTTTCCCAG GTGCAGCTGA AGGAGTCAGG ACCTGGCCTG GTGGCGCCCT |
| 101 | CACAGAGCCT GTCCATCACA TGCACCGTCT CAGGGTTCTC ATTAACCGGC |
| 151 | TATGGTGTAA ACTGGGTTCG CCAGCCTCCA GGAAAGGGTC TGGAGTGGCT |
| 201 | GGGAATAATT TGGGGTGATG GAAGCACAGA CTATAATTCA GCTCTCAAAT |
| 251 | CCAGACTGAT CATCAACAAG GACAACTCCA AGAGCCAAGT TTTCTTAAAA |
| 301 | ATGAACAGTC TGCAAACTGA TGACACAGCC AGGTACTTCT GTGCCAGAGA |
| 351 | TAAGACTTTT TACTACGATG GTTTCTACAG GGGCAGGATG GACTACTGGG |
| 401 | GTCAAGGAAC CTCAGTCACC GTCTCCTCA |

An exemplary amino acid sequence for the heavy chain variable domain includes:

(SEQ ID NO:136)

QVQLKESGPGL VAPSQSLSIT CTVSGFSLTG YGVNWVRQPP
GKGLEWLGII WGDGSTDYNS ALKSRLIINK DNSKSQVFLK
MNSLQTDDTA RYFCARDKTF YYDGFYRGRM DYWGQGTSVT VSS

CDRs are underlined. The amino acid sequence optionally is preceded by a leader sequence, e.g., MAVLALLFCL VTFPSCILS (SEQ ID NO:137).

An exemplary nucleotide sequence encoding the light chain variable domain includes:

| (SEQ ID NO:138) | |
|---|---|
| 1 | ATGAACACGA GGGCCCCTGC TGAGTTCCTT GGGTTCCTGT TGCTCTGGTT |
| 51 | TTTAGGTGCC AGATGTGATG TCCAGATGAT TCAGTCTCCA TCCTCCCTGT |
| 101 | CTGCATCTTT GGGAGACATT GTCACCATGA CTTGCCAGGC AAGTCAGGGC |
| 151 | ACTAGCATTA ATTTAAACTG GTTTCAGCAA AAACCAGGGA AAGCTCCTAA |
| 201 | GCTCCTGATC TTTGGTGCAA GCAACTTGGA AGATGGGGTC CCATCAAGGT |
| 251 | TCAGTGGCAG TAGATATGGG ACAAATTTCA CTCTCACCAT CAGCAGCCTG |
| 301 | GAGGATGAAG ATATGGCAAC TTATTTCTGT CTACAGCATA GTTATCTCCC |
| 351 | GTGGACGTTC GGTGGCGGCA CCAAACTGGA AATCAAA |

An exemplary amino acid sequence for the light chain variable domain includes:

(SEQ ID NO:139)

DVQMIQSP SSLSASLGDI VTMTCQASQG TSINLNWFQQ
KPGKAPKLLI FGASNLEDGV PSRFSGSRYG TNFTLTISSL
EDEDMATYFC LQHSYLPWTF GGGTKLEIK

CDRs are underlined. The amino acid sequence optionally is preceded by a leader sequence, e.g., MNTRAPAEFLGFLLLWFLGARC (SEQ ID NO:140).

### Example 4

### Fc Sequences

The Ser at position #1 of SEQ ID NO:128 represents amino acid residue #119 in a first exemplary full length antibody numbering scheme in which the Ser is preceded by residue #118 of a heavy chain variable domain. In the first exemplary full length antibody numbering scheme, mutated amino acids are at numbered 234 and 237, and correspond to positions 116 and 119 of SEQ ID NO:128. Thus, the following sequence represents an Fc domain with two mutations: L234A and G237A, according to the first exemplary full length antibody numbering scheme. Mus musculus (SEQ ID NO:128)

The following is another exemplary human Fc domain sequence:

(SEQ ID NO:141)

STKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQ
QGNVFSCSVMHEALHNHYTQKSLSLSPGK

Other exemplary alterations that can be used to decrease effector function include L234A; L235A), (L235A; G237A), and N297A.

### Example 5

### IL-13 and IgE in Mice

IL-13 is involved in the production of IgE, an important mediator of atopic disease. Mice deficient in IL-13 had partial reductions in serum IgE and mast cell IgE responses, whereas mice lacking the natural IL-13 binding agent, IL-13Rα2−/−, had enhanced levels of IgE and IgE effector function.

BALB/c female mice were obtained from Jackson Laboratories (Bar Harbor, Me.). IL-13Rα2−/− mice are described, e.g., in Wood et al. (2003) J. Exp. Med. 197:703-9. Mice deficient in IL-13 are described, e.g., in McKenzie et al. (1998) Immunity 9:423-32. All mutant strains were on the BALB/c background.

Serum IgE levels were measured by ELISA. ELISA plates (MaxiSorp; Nunc, Rochester, N.Y.) were coated overnight at 4° C. with rat anti-mouse IgE (BD Biosciences, San Diego, Calif.). Plates were blocked for 1 hour at room temperature with 0.5% gelatin in PBS, washed in PBS containing 0.05% TWEEN®-20 (PBS-Tween), and incubated for six hours at room temperature with purified mouse IgE (BD Biosciences) as standards or with serum dilutions. Binding was detected with biotinylated anti-mouse IgE (BD Biosciences) using mouse IgG (Sigma-Aldrich, St. Louis, Mo.) as a blocker. Binding was detected with peroxidase-linked streptavidin (Southern Biotechnology Associates, Inc., Birmingham, Ala.) and SURE BLUE™ substrate (KPL Inc., Gaithersburg, Md.).

In order to investigate the requirement for IL-13 to support resting IgE levels in naive mice, serum was examined in the absence of specific immunization from wild-type mice and from mice genetically deficient in IL-13 and IL-13Rα2. Mice deficient in IL-13 had virtually undetectable levels of serum IgE. In contrast, mice lacking the inhibitory receptor IL-13Rα2 displayed elevated levels of serum IgE. These results demonstrate that blocking IL-13 can be useful for treating or preventing atopic disorders.

### Example 6

### IL-13 and Atopic Disorders

The ability of MJ2-7 to inhibit the bioactivity of native human IL-13 (at 1 ng/ml) was evaluated in an assay for STAT6 phosphorylation. MJ2-7 inhibited the activity of native human IL-13 with an IC₅₀ of about 0.293 nM in this assay. An antibody with the murine heavy chain of MJ2-7 and a humanized light chain inhibited the activity of native human IL-13 with an IC₅₀ of about 0.554 nM in this assay.

The ability of MJ2-7 to inhibit non-human primate IL-13 (at 1 ng/ml) was evaluated in an assay for CD23 expression. The MJ2-7 inhibited the activity of non-human primate IL-13 with an IC₅₀ of about 0.242 nM in this assay. An antibody with the murine heavy chain of MJ2-7 and a humanized light chain inhibited the activity of non-human primate IL-13 with an IC₅₀ of about 0.308 nM in this assay.

### Example 7

### Nucleotide and Amino Acid Sequences of Mouse MJ 2-7 Antibody

The nucleotide sequence encoding the heavy chain variable region (with an optional leader) is as follows:

| (SEQ ID NO:142) | |
|---|---|
| 1 | ATGAAATGCA GCTGGGTTAT CTTCTTCCTG ATGGCAGTGG TTACAGGGGT |
| 51 | CAATTCAGAG GTTCAGCTGC AGCAGTCTGG GGCAGAGCTT GTGAAGCCAG |
| 101 | GGGCCTCAGT CAAGTTGTCC TGCACAGGTT CTGGCTTCAA CATTAAAGAC |
| 151 | ACCTATATAC ACTGGGTGAA GCAGAGGCCT GAACAGGGCC TGGAGTGGAT |
| 201 | TGGAAGGATT GATCCTGCGA ATGATAATAT TAAATATGAC CCGAAGTTCC |
| 251 | AGGGCAAGGC CACTATAACA GCAGACACAT CCTCCAACAC AGCCTACCTA |
| 301 | CAGCTCAACA GCCTGACATC TGAGGACACT GCCGTCTATT ACTGTGCTAG |
| 351 | ATCTGAGGAA AATTGGTACG ACTTTTTTGA CTACTGGGGC CAAGGCACCA |
| 401 | CTCTCACAGT CTCCTCA |

The amino acid sequence of the heavy chain variable region with an optional leader (underscored) is as follows:

| (SEQ ID NO:143) | |
|---|---|
| 1 | MKCSWVIFFL MAVVTGVNSE VQLQQSGAEL VKPGASVKLS CTGSGFNIKD |
| 51 | TYIHWVKQRP EQGLEWIGRI DPANDNIKYD PKFQGKATIT ADTSSNTAYL |
| 101 | QLNSLTSEDT AVYYCARSEE NWYDFFDYWG QGTTLTVSS |

The nucleotide sequence encoding the light chain variable region is as follows:

| (SEQ ID NO:144) | |
|---|---|
| 1 | ATGAAGTTGC CTGTTAGGCT GTTGGTGCTG ATGTTCTGGA TTCCTGCTTC |
| 51 | CAGCAGTGAT GTTTTGATGA CCCAAACTCC ACTCTCCCTG CCTGTCAGTC |
| 101 | TTGGAGATCA AGCCTCCATC TCTTGCAGGT CTAGTCAGAG CATTGTACAT |
| 151 | AGTAATGGAA ACACCTATTT AGAATGGTAC CTGCAGAAAC CAGGCCAGTC |
| 201 | TCCAAAGCTC CTGATCTACA AAGTTTCCAA CCGATTTTCT GGGGTCCCAG |
| 251 | ACAGGTTCAG TGGCAGTGGA TCAGGGACAG ATTTCACACT CAAGATTAGC |
| 301 | AGAGTGGAGG CTGAGGATCT GGGAGTTTAT TACTGCTTTC AAGGTTCACA |
| 351 | TATTCCGTAC ACGTTCGGAG GGGGGACCAA GCTGGAAATA AAA |

The amino acid sequence of the light chain variable region with an optional leader (underscored) is as follows:

| (SEQ ID NO:145) | |
|---|---|
| 1 | MKLPVRLLVL MFWIPASSSD VLMTQTPLSL PVSLGDQASI SCRSSQSIVH |
| 51 | SNGNTYLEWY LQKPGQSPKL LIYKVSNRFS GVPDRFSGSG SGTDFTLKIS |
| 101 | RVEAEDLGVY YCFQGSHIPY TFGGGTKLEI K |

### Example 8

### Nucleotide and Amino Acid Sequences of Exemplary First Humanized Variants of the MJ 2-7 Antibody

Humanized antibody Version 1 (V1) is based on the closest human germline clones. The nucleotide sequence of hMJ 2-7 V1 heavy chain variable region (hMJ 2-7 VH V1) (with a sequence encoding an optional leader sequence) is as follows:

| (SEQ ID NO:146) | |
|---|---|
| 1 | ATGGATTGGA CCTGGCGCAT CCTGTTCCTG GTGGCCGCTG CCACCGGCGC |
| 51 | TCACTCTCAG GTGCAGCTGG TGCAGTCTGG CGCCGAGGTG AAGAAGCCTG |
| 101 | GCGCTTCCGT GAAGGTGTCC TGTAAGGCCT CCGGCTTCAA CATCAAGGAC |
| 151 | ACCTACATCC ACTGGGTGCG GCAGGCTCCC GGCCAGCGGC TGGAGTGGAT |
| 201 | GGGCCGGATC GATCCTGCCA ACGACAACAT CAAGTACGAC CCCAAGTTTC |
| 251 | AGGGCCGCGT GACCATCACC CGCGATACCT CCGCTTCTAC CGCCTACATG |
| 301 | GAGCTGTCTA GCCTGCGGAG CGAGGATACC GCCGTGTACT ACTGCGCCCG |
| 351 | CTCCGAGGAG AACTGGTACG ACTTCTTCGA CTACTGGGGC CAGGGCACCC |
| 401 | TGGTGACCGT GTCCTCT |

The amino acid sequence of the heavy chain variable region (hMJ 2-7 V1) is based on a CDR grafted to DP-25, VH-I, 1-03. The amino acid sequence with an optional leader (first underscored region; CDRs based on AbM definition shown in subsequent underscored regions) is as follows:

| (SEQ ID NO:147) | |
|---|---|
| 1 | MDWTWRILFL VAAATGAHS - Q VQLVQSGAEV KKPGASVKVS CKASGFNIKD |
| 51 | TYIHWVRQAP GQRLEWMGRI DPANDNIKYD PKFQGRVTIT RDTSASTAYM |
| 101 | ELSSLRSEDT AVYYCARSEE NWYDFFDYWG QGTLVTVSSG ESCR |

The nucleotide sequence of the hMJ 2-7 V1 light chain variable region (hMJ 2-7 VL V1) (with a sequence encoding an optional leader sequence) is as follows:

| (SEQ ID NO:148) | |
|---|---|
| 1 | ATGCGGCTGC CCGCTCAGCT GCTGGGCCTG CTGATGCTGT GGGTGCCCGG |
| 51 | CTCTTCCGGC GACGTGGTGA TGACCCAGTC CCCTCTGTCT CTGCCCGTGA |
| 101 | CCCTGGGCCA GCCCGCTTCT ATCTCTTGCC GGTCCTCCCA GTCCATCGTG |
| 151 | CACTCCAACG GCAACACCTA CCTGGAGTGG TTTCAGCAGA GACCCGGCCA |
| 201 | GTCTCCTCGG CGGCTGATCT ACAAGGTGTC CAACCGCTTT TCCGGCGTGC |
| 251 | CCGATCGGTT CTCCGGCAGC GGCTCCGGCA CCGATTTCAC CCTGAAGATC |
| 301 | AGCCGCGTGG AGGCCGAGGA TGTGGGCGTG TACTACTGCT TCCAGGGCTC |
| 351 | CCACATCCCT TACACCTTTG GCGGCGGAAC CAAGGTGGAG ATCAAG |

This version is based on a CDR graft to DPK18, V kappaII. The amino acid sequence of hMJ 2-7 V1 light chain variable region (hMJ 2-7 VL V1) (with optional leader as first underscored region; CDRs based on AbM definition in subsequent underscored regions) is as follows:

| (SEQ ID NO:149) | |
|---|---|
| 1 | MRLPAQLLGL LMLWVPGSSG -DVVMTQSPLS LPVTLGQPAS ISCRSSQSIV |
| 51 | HSNGNTYLEW FQQRPGQSPR RLIYKVSNRF SGVPDRFSGS GSGTDFTLKI |
| 101 | SRVEAEDVGV YYGFQGSHIP YTFGGGTKVE IK |

### Example 9

### Nucleotide and Amino Acid Sequences of Exemplary Second Humanized Variants of the MJ 2-7 Antibody

The following heavy chain variable region is based on a CDR graft to DP-54, VH-3, 3-07. The nucleotide sequence of hMJ 2-7 Version 2 (V2) heavy chain variable region (hMJ 2-7 VH V2) (with a sequence encoding an optional leader sequence) is as follows:

| (SEQ ID NO:150) | |
|---|---|
| 1 | ATGGAGCTGG GCCTGTCTTG GGTGTTCCTG GTGGCTATCG TGGAGGGCGT |
| 51 | GCAGTGCGAG GTGCAGCTGG TGGAGTCTGG CGGCGGACTG GTGCAGCCTG |
| 101 | GCGGCTCTCT GCGGCTGTCT TGCGCCGCTT CCGGCTTCAA CATCAAGGAC |
| 151 | ACCTACATGC ACTGGGTGCG GCAGGCTCCG GGCAAGGGCC TGGAGTGGGT |
| 201 | GGCCCGGATC GATCCTGCCA ACGACAACAT CAAGTACGAC CCCAAGTTCC |
| 251 | AGGGCCGGTT CACCATCTCT CGCGACAACG CCAAGAACTC CCTGTACCTC |
| 301 | CAGATGAACT CTCTGCGCGC CGAGGATACC GCCGTGTACT ACTGCGCCCG |
| 351 | GAGCGAGGAG AACTGGTACG ACTTCTTCGA CTACTGGGGG CAGGGGACCC |
| 401 | TGGTGACCGT GTCCTCT |

The amino acid sequence of hMJ 2-7 V2 heavy chain variable region (hMJ 2-7 VH V2) with an optional leader (first underscored region; CDRs based on AbM definition shown in subsequent underscored regions) is as follows:

| | |
|---|---|
| 1 | MELGLSWVFL VAILEGVQC- E VQLVESGGGL VQPGGSLRLS CAASGFNIKD |
| 51 | TYIHWVRQAP GKGLEWVARI DPANDNIKYD PKFQGRFTIS RDNAKNSLYL |
| 101 | QMNSLRAEDT AVYYCARSEE NWYDFFDYWG QGTLVTVSS |

The hMJ 2-7 V2 light chain variable region was based on a CDR graft to DPK9, V kappaI, 02. The nucleotide sequence of hMJ 2-7 V2 light chain variable region (hMJ 2-7 VL V2) (with a sequence encoding an optional leader sequence) is as follows:

| (SEQ ID NO:152) | |
|---|---|
| 1 | ATGGATATGC GCGTGCCCGC TCAGCTGCTG GGCCTGCTGC TGCTGTGGCT |
| 51 | GCGCGGAGCC CGCTGCGATA TCCAGATGAC CCAGTCCCCT TCTTCTCTGT |
| 101 | CCGCCTCTGT GGGCGATCGC GTGACCATCA CCTGTCGGTC CTCCCAGTCC |
| 151 | ATCGTGCACT CCAACGGCAA CACCTACCTG GAGTGGTATC AGCAGAAGCC |
| 201 | CGGCAAGGCC CCTAAGCTGC TGATCTACAA GGTGTCCAAC CGCTTTTCCG |
| 251 | GCGTGCCTTC TCGGTTCTCC GGCTCCGGCT CCGGCACCGA TTTCACCCTG |
| 301 | ACCATCTCCT CCCTCCAGCC CGAGGATTTC GCCACCTACT ACTGCTTCCA |
| 351 | GGGCTCCCAC ATCCCTTACA CCTTTGGCGG CGGAACCAAG GTGGAGATCA |
| 401 | AGCGT |

The amino acid sequence of the light chain variable region of hMJ 2-7 V2 light chain variable region (hMJ 2-7 VL V2) (with optional leader peptide underscored and CDRs based on AbM definition shown in subsequent underscored regions) is as follows:

| (SEQ ID NO:153) | |
|---|---|
| 1 | MDMRVPAQLL GLLLLWLRGA RC -DIQMTQSP SSLSASVGDR VTITCRSSQS |
| 51 | IVHSNGNTYL EWYQQKLPGKA PKLLIYKVSN RFSGVPSRFS GSGSGTDFTL |
| 101 | TISSLQPEDF ATYYCFQGSH IPYTFGGGTK VEIKR |

Additional humanized versions of MJ 2-7 V2 heavy chain variable region were made. These versions included backmutations that have murine amino acids at selected framework positions.

The nucleotide sequence encoding the heavy chain variable region “Version 2.1” or V2.1 with the back mutations V48I,A29G is as follows:

| (SEQ ID NO:154) | |
|---|---|
| 1 | GAGGTGCAGC TGGTGGAGTC TGGCGGCGGA CTGGTGCAGC CTGGCGGCTC |
| 51 | TCTGCGGCTG TCTTGCGCCG CTTCCGGCTT CAACATCAAG GACACCTACA |
| 101 | TCCACTGGGT GCGGCAGGCT CCCGGCAAGG GCCTGGAGTG GATCGGCCGG |
| 151 | ATCGATCCTG CCAACGACAA CATCAAGTAC GACCCCAAGT TCCAGGGCCG |
| 201 | GTTCACCATC TCTCGCGACA ACGCCAAGAA CTCCCTGTAC CTCCAGATGA |
| 251 | ACTCTCTGCG CGCCGAGGAT ACCGCCGTGT ACTACTGCGC CCGGAGCGAG |
| 301 | GAGAACTGGT ACGACTTCTT CGACTACTGG GGCCAGGGCA CCCTGGTGAC |
| 351 | CGTGTCCTCT |

The amino acid sequence of the heavy chain variable region of V2.1 (CDRs based on AbM definition shown in subsequent underscored regions) is as follows:

| (SEQ ID NO:155) | |
|---|---|
| 1 | EVQLVESGGG LVQPGGSLRL SCAASGFNIK DTYIHWVRQA PGKGLEWIGR |
| 51 | IDPANDNIKY DPKFQGRFTI SRDNAKNSLY LQMNSLRAED TAVYYCARSE |
| 101 | ENWYDFFDYW GQGTLVTVSS |

The nucleotide sequence encoding the heavy chain variable region V2.2 with the back mutations (R67K;F68A) is as follows:

| (SEQ ID NO:156) | |
|---|---|
| 1 | GAGGTGCAGC TGGTGGAGTC TGGCGGCGGA CTGGTGCAGC CTGGCGGCTC |
| 51 | TCTGCGGCTG TCTTGCGCCG CTTCCGGCTT CAACATCAAG GACACCTACA |
| 101 | TCCACTGGGT GCGGCAGGGT CCCGGCAAGG GCCTGGAGTG GGTGGGCCGG |
| 151 | ATCGATCCTG CGAACGACAA CATCAAGTAC GACCCCAAGT TCCAGGGCAA |
| 201 | GGCCACCATG TCTCGCGACA ACGGCAAGAA CTCCCTGTAC CTCCAGATGA |
| 251 | ACTCTCTGCG CGCCGAGGAT ACCGCCGTGT ACTACTGCGC CCGGAGCGAG |
| 301 | GAGAACTGGT ACGACTTCTT CGACTACTGG GGCCAGGGCA CCCTGGTGAC |
| 351 | CGTGTCCTGT |

The amino acid sequence of the heavy chain variable region of V2.2 (CDRs based on AbM definition shown in subsequent underscored regions) is as follows:

| (SEQ ID NO:157) | |
|---|---|
| 1 | EVQLVESGGG LVQPGGSLRL SCAASGFNIK DTYIHWVRQA PGKGLEWVAR |
| 51 | IDPANDNIKY DPKFQGKATI SRDNAKNSLY LQMNSLRAED TAVYYCARSE |
| 102 | ENWYDFFDYW GQGTLVTVSS |

The nucleotide sequence encoding the heavy chain variable region V2.3 with the back mutations (R72A):

| (SEQ ID NO:158) | |
|---|---|
| 1 | GAGGTGCAGC TGGTGGAGTC TGGCGGCGGA CTGGTGCAGC CTGGCGGCTC |
| 51 | TCTGCGGCTG TCTTGCGCCG CTTCCGGCTT CAACATCAAG GACACCTACA |
| 101 | TCCACTGGGT GCGGCAGGCT CCCGGCAAGG GCCTGGAGTG GGTGGCCCGG |
| 151 | ATCGATCCTG CCAACGACAA CATCAAGTAC GACCCCAAGT TCCAGGGCCG |
| 201 | GTTCACCATC TCTGCCGACA ACGCCAAGAA CTCCCTGTAC CTCCAGATGA |
| 251 | ACTCTCTGCG CGCCGAGGAT ACCGCCGTGT ACTACTGCGC CCGGAGCGAG |
| 301 | GAGAACTGGT ACGACTTCTT CGACTACTGG GGCCAGGGCA CCCTGGTGAC |
| 351 | CGTGTCCTGT |

The amino acid sequence of the heavy chain variable region of V2.3 (CDRs based on AbM definition shown in subsequent underscored regions) is as follows:

| (SEQ ID NO:159) | |
|---|---|
| 1 | EVQLVESGGG LVQPGGSLRL SCAASGFNIK DTYIHWVRQA PGKGLEWVAR |
| 51 | IDPANDNIKY DPKFQGRFTI SADNAKNSLY LQMNSLRAED TAVYYCARSE |
| 103 | ENWYDFFDYW GQGTLVTVSS |

The nucleotide sequence encoding the heavy chain variable region V2.4 with the back mutations (A49G) is as follows:

| (SEQ ID NO:160) | |
|---|---|
| 1 | GAGGTGCAGC TGGTGGAGTC TGGCGGCGGA CTGGTGCAGC CTGGCGGCTC |
| 51 | TCTGCGGCTG TCTTGCGCCG CTTCCGGCTT CAACATCAAG GACACCTACA |
| 101 | TCCAGTGGGT GCGGCAGGCT CCCGGCAAGG GCCTGGAGTG GGTGGGCCGG |
| 151 | ATCGATCCTG CCAACGACAA CATCAAGTAC GACCCCAAGT TCCAGGGCCG |
| 201 | GTTCACCATC TCTCGCGACA ACGCCAAGAA CTCCCTGTAC CTCCAGATGA |
| 251 | ACTCTCTGCG CGCCGAGGAT ACCGCCGTGT ACTACTGCGC CCGGAGCGAG |
| 301 | GAGAACTGGT ACGACTTCTT CGACTACTGG GGCCAGGGCA CCCTGGTGAC |
| 351 | CGTGTCGTCT |

The amino acid sequence of the heavy chain variable region of V2.4 (CDRs based on AbM definition shown in subsequent underscored regions) is as follows:

| (SEQ ID NO:161) | |
|---|---|
| 1 | EVQLVESGGG LVQPGGSLRL SCAASGFNIK DTYIHWVRQA PGKGLEWVGR |
| 51 | IDPANDNIKY DPKFQGRFTI SRDNAKNSLY LQMNSLRAED TAVYYCARSE |
| 104 | ENWYDFFDYW GQGTLVTVSS |

The nucleotide sequence encoding the heavy chain variable region V2.5 with the back mutations (R67K;F68A;R72A) is as follows:

| (SEQ ID NO:162) | |
|---|---|
| 1 | GAGGTGCAGC TGGTGGAGTC TGGCGGCGGA CTGGTGCAGC CTGGCGGCTC |
| 51 | TCTGCGGCTG TCTTGCGCCG CTTCCGGCTT CAACATCAAG GACACCTACA |
| 101 | TCCACTGGGT GCGGCAGGCT CCCGGCAAGG GCCTGGAGTG GGTGGCCCGG |
| 151 | ATCGATCCTG CCAACGACAA CATCAAGTAC GACCCGAAGT TCCAGGGCAA |
| 201 | GGCCACCATC TCTGCCGACA ACGCCAAGAA CTCCCTGTAC CTCCAGATGA |
| 251 | ACTCTCTGCG CGCCGAGGAT ACCGCCGTGT ACTACTGCGC CCGGAGCGAG |
| 301 | GAGAACTGGT ACGACTTTCTT CGACTACTGG GGCCAGGGCA CCCTGGTGAC |
| 352 | CGTGTCCTCT |

The amino acid sequence of the heavy chain variable region of V2.5 (CDRs based on AbM definition shown in subsequent underscored regions) is as follows:

| (SEQ ID NO:163) | |
|---|---|
| 1 | EVQLVESGGG LVQPGGSLRL SCAASGFNIK DTYIHWVRQA PGKGLEWVAR |
| 51 | IDPANDNIKY DPKFQGKATI SADNAKNSLY LQMNSLRAED TAVYYCARSE |
| 105 | ENWYDFFDYW GQGTLVTVSS |

The nucleotide sequence encoding the heavy chain variable region V2.6 with the back mutations (V48I;A49G;R72A) is as follows:

| (SEQ ID NO:164) | |
|---|---|
| 1 | GAGGTGCAGC TGGTGGAGTC TGGCGGCGGA CTGGTGCAGC CTGGCGGCTC |
| 51 | TCTGCGGCTG TCTTGCGCCG CTTCCGGCTT CAACATCAAG GACACCTACA |
| 101 | TCCACTGGGT GCGGCAGGCT CCCGGCAAGG GCCTGGAGTG GATCGGCCGG |
| 151 | ATCGATCCTG CCAACGACAA CATCAAGTAC GACCCCAAGT TCCAGGGCCG |
| 201 | GTTCACCATC TCTGCCGACA ACGCCAAGAA CTCCCTGTAC CTCCAGATGA |
| 251 | ACTCTCTGCG CGCCGAGGAT ACCGCCGTGT ACTACTGCGC CCGGAGCGAG |
| 301 | GAGAACTGGT ACGACTTCTT CGACTACTGG GGCCAGGGCA CCCTGGTGAC |
| 351 | CGTGTCCTCT |

The amino acid sequence of the heavy chain variable region of V2.6 (CDRs based on AbM definition shown in subsequent underscored regions) is as follows:

| (SEQ ID NO:165) | |
|---|---|
| 1 | EVQLVESGGG LVQPGGSLRL SCAASGFNIK DTYIHWVRQA PGKGLEWIGR |
| 51 | IDPANDNIKY DPKFQGRFTI SADNAKNSLY LQMNSLRAED TAVYYCARSE |
| 106 | ENWYDFFDYW GQGTLVTVSS |

The nucleotide sequence encoding the heavy chain variable region V2.7 with the back mutations (A49G;R72A) is as follows:

| (SEQ ID NO:166) | |
|---|---|
| 1 | GAGGTGCAGC TGGTGGAGTC TGGCGGCGGA CTGGTGCAGC CTGGCGGCTC |
| 51 | TCTGCGGCTG TCTTGCGCCG CTTCCGGCTT CAACATCAAG GACACCTACA |
| 101 | TCCACTGGGT GCGGCAGGCT CCCGGCAAGG GCCTGGAGTG GGTGGGCCGG |
| 151 | ATCGATCCTG CCAACGACAA CATCAAGTAC GACCCCAAGT TCCAGGGCCG |
| 201 | GTTCACCATC TCTGCCGACA ACGCCAAGAA CTCCCTGTAC CTCCAGATGA |
| 251 | ACTCTCTGCG CGCCGAGGAT ACCGCCGTGT ACTACTGCGC CCGGAGCGAG |
| 301 | GAGAACTGGT ACGACTTCTT CGACTACTGG GGCCAGGGCA CCCTGGTGAC |
| 351 | CGTGTCCTCT |

The amino acid sequence of the heavy chain variable region of V2.7 (CDRs based on AbM definition shown in subsequent underscored regions) is as follows:

(SEQ ID NO:167)

| | |
|---|---|
| 1 | EVQLVESGGG LVQPGGSLRL SCAASGFNIK DTYIHWVRQA PGKGLEWVGR |
| 51 | IDPANDNIKY DPKFQGRFTI SADNAKNSLY LQMNSLRAED TAVYYCARSE |
| 107 | ENWYDFFDYW GQGTLVTVSS |

The nucleotide sequence encoding the heavy chain variable region V2.8 with the back mutations (L79A) is as follows:

| (SEQ ID NO:168) | |
|---|---|
| 1 | GAGGTGCAGC TGGTGGAGTC TGGCGGCGGA CTGGTGCAGC CTGGCGGCTC |
| 51 | TCTGCGGCTG TCTTGCGCCG CTTCCGGCTT CAACATCAAG GACACCTACA |
| 101 | TCCACTGGGT GCGGCAGGCT CCCGGCAAGG GCCTGGAGTG GGTGGCCCGG |
| 151 | ATCGATCCTG CCAACGACAA CATCAAGTAC GACCCCAAGT TCCAGGGCCG |
| 201 | GTTCACCATC TCTCGCGACA ACGCCAAGAA CTCCGCCTAC CTCCAGATGA |
| 251 | ACTCTCTGCG CGCCGAGGAT ACCGCCGTGT ACTACTGCGC CCGGAGCGAG |
| 301 | GAGAACTGGT ACGACTTCTT CGACTACTGG GGCCAGGGCA CCCTGGTGAC |
| 351 | CGTGTCCTCT |

The amino acid sequence of the heavy chain variable region of V2.8 (CDRs based on AbM definition shown in subsequent underscored regions) is as follows:

| (SEQ ID NO:169) | |
|---|---|
| 1 | EVQLVESGGG LVQPGGSLRL SCAASGFNIK DTYIHWVRQA PGKGLEWVAR |
| 51 | IDPANDNIKY DPKFQGRFTI SRDNAKNSAY LQMNSLRAED TAVYYCARSE |
| 108 | ENWYDFFDYW GQGTLVTVSS |

The nucleotide sequence encoding the heavy chain variable region V2.10 with the back mutations (A49G;R72A;L79A) is as follows:

| (SEQ ID NO:170) | |
|---|---|
| 1 | GAGGTGCAGC TGGTGGAGTC TGGCGGCGGA CTGGTGCAGC CTGGCGGCTC |
| 51 | TCTGCGGCTG TCTTGCGCCG CTTCCGGCTT CAACATCAAG GACACCTACA |
| 101 | TCCACTGGGT GCGGCAGGCT CCCGGCAAGG GCCTGGAGTG GGTGGGCCGG |
| 151 | ATCGATCCTG CCAACGACAA CATCAAGTAC GACCCCAAGT TCCAGGGCCG |
| 201 | GTTCACCATC TCTGCCGACA ACGCCAAGAA CTCCGCCTAC CTCCAGATGA |
| 251 | ACTCTCTGCG CGCCGAGGAT ACCGCCGTGT ACTACTGCGC CCGGAGCGAG |
| 301 | GAGAACTGGT ACGACTTCTT CGACTACTGG GGCCAGGGCA CCCTGGTGAC |
| 351 | CGTGTCCTCT |

The amino acid sequence of the heavy chain variable region of V2.10 (CDRs based on AbM definition shown in subsequent underscored regions) is as follows:

| (SEQ ID NO:171) | |
|---|---|
| 1 | EVQLVESGGG LVQPGGSLRL SCAASGFNIK DTYIHWVRQA PGKGLEWVGR |
| 51 | IDPANDNIKY DPKFQGRFTI SADNAKNSAY LQMNSLRAED TAVYYCARSE |
| 109 | ENWYDFFDYW GQGTLVTVSS |

The nucleotide sequence encoding the heavy chain variable region V2.11 with the back mutations (V48I;A49G;R72A;L79A) is as follows:

| (SEQ ID NO:172) | |
|---|---|
| 1 | GAGGTGCAGC TGGTGGAGTC TGGCGGCGGA CTGGTGCAGC CTGGCGGCTC |
| 51 | TCTGCGGCTG TCTTGCGCCG CTTCCGGCTT CAACATCAAG GACACCTACA |
| 101 | TCCACTGGGT GCGGCAGGCT CCCGGCAAGG GCCTGGAGTG GATCGGCCGG |
| 151 | ATCGATCCTG CCAACGACAA CATCAAGTAC GACCCCAAGT TCCAGGGCCG |
| 201 | GTTCACCATC TCTGCCGACA ACGCCAAGAA CTCCGCCTAC CTCCAGATGA |
| 251 | ACTCTCTGCG CGCCGAGGAT ACCGCCGTGT ACTACTGCGC CCGGAGCGAG |
| 301 | GAGAACTGGT ACGACTTCTT CGACTACTGG GGCCAGGGCA CCCTGGTGAC |
| 351 | CGTGTCCTCT |

The amino acid sequence of the heavy chain variable region of V2.11 (CDRs based on AbM definition shown in subsequent underscored regions) is as follows:

| (SEQ ID NO:173) | |
|---|---|
| 1 | EVQLVESGGG LVQPGGSLRL SCAASGFNIK DTYIHWVRQA PGKGLEWIGR |
| 51 | IDPANDNIKY DPKFQGRFTI SADNAKNSAY LQMNSLRAED TAVYYCARSE |
| 110 | ENWYDFFDYW GQGTLVTVSS |

The nucleotide sequence encoding the heavy chain variable region V2.16 with the back mutations (V48I;A49G;R72A) is as follows:

| (SEQ ID NO:174) | |
|---|---|
| 1 | GAGGTGCAGC TGGTGGAGTC TGGCGGCGGA CTGGTGCAGC CTGGCGGCTC |
| 51 | TCTGCGGCTG TCTTGCACCG GCTCCGGCTT CAACATCAAG GACACCTACA |
| 101 | TCCACTGGGT GCGGCAGGCT CCCGGCAAGG GCCTGGAGTG GATCGGCCGG |
| 151 | ATCGATCCTG CCAACGACAA CATCAAGTAC GACCCCAAGT TCCAGGGCCG |
| 201 | GTTCACCATC TCTGCCGACA ACGCCAAGAA CTCCCTGTAC CTCCAGATGA |
| 251 | ACTCTCTGCG CGCCGAGGAT ACCGCCGTGT ACTACTGCGC CCGGAGCGAG |
| 301 | GAGAACTGGT ACGACTTCTT CGACTACTGG GGCCAGGGCA CCCTGGTGAC |
| 351 | CGTGTCCTCT |

The amino acid sequence of the heavy chain variable region of V2.16 (CDRs based on AbM definition shown in subsequent underscored regions) is as follows:

| (SEQ ID NO:175) | |
|---|---|
| 1 | EVQLVESGGG LVQPGGSLRL SCTGSGFNIK DTYIHWVRQA PGKGLEWIGR |
| 51 | IDPANDNIKY DPKFQGRFTI SADNAKNSLY LQMNSLRAED TAVYYCARSE |
| 111 | ENWYDFFDYW GQGTLVTVSS |

The following is the amino acid sequence of a humanized MH 2-7 V2.11 IgG1 with a mutated CH2 domain:

(SEQ ID NO:176)

EVQLVESGGGLVQPGGSLRLSCAASGFNIKDTYIHWVRQAPGKGLEWIGR
IDPANDNIKYDPKFQGRFTISADNAKNSAYLQMNSLRAEDTAVYYCARSE
ENWYDFFDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPEALGALPSVFLFPPK
PKDTLMISRTPEVTCVVVDVSHEDPEVKFNWYVDGVEVHNAKTKPREEQY
NSTYRVVSVLTVLHQDWLNGKEYKCKVSKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK

The variable domain is at amino acids 1-120; CH1 at 121-218; hinge at 219-233; CH2 at 234-343; and CH3 at 344-450. The light chain includes the following sequence with variable domain at 1-133.

(SEQ ID NO:177)

DIQMTQSPSSLSASVGDRVTITCRSSQSIVHSNGNTYLEWYQQKPGKAPK
LLIYKVSNRFSGVPSRFSGSGSGTDFTLTISSLQPEDFATYYCFQGSHIP
YTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

### Example 10

### Functional Assays of Exemplary Variants of MJ2-7

The ability of the MJ2-7 antibody and humanized variants was evaluated to inhibit human IL-13 in assays for IL-13 activity.

### STAT6 Phosphorylation Assay.

HT-29 human colonic epithelial cells (ATCC) were grown as an adherent monolayer in McCoy's 5A medium containing 10% FBS, Pen-Strep, glutamine, and sodium bicarbonate. For assay, the cells were dislodged from the flask using trypsin, washed into fresh medium, and distributed into 12×75 mm polystyrene tubes. Recombinant human IL-13 (R&D Systems, Inc.) was added at concentrations ranging from 100-0.01 ng/ml. For assays testing the ability of antibody to inhibit the IL-13 response, 1 ng/ml recombinant human IL-13 was added along with dilutions of antibody ranging from 500-0.4 ng/ml. Cells were incubated in a 37° C. water bath for 30-60 minutes, then washed into ice-cold PBS containing 1% BSA. Cells were fixed by incubating in 1% paraformaldehyde in PBS for 15 minutes at 37° C., then washed into PBS containing 1% BSA. To permeabilize the nucleus, cells were incubated overnight at −20° C. in absolute methanol. They were washed into PBS containing 1% BSA, then stained with ALEXA™ Fluor 488-labeled antibody to STAT6 (BD Biosciences). Fluorescence was analyzed with a FACSCAN™ and CELLQUEST™ software (BD Biosciences).

### CD23 Induction on Human Monocytes

Mononuclear cells were isolated from human peripheral blood by layering over HISTOPAQUE® (Sigma). Cells were washed into RPMI containing 10% heat-inactivated FCS, 50 U/ml penicillin, 50 mg/ml streptomycin, 2 mM L-glutamine, and plated in a 48-well tissue culture plate (Costar/Corning). Recombinant human IL-13 (R&D Systems, Inc.) was added at dilutions ranging from 100-0.01 ng/ml. For assays testing the ability of antibody to inhibit the IL-13 response, 1 ng/ml recombinant human IL-13 was added along with dilutions of antibody ranging from 500-0.4 ng/ml. Cells were incubated overnight at 37° C. in a 5% CO₂ incubator. The next day, cells were harvested from wells using non-enzymatic Cell Dissociation Solution (Sigma), then washed into ice-cold PBS containing 1% BSA. Cells were incubated with phycoerythrin (PE)-labeled antibody to human CD23 (BD Biosciences, San Diego, Calif.), and CyChrome-labeled antibody to human CD11b (BD Biosciences). Monocytes were gated based on high forward and side light scatter, and expression of CD11b. CD23 expression on monocytes was determined by flow cytometry using a FACSCAN™ (BD Biosciences), and the percentage of CD23⁺ cells was analyzed with CELLQUEST™ software (BD Biosciences).

### TF-1 Cell Proliferation

TF-1 cells are a factor-dependent human hemopoietic cell line requiring interleukin 3 (IL-3) or granulocyte/macrophage colony-stimulating factor (GM-CSF) for their long-term growth. TF-1 cells also respond to a variety of other cytokines, including interleukin 13 (IL-13). TF-1 cells (ATCC) were maintained in RPMI medium containing 10% heat-inactivated FCS, 50 U/ml penicillin, 50 mg/ml streptomycin, 2 mM L-glutamine, and 5 ng/ml recombinant human GM-CSF (R&D Systems). Prior to assay, cells were starved of GM-CSF overnight. For assay, TF-1 cells were plated in duplicate at 5000 cells/well in 96-well flat-bottom microtiter plates (Costar/Corning), and challenged with human IL-13 (R&D Systems), ranging from 100-0.01 ng/ml. After 72 hours in a 37° C. incubator with 5% CO₂, the cells were pulsed with 1 μCi/well ³H-thymidine (Perkin Elmer/New England Nuclear). They were incubated an additional 4.5 hours, then cells were harvested onto filter mats using a TOMTEK™ harvester. ³H-thymidine incorporation was assessed by liquid scintillation counting.

### Tenascin Production Assay

BEAS-2B human bronchial epithelial cells (ATCC) were maintained BEGM media with supplements (Clonetics). Cells were plated at 20,000 per well in a 96-well flat-bottom culture plate overnight. Fresh media is added containing IL-13 in the presence or absence of the indicated antibody. After overnight incubation, the supernatants are harvested, and assayed for the presence of the extracellular matrix component, tenascin C, by ELISA. ELISA plates are coated overnight with 1 ug/ml of murine monoclonal antibody to human tenascin (IgG1, k; Chemicon International) in PBS. Plates are washed with PBS containing 0.05% TWEEN®-20 (PBS-Tween), and blocked with PBS containing 1% BSA. Fresh blocking solution was added every 6 minutes for a total of three changes. Plates were washed 3× with PBS-Tween. Cell supernatants or human tenascin standard (Chemicon International) were added and incubated for 60 minutes at 37° C. Plates were washed 3× with PBS-Tween. Tenascin was detected with murine monoclonal antibody to tenascin (IgG2a, k; Biohit). Binding was detected with HRP-labeled antibody to mouse IgG2a, followed by TMB substrate. The reaction was stopped with 0.01 N sulfuric acid. Absorbance was read at 450 nm.

The HT 29 human epithelial cell line can be used to assay STAT6 phosphorylation. HT 29 cells are incubated with 1 ng/ml native human IL-13 crude preparation in the presence of increasing concentrations of the test antibody for 30 minutes at 37° C. Western blot analysis of cell lysates with an antibody to phosphorylated STAT6 can be used to detect dose-dependent IL 13-mediated phosphorylation of STAT6. Similarly, flow cytometric analysis can detect phosphorylated STAT6 in HT 29 cells that were treated with a saturating concentration of IL-13 for 30 minutes at 37° C., fixed, permeabilized, and stained with an ALEXA™ Fluor 488-labeled mAb to phospho-STAT6. An exemplary set of results is set forth in the Table 1. The inhibitory activity of V2.11 was comparable to that of sIL-13Rα2-Fc.

TABLE 1

| | | | Expression | Native hIL-13 |
|---|---|---|---|---|
| Construct | | Backmutations | μg/ml/ | STAT6 assay |
| VH | VL | VH | COS; 48 h | IC 50, nM |
| V2.0 | V2 | None, CDR grafted | 8-10 | >100 |
| | CDR graft | | | |
| V2.1 | V2 | V48I; A49G | 9-14 | 2.8 |
| V2.2 | V2 | R67K; F68A | 5-6 | >100 |
| V2.3 | V2 | R72A | 8-9 | 1.67-2.6 |
| V2.4 | V2 | A49G | 10 | 17.5 |
| V2.5 | V2 | R67K; F68A; R72A | 4-5 | 1.75 |
| V2.6 | V2 | V48I; A49G: R72A | 11-12 | 1.074-3.37 |
| V2.7 | V2 | A49G; R72A | 10-11 | 1.7 |
| V2.11 | V2 | V48I; A49G: | 24 | 0.25-0.55 |
| | | R72A: L79A | | |

### Example 11

### Binding Interaction Site Between IL-13 and IL-13Rα1

A complex of IL-13, the extracellular domain of IL-13Rα1 (residues 27-342 of SEQ ID NO:125), and an antibody that binds human IL-13 was studied by x-ray crystallography. See, e.g., U.S. Ser. No. 07/004,8785. Two points of substantial interaction were found between IL-13 and IL-13Rα1. The interaction between Ig domain 1 of IL-13Rα1 and IL-13 results in the formation of an extended beta sheet spanning the two molecules. Residues Thr88 [Thr107], Lys89 [Lys108], Ile90 [Ile109], and Glu91 [Glu110] of IL-13 (SEQ ID NO:124, mature sequence [full-length sequence (SEQ ID NO:178)]) form a beta strand that interacts with residues Lys76, Lys77, Ile78 and Ala79 of the receptor (SEQ ID NO:125). Additionally, the side chain of Met33 [Met52] of IL-13 (SEQ ID NO:124 [SEQ ID NO:178]) extends into a hydrophobic pocket that is created by the side chains of these adjoining strands.

The predominant feature of the interaction with Ig domain 3 is the insertion of a hydrophobic residue (Phe107 [Phe126]) of IL-13 (SEQ ID NO:124 [SEQ ID NO:178]) into a hydrophobic pocket in Ig domain 3 of the receptor IL-13Rα1. The hydrophobic pocket of IL-13Rα1 is formed by the side chains of residues Leu319, Cys257, Arg256, and Cys320 (SEQ ID NO:125). The interaction with Phe107 [Phe126] of IL-13 (SEQ ID NO:124 [SEQ ID NO:178]) results in an extensive set of van der Waals interactions between amino acid residues Ile254, Ser255, Arg256, Lys318, Cys320, and Tyr321 of IL-13Rα1 (SEQ ID NO:125) and amino acid residues Arg11 [Arg30], Glu12 [Glu31], Leu13 [Leu32], Ile14 [Ile33], Glu15 [Ile34], Lys104 [Lys123], Lys105 [Lys124], Leu106 [Leu125], Phe107 [Phe126], and Arg108 [Arg 127] of IL-13 (SEQ ID NO:124 [SEQ ID NO:178]). These results demonstrate that an IL-13 binding agent that binds to the regions of IL-13 involved in interaction with IL-13Rα1 can be used to inhibit IL-13 signaling.

### Example 12

### Expression of Humanized MJ 2-7 Antibody in COS Cells

To evaluate the production of chimeric anti-NHP IL13 antibodies in the mammalian recombinant system, the variable regions of mouse MJ 2-7 antibody were subcloned into a pED6 expression vector containing human kappa and IgG1mut constant regions. Monkey kidney COS-1 cells were grown in DME media (Gibco) containing 10% heat-inactivated fetal bovine serum, 1 mM glutamine and 0.1 mg/ml Penicillin/Streptomycin. Transfection of COS cells was performed using TRANSITIT™-LT1 Transfection reagent (Mirus) according to the protocol suggested by the reagent supplier. Transfected COS cells were incubated for 24 hours at 37° C. in the presence of 10% CO₂, washed with sterile PBS, and then grown in serum-free media R1CD1 (Gibco) for 48 hours to allow antibody secretion and accumulation in the conditioned media. The expression of chMJ 2-7 antibody was quantified by total human IgG ELISA using purified human IgG1/kappa antibody as a standard.

The production of chimeric MJ 2-7 antibody in COS cells was significantly lower then the control chimeric antibody (Table 2). Therefore, optimization of Ab expression was included in the MJ 2-7 humanization process. The humanized MJ 2-7 V1 was constructed by CDR grafting of mouse MJ 2-7 heavy chain CDRs onto the most homologous human germline clone, DP 25, which is well expressed and represented in typical human antibody response. The CDRs of light chain were subcloned onto human germline clone DPK 18 in order to generate huMJ 2-7 V1 VL. The humanized MJ 2-7 V2 was made by CDR grafting of CDRs MJ 2-7 heavy chain variable region onto DP54 human germline gene framework and CDRs of MJ 2-7 light chain variable region onto DPK9 human germline gene framework. The DP 54 clone belongs to human VH III germline subgroup and DPK9 is from the V kappa I subgroup of human germline genes. Antibody molecules that include VH III and V kappa I frameworks have high expression level in E. coli system and possess high stability and solubility in aqueous solutions (see, e.g., Stefan Ewert et al., J. Mol. Biol. (2003), 325; 531-553, Adrian Auf et al., Methods (2004) 34:215-224). We have used the combination of DP54/DPK9 human frameworks in the production of several recombinant antibodies and have achieved a high expression of antibody (>20 μg/ml) in the transient COS transfection experiments.

TABLE 2

| mAb | Expression, μg/ml |
|---|---|
| 3D6 | 10.166 |
| Ch MJ 2-7 pED6 (1) | 2.44 |
| Ch MJ 2-7 pED6 (2) | 2.035 |
| h12A11 V2 | 1.639 |

The CDR grafted MJ 2-7 V1 and V2 VH and VL genes were subcloned into two mammalian expression vector systems (pED6kappa/pED6 IgG1mut and pSMEN2kappa/pSMED2IgG1mut), and the production of humanized MJ 2-7 antibodies was evaluated in transient COS transfection experiments as described above. In the first set of the experiments the effect of various combinations of huMJ 2-7 VL and VH on the antibody expression was evaluated (Table 3). Changing of MJ 2-7 VL framework regions to DKP9 increased the antibody production 8-10 fold, whereas VL V1 (CDR grafted onto DPK 18) showed only a moderate increase in antibody production. This effect was observed when humanized VL was combined with chimeric MJ 2-7 VH and humanized MJ 2-7 V1 and V2. The CDR grafted MJ 2-7. V2 had a 3-fold higher expression level then CDR grafted MJ 2-7 V1 in the same assay conditions.

TABLE 3

| mAb | Expression, μg/ml |
|---|---|
| ChMJ 2-7 | 1.83 |
| hVH V1/mVL | 3.04 |
| hVH V1/hVL V1 | 6.34 |
| hVH V1/hVL V2 | 15.4 |
| HVH-V2/mVL | 0.2 |
| mVH/hVL-V2 | 18.41 |
| hVH-V2/hVL-V1 | 5.13 |
| hVH-V2/hVL-V2 | 10.79 |

Similar experiments were performed with huMJ 2-7 V2 containing back mutations in the heavy chain variable regions (Table 4). The highest expression level was detected for huMJ 2-7 V2.11 that retained the antigen binding and neutralization properties of mouse MJ 2-7 antibody. Introduction of back mutations at the positions 48 and 49 (V48I and A49G) increased the production of huMJ 2-7 V2 antibody in COS cells, whereas the back mutations of amino acids at the positions 23, 24, 67 and 68 (A23T; A24G; R67K and F68A) had a negative impact on antibody expression.

TABLE 4

| mAb | Expression, μg/ml |
|---|---|
| V2 | 8.27 |
| V2.1 | 12.1 |
| V2.2 | 5.29 |
| V2.3 | 9.60 |
| V2.4 | 8.20 |
| V2.5 | 6.05 |
| V2.6 | 11.3 |
| V2.10 | 9.84 |
| V2.11 | 14.85 |
| V2.16 | 1.765 |

### Example 13

### Evaluation of Antigen Binding Properties of Humanized MJ 2-7 Antibodies by NHP IL-13 FLAG ELISA

The ability of fully humanized MJ 2-7 mAb (V1, V2 v2) to compete with biotinylated mouse MJ 2-7 Ab for binding to NHP IL-13-FLAG was evaluated by ELISA. The microtiter plates (Costar) were coated with 1 μg/ml of anti-FLAG monoclonal antibody M2 (Sigma). The FLAG NHP IL-13 protein at concentration of 10 ng/ml was mixed with 10 ng/ml of biotin labeled mouse MJ 2-7 antibody and various concentrations of unlabeled mouse and humanized MJ 2-7 antibody. The mixture was incubated for 2 hours at room temperature and then added to the anti-FLAG antibody-coated plate. Binding of FLAG NHP-IL-13/bioMJ2-7 Ab complexes was detected with streptavidin-HRP and 3,3′,5,5′-tetramethylbenzidine (TMB). The humanized MJ 2-7 V2 significantly lost activity whereas huMJ 2-7 V2.11 completely restored the antigen binding activity and was capable of competing with biotinylated MJ 2-7 mAb for binding to FLAG-NHP IL-13. BIACORE™ analysis also confirmed that NHP IL-13 had rapid binding to and slow dissociation to immobilized h1uMJ 2-7 v2.11.

### Example 14

### Molecular Modeling of Humanized MJ2-7 V2VH

Structure templates for modeling humanized MJ2-7 heavy chain version 2 (MJ2-7 V2VH) were selected based on BLAST homology searches against Protein Data Bank (PDB). Besides the two structures selected from the BLAST search output, an additional template was selected from an in-house database of protein structures. Model of MJ2-7 V2VH was built using the three template structures 1JPS (co-crystal structure of human tissue factor in complex with humanized Fab D3h44), 1N8Z (co-crystal structure of human Her2 in complex with Herceptin Fab) and F13.2 (IL-13 in complex with mouse antibody Fab fragment) as templates and the Homology module of InsightII (Accelrys, San Diego). The structurally conserved regions (SCRs) of 1JPS, 1N8Z and F13.2 (available from 16163-029001) were determined based on the Cα distance matrix for each molecule and the template structures were superimposed based on minimum RMS deviation of corresponding atoms in SCRs. The sequence of the target protein MJ2-7 V2VH was aligned to the sequences of the superimposed templates proteins and coordinates of the SCRs were assigned to the corresponding residues of the target protein. Based on the degree of sequence similarity between the target and the templates in each of the SCRs, coordinates from different templates were used for different SCRs. Coordinates for loops and variable regions not included in the SCRs were generated by Search Loop or Generate Loop methods as implemented in Homology module. Briefly, Search Loop method scans protein structures that would fit properly between two SCRs by comparing the Ca distance matrix of flanking SCR residues with a pre-calculated matrix derived from protein structures that have the same number of flanking residues and an intervening peptide segment of a given length. Generate Loop method that generate atom coordinates de novo was used in those cases where Search Loops did not produce desired results. Conformation of amino acid side chains was kept the same as that in the template if the amino acid residue was identical in the template and the target. However, a conformational search of rotamers was done and the energetically most favorable conformation was retained for those residues that are not identical in the template and target. This was followed by Splice Repair that sets up a molecular mechanics simulation to derive proper bond lengths and bond angles at junctions between two SCRs or between SCR and a variable region. Finally the model was subjected to energy minimization using Steepest Descents algorithm until a maximum derivative of 5 kcal/(mol Å) or 500 cycles and Conjugate Gradients algorithm until a maximum derivative of 5 kcal/(mol Å) or 2000 cycles. Quality of the model was evaluated using ProStat/Struct_Check command.

Molecular model of mouse MJ2-7 VH was built by following the procedure described for humanized MJ2-7 V2VH except the templates used were 1QBL and 1QBM, crystal structures for horse anti-cytochrome c antibody FabE8.

Potential differences in CDR-Framework H-bonds predicted by the models

hMJ2-7 V2VH:G26-hMJ2-7 V2VH:A24

hMJ2-7 V2VH:Y109-hMJ2-7 V2VH:S25

mMJ2-7 VH:D61-mMJ2-7 VH:148

mMJ2-7 VH:K₆₃-mMJ2-7 VH:E46

mMJ2-7 VH:Y109-mMJ2-7 VH:R98

These differences suggested the following optional back mutations: A23T, A24G and V48I.

Other optional back mutations suggested based on significant RMS deviation of individual amino acids and differences in amino acid residues adjacent to these are: G9A, L115T and R87T.

### Example 15

### IL-13 Neutralization Activity of MJ2-7 and C65

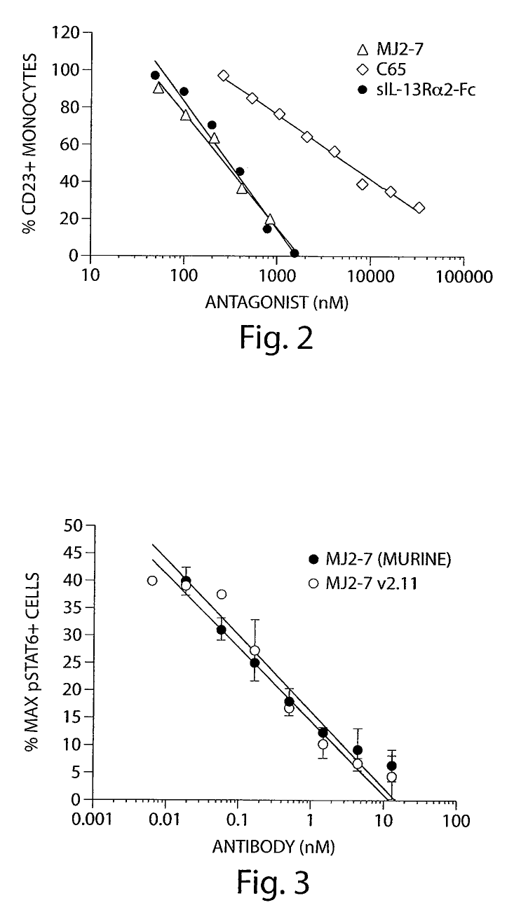

The IL-13 neutralization capacities of MJ2-7 and C65 were tested in a series of bioassays. First, the ability of these antibodies to neutralize the bioactivity of NHP IL-13 was tested in a monocyte CD23 expression assay. Freshly isolated human PBMC were incubated overnight with 3 ng/ml NHP IL-13 in the presence of increasing concentrations of MJ2-7, C65, or sIL-13Rα2-Fc. Cells were harvested, stained with CYCHROME™-labeled antibody to the monocyte-specific marker, CD11b, and with PE-labeled antibody to CD23. In response to IL-13 treatment, CD23 expression is up-regulated on the surface of monocytes, which were gated based on expression of CD11b. MJ2-7, C65, and sIL13Rα2-Fc all were able to neutralize the activity of NHP IL-13 in this assay. The potencies of MJ2-7 and sIL-13Rα2-Fc were equivalent. C65 was approximately 20-fold less active (FIG. 2).

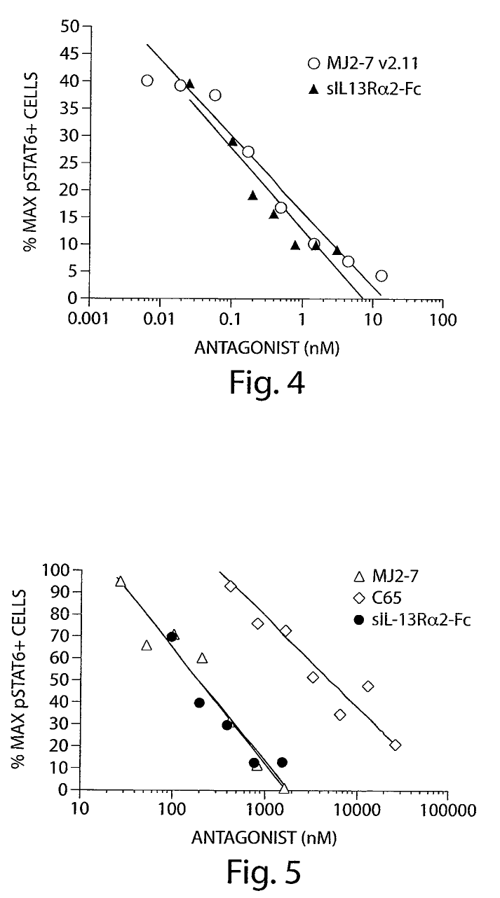

In a second bioassay, the neutralization capacities of MJ2-7 and C65 for native human IL-13 were tested in a STAT6 phosphorylation assay. The HT-29 epithelial cell line was incubated with 0.3 ng/ml native human IL-13 in the presence of increasing concentrations of MJ2-7, C65, or sIL-13Rα2-Fc, for 30 minutes at 37° C. Cells were fixed, permeabilized, and stained with ALEXA™ Fluor 488-labeled antibody to phosphorylated STAT6. IL-13 treatment stimulated STAT6 phosphorylation. MJ2-7, C65, and sIL13Rα2-Fc all were able to neutralize the activity of native human IL-13 in this assay (FIG. 3). The IC50's for the murine MJ-27 antibody and the humanized form (V2.11) were 0.48 nM and 0.52 nM respectively. The potencies of MJ2-7 and sIL-13Rα2-Fc were approximately equivalent. The IC₅₀ for sIL-13Ra2-Fc was 0.33 nM (FIG. 4). C65 was approximately 20-fold less active (FIG. 5).

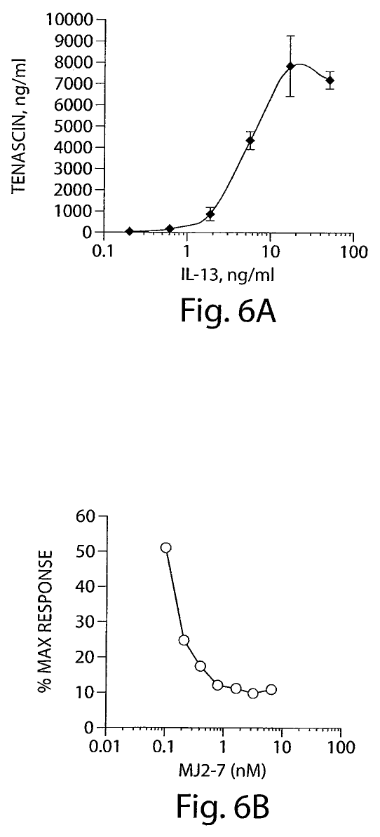

In a third bioassay, the ability of MJ2-7 to neutralize native human IL-13 was tested in a tenascin production assay. The human BEAS-2B lung epithelial cell line was incubated overnight with 3 ng/ml native human IL-13 in the presence of increasing concentrations of MJ2-7. Supernatants were harvested and tested for production of the extracellular matrix protein, tenascin C, by ELISA (FIG. 6A). MJ2-7 inhibited this response with IC₅₀ of approximately 0.1 nM (FIG. 6B).

These results demonstrate that MJ2-7 is an effective neutralizer of both NHP IL-13 and native human IL-13. The IL-13 neutralization capacity of MJ2-7 is equivalent to that of sIL-13Rα2-Fc. MJ1-65 also has IL-13 neutralization activity, but is approximately 20-fold less potent than MJ2-7.

### Example 16

### Epitope Mapping of MJ2-7Antibody by SPR

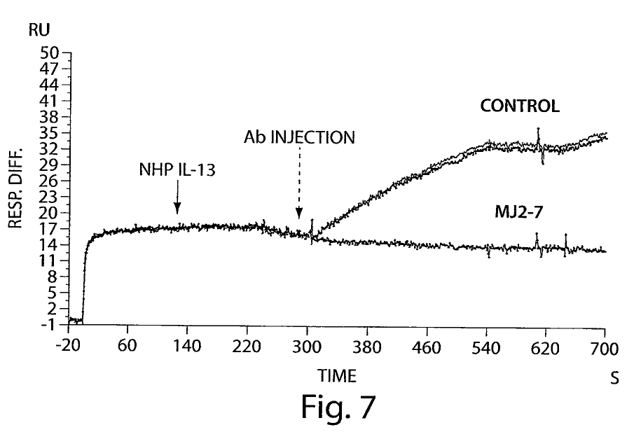

sIL-13Rα2-Fc was directly coated onto a CM5 chip by standard amine coupling. NHP-IL-13 at 100 nM concentration was injected, and its binding to the immobilized IL-13Rα2-Fc was detected by BIACORE™. An additional injection of 100 nM of anti IL-13 antibodies was added, and changes in binding were monitored. MJ2-7 antibody did not bind to NHP-IL-13 when it was in a complex with hu IL-13Rα2, whereas a positive control anti-IL-13 antibody did (FIG. 7). These results indicate that hu IL-13Rα2 and MJ2-7 bind to the same or overlapping epitopes of NHP IL-13.

### Example 17

### Measurement of Kinetic Rate Constants for the Interaction Between NHP-IL-13 and Humanized MJ2-7 V2-11 Antibody

To prepare the biosensor surface, goat anti-human IgG Fc specific antibody was immobilized onto a research-grade carboxy methyl dextran chip (CM5) using amine coupling. The surface was activated with a mixture of 0.1 M 1-ethyl-3-(3-dimethylaminopropyl) carbodiimide (EDC) and 0.05 M N-Hydroxysuccinimide (NHS). The capturing antibody was injected at a concentration of 10 μg/ml in sodium acetate buffer (pH 5.5). Remaining activated groups were blocked with 1.0 M ethanolamine (pH 8.0). As a control, the first flow cell was used as a reference surface to correct for bulk refractive index, matrix effect, s and non-specific binding, the second, third and fourth flow cells were coated with the capturing molecule.

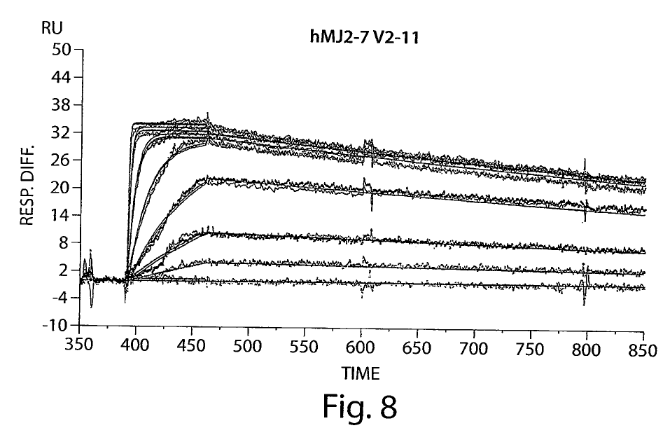

For kinetic analysis, the monoclonal antibody hMJ2-7 V2-11 was captured onto the anti IgG antibody surface by injecting 40 μl of a 1 μg/ml solution. The net difference between the baseline and the point approximately 30 seconds after completion of injection was taken to represent the amount of target bound. Solutions of NHP-IL-13 at 600, 200, 66.6, 22.2, 7.4, 2.5, 0.8, 0.27, 0.09 and 0 nM concentrations were injected in triplicate at a flow rate of 1001 per min for 2 minutes, and the amount of bound material as a function of time was recorded (FIG. 8). The dissociation phase was monitored in HBS/EP buffer (10 mM HEPES, pH 7.4, containing 150 mM NaCl, 3 mM EDTA and 0.005% (v/v) Surfactant P20) for 5 minutes at the same flow rate followed by two 5 μl injections of glycine, pH 1.5, to regenerate a fully active capturing surface. All kinetic experiments were done at 22.5° C. in HBS/EP buffer. Blank and buffer effects were subtracted for each sensorgram using double referencing.

The kinetic data were analyzed using BIAEVALUATION™ software 3.0.2 applied to a 1:1 model. The apparent dissociation (kd) and association (ka) rate constants were calculated from the appropriate regions of the sensorgrams using a global analysis. The affinity constant of the interaction between antibody and NHP IL-13 was calculated from the kinetic rate constants by the following formula: Kd=kd/ka. These results indicate that huMJ2-7 V2-11 has on and off-rates of 2.05×10⁷ M⁻¹ s⁻¹ and 8.89×10⁻⁴ 1/s, respectively, resulting in an antibody with 43 pM affinity for NHP-IL-13.

### Example 18

### Inhibitory Activity of MJ2-7 Humanization Intermediates in Bioassays

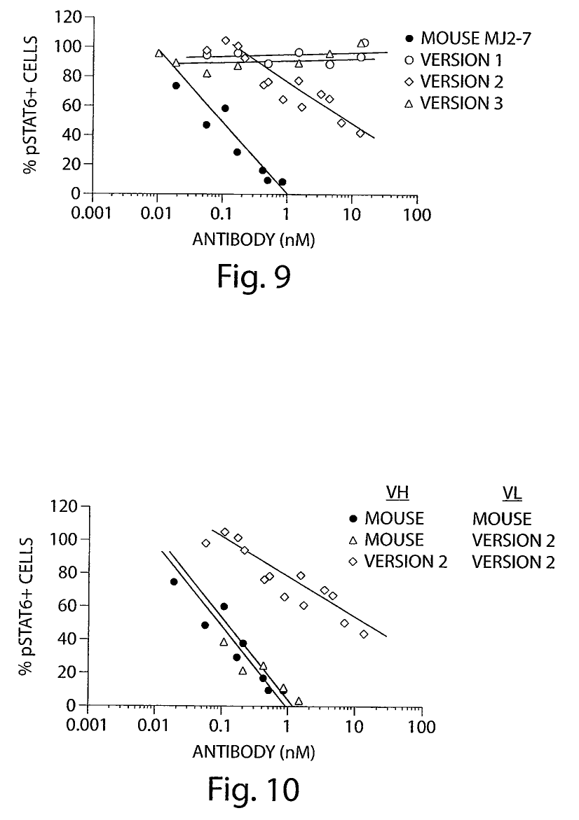

The inhibitory activity of various intermediates in the humanization process was tested by STAT6 phosphorylation and tenascin production bioassays. A sub-maximal level of NHP IL-13 or native human IL-13 crude preparation was used to elicit the biological response, and the concentration of the humanized version of MJ2-7 required for half-maximal inhibition of the response was determined. Analysis hMJ2-7 V1, hMJ2-7 V2 and hMJ2-7 V3, expressed with the human IgG1, and kappa constant regions, showed that Version 2 retained neutralization activity against native human IL-13. This concentration of the Version 2 humanized antibody required for half-maximal inhibition of native human IL-13 bioactivity was approximately 110-fold greater than that of murine MJ2-7 (FIG. 9). Analysis of a semi-humanized form, in which the V1 or V2 VL was combined with murine MJ2-7 VH, demonstrated that the reduction of native human IL-13 neutralization activity was not due to the humanized VL, but rather to the VH sequence (FIG. 10). Whereas the semi-humanized MJ2-7 antibody with VL V1 only partially retained the neutralization activity the version with humanized VL V2 was as active as parental mouse antibody. Therefore, a series of back-mutations were introduced into the V1 VH sequence to improve the native human IL-13 neutralization activity of murine MJ2-7.

### Example 19

### MJ2-7 Blocks IL-13 Interaction with IL-13Rα1 and IL-13Rα2

MJ2-7 is specific for the C-terminal 19-mer of NHP IL-13, corresponding to amino acid residues 114-132 of the immature protein (SEQ ID NO:24), and residues 95-113 of the mature protein (SEQ ID NO:14). For human IL-13, this region, which forms part of the D alpha-helix of the protein, has been reported to contain residues important for binding to both IL-13Rα1 and IL-13Rα2. Analysis of human IL-13 mutants identified the A, C, and D-helices as containing important contacts site for the IL-13Rα1/IL-4Rα signaling complex (Thompson and Debinski (1999) J. Biol. Chem. 274: 29944-50). Alanine scanning mutagenesis of the D-helix identified residues K123, K124, and R127 (SEQ ID NO:24) as responsible for interaction with IL-13Rα2, and residues E110, E128, and L122 as important contacts for IL-13Rα1 (Madhankmuar et al. (2002) J. Biol. Chem. 277: 43194-205). High resolution solution structures of human IL-13 determined by NMR have predicted the IL-13 binding interactions based on similarities to related ligand-receptor pairs of known structure. These NMR studies have supported a key role for the IL-13 A and D-helices in making important contacts with IL-13Rα1 (Eisenmesser et al. (2001) J. Mol. Biol. 310:231-241; Moy et al. (2001) J. Mol. Biol. 310:219-230). Binding of MJ2-7 to this epitope located in the C-terminal, D-helix of IL-13 was predicted to disrupt interaction of IL-13 with IL-13Rα1 and IL-13Rα2.

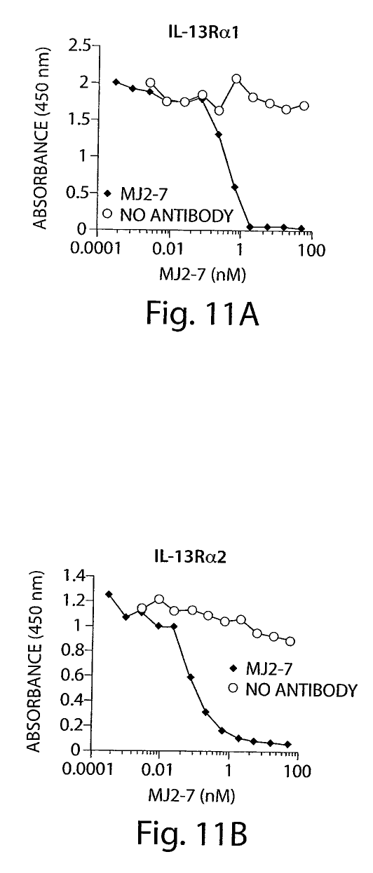

The ability of MJ2-7 to inhibit binding of NHP IL-13 to IL-13Rα1 and IL-13Rα2 was tested by ELISA. Recombinant soluble forms of human IL-13Rα1-Fc and IL-13Rα2-Fc were coated onto ELISA plates. FLAG-tagged NHP IL-13 was added in the presence of increasing concentrations of MJ2-7. Results showed that MJ2-7 competed with both soluble receptor forms for binding to NHP IL-13 (FIGS. 11A and 11B). This provides a basis for the neutralization of IL-13 bioactivity by MJ2-7.

### Example 20

### The MJ 2-7 Light Chain CDRs Contribute to Antigen Binding

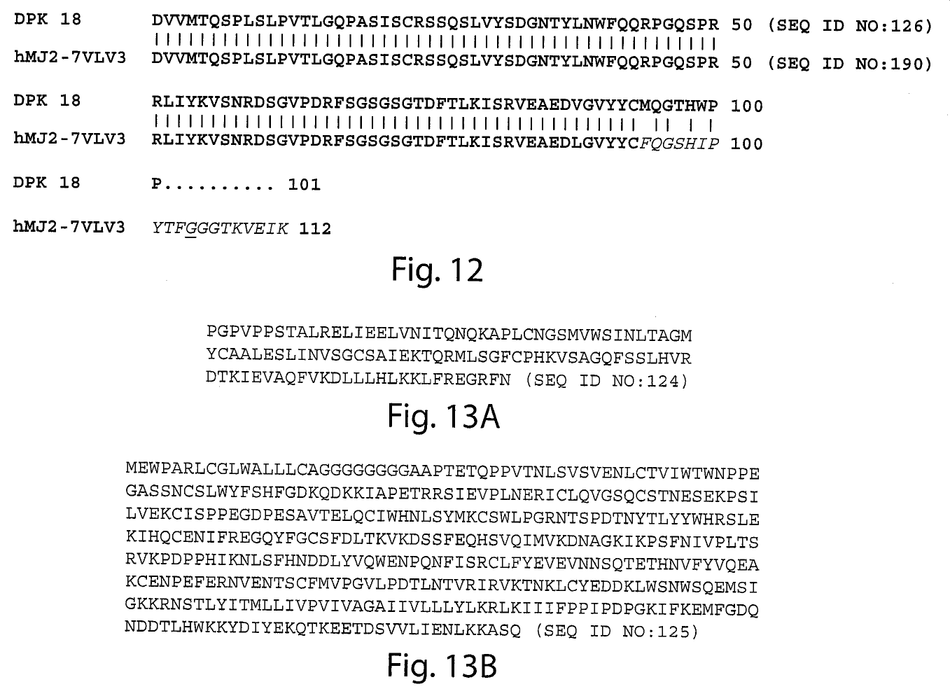

To evaluate if all three light chain CDR regions are required for the binding of MJ 2-7 antibody to NHP IL-13, two additional humanized versions of MJ 2-7 VL were constructed by CDR grafting. The VL version 3 was designed based on human germline clone DPK18, contained CDR1 and CDR2 of the human germline clone and CDR3 from mouse MJ2-7 antibody (FIG. 12). In the second construct (hMJ 2-7 V4), only CDR1 and CDR2 of MJ 2-7 antibody were grafted onto DPK 18 framework, and CDR3 was derived from irrelevant mouse monoclonal antibody.

The humanized MJ 2-7 V3 and V4 were produced in COS cells by combining hMJ 2-7 VH V1 with hMJ 2-7 VL V3 and V4. The antigen binding properties of the antibodies were examined by direct NHP IL-13 binding ELISA. The hMJ 2-7 V4 in which MJ 2-7 light chain CDR3 was absent retained the ability to bind NHP IL-13, whereas V3 that contained human germline CDR1 and CDR2 in the light chain did not bind to immobilized NHP IL-13. These results demonstrate that CDR1 and CDR2 of MJ 2-7 antibody light chain are most likely responsible for the antigen binding properties of this antibody.

Nucleotide sequence of hMJ 2-7 VL V3

| (SEQ ID NO:189) | |
|---|---|
| 1 | ATGCGGCTGC CCGCTCAGCT GCTGGGCCTG CTGATGCTGT GGGTGCCCGG |
| 51 | CTCTTCCGGC GACGTGGTGA TGACCCAGTC CCCTCTGTCT CTGCCCGTGA |
| 101 | CCCTGGGCCA GCCCGCTTCT ATCTCTTGCC GGTCCTCCCA GTCCCTGGTG |
| 151 | TACTCCGACG GCAACACCTA CCTGAACTGG TTCCAGCAGA GACCCGGCCA |
| 201 | GTCTCCTCGG CGGCTGATCT ACAAGGTGTC CAACCGCTTT TCCGGCGTGC |
| 251 | CCGATCGGTT CTCCGGCTCC GGCAGCGGCA CCGATTTCAC CCTGAAGATC |
| 301 | AGCCGCGTGG AGGCCGAGGA TGTGGGCGTG TACTACTGCT TCCAGGGCTC |
| 351 | CCACATCCCT TACACCTTTG GCGGCGGAAC CAAGGTGGAG ATCAAG |

Amino acid sequence of hMJ 2-7 VL V3

(SEQ ID NO:190)

MRLPAQLLGLLMLWVPGSSG-DVVMTQSPLSLPVTLGQPASISCRSSQSL
VYSDGNTYLNWFQQRPGQSPRRLIYKVSNRFSGVPDRFSGSGSGTDFTLK
ISRVEAEDVGVYYCFQGSHIPYTFGGGTKVEIK

Nucleotide sequence of hMJ 2-7 VL V4

(SEQ ID NO:191)

GATGTTGTGATGACCCAATCTCCACTCTCCCTGCCTGTCACTCCTGGAGA
GCCAGCCTCCATCTCTTGCAGATCTAGTCAGAGCATTGTGCATAGTAATG
GAAACACCTACCTGGAATGGTACCTGCAGAAACCAGGCCAGTCTCCACAG
CTCCTGATCTACAAAGTTTCCAACCGATTTTCTGGGGTCCCAGACAGGTT
CAGTGGCAGTGGATCAGGGACAGATTTCACACTCAAGATCAGCAGAGTGG
AGGCTGAGGATGTGGGAGTTTATTACTGCTTTCAAAGTTCACATGTTCCT
CTCACCTTCGGTCAGGGGACCAAGCTGGAGATCAAA

Amino acid sequence of hMJ 2-7 VL V4

(SEQ ID NO: 192)

DVVMTQSPLS LPVTPGEPAS ISCRSSQSIV HSNGNTYLEW
YLQKPGQSPQ LLIYKVSNRF SGVPDRFSGS GSGTDFTLKISRVEAED
VGV YYCFQSSHVP LTFGQGTKLE IK

### Example 21

### Neutralizing Activities of Anti-IL13 Antibodies in Cynomolgus Monkey Model

The efficacy of an IL-13 binding agent (e.g., an anti-IL13 antibody) in neutralizing one or more IL-13-associated activities in vivo can be tested using a model of antigen-induced airway inflammation in cynomolgus monkeys naturally allergic to Ascaris suum. These assays can be used to confirm that the binding agent effectively reduces airway eosinophilia in allergic animals challenged with an allergen. In this model, challenge of an allergic monkey with Ascaris suum antigen results in one or more of the following: (i) an influx of inflammatory cells, e.g., eosinophils into the airways; (ii) increased eotaxin levels; (iii) increase in Ascaris-specific basophil histamine release; and/or (iv) increase in Ascaris-specific IgE titers.

To test the ability of an anti-IL-13 antibody to prevent the influx of inflammatory cells, the antibody can be administered 24 hours prior to challenge with Ascaris suum antigen. On the day of challenge, a baseline bronchoalveolar lavage (BAL) sample can be obtained from the left lung. Ascaris suum antigen can be instilled intratracheally into the right lung. Twenty-four hours later, the right lung is ravaged, and the BAL fluid from animals treated intravenously with the antibody were compared to BAL fluid from untreated animals. If the antibody reduces airway inflammation, an increase in percent BAL eosinophils may be observed among the untreated group, but not for the antibody-treated group.

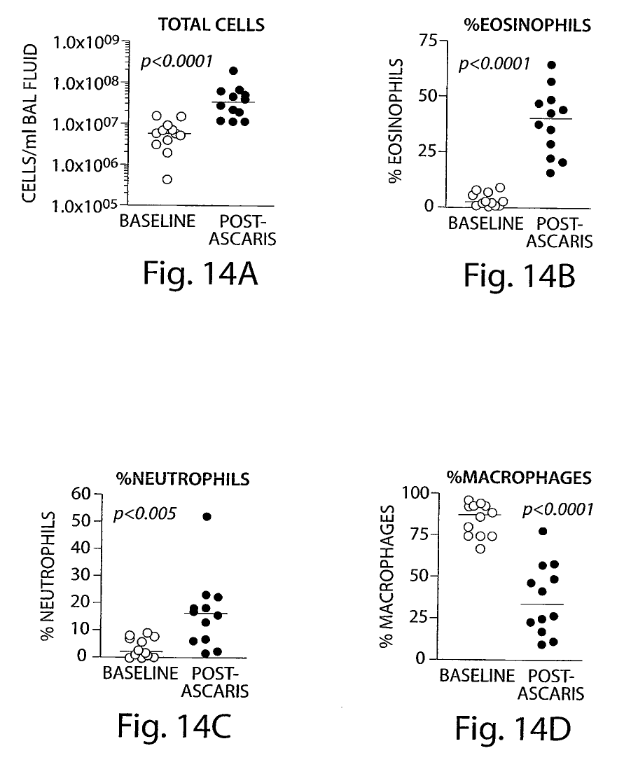

FIGS. 14A-14D depict an increase in the total number of cells and percentage of inflammatory cells, for example, eosinophils (FIG. 14B), neutrophils (FIG. 14C) and macrophages (FIG. 14D) 24-hours following airway challenge with Ascaris. A statistically significant increase in the percentage of inflammatory cells was observed 24 hours after challenge compared to the baseline values.

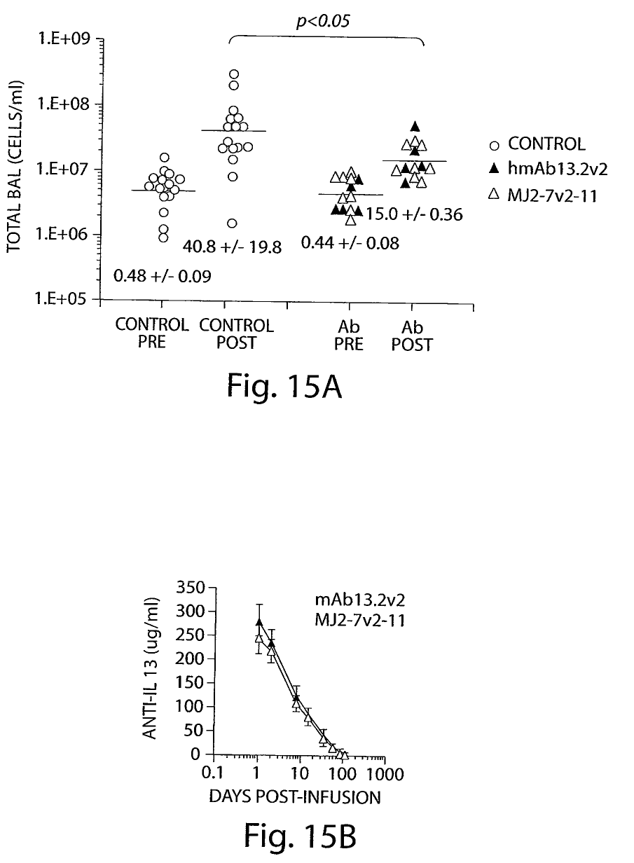

Anti-IL13 antibodies (humanized MJ2-7v.2-11 and humanized mAb13.2v.2) were administered to cynomolgus monkeys 24 hours prior to challenge with Ascaris suum antigen. (mAb 13.2 and its humanized form hmAb13.2v2 were described in commonly owned PCT application WO 05/123126, the contents of which are incorporated herein by reference in their entirety). Control monkeys were treated with saline. 10 mg/kg of hMJ2-7v2-11, hmAb13.2v2, or irrelevant human Ig (IVIG) were administered intravenously. The following day, prechallenged BAL samples from control and treated monkeys (referred to in FIG. 15A as “control pre” and “Ab pre”) were collected from the left lung of the monkeys. The monkeys were treated with 0.75 micrograms of Ascaris suum antigen intratracheally into the right lung. Twenty-four hours post-challenge, BAL samples were collected from the right lung of control and treated monkeys, and assayed for cellular infiltrate (referred to in FIG. 15B as “control post” and “Ab post,” respectively). BAL samples collected from antibody-treated monkeys showed a statistically significant reduction in the total number of cell infiltrate compared to control animals (FIG. 15A). Control samples are represented in FIG. 15A as circles, hmAb13.2v2- and hMJ2-7v2-11-treated samples are shown as dark and light triangles, respectively. hMJ2-7v2-11 and hmAb13.2v2 showed comparable efficacy in this model. FIG. 15B shows a linear graph depicting the concentration of either hMJ2-7v2-11 or hmAb13.2v2 with respect to days post-Ascaris infusion. A comparable decrease kinetics is detected for both antibodies.

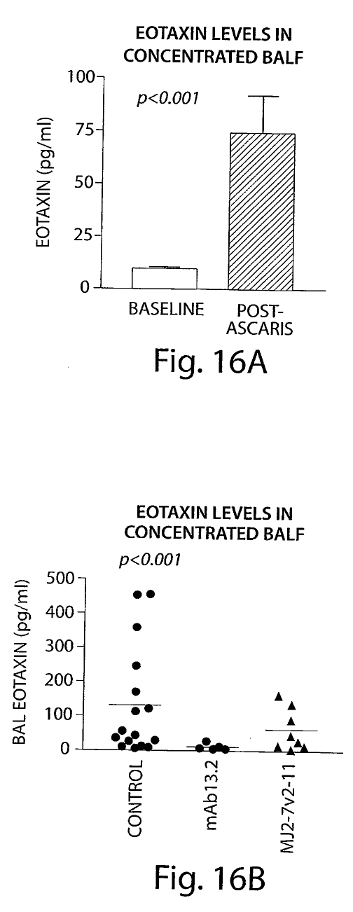

Eotaxin levels were significantly increased 24 hours following Ascaris challenge (FIG. 16A). Both hMJ2-7v2-11 and hmAb13.2v2 reduced eotaxin levels detected in BAL fluids from cynomolgus monkeys 24 hours after to challenge with Ascaris suum antigen, compared to saline treated controls.

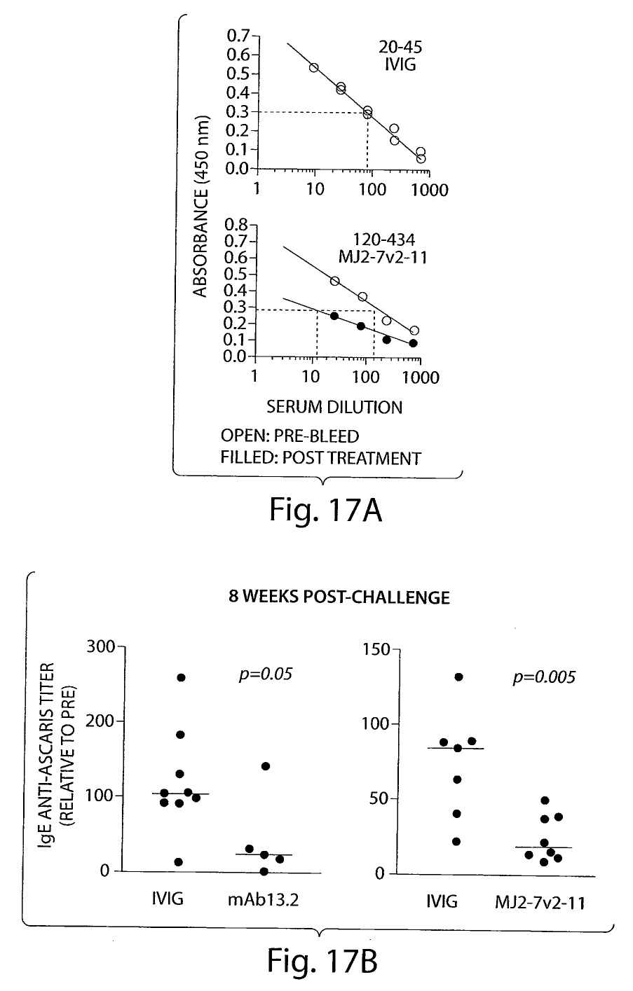

Cynomolgus monkeys sensitized to Ascaris suum develop IgE to Ascaris antigen. The IgE binds to FcεRI on circulating basophils, such that in vitro challenge of peripheral blood basophils with Ascaris antigen induces degranulation and release of histamine. Repeated antigen exposure boosts basophil sensitization, resulting in enhanced histamine release responses. To test the effects of hMJ2-7v2-11 and hmAb13.2v2 in IgE- and basophil levels, cynomolgus monkeys dosed with humanized hMJ2-7v.2, hmAb13.2v2, irrelevant Ig (IVIG), or saline, as described above, were bled 8 weeks post-Ascaris challenge, and levels of total and Ascaris-specific IgE in plasma were determined by ELISA. FIG. 17A shows a linear graph of the changes in absorbance with respect to dilution of samples obtained pre- and 8-weeks post-challenge from animals treated with IVIG or hMJ2-7v2-11. Open-circles represent pre-bleed measurements; filled circles represent post-treatment measurements. A significant reduction in absorbance was detected in post-challenged samples treated with hMJ2-7v2-11 relative to the pre-challenge values in all dilutions assayed FIG. 17A depicts representative examples showing no change in Ascaris-specific IgE titer in an individual monkey treated with irrelevant Ig (IVIG; animal 20-45; top panel), and decreased titer of Ascaris-specific IgE in an individual monkey treated with hMJ2-7v2-11 (animal 120-434; bottom panel).

Animals treated with either humanized hMJ2-7v.2-11 or hmAb13.2v2 showed a significant reduction in levels of circulating IgE-specific for Ascaris in cynomolgus monkey sera (FIG. 17B). There was no significant change in total IgE titer for any of the treatment groups. FIG. 17A shows a linear graph of the changes in absorbance with respect to dilution of samples obtained pre- and 8-weeks post-challenge from animals treated with IVIG or hMJ2-7v2-11. Open-circles represent pre-bleed measurements; filled circles represent post-treatment measurements. A significant reduction in absorbance was detected in post-challenged samples treated with hMJ2-7v2-11 relative to the pre-challenge values in all dilutions assayed. The designations “20-45” and “120-434” refer to the cynomolgus monkey identification number.

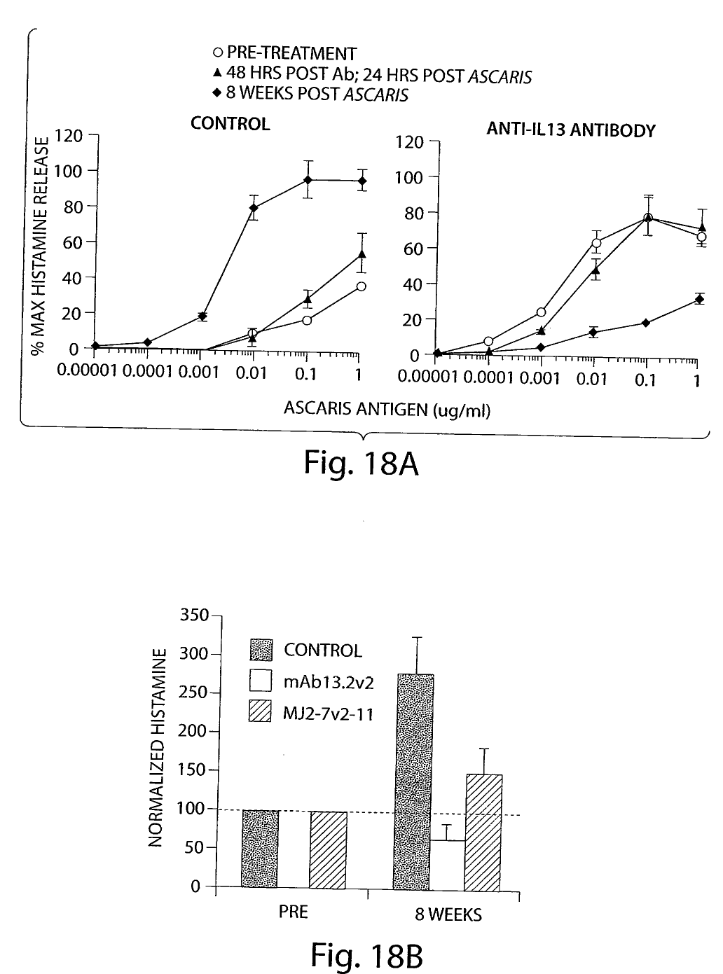

To evaluate the effects of anti-IL13 antibodies on basophil histamine release, the animals were bled at 24 hours and 8 weeks post-Ascaris challenge. Whole blood was challenged with Ascaris antigen for 30 minutes at 37° C., and histamine released into the supernatant was quantitated by ELISA (Beckman Coulter, Fullerton, Calif.). As shown in FIGS. 18A-18B, the control animals demonstrated increased levels of Ascaris-induced basophil histamine release particularly 8 weeks following antigen challenge (represented by the diamonds in FIG. 18A and left-hand bar in FIG. 18B). In contrast, the animals treated with either humanized hMJ2-7v.2-11 or hmAb13.2v2 did not show this increase in basophil sensitization in response to Ascaris 8 weeks after challenge (FIGS. 18A-18B). The majority of individual animals treated with humanized hMJ2-7v.2-11 or hmAb13.2v2 showed either a decrease (example in FIG. 18A) or no change in basophil histamine release 8 weeks post-challenge compared to pre- or 24 hour post-challenge. Thus, a single administration of the humanized anti-IL13 antibody had a lasting effect in modifying histamine release in this model.

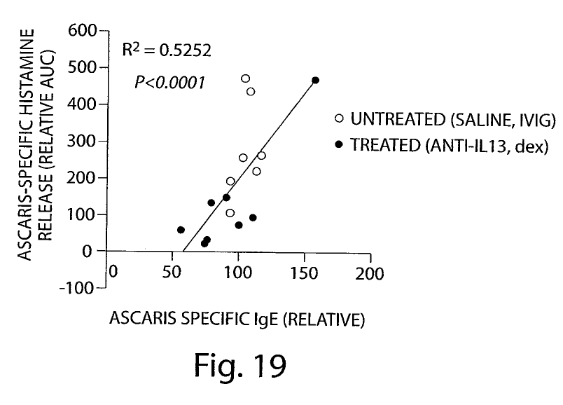

FIG. 19 depicts the correlation between Ascaris-specific histamine release and Ascaris-specific IgE levels. Higher values were detected in control samples (saline- or IVIG-treated samples) (light blue circles) compared to anti-IL13 antibody- or dexamethasone (dex)-treated (dark red circles). Humanized anti-IL13 antibody (humanized mAb13.2v.2) administered i.v. 24 hours prior to Ascaris challenge, or dexamethasone administered intramuscular in two injections each one at a concentration of 1 mg/kg 24 hours and 30 mins. prior to Ascaris challenge. Twenty four hours post-challenge, BAL lavage was collected from the right lung and assayed for histamine release and IgE levels.

The results shown herein demonstrated that pretreatment of cynomolgus monkeys with either MJ2-7 or mAb13.2 reduced airway inflammation induced by Ascaris suum antigen at comparable levels as detected by cytokine levels in BAL samples, serum levels of Ascaris-specific IgE's and basophil histamine release in response to Ascaris challenge in vitro.

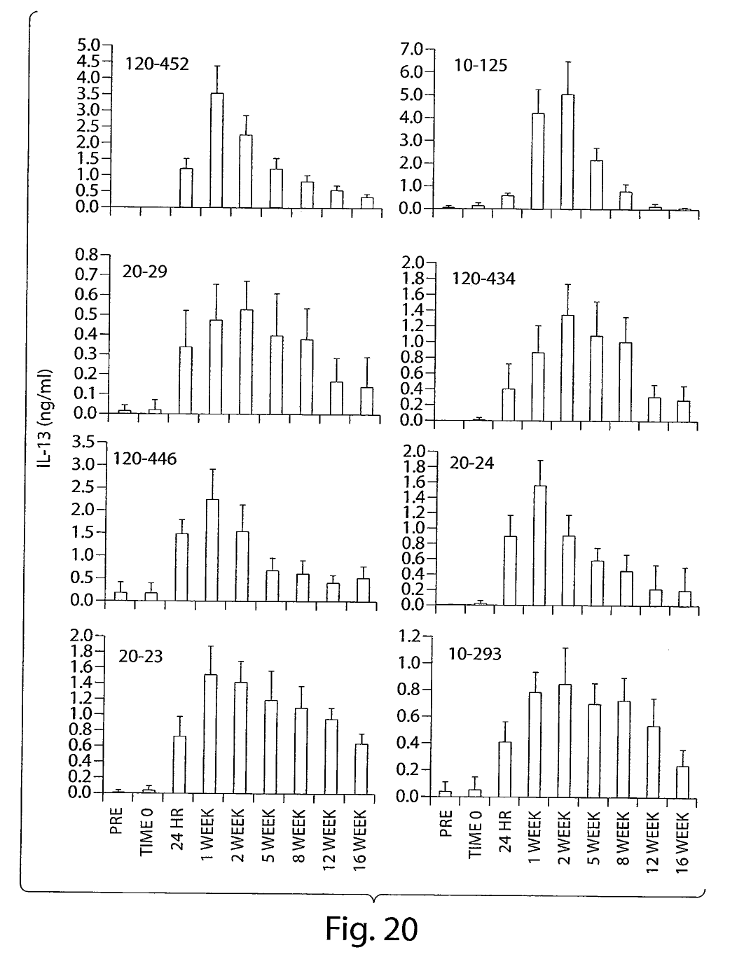

FIG. 20 is a series of bar graphs depicting the increases in serum IL-13 levels in individual cynomolgus monkeys treated with humanized MJ2-7 (hMJ2-7v2-11). The label in each panel (e.g., 120-452) corresponds to the monkey identification number. The “pre” sample was collected prior to administration of the antibody. The time “0” was collected 24-hours post-antibody administration, but prior to Ascaris challenge. The remaining time points were post-Ascaris challenge. The assays used to detect IL-13 levels are able to detect IL-13 in the presence of hMJ2-7v2-11 or hmAb13.2v2 antibodies. More specifically, ELISA plates (MaxiSorp; Nunc, Rochester, N.Y.), were coated overnight at 4° C. with 0.5 ug/ml mAb13.2 in PBS. Plates were washed in PBS containing 0.05% Tween-20 (PBS-Tween). NHP IL-13 standards, or serum dilutions from cynomolgus monkeys, were added and incubated for 2 hours at room temperature. Plates were washed, and 0.3 ug/ml biotinylated MJ1-64 (referred to herein as C65 antibody) was added in PBS-Tween. Plates were incubated 2 hours, room temperature, washed, and binding detected using HRP-streptavidin (Southern Biotechnology Associates) and Sure Blue substrate (Kirkegaard and Perry Labs). For detection of IL-13 in the presence of mAb13.2, the same protocol was followed, excepts that ELISA plates were coated with 0.5 ug/ml MJ2-7.

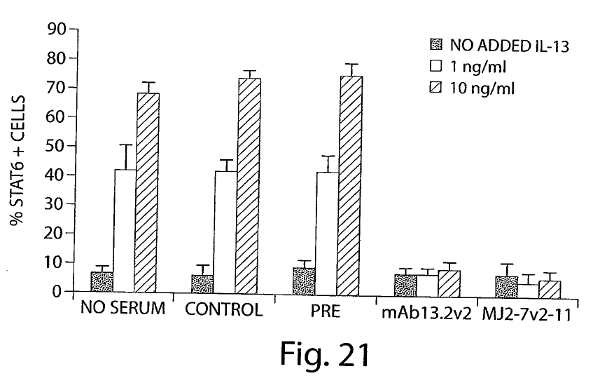

FIG. 21 shows data demonstrating that sera from cynomolgus monkeys treated with anti-IL13 antibodies have residual IL-13 neutralization capacity at the concentrations of non-human primate IL-13 tested. FIG. 21 is a bar graph depicting the STAT6 phosphorylation activity of non-human primate IL-13 at 0, 1, or 10 ng/ml, either in the absence of serum (“no serum”); the presence of serum from saline or IVIG-treated animals (“control”); or in the presence of serum from anti-IL13 antibody-treated animals, either before antibody administration (“pre”), or 1-2 weeks post-administration of the indicated antibody. Serum was tested at 1:4 dilution. A humanized version of MJ2-7 (MJ2-7v.2-11) was used in this study. Assays for measuring STAT6 phosphorylation are disclosed herein.

FIG. 22 are linear graphs showing that levels of non-human primate IL-13 trapped by humanized MJ2-7 (hMJ2-7v2-11) at a 1-week time point in cynomolgus monkey serum correlate with the level of inflammation measured in the BAL fluids post-Ascaris challenge. Such correlation supports that detection of serum IL-13 (either unbound or bound to an anti-IL13 antibody) as a biomarker for detecting subjects having inflammation. Subjects having more severe inflammation showed higher levels of serum IL-13. Although levels of unbound IL-13 are typically difficult to quantitate, the assays disclosed herein above in FIG. 20 provides a reliable assay for measuring IL-13 bound to an anti-IL-13 antibody.

### Example 22

### Effects of Humanized Anti-IL-13 Antibodies on Airway Inflammation, Lung Resistance, and Dynamic Lung Compliance Induced by Administration of Human IL-13 to Mice

Murine models of asthma have proved invaluable tools for understanding the role of IL-13 in this disease. The use of this model to evaluate in vivo efficacies of the IMA antibody series (humanized 13.2v.2 and humanized MJ2-7v.2-11) was initially hampered by the inability of these antibodies to cross react with rodent IL-13. This limitation was circumvented herein by administering human recombinant IL-13 to mice. Human IL-13 is capable of binding to the murine IL-13 receptor, and when administered exogenously induces airway inflammation, hyperresponsiveness, and other correlates of asthma.

In non-human primates, the IL-13 epitope recognized by humanized MJ2-7v.2-11 includes a GLN at position 110. In humans, however, position 110 is a polymorphic variant, typically with ARG replacing GLN (e.g., R110). The R110Q polymorphic variant is widely associated with increased prevalence of atopic disease.

In this example, recombinant human R110Q IL-13 was expressed in E. coli and refolded. Antibody 13.2 (IgG1, k) was cloned from BALB/c mice immunized with human IL-13, and the humanized version of this antibody is designated humanized 13.2v.2 (or h13.2v.2). Antibody MJ2-7 (IgG1, k) was cloned from BALB/c mice immunized with the N-terminal 19 amino acids of nonhuman primate IL-13, and the humanized version of this antibody is designated humanized MJ2-7v.2-11 (or hMJ2-7v.2-11). Both antibodies were formulated in 10 mM L-histidine, pH 6, containing 5% sucrose. Carimune NH immune globulin intravenous (human IVIG) (ZLB Bioplasma Inc., Switzerland) was purified by Protein A chromatography and formulated in 10 mM L-histidine, pH 6, containing 5% sucrose.

To analyze the mouse lung response to the presence of recombinant human R110Q IL-13, BABL/c female mice were treated with 5 μg of recombinant human R110Q IL-13 (e.g., approximately 250 μg/kg), or an equivalent volume of saline (20 μL), administered intratracheally on days 1, 2, and 3. On day 4, animals were tested for signs of airway resistance (RI) and compliance (Cdyn) in response to increasing doses of nebulized methacholine. Briefly, anesthetized and tracheostomized mice were placed into whole body plethysmographs, each with a manifold built into the head plate of the chamber, with ports to connect to the trachea, to the inspiration and expiration ports of a ventilator, and to a pressure transducer, monitoring the tracheal pressure. A pneumotachograph in the wall of each plethysmograph monitored the airflow into and out of the chamber, due to the thoracic movement of the ventilated animal. Animals were ventilated at a rate of 150 breaths/min and a tidal volume of 150 ml. Resistance computations were derived from the tracheal pressure and airflow signals, using an algorithm of covariance.

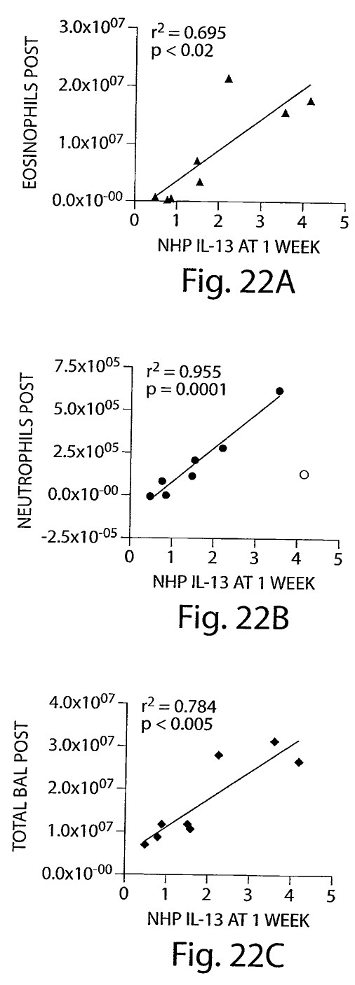

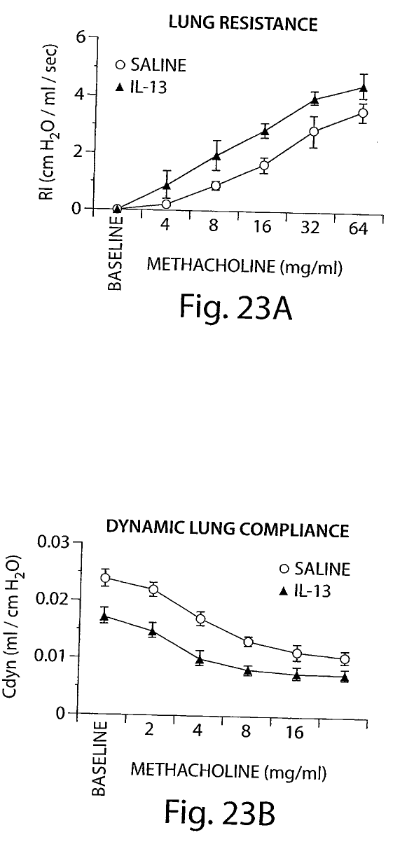

As shown in FIGS. 23A-23B, intratracheal administration of recombinant human R110Q IL-13 elicited increased lung resistance and decreased dynamic compliance in response to methacholine challenge. These observations were not, however, accompanied by strong lung inflammation.

To enhance, the lung inflammatory response in mice, 5 μg of recombinant human R110Q IL-13, or an equivalent volume (50 μL) of saline, was administered to C57BL/6 mice intranasally on days 1, 2, and 3. Animals were sacrificed on day 4 and bronchoalveolar lavage (BAL) fluid collected. Pre-analysis, BAL was filtered through a 70 μm cell strainer and centrifuged at 2,000 rpm for 15 minutes to pellet cells. Cell fractions were analyzed for total leukocyte count, spun onto microscope slides (Cytospin; Pittsburgh, Pa.), and stained with Diff-Quick (Dade Behring, Inc. Newark Del.) for differential analysis. IL-6, TNFα, and MCP-1 levels were determined by cytometric bead array (CBA; BD Pharmingen, San Diego, Calif.). The limits of assay sensitivity were 1 pg/ml for IL-6, and 5 pg/ml for TNFα and MCP-1.

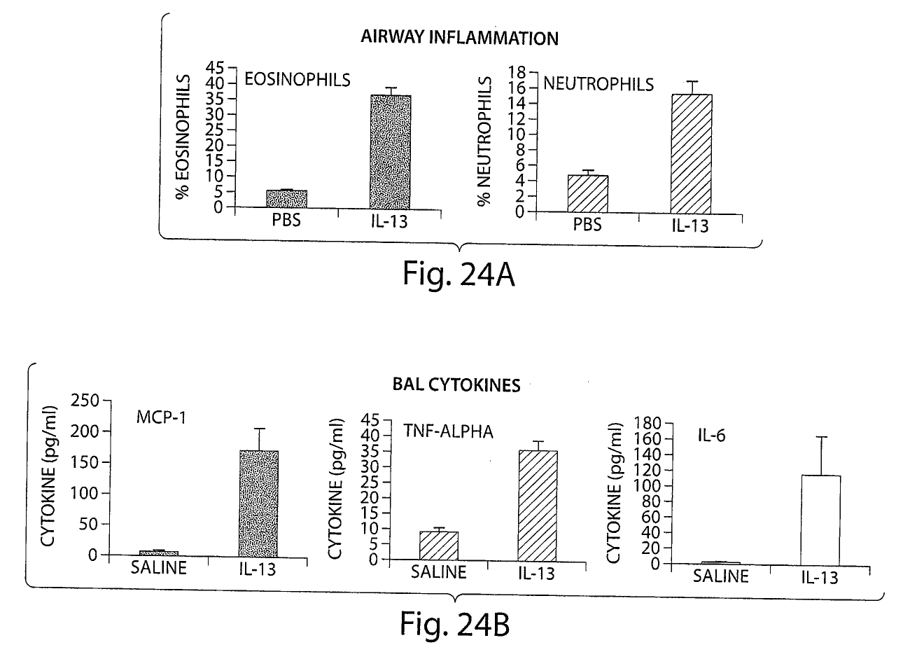

As shown in FIG. 24A, intranasal administration of recombinant human R100Q IL-13 induced a strong airway inflammatory response, as indicated by elevated eosinophil and neutrophil infiltration into BAL. Cell infiltrates consisted primarily of eosinophils (e.g., approximately 40%). As shown in FIG. 24B, intranasal administration of recombinant human R110Q IL-13 also significantly increased the levels of several cytokines in BAL including, for example, MCP-1, TNF-α, and IL-6.

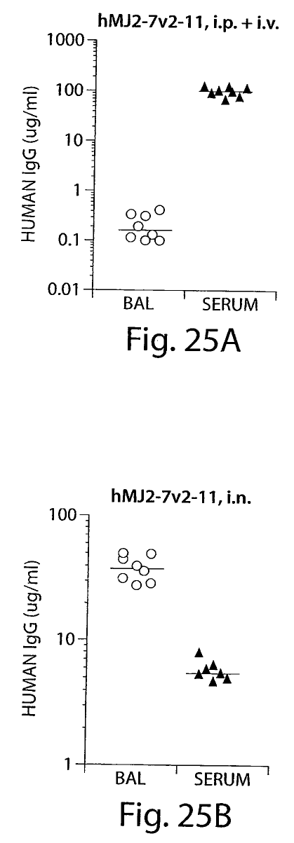

To determine the best delivery method for humanized MJ2-7v.2-11, antibody levels in BAL and serum were analyzed following intraperitoneal and intravenous, or intranasal administration following treatment with recombinant human R110Q IL-13 administered intranasally or intratracheally. Briefly, BALB/c female mice were administered 5 μg of recombinant human R110Q IL-13 or an equivalent volume of saline intratracheally on days 1, 2, and 3. On day 0, and 2 hours prior to administering each IL-13 dose, mice were treated with 500 μg humanized MJ2-7v.2administered intravenously on day 0, and by IP on days 1, 2, and 3 (FIG. 25A). Alternatively, 500 μg of humanized MJ2-7v.2-11 were administered intranasally on days 0, 1, 2, and 3. Total human IgG was measured by ELISA, as follows: ELISA plates (MaxiSorp; Nunc, Rochester, N.Y.) were coated overnight at 4° C. with 1:1500 dilution of goat anti-human Ig (M+G+A) Fc (ICN-Cappel, Costa Mesa, Calif.) at 50 μl/well in 25 mM carbonate-bicarbonate buffer, pH 9.6. Plates were blocked for 1 hour at room temperature with 0.5% gelatin in PBS, washed in PBS containing 0.05% Tween-20 (PBS-Tween). Humanized MJ2-7v.2-11 standard or 6×1:2 dilutions of sheep serum starting at 1:500-1:50,000 were added and incubated for 2 hours at room temperature. Plates were washed with PBS-Tween, and a 1:5000 dilution of biotinylated mouse anti-human IgG (Southern Biotechnology Associates) was incubated for 2 hours at room temperature. Plates were washed with PBS-Tween, and binding was detected with peroxidase-linked streptavidin (Southern Biotechnology Associates) and Sure Blue substrate (KPL Inc.). Assay sensitive was 0.5 ng/ml human IgG.

FIG. 25A shows elevated levels of human IgG in serum compared to BAL following intraperitoneal and intravenous administration of the humanized MJ2-7v.2-11 antibody. As shown in FIG. 25B, total IgG levels in μg/ml were significantly higher in BAL than serum levels following intranasal administration of humanized MJ2-7v.2-11 antibody.

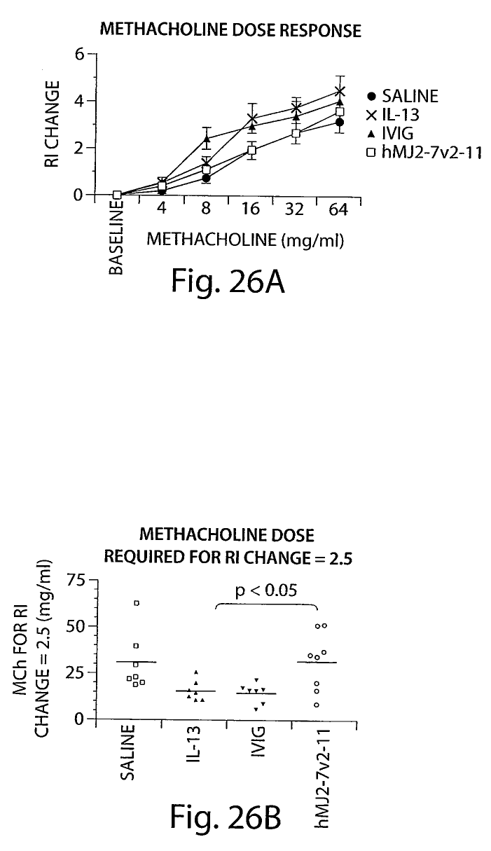

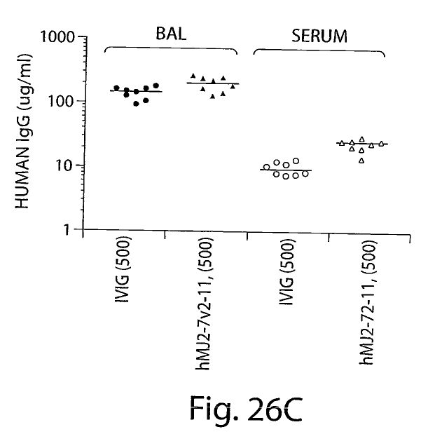

To determine if the humanized MJ2-7v.2-11 antibody was capable of modulating the above observed lung function and inflammatory response, airway hyperresponsiveness was induced by intratracheal administration of 5 μg recombinant human R100Q IL-13 or an equivalent volume (20 μL) of saline on days 1, 2, and 3. On day 0, and 2 hours before administering each dose of recombinant human R110Q IL-13, animals were treated with 500 μg of humanized MJ2-7v.2-11, 500 μg dose of IVIG, or an equivalent volume of saline, administered intranasally. Animals were tested on day 4 for airway resistance (RI) and compliance (Cdyn) in response to increasing doses of nebulized methacholine, as described above. Humanized MJ2-7v.2 and IVIG levels in BAL and serum were analyzed by ELISA, as described above. As shown in FIGS. 26A-26B, humanized MJ2-7v.2-11 effectively reduced the asthmatic response, resulting in a significant reduction in the dose of methacholine required to achieve half-maximal degree of lung resistance. In contrast, an equivalent dose of IVIG had no effect. Changes in dynamic lung compliance were not apparent under these conditions. As shown in FIG. 26C, BAL IgG antibody levels were approximately 10-20 times higher than serum levels.

To determine if humanized MJ2-7v.2-11 anti-IL-13 antibody administration promoted an increase in the circulating levels of IL-13, BAL and sera were assayed for IL-13 levels by ELISA, as follows: Briefly, BALB/c female mice were treated as described for FIG. 26A-26B. ELISA plates (Nunc Maxi-Sorp) were coated overnight with 50 μl/well mouse anti-IL-13 antibody, mAb 13.2, diluted to 0.5 mg/ml in PBS. Plates were washed 3 times with PBS containing 0.05% Tween-20 (PBS-Tween) and blocked for 2 hours at room temperature with 0.5% gelatin in PBS. Plates were then washed and human IL-13 standard (Wyeth, Cambridge, Mass.), or dilutions of mouse serum (serial 3× dilutions starting at 1:4) were added, in PBS-Tween containing 2% fetal calf serum (FCS). Plates were incubated for a further 4 hours at room temperature, and washed. Biotinylated mouse anti-human IL-13 antibody, C65, was added at 0.3 μg/ml in PBS-Tween. Plates were incubated for 1-2 hours at room temperature, washed, then incubated with HRP-streptavidin (Southern Biotechnology Associates, Birmingham, Ala.) for 1 hour at room temperature. Color was developed using Sure Blue peroxidase substrate (KPL, Gaithersburg, Md.), and the reaction stopped with 0.01M sulfuric acid. Absorbance was read at 450 nm in read in a SpectraMax plate reader (Molecular Devices Corp., Sunnyvale, Calif.). Serum IL-13 levels were determined by reference to a human IL-13 standard curve, which was independently established for each plate.

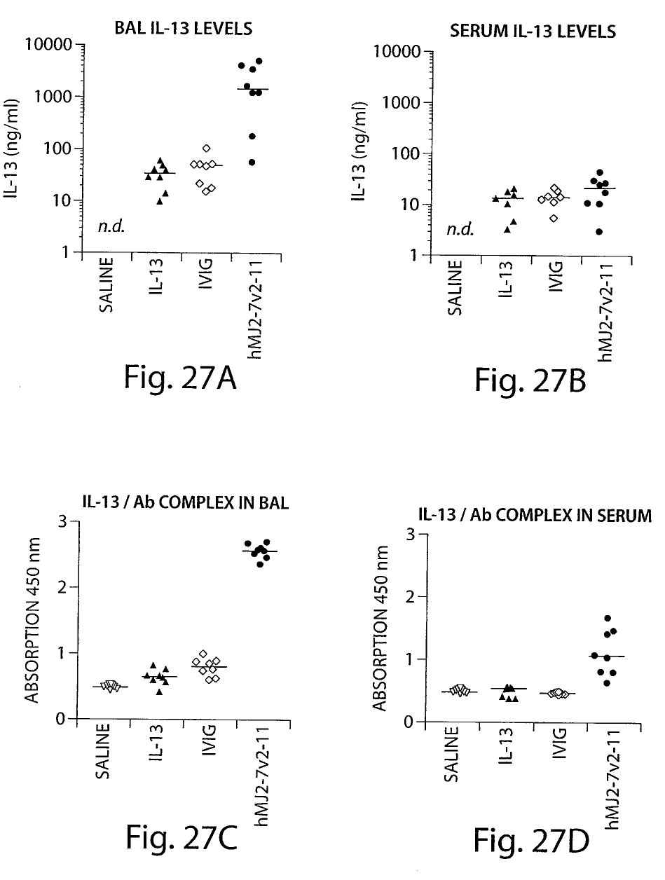

As shown in FIGS. 27A-27B, consistent with FIG. 26C, IL-13 levels were elevated in BAL of antibody-treated mice, but not serum. In addition, we observed that IL-13 isolated from these samples had no detectable biological activity (data not shown). To determine if this observed lack of IL-13 biological activity was due to IL-13 and humanized MJ2-7v.2-11 complex formation, an ELISA was developed to specifically detect IL-13 and humanized MJ2-7v.2-11 in complex. Briefly, ELISA plates (Nunc Maxi-Sorp) were coated overnight with 50 μl/well mouse anti-IL-13 antibody, mAb13.2, diluted to 0.5 mg/ml in PBS. Plates were washed 3 times with PBS containing 0.05% Tween-20 (PBS-Tween) and blocked for 2 hours at room temperature with 0.5% gelatin in PBS. Plates were then rewashed, and human IL-13 standard (Wyeth, Cambridge, Mass.), or dilutions of mouse serum (serial 3× dilutions starting at 1:4) were added, in PBS-Tween containing 2% fetal calf serum (FCS). Plates were subsequently incubated for 4 hours at room temperature. Biotinylated anti-human IgG (Fc specific) (Southern Biotechnology Associates, Birmingham, Ala.) diluted 1:5000 in PBS-Tween was then added. Plates were incubated for 1-2 hours at room temperature, washed, and finally incubated with HRP-streptavidin (Southern Biotechnology Associates, Birmingham, Ala.) for 1 hour at room temperature. Color was developed using Sure Blue peroxidase substrate (KPL, Gaithersburg, Md.), and the reaction stopped with 0.01M sulfuric acid. Absorbance was read at 450 nm in read in a SpectraMax plate reader (Molecular Devices Corp., Sunnyvale, Calif.).

As shown in FIGS. 27D-27E, IL-13 and humanized MJ2-7v.2-11 complexes were recovered from BAL and serum of mice in this model. This observation indicates that humanized MJ2-7v.2-11 is capable of binding IL-13 in vivo, and that this interaction may negate IL-13 biological activity.

The effects of humanized MJ2-7v.2-11 on human IL-13-mediated lung inflammation and cytokine production were tested in mice, and compared with a second antibody, humanized 13.2v.2, as follows. Briefly, C57BL/6 female mice (10/group) were treated with 5 μg of recombinant human R100Q IL-13 (e.g., approximately 250 μg/kg), or an equivalent volume (50 μl) of saline, on days 1, 2, and 3, administered intranasally. On day 0, and 2 hours before administering each dose of IL-13, mice were given intranasal doses of 500 μg, 100 μg, or 20 μg of humanized MJ2-7v.2-11 or humanized 13.2v.2. Control groups received 500 μg IVIG, or an equivalent volume of saline. Animals were sacrificed on day 4, and BAL collected. Eosinophil and neutrophil infiltration into BAL were determined by differential cell count and expressed as a percentage.

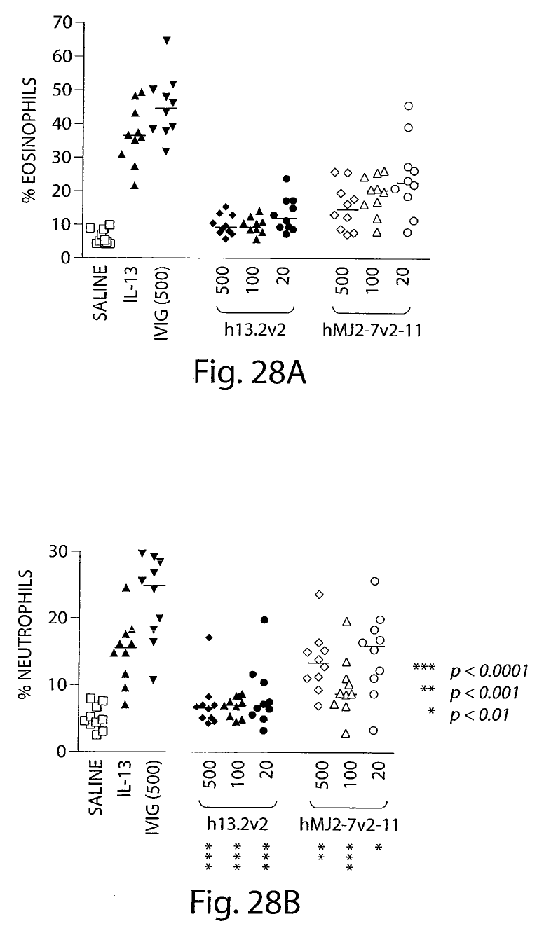

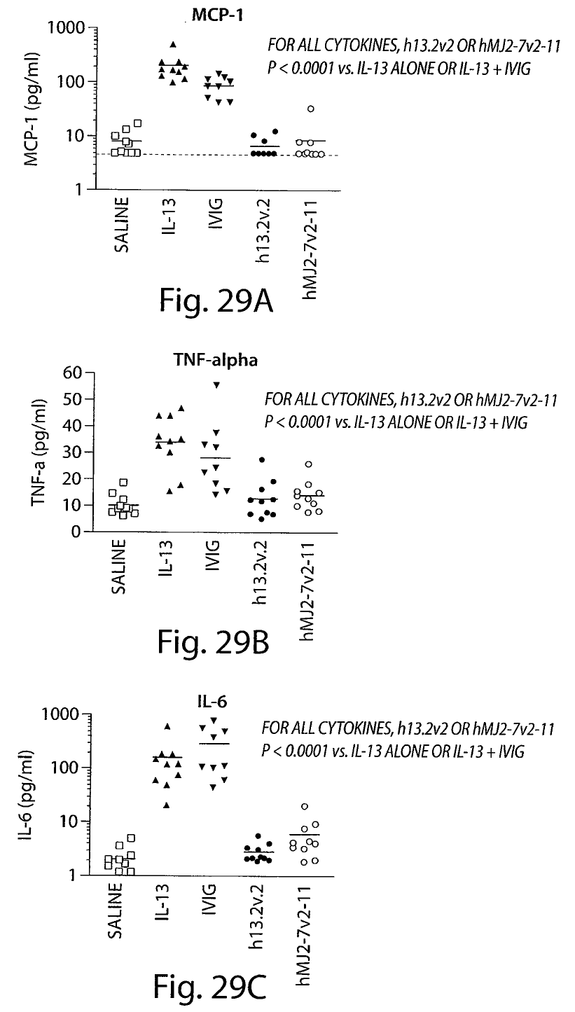

As shown in FIGS. 28A-28B, consistent with FIG. 24A, recombinant human R110Q IL-13 treatment evoked an increase in eosinophil and neutrophil infiltration levels. Interestingly, humanized MJ2-7v.2-11 and humanized 13.2v.2 significantly reduced eosinophil (FIG. 28A) and neutrophil (FIG. 28B) infiltration compared to controls (e.g., saline, IL-13, IVIG). As shown in FIG. 29A-29C, HMJ2-7V2-11 and humanized MJ2-7v.2-11 also abrogated increases in MCP-1, TNF-α, and IL-6 cytokine levels.

To confirmation that BAL cytokine levels accurately represent the degree of inflammation C57BL/6 female mice were treated with 5 μg of recombinant human R110Q IL-13 (e.g., approximately 250 μg/kg) or an equivalent volume (50 μl) of saline on days 1, 2, and 3, administered intranasally. On day 0, and 2 hours before administering each dose of IL-13, mice were given intranasal doses of 500, 100, or 20 μg of humanized MJ2-7v.2-11. On day 4, animals were sacrificed and BAL collected. Humanized MJ2-7v.2-11 antibody levels in BAL were determined by ELISA, as described above. BAL IL-6 levels were determined by cytometric bead array. Eosinophil percentages were determined by differential cell counting.

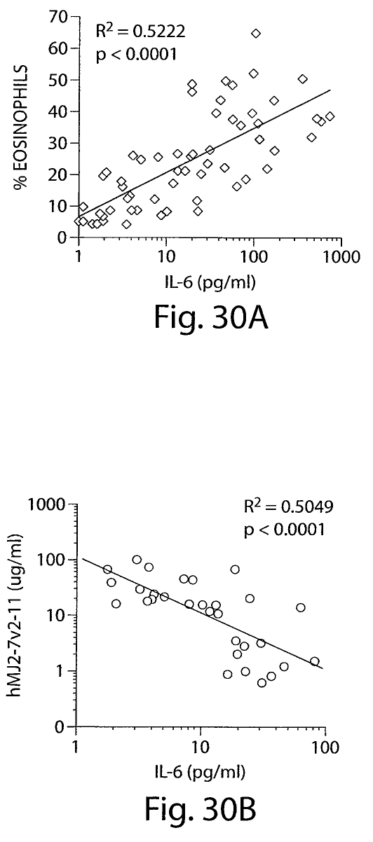

As shown in FIGS. 30A-30B, IL-6 BAL cytokine levels were related to the degree of inflammation. Furthermore, higher levels of humanized MJ2-7v.2-11 in BAL fluid inversely correlated with cytokine concentration, strongly implying a treatment effect.

The levels of antibody required to reduce IL-13 bioactivity in vivo in this model were high. The best efficacy was seen at a dose of 500 μg antibody, corresponding to approximately 25 mg/kg in the mouse. This high dose requirement for antibody is most likely a consequence of the high levels of IL-13 (5 μg/dose×3 doses) used to elicit lung responses. Interestingly, good neutralization of in vivo IL-13 bioactivity was seen only when humanized MJ2-7v.2-11 was administered intranasally, and not when the antibody was administered via intravenous or intraperitoneal. Distribution studies showed that following intravenous and intraperitoneal dosing, high levels of antibody were recovered in serum at the time of sacrifice, but very low levels were found in BAL. In contrast, following intranasal dosing, comparable levels of antibody were found in serum and in BAL. Thus, levels of humanized MJ2-7v.2-11 in BAL fluid were approximately 100-fold higher following intranasal dosing than intravenous and intraperitoneal dosing. The observation that intranasal dosing was efficacious but intravenous and intraperitoneal dosing was not indicates that in this model, the site of antibody action was the lung. This site of action is expected based on the intratracheal or intranasal delivery route of IL-13, and was confirmed by the observation that antibody trapped IL-13 in the BAL fluid, but very little antibody/IL-13 complex was seen in the serum.

In conclusion, these findings further support the IL-13 neutralization activity of humanized MJ2-7v.2-11 in vivo.

### Example 23

### Effects of IL-13 and/or IL-4 Neutralization at the Time of Allergen Challenge on Allergen-Specific IgE Titer

IL-13 and IL-4 drive the production of IgE, an important mediator of allergic disease (Oettgen, H. C. (2000) Curr Opin Immunol 12:618-623; Wynn, T. A. (2003) Anuu Rev. Immunol. 21:425-456). The effects of a single administration of IL-4 or IL-13 antagonist, delivered 24 hours prior to challenge, on allergen-specific IgE levels were examined. These questions were addressed using a standard murine OVA sensitization and challenge model.

Female Balb/c mice between 6 and 8 weeks of age were purchased from Jackson Laboratory. Mice were housed in environmentally controlled, pathogen-free conditions for 2 weeks before the study and for the duration of the experiments. All procedures were reviewed and approved by the Institutional Animal Care and Use Committee at Wyeth Research.

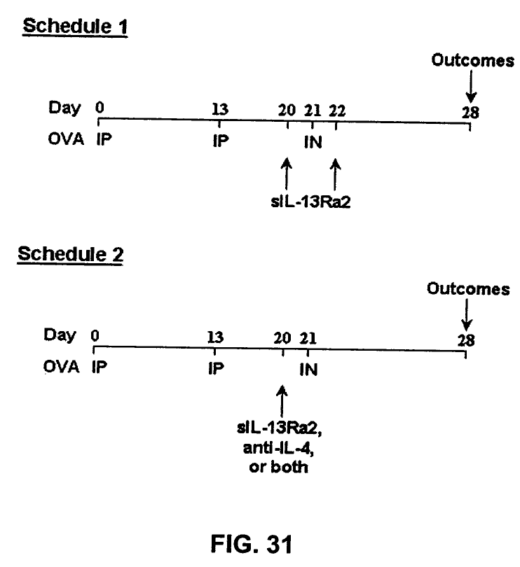

Groups of mice were immunized by intraperitoneal injections with 200 μl solution containing 20 μg OVA (grade V, Sigma-Aldrich, St Louis, Mo.) emulsified with 4 mg aluminum hydroxide/magnesium hydroxide (ImjectAlum; Pierce, Rockford, Ill.) in PBS on days 0 and 13 (FIG. 31). Sensitized mice were administered 200 μg/dose soluble murine IL-13Rα2.IgG fusion protein (sIL-13Rα2.Fc; Wyeth Research) or 200 μg/dose rat anti-mouse IL-4 monoclonal antibody (clone 30340; rat IgG1 anti-mouse IL-4; R&D Systems, Minneapolis, Minn.), by intraperitoneal injection one day before challenge. Control animals received mouse IgG2a (Wyeth Research) or purified rat IgG1 (Wyeth Research). Some groups were treated with sIL-13Rα2.Fc or control one day before and one day after challenge. On day 21, the mice were anesthetized with isoflurane solution (Henry Schein, Melville, N.Y.) using an Impac6 system (VetEquip, Pleasanton, Calif.) and challenged intranasally with 20 μg OVA/mouse in 50 μl PBS.

Mice were sacrificed on day 28 and blood collected by cardiac puncture. Serum was obtained by use of gel barrier with clotting activator tubes (CapiJect; Terumo Medical, Somerset, N.J.).

To assay IgE titers, ELISA plates (MaxiSorp; Nunc) were coated with rat anti-mouse IgE (BD Biosciences, San Jose, Calif.). Plates were blocked with 0.5% gelatin in PBS for 1 hour; washed in PBS containing 0.05% Tween-20 (PBS-Tween); incubated 6 hours at room temperature with purified mouse IgE (BD Biosciences) as standard, or dilutions of serum, in the presence of mouse IgG (Sigma-Aldrich, St. Louis, Mo.) as blocker. The assay was developed using peroxidase-linked streptavidin (Southern Biotechnology Associates, Birmingham, Ala.) and TMB-substrate solution (SureBlue; Kirkegaard & Perry Laboratories, Gaithersberg, Md.). For determination of OVA-specific IgE or IgG subtypes, plates were coated overnight with OVA (Sigma-Aldrich). Bound IgE was quantitated with biotinylated rat anti-mouse IgE (BD Biosciences) in the presence of mouse IgG blocking agent (Sigma-Aldrich). Bound IgG1 was quantitated with biotinylated rat anti-mouse IgG1 or rat anti-mouse IgG3 (BD Biosciences). Total IgE concentrations were determined by reference to a standard curve of purified mouse IgE (BD Biosciences). The limit of detection was 2 ng/ml. OVA-specific Ig titer was quantitated as the serum dilution required to reach a given absorbance value, relative to a reference standard. The limit of detection was a relative titer of 0.5. Serial dilutions of serum were run in each assay, with each sample run in at least three separate assays.

For each test, average values for replicate determinations from each animal were included. Groups of 20 animals were run in each assay. Data were analyzed using GraphPad Prism software. All reported p values were determined by unpaired Student's t test.

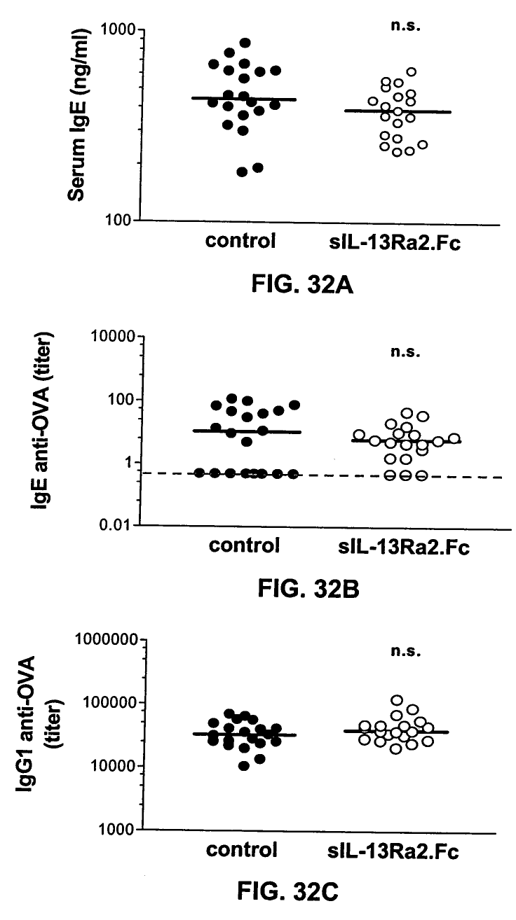

To address the requirement for IL-13 in driving IgE production in response to allergen challenge, IL-13 antagonist (sIL-13Rα2.Fc) was administered to OVA-immunized mice 24 hours before and 24 hours after intranasal challenge with the antigen. As outlined in FIG. 31, mice were immunized i.p. with OVA/alum on day 0, boosted with OVA/alum on day 13, and challenged intranasally on day 21. sIL-13Rα2.Fc (200 μg) was administered i.p. on both days 20 and 22. Animals were sacrificed on day 28, and blood collected into serum separator tubes. Total serum IgE was quantitated by ELISA. There was no difference in total IgE titer in animals treated with sIL-13Rα2.Fc as compared to those given control mouse IgG2a (FIG. 32A). Animals treated both before and after challenge with the IL-13 antagonist had reduced OVA-specific IgE titer as compared to animals treated with the isotype control, but this difference failed to reach statistical significance because of the presence of several animals in the control group with no detectable titer of OVA-specific IgE (FIG. 32B). There was no significant difference in titers of OVA-specific IgG1 (FIG. 32C).

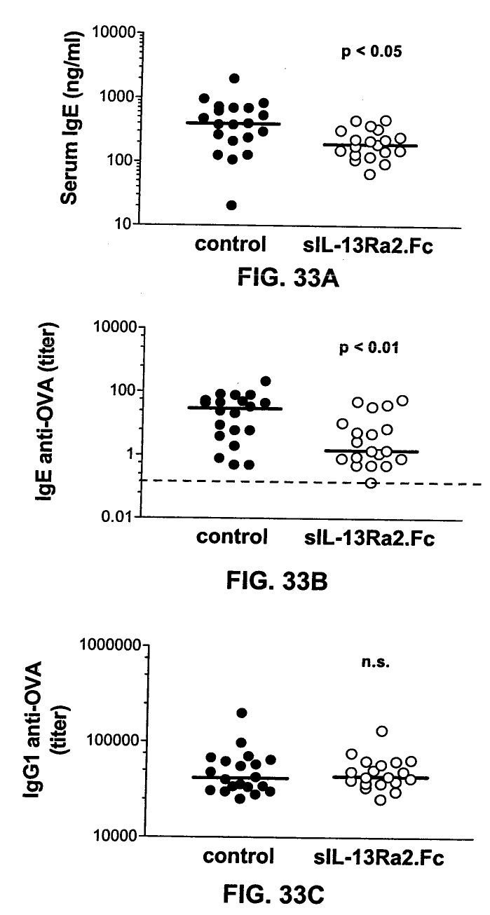

Because there was a trend toward reduced titers of OVA-specific IgE in animals treated with sIL-13Rα2.Fc both before and after challenge, we evaluated the effectiveness of a single administration of sIL-13Rα2.Fc, given 24 hours before challenge. Total serum IgE concentration was reduced in the mice treated with sIL-13Rα2.Fc as compared to those given IgG2a control (p<0.05; FIG. 33A). OVA-specific IgE titer was also reduced following a single administration of sIL-13Rα2.Fc p<0.01; FIG. 33B). There was no change in titer of OVA-specific IgG1.

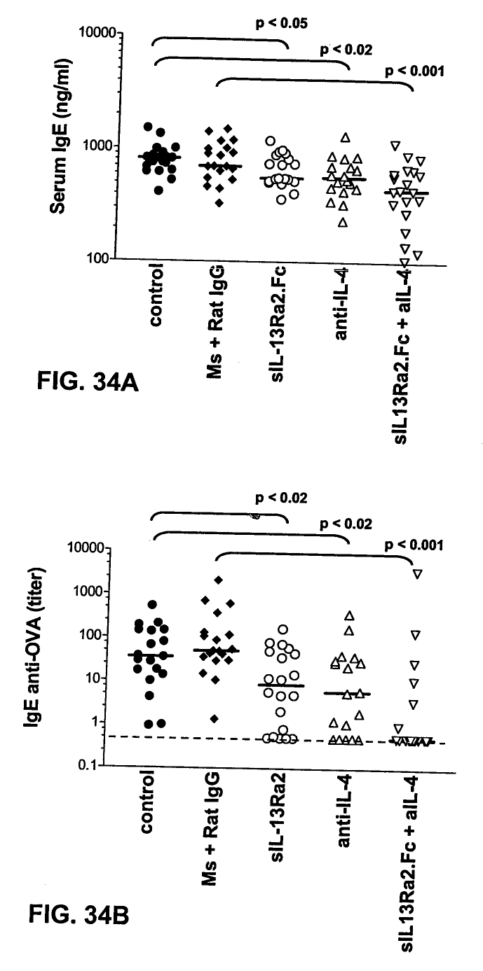

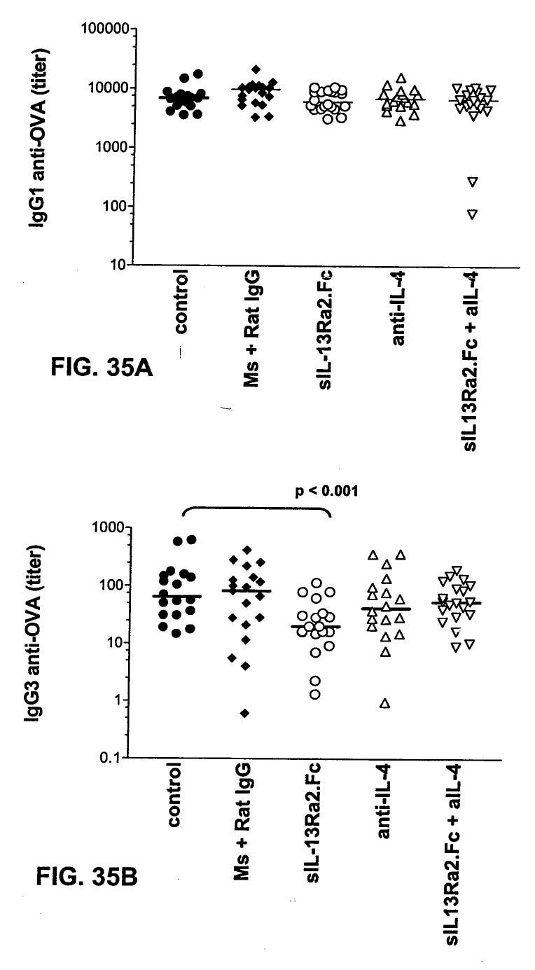

To evaluate whether IL-4 neutralization could affect the IgE response to OVA challenge in a similar way to IL-13 neutralization, mice were given a single dose of 200 μg anti-IL-4 i.p., 24 hours pre-challenge. An additional group of mice was treated with a combination of sIL-13Rα2.Fc and anti-IL-4 (200 μg each). Neutralization of either IL-13 (p<0.05) or IL-4 (p<0.02) produced a significant reduction in total serum IgE titer (FIG. 34A). OVA-specific IgE titers were also significantly reduced following treatment with either anti-IL-4 (p<0.02) or sIL-13Rα2.Fc (p<0.02) (FIG. 34B). OVA-specific IgG1 titers were unaffected by either treatment (FIG. 35A). OVA-specific IgG3 titers were also measured in this study and showed a significant reduction with IL-13 antagonist p<0.001), but not with anti-IL-4 treatment (FIG. 35B).

Administration of sIL-13Rα2.Fc together with anti-IL-4 produced a greater reduction in total serum IgE titer than that produced by either agent alone (p<0.001) (FIG. 34A). Similarly, OVA-specific IgE titers were reduced to a greater extent following treatment with sIL-3Rα2.Fc and anti-IL-4 than was seen by blocking either cytokine alone (p<0.001) (FIG. 34B). Mice treated with the combination of sIL-13Rα2.Fc and anti-IL-4 did not differ in titers of OVA-specific IgG1 (FIG. 35A) or OVA-specific IgG3 (FIG. 35B) compared to control animals.

Several studies have examined the utility of IL-4 or IL-13 neutralization, delivered throughout the course of OVA immunization and/or challenge, in modulating IgE responses (Zhou, C. Y. et al. (1997) J Asthma 34:195-201; Yang, G. et al. (2004) Cytokine 28:224-232). Although this treatment paradigm is effective, studies in the NHP model, discussed herein, indicate that effective IL-13 neutralization could have a lasting impact on IgE responses. Therefore, the requirement for multiple administrations of an IL-4 or IL-13 neutralizing agent was addressed in a mouse model. We determined whether, under optimal conditions of sensitization and challenge, a single treatment with IL-4 or IL-13 neutralizing agent could effectively modulate IgE responses to antigen.

sIL-13Rα2.Fc is a potent IL-13 antagonist, that has been shown to block lung inflammation, AHR, and mucus production in animal models of asthma (Wills-Karp, M. et al. (1998) Science 282:2258-2261). In previous studies addressing its effects on IgE production, mice were given two rounds of lung challenge with OVA either 10 days (Wills-Karp, M. et al. (1998) supra) or 6 weeks (Taube, C. et al. (2002) J. Immunol. 169:6482-6489) following the initial challenge. sIL-13Rα2.Fc delivered only at the time of secondary allergen challenge did not alter the serum titer of OVA-specific IgE (Wills-Karp, M. et al. (1998) supra, Taube, C. et al. (2002) supra). The lack of effect on IgE titer was not surprising given the robust IgE response seen with a secondary challenge (Karp, M. et al. (1998) supra). Consistent with this, delivery of several doses of IL-13 antagonist, beginning at the initial challenge, has been more effective. Serum levels of allergen-specific IgE, but not IgG1, were reduced when antibody to IL-13 was administered prior to each of 5 weekly intranasal challenges with OVA in a chronic asthma model (Zhou, C. Y. et al. (1997) supra).

To address whether a single dosing paradigm with IL-13 neutralizing agent would affect specific IgE production in mice, sIL-13Rα2.Fc was administered before intranasal challenge with OVA. Mice were sensitized with OVA/alum on days 0 and 13, then given a single intranasal challenge with OVA on day 21. Results showed that a single administration of sIL-13Rα2.Fc, delivered 24 hours before challenge, reduced titers of OVA-specific IgE at the time of sacrifice, on day 28. Titers of OVA-specific IgG1 were not affected. Total serum IgE concentrations were also reduced in most experiments. Interestingly, delivery of two doses of sIL-13Rα2.Fc, at 24 hours before and 24 hours after challenge, did not improve the efficacy of this treatment.

To compare the efficacy of IL-13 and IL-4 neutralization, groups of mice were sensitized and challenged with OVA as described above, and treated 24 hours before challenge either with sIL-13Rα2.Fc, antibody to IL-4, or both sIL-13Rα2.Fc and anti-IL-4. Treatment with either sIL-13Rα2.Fc or anti-IL-4 significantly reduced titers of OVA-specific IgE. Total serum IgE concentration was also significantly, reduced. Administration of both sIL-3Rα2.Fc and anti-IL-4 produced a greater magnitude of change in OVA-specific titer and in total serum IgE concentration than was seen with either treatment alone. These effects appeared specific for IgE, however, as neither OVA-specific IgG1 nor OVA-specific IgG3 titers were affected by the combined treatment with sIL-13Rα2.Fc and anti-IL-4.

These findings support the observations from NHP studies, that delivery of an IL-13 neutralizing agent in single administration prior to allergen challenge can reduce the IgE response to allergen. An IL-4 neutralizing agent can have similar activity. Neutralization of both IL-4 and IL-13 had a more potent effect on reduction of IgE responses than neutralization of either cytokine alone. These findings emphasize the critical requirement for IL-4 and IL-13 at the time of allergen challenge.

Those skilled in the art will recognize, or be able to ascertain using no more than routine experimentation, many equivalents of the specific embodiments described herein described herein. Other embodiments are within the following claims.

What is claimed is:

1. A method of treating or preventing an IL-13-associated disorder or condition in a subject, comprising administering to the subject, as a single treatment interval, one or more of an IL-13 antagonist or an IL-4 antagonist in an amount effective to reduce or delay the onset or recurrence of one or more symptoms of the disorder or condition.

2. The method of claim 1, wherein the single treatment interval is a single dose of the IL-13 antagonist alone or in combination with the IL-4 antagonist.

3. The method of claim 1, wherein single treatment interval consists essentially of two or three doses of the IL-13 antagonist alone or in combination with the IL-4 antagonist within one week or less from the initial dose.

4. The method of claim 1, wherein the administration of the one or more of the IL-13 antagonist or the IL-4 antagonist occurs prior to any detectable manifestation of the symptoms of the disorder or condition.

5. The method of claim 1, wherein the administration of the one or more of the IL-13 antagonist or the IL-4 antagonist occurs after a partial manifestation of the symptoms of the disorder or condition.

6. The method of claim 1, wherein the one or more of the IL-13 antagonist or the IL-4 antagonist is administered to the subject prior to exposure to an agent that triggers or exacerbates the IL-13-associated disorder or condition.

7. The method of claim 6, wherein the one or more of the IL-13 antagonist or IL-4 antagonist is administered prior to seasonal exposure to an allergen.

8. The method of claim 4, wherein the one or more of the IL-13 antagonist or the IL-4 antagonist is administered prior to the recurrence of a flare or episode of the IL13-associated disorder or condition.

9. The method of claim 1, wherein the one or more of the IL-13 antagonist or the IL-4 antagonist is administered anywhere between 1 to 5 days before or after exposure to the triggering or exacerbating agent.

10. The method of claim 6, wherein the agent that triggers or exacerbates the IL-13-associated disorder is selected from the group consisting of an allergen, a pollutant, a toxic agent, an infection and stress.

11. The method of claim 1, wherein the symptoms of the IL-13 associated disorder or condition comprise one or more of: increased IgE levels, increase histamine release, increase eotaxin levels, or a respiratory symptom.

12. The method of claim 11, wherein the respiratory symptom comprises one or more of: difficulty breathing, wheezing, coughing, shortness of breath or difficulty performing normal daily activities.

13. The method of claim 1, wherein the subject is a human adult, an adolescent, or a child having, or at risk of having, the IL-13 associated disorder or condition.

14. The method of claim 1, wherein the IL-13-associated disorder or condition is an inflammatory, a respiratory, an allergic, or an autoimmune disorder or condition.

15. The method of claim 1, wherein the IL-13-associated disorder or condition is chosen from one or more of: IgE-related disorders, atopic disorders, atopic dermatitis, urticaria, eczema, allergic rhinitis allergic enterogastritis, asthma, chronic obstructive pulmonary disease (COPD), conditions involving airway inflammation, eosinophilia, fibrosis and excess mucus production, autoimmune conditions of the skin, atopic dermatitis, inflammatory bowel disease (IBD), ulcerative colitis, Crohn's disease, cirrhosis, hepatocellular carcinoma, scleroderma, tumors, cancers, leukemia, glioblastoma, lymphoma, viral infections, or fibrosis of the liver.

16. The method of claim 1, wherein the one or more of the IL-13 antagonist or the IL-4 antagonist inhibits or reduces one or more biological activities of IL-13 or IL-4, or an IL-13 receptor or an IL-4 receptor chosen from one or more of: induction of CD23 expression, production of IgE by human B cells, phosphorylation of a transcription factor, activation of STAT6 protein, antigen-induced eosinophilia in vivo; antigen-induced bronchoconstriction in vivo, or drug-induced airway hyperreactivity in vivo.

17. The method of claim 1, wherein the one or more of the IL-13 antagonist or the IL-4 antagonist is an antibody molecule that binds to IL-13, IL-13R, IL-4 or IL-4Rα; a soluble form of the IL-13R or the IL-4Rα; an IL-13 or IL-4 mutein that binds to the corresponding receptor, but does not substantially activate the receptor; a small molecule inhibitor of STAT6; a peptide inhibitor; or an inhibitor of nucleic acid expression.

18. The method of claim 21, wherein the IL-13R is an IL-13Rα2 or an IL-13Rα1.

19. The method of claim 21, wherein the antibody molecule binds to IL-13 with a K_{D} of less than 10⁻⁷ M, and has one or more of the following properties:
(a) the heavy chain immunoglobulin variable domain comprises a heavy chain CDR3 that differs by fewer than 3 amino acid substitutions from a heavy chain CDR3 of monoclonal antibody MJ2-7 (SEQ ID NO:17), mAb 13.2 (SEQ ID NO:196) or C65 (SEQ ID NO:123);
(b) the light chain immunoglobulin variable domain comprises a light chain CDR1 that differs by fewer than 3 amino acid substitutions from a corresponding light chain CDR of monoclonal antibody MJ2-7 (SEQ ID NO:18), mAb 13.2 (SEQ ID NO:197) or C65 (SEQ ID NO:118);
(c) the heavy chain immunoglobulin variable domain comprises a an amino acid sequence encoded by a nucleotide sequence that hybridizes under high stringency conditions to the complement of the nucleotide sequence encoding a heavy chain variable domain of V2.1 (SEQ ID NO:71), V2.3 (SEQ ID NO:73), V2.4 (SEQ ID NO:74), V2.5 (SEQ ID NO:75), V2.6 (SEQ ID NO:76), V2.7 (SEQ ID NO:77), V2.11 (SEQ ID NO:80), ch13.2 (SEQ ID NO:204), h13.2v1 (SEQ ID NO:205), h13.2v2 (SEQ ID NO:206) or h13.2v3 (SEQ ID NO:207);
(d) the light chain immunoglobulin variable domain comprises an amino acid sequence encoded by a nucleotide sequence that hybridizes under high stringency conditions to the complement of the nucleotide sequence encoding a light chain variable domain of V2.11 (SEQ ID NO:36) or h13.2v2 (SEQ ID NO:212);
(e) the heavy chain immunoglobulin variable domain comprises an amino acid sequence that is at least 90% identical to the amino acid sequence of the heavy chain variable domain of V2.1 (SEQ ID NO:71), V2.3 (SEQ ID NO:73), V2.4 (SEQ ID NO:74), V2.5 (SEQ ID NO:75), V2.6 (SEQ ID NO:76), V2.7 (SEQ ID NO:77), V2.11 (SEQ ID NO:80); ch13.2 (SEQ ID NO:208), h13.2v1 (SEQ ID NO:209), h13.2v2 (SEQ ID NO:210) or h13.2v3 (SEQ ID NO:211);
(f) the light chain immunoglobulin variable domain sequence is at least 90% identical a light chain variable domain of V2.11 (SEQ ID NO:36) or h13.2v2 (SEQ ID NO:212);
(g) the antibody molecule competes with mAb MJ2-7, mAb13.2 or C65 for binding to human IL-13;
(h) the antibody molecule contacts one or more amino acid residues from IL-13 selected from the group consisting of residues 116, 117, 118, 122, 123, 124, 125, 126, 127, and 128 of SEQ ID NO:24 or SEQ ID NO:178,
(i) the antibody molecule contacts one or more residues from IL-13 selected from the group consisting of residues 81-93 and 114-132 of human IL-13 (SEQ ID NO:194), or selected from the group consisting of: Glutamate at position 68 [49], Asparagine at position 72 [53], Glycine at position 88 [69], Proline at position 91 [72], Histidine at position 92 [73], Lysine at position 93 [74], and Arginine at position 105 [86] of SEQ ID NO:194 [position in mature sequence; SEQ ID NO:195];
(j) the heavy chain variable domain sequence has the same canonical structure as mAb MJ2-7, mAb 13.2 or C65 in hypervariable loops 1, 2 and/or 3;
(k) the light chain variable domain sequence has the same canonical structure as mAb MJ2-7, mAb 13.2 or C65 in hypervariable loops 1, 2 and/or 3; and
(l) the heavy chain variable domain sequence and/or the light chain variable domain sequence has FR1, FR2, and FR3 framework regions from VH segments encoded by germline genes DP-54 and DPK-9 respectively or a sequence at least 95% identical to VH segments encoded by germline genes DP-54 and DPK-9; and
(m) confers a post-injection protective effect against exposure to Ascaris antigen in a sheep model at least 6 weeks after injection.

20. The method of claim 1, wherein the one or more IL-13 antagonist or the IL-4 antagonist are administered in combination simultaneously or sequentially.

21. The method of claim 27, wherein the one or more IL-13 antagonist or the IL-4 antagonist are co-formulated.

22. The method of claim 27, wherein the one or more IL-13 antagonist or the IL-4 antagonist are administered in combination with other therapeutic agents chosen from one or more of: inhaled steroids, beta-agonists, antagonists of leukotrienes or leukotriene receptors, IgE inhibitors, PDE4 inhibitors, xanthines, anticholinergic drugs, IL-5 inhibitors, eotaxin/CCR3 inhibitors or anti-histamines.

23. A composition or a dose-formulation comprising an IL-13 antagonist and an IL-4 antagonist, wherein the IL4 antagonist is selected from the group consisting of an antibody molecule that binds to IL-4 or IL-4Rα; a soluble form of IL-4Rα; an IL-4 mutein; a small molecule inhibitor of STAT6; a peptide inhibitor; or an inhibitor of nucleic acid expression, and the IL-13 antagonist is an antibody molecule competes with mAb MJ2-7, mAb13.2 or C65 for binding to human IL-13, or a soluble fragment of an IL-13Rα2.

24. A method for detecting the presence of IL-13 in a sample in vitro, comprising
providing a first anti-IL-13 antibody molecule immobilized to a support;
providing a sample obtained from a subject after exposure of the subject to a second anti-IL-13 antibody molecule;
contacting the sample with the first anti-IL-13 antibody, under conditions that allow binding of the IL-13 to the immobilized first anti-IL-13 antibody molecule to occur; and
detecting IL-13 in the sample relative to a reference value,
wherein the first and second anti-IL13 antibodies bind to different epitopes on IL-13.

25. The method of claim 31, wherein the first anti-IL-13 antibody molecule binds to substantially free IL-13, and does not substantially bind to IL-13 bound to the second anti-IL-13 antibody molecule.

26. The method of claim 31, wherein the first anti-IL-13 antibody molecule binds to substantially free IL-13 and IL-13 bound to a second anti-IL-13 antibody molecule.

27. The method of claim 31, wherein the detecting of the presence of IL-13 bound to the immobilized first anti-IL-13 antibody molecule is carried out using a labeled third anti-IL-13 antibody molecule, or a labeled agent that recognizes the complex of IL-13 first or second antibody molecule.

28. The method of claim 31, wherein a change in the level of IL-13 bound to the first anti-IL-13 antibody molecule in the sample relative to a control sample is indicative of the presence of the IL-13 in the sample

29. The method of claim 35, wherein the change is an increase in the level of IL-13 in the sample relative to a predetermined level, wherein said increase is indicative of increased inflammation in the lung.

30. A method for evaluating the efficacy of an anti-IL-13 antibody molecule, in reducing pulmonary inflammation in a subject, comprising:
detecting the levels of IL-13 unbound and bound to the anti-IL-13 antibody molecule in a sample according to the method of claim 24,
wherein a change in the levels of IL-13 unbound relative to a reference sample is indicative of the efficacy of the anti-IL-13 antibody molecule.

31. The method of claim 30, further comprising evaluating a change in one or more of eotaxin levels in a sample, histamine release by basophils, IgE-titers, or evaluating changes in the symptoms of the subject.

32. The method of claim 31, wherein a reduction in the levels of IL-13 unbound relative to the anti-IL-13 antibody molecule, or an increase in the level of IL-13 bound to the antibody molecule is indicative that the anti-IL-13 antibody molecule is effectively reducing lung inflammation in the subject.
